(19) United States
(12) Patent Application Publication
(10) Pub. No.: US 2023/0406878 A1
(43) Pub. Date: Dec. 21, 2023

(54) OLIGONUCLEOTIDES, REAGENTS, AND PREPARATION THEREOF

(71) Applicant: BIOGEN MA INC., Cambridge, MA (US)

(72) Inventors: Wuming Yan, Somerville, MA (US); Xuan Zhou, Cambridge, MA (US); Xianglin Shi, Cambridge, MA (US); Firoz Antia, Cambridge, MA (US); William F. Kiesman, Wayland, MA (US); Yannick Fillon, Cambridge, MA (US)

(21) Appl. No.: 18/036,459

(22) PCT Filed: Nov. 10, 2021
(86) PCT No.: PCT/US2021/058786

§ 371 Date: May 11, 2023

Related U.S. Application Data
(60) Provisional application No. 63/112,281, filed on Nov. 11, 2020.

(51) Int. Cl. C07H 21/02; C07F 7/18; C07D 249/06; C07D 295/192; C07H 23/00; C07H 21/04

(52) U.S. Cl. CPC C07H 21/02; C07F 7/1804; C07D 249/06; C07D 295/192; C07H 23/00; C07H 21/04

(57) ABSTRACT

The present disclosure describes novel reagents and processes for preparing oligonucleotides, which have two or more nucleotides. In one embodiment, the reagent is represented by Formula I′ or B.

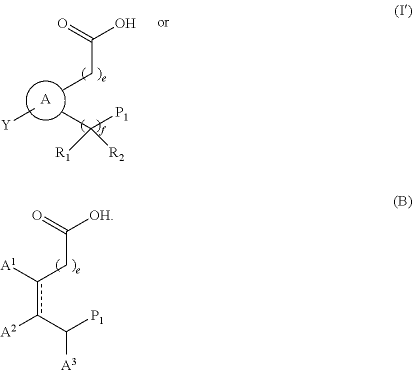

### RELATED APPLICATION

This application claims the benefit of the filing date, under 35 U.S.C. § 119(e), of U.S. Provisional Application No. 63/112,281, filed on Nov. 11, 2020, the entire contents of which is incorporated herein by reference.

### FIELD OF THE INVENTION

The present invention relates to oligonucleotides, reagents, and methods for preparing oligonucleotides.

### BACKGROUND

Oligonucleotides are short DNA or RNA oligomers that can be chemically synthesized for a wide range of applications. Recent developments in utilizing synthetic oligonucleotides as therapeutic agents have increased demand for synthetic methods that can produce oligonucleotides in large quantities with high efficiency and purity.

Traditionally, oligonucleotides are synthesized by a solid phase automated synthesizer utilizing phosphoramidite chemistry, limited to a scale of less than 2 moles. Thus, the solid phase synthesis is insufficient for the production of materials needed for clinical development and commercialization of oligonucleotide drugs in large indications. In addition, the solid phase synthesis often requires the use of excess reagents and consequently increases the cost associated with the production of the target oligonucleotides.

Hence, there is a need for novel reagents and robust methods for synthesizing oligonucleotides that are suitable for large scale manufacturing process with high efficiency and purity.

### SUMMARY OF THE INVENTION

One aspect of the present disclosure is directed to a compound of Formula I′ or B:

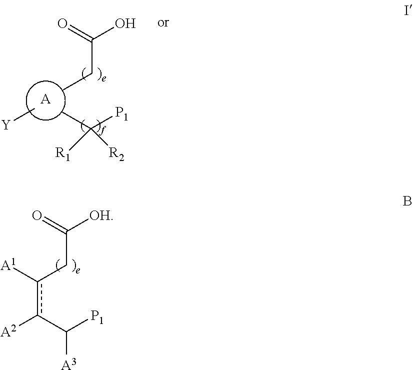

or a salt thereof; wherein ring A, A¹, A², A³, R₁, R₂, P₁, Y, e, and f are defined below.

One aspect of the present disclosure is directed to a nucleotide or an oligonucleotide represented by Formula III or IIIP,

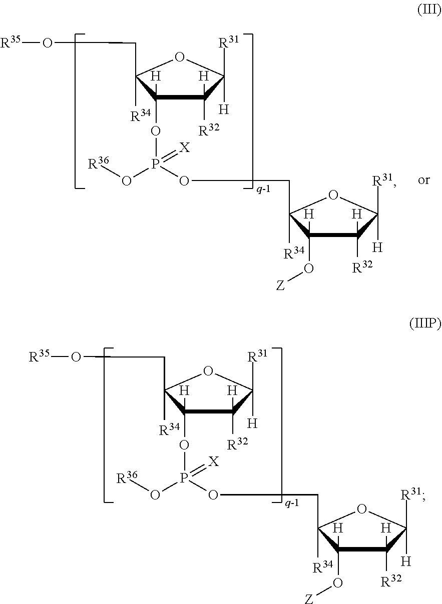

or a salt thereof, wherein R³¹, R³², R³⁴, R³⁵, R³⁶, q, X, and Z are defined below.

One aspect of the present disclosure is directed to a nucleotide or an oligonucleotide represented by Formula III′ or IIIP′,

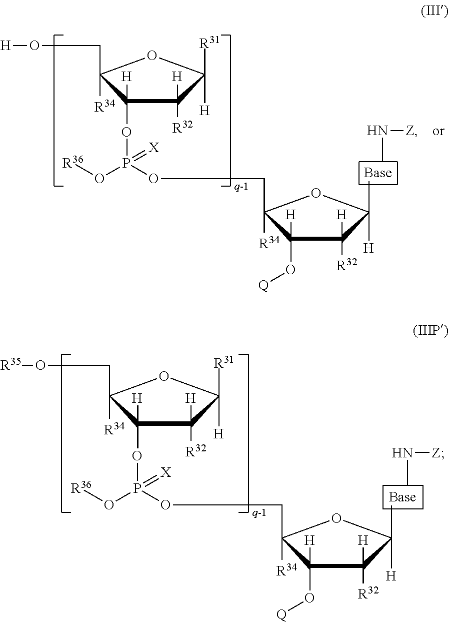

or a salt thereof, wherein R³¹, R³², R³⁴, R³⁵, R³⁶, q, Q, X, [symbol] and Z are defined below.

One aspect of the present disclosure is directed to a process for preparing an oligonucleotide fragment of formula (V),

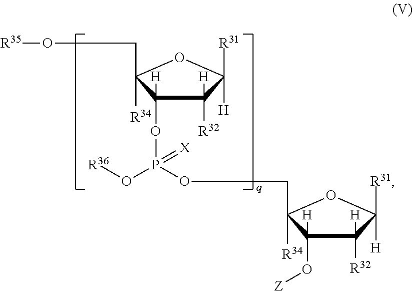

or a salt thereof, comprising the steps of:
1) deprotecting a compound of formula (VA):

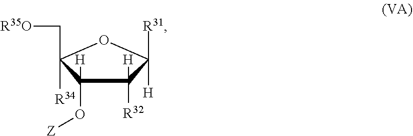

or a salt thereof, to form a compound of formula (VB):

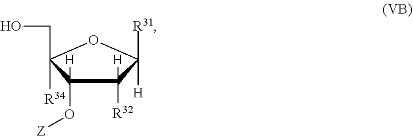

or a salt thereof;
2) reacting the compound of formula (VB), or a salt thereof, with a compound of formula (VC):

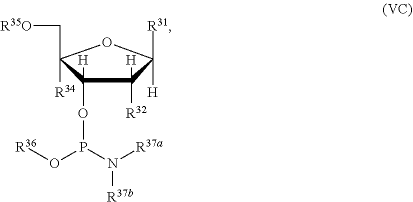

or a salt thereof, to form a compound of formula (VD),

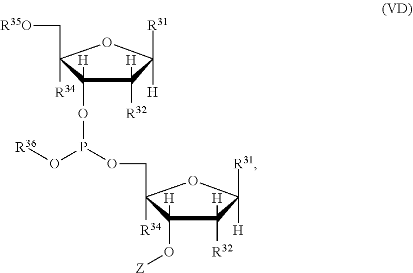

or a salt thereof;
3) sulfurizing or oxidizing the compound of formula (VD), or a salt thereof, with a sulfurization or oxidation agent to form a compound of formula (VE):

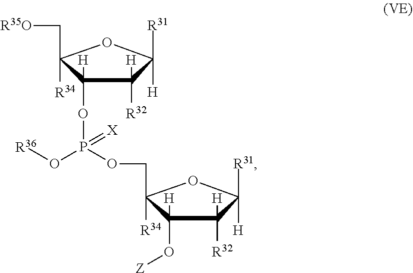

or a salt thereof;
4) deprotecting the compound of formula (VE), or a salt thereof to form a compound of formula (VF):

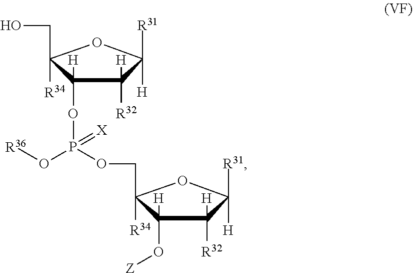

or a salt thereof;
5) when q is equal or greater than 2, starting with the compound of formula (VF), repeating steps 2), 3) and 4) for q-2 times, followed by steps 2) and 3) to yield the fragment of formula (V), or a salt thereof; wherein R³¹, R³², R³⁴, R³⁵, R³⁶, q, X, and Z are defined below.

One aspect of the present disclosure is directed to a process for preparing an oligonucleotide fragment of formula(V′),

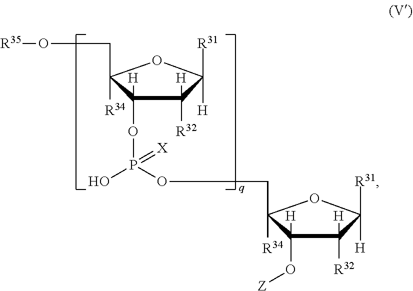

or a salt thereof, comprising the steps of:
1) deprotecting a compound of formula (VA):

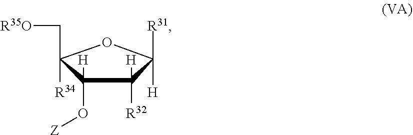

or a salt thereof, to form a compound of formula (VB):

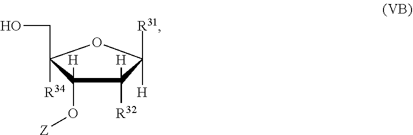

or a salt thereof;
2) reacting the compound of formula (VB), or a salt thereof, with a compound of formula (VC′):

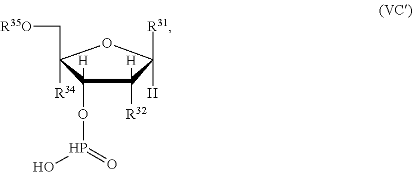

or a salt thereof, to form a compound of formula (VD′),

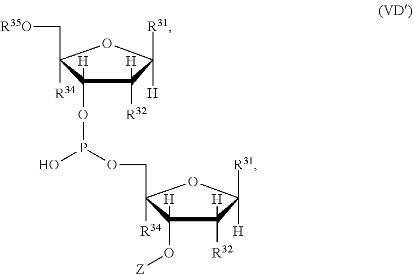

or a salt thereof;
3) sulfurizing or oxidizing the compound of formula (VD′), or a salt thereof, with a sulfurization or oxidation agent to form a compound of formula (VE′):

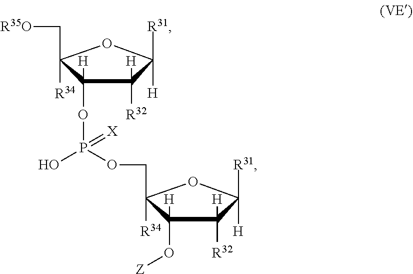

or a salt thereof;
4) deprotecting the compound of formula (VE′), or a salt thereof to form a compound of formula (VF′):

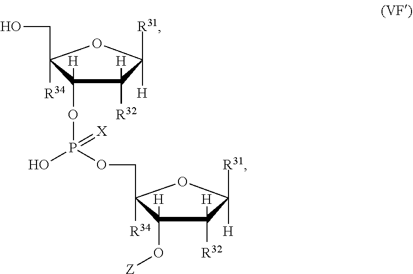

or a salt thereof;
5) when q is equal or greater than 2, starting with the compound of formula (VF′), repeating steps 2), 3) and 4) for q-2 times, followed by steps 2) and 3) to yield the fragment of formula (V′), or a salt thereof, wherein R³¹, R³², R³⁴, R³⁵, q, X, and Z are defined below.

One aspect of the present disclosure is directed to a process for preparing an oligonucleotide fragment of formula (V-C1) or (V-C2),

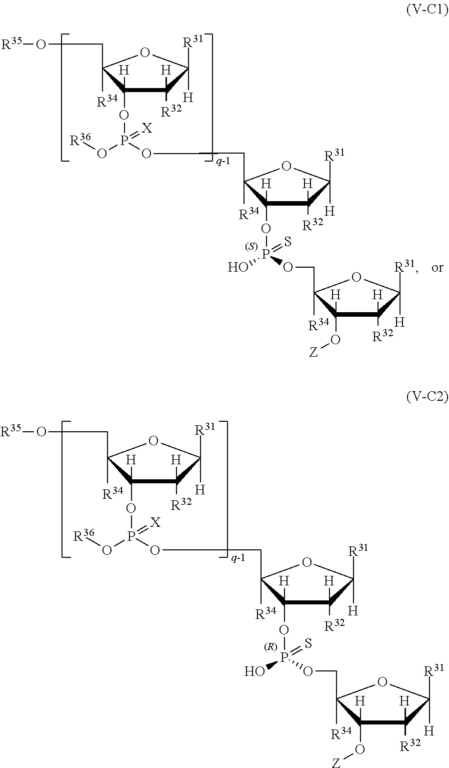

or a salt thereof, comprising the steps of:
1) reacting the compound of formula (VB),

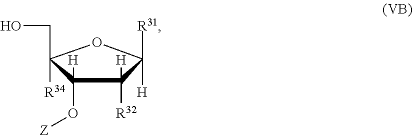

or a salt thereof, with a compound of formula (V-CR1) or (V-CR2),

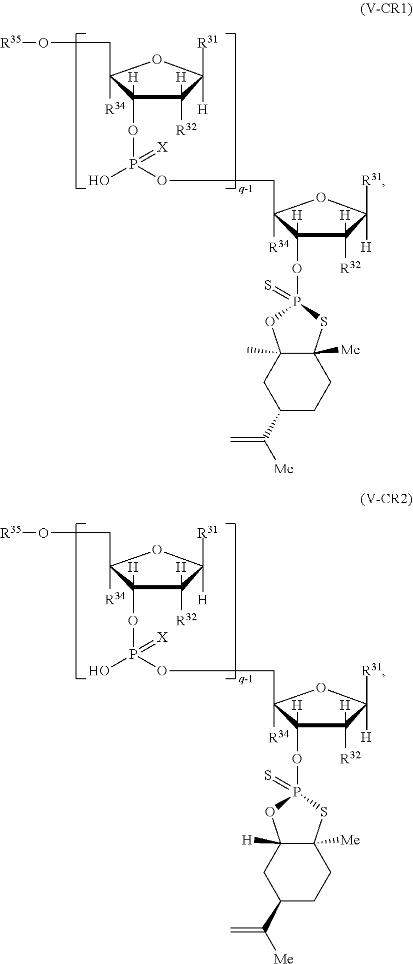

or a salt thereof, and a base, to form a compound of formula (V-C1) or (V-C2), wherein R³¹, R³², R³⁴, R³⁵, q, X, and Z are defined below.

One aspect of the present disclosure is directed to a process for preparing an oligonucleotide fragment of formula (V-C1) or (V-C2),

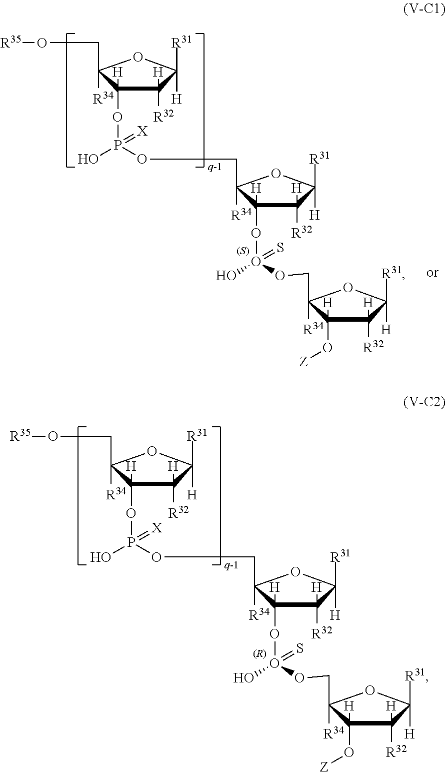

or a salt thereof, comprising the steps of:
1) reacting the compound of formula (VB),

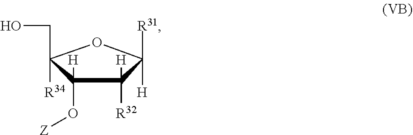

or a salt thereof, with a reagent of formula (VR1) or (VR2),

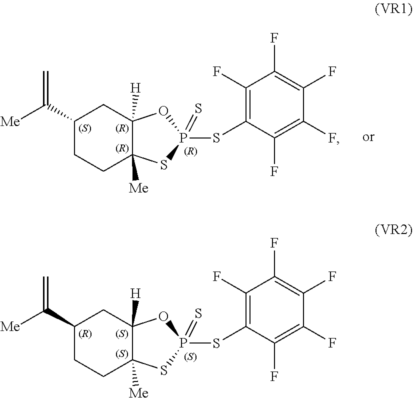

to form a compound of formula (V-CR3) or (V-CR4),

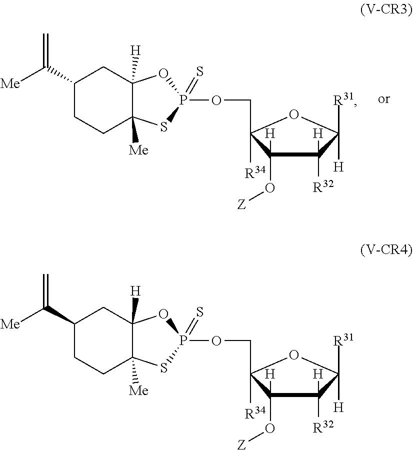

or a salt thereof;
2) reacting the compound of formula (V-CR3) or (V-CR4), or a salt thereof, with a compound of formula (VG):

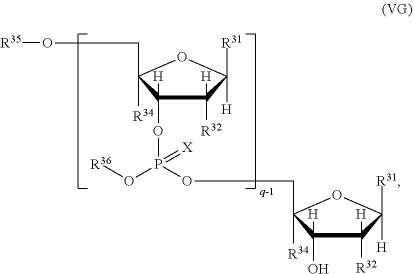

or a salt thereof, and a base, to form the compound of formula (V-C1) or (V-C2), wherein R³¹, R³², R³⁴, R³⁵, R³⁶, q, X, and Z are defined below.

One aspect of the present disclosure is directed to a process for preparing an oligonucleotide fragment of formula (VBZ),

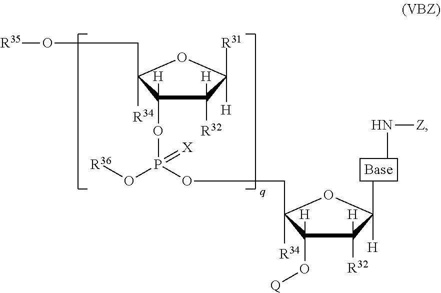

or a salt thereof, comprising the steps of:
1) reacting the compound of formula (VBZ-1),

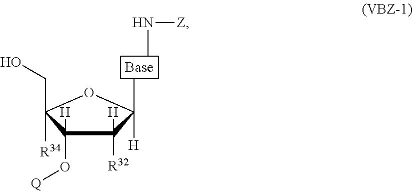

or a salt thereof, with a compound of formula (VBZ-2):

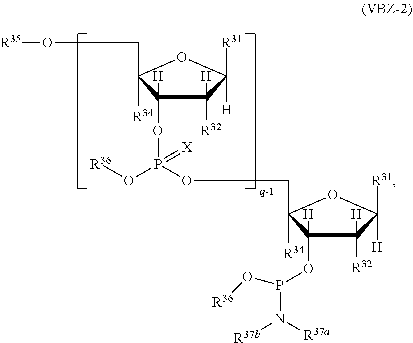

or a salt thereof, to form a compound of formula (VBZ-3),

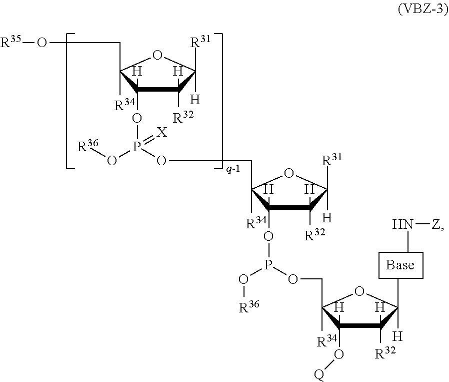

or a salt thereof;
3) sulfurizing or oxidizing the compound of formula (VBZ-3), or a salt thereof, with a sulfurization or oxidation agent to form the compound of formula (VBZ), or a salt thereof; wherein R³¹, R³², R³⁴, R³⁵, R³⁶, q, X, and Z are defined below.

One aspect of the present disclosure is directed to a process for preparing an oligonucleotide fragment of formula (V),

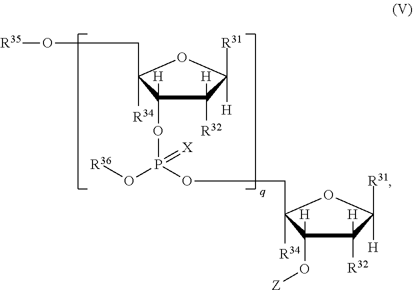

or a salt thereof, comprising the steps of:
a) coupling a nucleotide of formula (V-1):

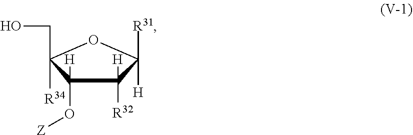

or a salt thereof, with an oligonucleotide fragment of formula (V-2):

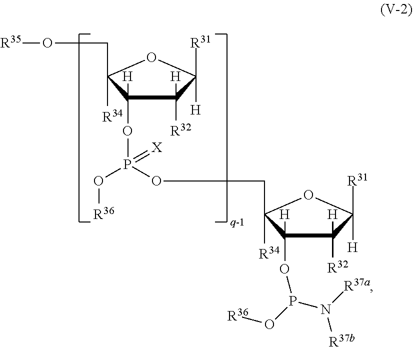

or a salt thereof, in a solution to form an oligonucleotide fragment of formula (V-3),

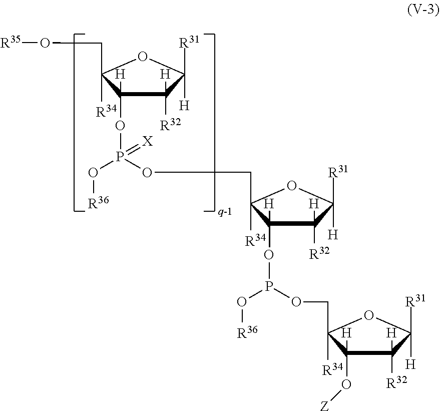

or a salt thereof; and
b) sulfurizing or oxidizing the oligonucleotide of formula (V-3), or a salt thereof, to form an oligonucleotide of formula (V):

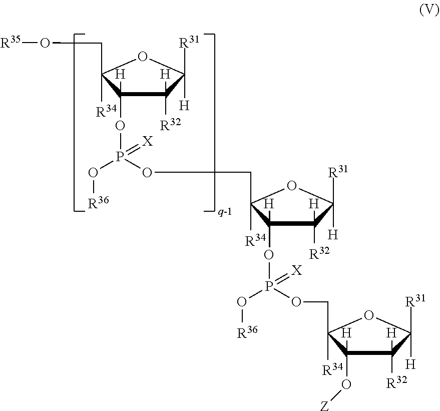

or a salt thereof; wherein R³¹, R³², R³⁴, R³⁵, R³⁶, R^{37a}, R^{37b}, q, X, and Z are defined below.

One aspect of the present disclosure is directed to a process for preparing an oligonucleotide fragment of formula (V*),

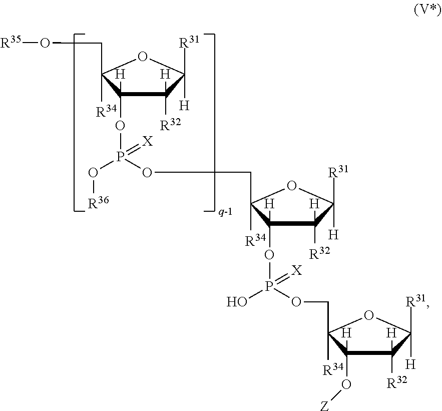

or a salt thereof, comprising the steps of:
a) coupling a nucleotide of formula (V-1):

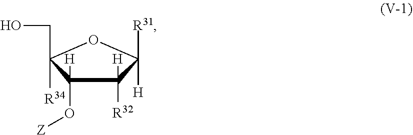

or a salt thereof, with an oligonucleotide fragment of formula (V-2′):

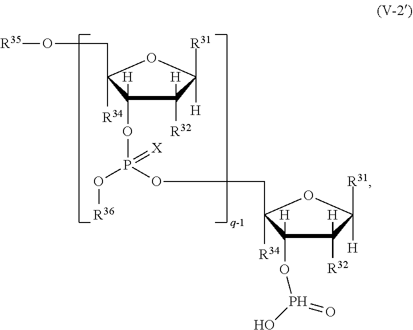

or a salt thereof, in a solution to form an oligonucleotide fragment of formula (V-3′),

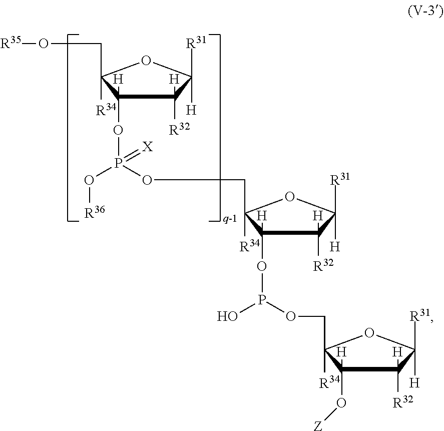

or a salt thereof; and
b) sulfurizing or oxidizing the oligonucleotide of formula (V-3′), or a salt thereof, to form the oligonucleotide of formula (V*) or a salt thereof;
   wherein R³¹, R³², R³⁴, R³⁵, R³⁶, q, X, and Z are defined below.

One aspect of the present disclosure is directed to a process for preparing a target oligonucleotide of formula (VI) or (VI-1),

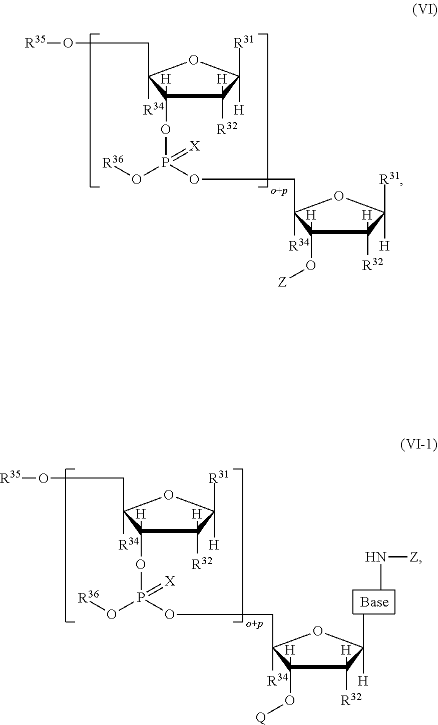

or a salt thereof, comprising
a) coupling an oligonucleotide fragment of formula (F1) or (F1-1):

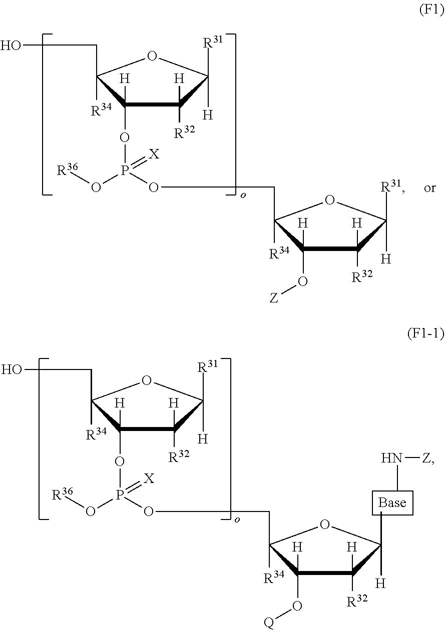

or a salt thereof,
with an oligonucleotide fragment of formula (F2):

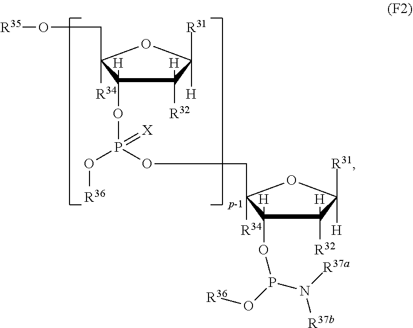

or a salt thereof, in a solution to form an oligonucleotide fragment of formula (F3) or (F3-1),

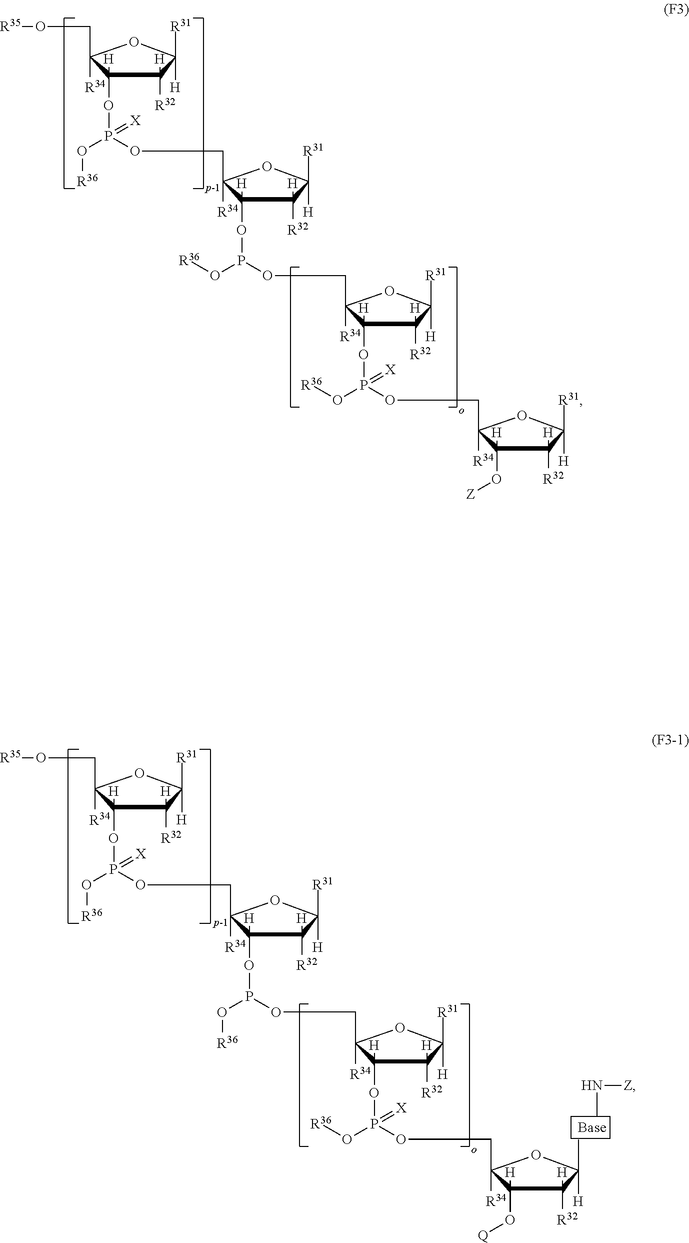

or a salt thereof; and
b) sulfurizing or oxidizing the oligonucleotide fragment of formula (F3) or (F3-1), or a salt thereof, to form the oligonucleotide of formula (VI) or (VI-1) or a salt thereof;
   wherein R³¹, R³², R³⁴, R³⁵, R³⁶, R^{37a}, R^{37b}, o, p, Q, X, [symbol] and Z are defined below.

One aspect of the present disclosure is directed to a process for preparing a target oligonucleotide of formula (VI′) or (VI′-1),

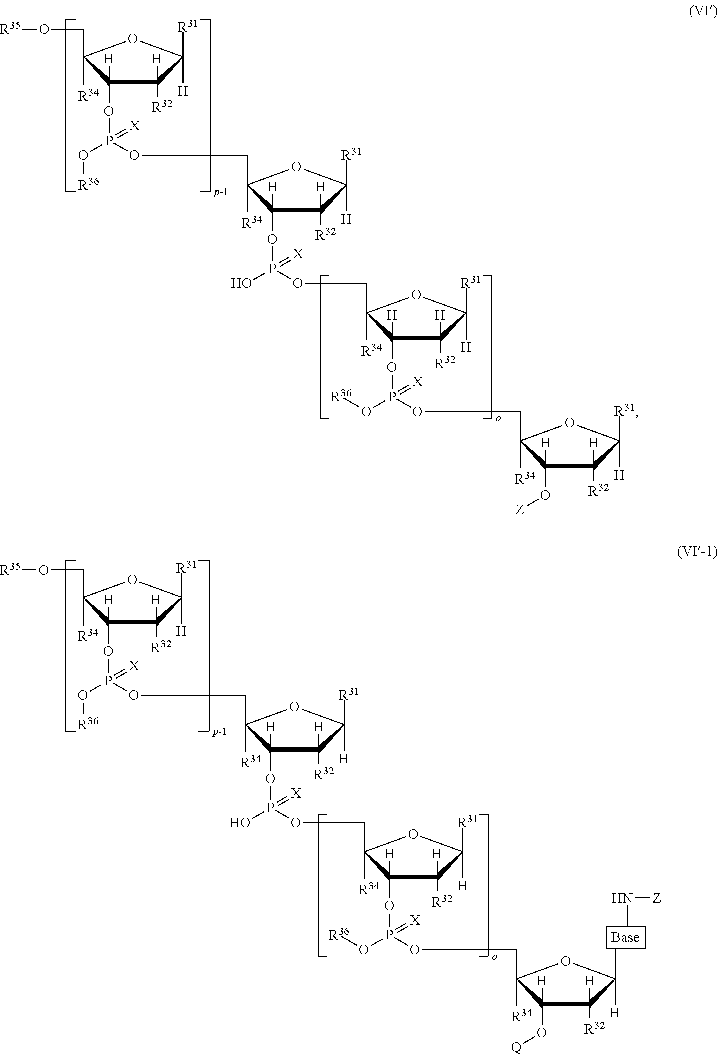

or a salt thereof, comprising
a) coupling an oligonucleotide fragment of formula (F1) or (F1-1):

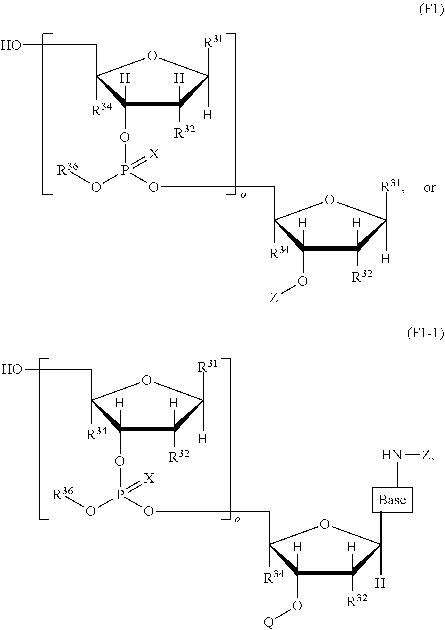

or a salt thereof, with an oligonucleotide fragment of formula (F2′):

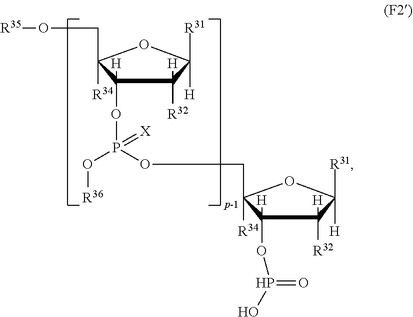

or a salt thereof, in a solution to form an oligonucleotide fragment of formula (F3′) or (F3′-1),

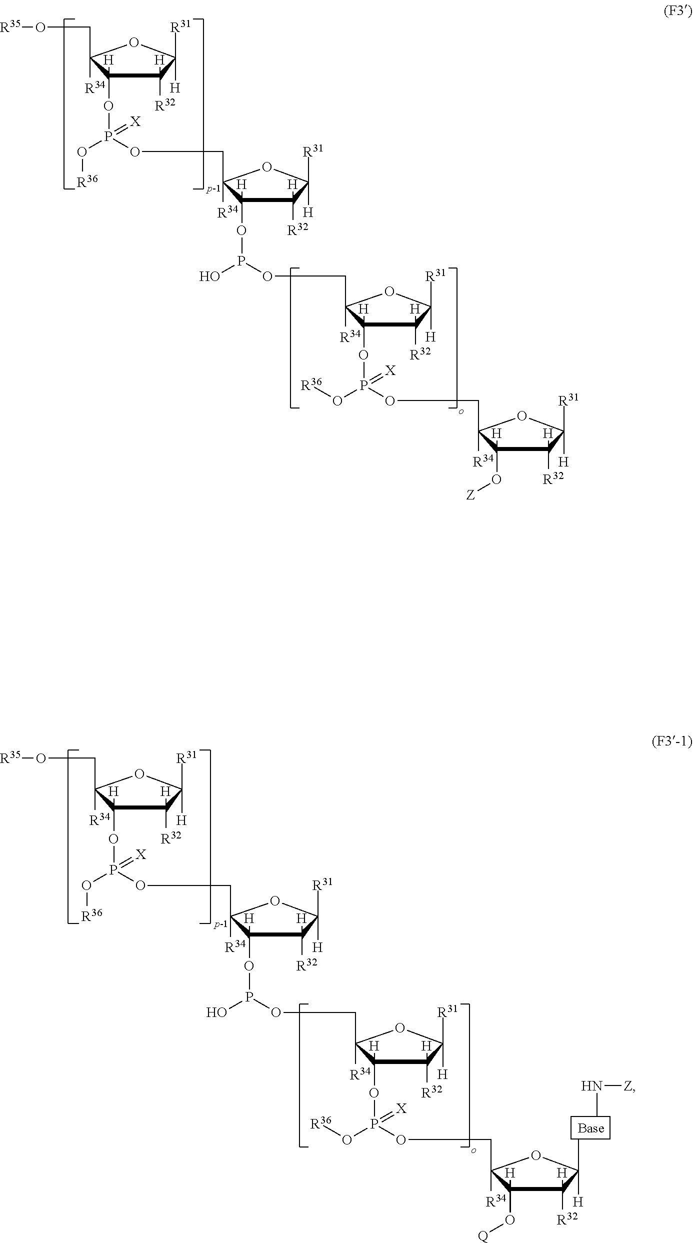

or a salt thereof; and
b) sulfurizing or oxidizing the oligonucleotide fragment of formula (F3′) or (F3′-1), or a salt thereof, to form the oligonucleotide of formula (VI′) or (VI′-1) or a salt thereof, wherein: R³, R³², R³⁴, R³, R³⁶, R^{37a}, R^{37b}, o, p, Q, X, [symbol] and Z are defined below.

### BRIEF DESCRIPTION OF THE FIGURES

FIG. 1 shows a retro-synthesis scheme for preparing oligonucleotide I.

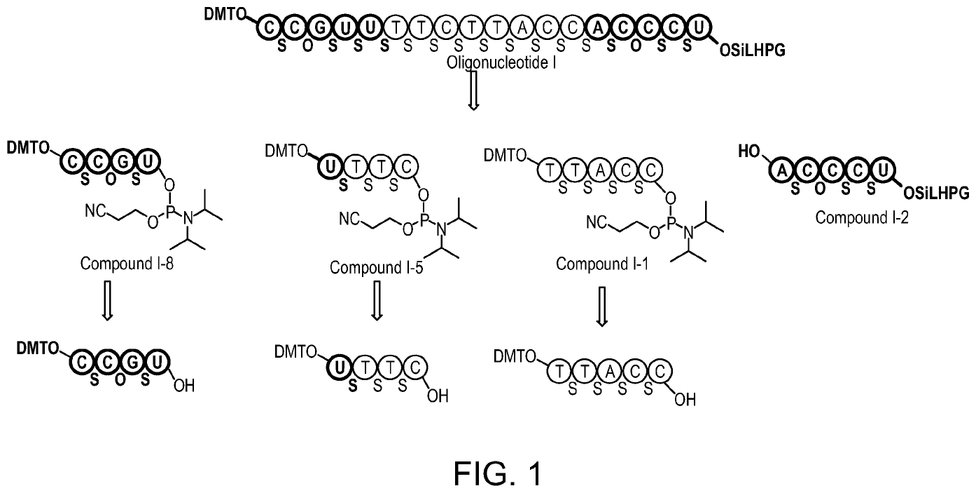

FIG. 2 shows a synthetic scheme for preparing oligonucleotide fragment A.

FIG. 3 shows a synthetic scheme for preparing oligonucleotide fragment B from reagent M19.

FIG. 4 shows a synthetic scheme for preparing oligonucleotide fragment C.

FIG. 5 shows a synthetic scheme for preparing oligonucleotide fragment D.

FIG. 6 shows a synthetic scheme for preparing oligonucleotide fragment E.

FIG. 7 shows a synthetic scheme for preparing oligonucleotide fragment F.

FIG. 8 shows a synthetic scheme for preparing oligonucleotide fragment J.

FIG. 9 shows a synthetic scheme for preparing oligonucleotide fragment K.

FIG. 10 shows a synthetic scheme for preparing oligonucleotide fragment O.

FIG. 11 shows synthetic scheme for preparing oligonucleotide fragment B from reagent M40.

FIG. 12 shows the reaction product and by-products of the one-pot procedure for the preparation of P═O linkage.

FIG. 13 shows a synthetic scheme for preparing oligonucleotide I on a large scale.

### DETAILED DESCRIPTION

Reagents for facilitating the preparation of oligonucleotides, especially in large scale are described. The synthetic processes based on reagents of the present disclosure produce protected target oligonucleotides on a large-scale with high purity without the need for chromatographic purification from the assembly of oligonucleotide fragments. Further, the protected target oligonucleotides can be easily deprotected selectively based on the conditions of the present disclosure. After deprotection and standard downstream purification, high purity ASO oligonucleotides suitable for therapeutic uses are obtained. Accordingly, the novel reagents and synthetic processes of the present disclosure provide great advantages over traditional preparation of oligonucleotides.

### Definitions

The term “nucleobase” means the heterocyclic base portion of a nucleoside. Nucleobases may be naturally occurring or may be modified. In certain embodiments, a nucleobase may comprise any atom or group of atoms capable of hydrogen bonding to a nucleobase of another nucleic acid. In particular, the nucleobase is a heterocyclic base, typically purines and pyrimidines. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable to incorporation into the compounds synthesized by the method described herein. In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

The term “nucleoside” means a compound comprising a heterocyclic base moiety and a sugar moiety, which can be modified at the 2′-end.

The term “nucleotide” means a nucleoside comprising a phosphate or thiophosphate or dithiophosphate linking group.

The term “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein, “target oligonucleotide” refers to the oligonucleotide product that can be prepared based on the reagents and the processes of the present disclosure. In certain embodiments, the target oligonucleotide comprises at least 10 or at least 15 nucleotides. In certain embodiments, the target oligonucleotide has 10 to 500, 15 to 500, 15 to 200, 15 to 100, 15 to 50, 15 to 40, 15 to 30 or 16 to 30 nucleotides.

As used herein, “oligonucleotide fragments” refers to short oligonucleotides that are assembled to make the target oligonucleotide. In certain embodiments, the oligonucleotide fragment has 3 to 10, 3 to 8, 3 to 6 or 4 to 6 nucleotides. In certain embodiments, the oligonucleotide fragment has 4 or 5 nucleotides.

As used herein, the term “alkyl” refers to a fully saturated branched or unbranched hydrocarbon moiety. In some embodiments, the alkyl comprises 1 to 30 carbon atoms, 1 to 20 carbon atoms, 1 to 16 carbon atoms, 1 to 10 carbon atoms, 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, isopropyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

As used herein, “carbocyclyl” refers to a saturated or unsaturated monocyclic, bicyclic or tricyclic (e.g., fused, bridged or spiro ring systems) ring system which has from 4- to 12-ring members, all of which are carbon. The term “carbocyclyl” encompasses cycloalkyl groups, cycloalkenyl group and aromatic groups (i.e., aryl). “Cycloalkyl” refers to completely saturated monocyclic hydrocarbon groups of 3-7 carbon atoms, including cyclopropyl, cyclobutyl, cyclpentyl, cyclohexyl and cyclopentyl; and “cycloalkyenyl” refers to unsaturated non-aromatic monocyclic hydrocarbon groups of 3-7 carbon atoms, including cyclpenteneyl, cyclohexenyl and cyclopentenyl.

The term “aryl” refers to monocyclic, bicyclic or tricyclic aromatic hydrocarbon groups having from 6 to 14 carbon atoms in the ring portion. In one embodiment, the term aryl refers to monocyclic and bicyclic aromatic hydrocarbon groups having from 6 to 10 carbon atoms. Representative examples of aryl groups include phenyl, naphthyl, fluorenyl, and anthracenyl.

The term “aryl” also refers to a bicyclic or tricyclic group in which at least one ring is aromatic and is fused to one or two non-aromatic hydrocarbon ring(s). Nonlimiting examples include tetrahydronaphthalene, dihydronaphthalenyl and indanyl.

The term “bridged ring system,” as used herein, is a ring system that has a carbocyclyl or heterocyclyl ring wherein two non-adjacent atoms of the ring are connected (bridged) by one or more (preferably from one to three) atoms selected from C, N, O, or S. A bridged ring system may have from 6-7 ring members.

The term “spiro ring system,” as used herein, is a ring system that has two rings each of which are independently selected from a carbocyclyl or a heterocyclyl, wherein the two ring structures having one ring atom in common. Spiro ring systems have from 5 to 7 ring members.

As used herein, the term “heterocyclyl” refers to a saturated or unsaturated, monocyclic or bicyclic (e.g., bridged or spiro ring systems) ring system which has from 3- to 7-ring members, or 3- to 6-ring members or 5- to 7-ring members, at least one of which is a heteroatom, and up to 4 (e.g., 1, 2, 3, or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. Unsaturated heterocyclic rings include heteroaryl rings. As used herein, the term “heteroaryl” refers to an aromatic 5 or 6 membered monocyclic ring system, having 1 to 4 heteroatoms independently selected from O, S and N, and wherein N can be oxidized (e.g., N(O)) or quaternized, and S can be optionally oxidized to sulfoxide and sulfone. In one embodiment, a heterocyclyl is a 3- to 7-membered saturated monocyclic or a 3- to 6-membered saturated monocyclic or a 5- to 7-membered saturated monocyclic ring. In one embodiment, a heterocyclyl is a 3- to 7-membered monocyclic or a 3- to 6-membered monocyclic or a 5- to 7-membered monocyclic ring. In another embodiment, a heterocyclyl is a 6 or 7-membered bicyclic ring. The heterocyclyl group can be attached at a heteroatom or a carbon atom. Examples of heterocyclyls include aziridinyl, oxiranyl, thiiranyl, oxaziridinyl, dioxiranyl, azetidinyl, oxetanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, dihydropyranyl, and heteroaryl rings including azirinyl, oxirenyl, thiirenyl, diazirinyl, azetyl, oxetyl, thietyl, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, azepinyl, oxepinyl, thiepinyl, diazepinyl, and thiazepinyl and the like. Examples of bicyclic heterocyclic ring systems include 3-azabicyclo[3.1.0]hexanyl, 3-azabicyclo[3.1.1]heptanyl, 2-azaspiro[3.3]heptanyl, 2-oxa-6-azaspiro[3.3]heptanyl, and 5-azaspiro[2.3]hexanyl.

“Halogen” or “halo” may be fluoro, chloro, bromo or iodo.

As used herein, a “hydroxyl protecting group” refers to a group that is suitable for protecting a hydroxyl group, —OH, from reacting with other reagents. Examples of hydroxyl protecting groups can be found in Greene, T W et al., Protective Groups in Organic Synthesis, 4th Ed., John Wiley and Sons (2007).

In certain embodiments, the hydroxyl protecting groups can be selected from, for example, acetyl (Ac); benzoyl (Bz); benzyl (Bn); β-methoxyethoxymethyl ether (MEM); methoxymethyl ether (MOM); methoxytrityl [(4-methoxyphenyl)diphenylmethyl, MMT); 4,4′-dimethoxytrityl (DMT); methoxyethyl (MOE); p-methoxybenzyl ether (PMB); methylthiomethyl ether; pivaloyl (Piv); tetrahydropyranyl (THP); tetrahydrofuran (THF); silyl ether (including, but not limited to, trimethylsilyl (TMS), tert-butyldiphenylsilyl (TBDPS), tert-butoxydiphenylsilyl (TBoDPS), triphenylsilyl (TPS), tert-butyldimethylsilyl (TBDMS), tri-iso-propylsilyloxymethyl (TOM), and triisopropylsilyl (TIPS) ethers); methyl ethers, and ethoxyethyl ethers (EE).

In certain embodiments, the hydroxyl protecting group protects the 3′-hydroxyl of a nucleoside (referred to as 3′-hydroxyl protecting group). In certain embodiments, the 3′-hydroxyl protecting groups include a silyl hydroxyl protecting group, such as trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl tri(trimethylsilyl)silyl, t-butylmethoxyphenylsilyl, and t-butoxydiphenylsilyl. In certain embodiments, the 3′-hydroxyl protecting group is TBDPS. In certain embodiments, the 3′-hydroxyl protecting group is a large hydrophobic protecting group (LHPG), such as those described herein.

The suffic “yl” added to the end of a chemical name indicates that the named moiety is bonded to the molecule at point. The suffix “ene” added to the end of a chemical name indicates that the named moiety is bonded to the molecule at two points.

In certain embodiments, the hydroxyl protecting group protects the 5′-hydroxyl of a nucleoside (referred to as 5′-hydroxyl protecting group). Exemplary 5′-hydoxyl groups include, but are not limited to those as described herein (e.g., R³⁵ in any of the aspects or embodiments). In a specific embodiment, 5′-hydoxyl protecting group is an acid-labile 4,4′-dimethoxytrityl (or bis-(4-methoxyphenyl)phenylmethyl) (DMT or DMTr) protecting group. In certain embodiments, the 5′-hydroxyl protecting group is a large hydrophobic protecting group (LHPG), such as those described herein.

As used herein, “selective precipitation” refers to a purification method that separates the desired product from one or more impurities in a solution by adding the solution to a solvent that precipitates out the product; while leaving the one or more impurities in the solution. Alternatively, the solvent can be added to the solution comprising the crude product and the one or more impurities to precipitate out the product. In certain embodiments, the desired compound or oligonucleotide of the present disclosure comprises a hydrophobic group (e.g., hydrophobic 3′-hydroxyl protecting group or hydrophobic 5′-hydroxyl protecting group (e.g., LHPG group described herein)) and the addition of a polar solvent (e.g. CH₃CN) into the solution containing the compound or oligonucleotide and one or more impurities to precipitate out the desired oligonucleotide. In certain embodiments, the desired compound or oligonucleotide of the present disclosure can be purified by adding a co-solvent or solvent mixture (e.g., heptane, tert-butylmethylether (TBME or MBTE), heptane/MBTE mixture (e.g. a heptane/MBTE mixture with volume ratio of heptane to MBTE in the range of 20:1 to 1:20, 9:1 to 1:9, or 4:1 to 1:4, or a heptane/MBTE mixture with heptane to MBTE volume ratio of 9:1, 4:1, 2:1, 1:1, 2:5, 1:2, 1:4 or 1:9) to a solution comprising the crude product and the one or more impurities in an organic solvent (e.g., dichloromethane (DCM) or ethylacetate (EtOAc)) to precipitate out the product. Alternatively, the solution comprising the crude product and the one or more impurities can be added to the non-polar or less polar solvent or solvent mixture to precipitate out the product. Suitable co-solvent can be determined based on the hydrophobicity of the product. In certain embodiments, the co-solvent is less polar than the organic solvent the product is dissolved in.

As used herein, “extraction” refers to a purification method that separates the desired product from one or more impurities in a solution by contacting the solution with a solvent that the product is soluble in; while the one or more impurities are insoluble. Alternatively, the solution containing the product and one or more impurities can be contacted with a solvent that the one or more impurities are soluble in; while the product is insoluble. In certain embodiments, the solution (e.g., a reaction mixture or a solution of crude product) containing the product and one or more impurities in an organic solvent (e.g., DCM, EtOAc or THF) or an organic solvent mixture can be contacted (extracted or washed) with water or an aqueous solution (e.g., NaHCO₃/H₂O solution or NaCl/H₂O solution) to remove hydrophilic impurities.

As used herein the term “base” refers to a substance that can produce hydroxide ion (OH⁻) in water solutions or a substance that can donate a pair of nonbonding electrons. Exemplary bases include, but are not limited to, alkaline hydroxide, alkaline earth hydroxide, alkylamines (e.g., tert-butylamine, sec-butylamine, trimethylamine, triethylamine, diisopropylethylamine, 2-methylpropan-2-amine), 8-diazabicyclo[5.4.0]undec-7-ene (DBU), imidazole, N-methylimidazole, pyridine and 3-picoline. As used herein, the term “salt” refers to an organic or inorganic salt of a compound, nucleotide or oligonucleotide described herein. In certain embodiments, the salt is a pharmaceutically acceptable salt thereof. The phrase “pharmaceutically acceptable” indicates that the substance or composition must be compatible chemically and/or toxicologically, with the other ingredients comprising a formulation, and/or the mammal being treated therewith. In certain embodiments, the salt of the compound, nucleotide or oligonucleotide described herein is a sodium salt, a potassium salt or an ammonium salt. In certain embodiments, the salt is a sodium salt or ammonium salt.

### 1. Reagent

In a first aspect, the present disclosure provides Reagents for facilitating the synthesis of oligonucleotides. In one embodiment, the Reagents of the disclosure serve as a protecting group protecting a 3′-hydroxyl group of a nucleotide/oligonucleotide fragment. In another embodiment, the nucleotide, oligonucleotide fragment, or target oligonucleotide which is protected by the Reagents of the disclosure can be selectively precipitated from the reaction mixture. As such, the nucleotide, oligonucleotide fragment, or target oligonucleotide is easily collected by filtration without chromatograph.

In a first embodiment of the first aspect, the present disclosure provides a compound of Formula I′ or B:

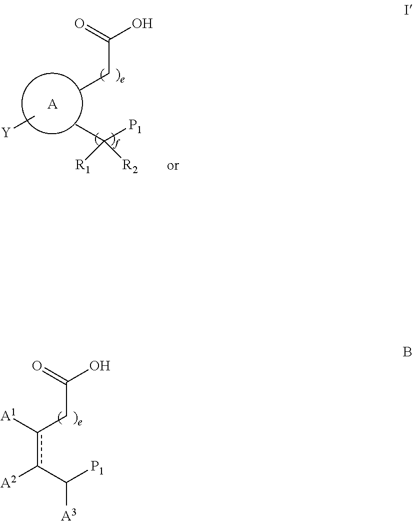

or a salt thereof, wherein:
one of A¹, A² and A³ is Y^{A} and the others are H;
[symbol] is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

In a second embodiment of the first aspect, the present disclosure provides a compound of formula I′

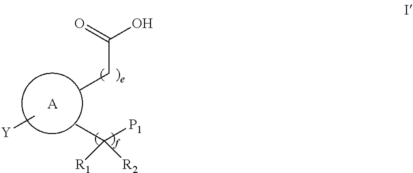

or a salt thereof, wherein:
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} is independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A} COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

In a third embodiment, the present disclosure provides a compound of formula B:

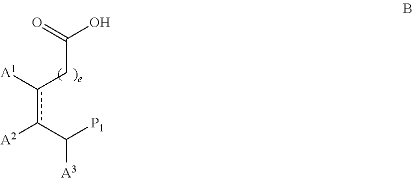

or a salt thereof. The remainder of the variables in formula B are described in the first embodiment.

In a fourth embodiment, the present disclosure provides a compound of formula B-1 or B-2:

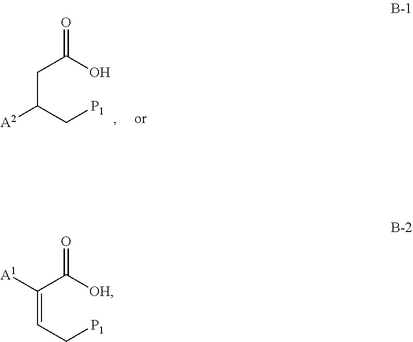

or a salt thereof. The remainder of the variables in formula B are described in the third embodiment.

In a fifth embodiment, the present disclosure provides a compound of formula I′ or B or a salt thereof, Y is a hydrophobic group comprising one or more aliphatic hydrocarbon group having 10 or more carbon atoms. The remainder of the variables in formula I′ or B are described in the first, second, third or fourth embodiment.

In a sixth embodiment, the present disclosure provides a compound of formula I′ or a salt thereof, wherein ring A is phenyl or naphthalenyl. The remainder of the variables in Formula I′ are described in the second and/or fifth embodiments.

In a seventh embodiment, the present disclosure provides a compound of formula I′ or B or a salt thereof, wherein P₁ is a silyl hydroxyl protecting group selected from the following:

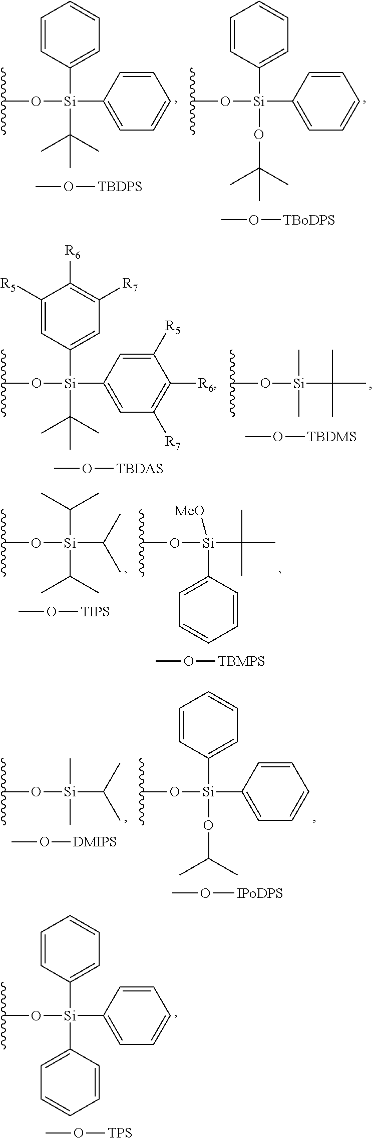

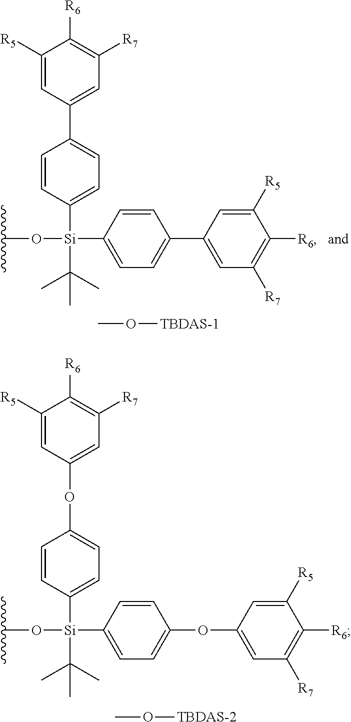

wherein

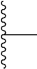

represents the point of attachment for P₁; and R₅, R₆ and R₇ are each independently H, C₁₋₃₀alkyl, or C₁₋₃₀alkoxy. The remainder of the variables in Formula I′ or B are described in any one of the first through sixth embodiments.

In an eighth embodiment, the present disclosure provides a compound of formula I′ or B or a salt thereof, wherein P₁ is selected from the group consisting of —O-TBDMS, —O-TIPS, —O-TBDPS, —O-TBoDPS, and —O-TBDAS:

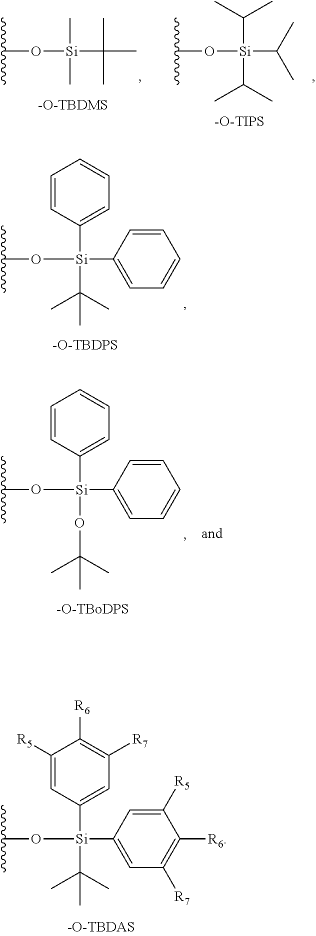

The remainder of the variables in Formula I′ are described in any one of the first through seventh embodiments.

In a ninth embodiment, the present disclosure provides a compound of formula I or Ia:

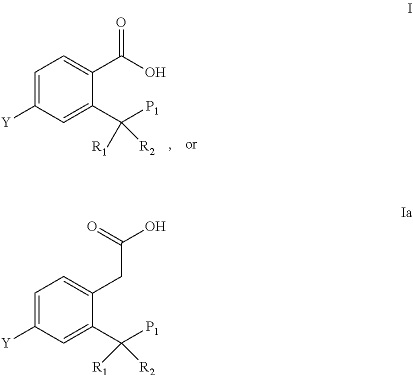

or a salt thereof;
wherein P₁ is selected from the group consisting of —O-TBDPS, —O-TBoDPS, and —O-TBDAS:

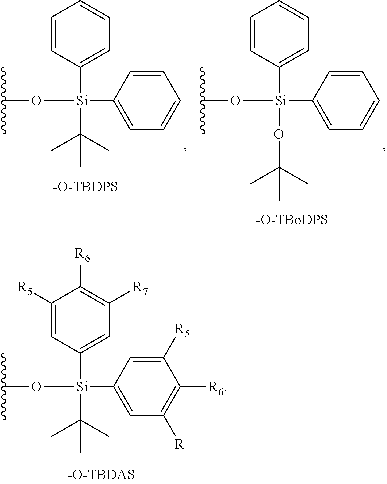

The remainder of the variables in Formula I or Ia are described in the first, second, and/or fifth through eighth embodiments.

In a tenth embodiment, the present disclosure provides a compound of Formulae I′, B, or Formula I or a salt thereof, wherein Y is represented by Formula A:

W—V—U—* (A)

wherein:
—* represents the point of attachment for Y;
W is represented by Formula A1, A2, A2-1, A2-2, A3, A3-1, or A3-2:

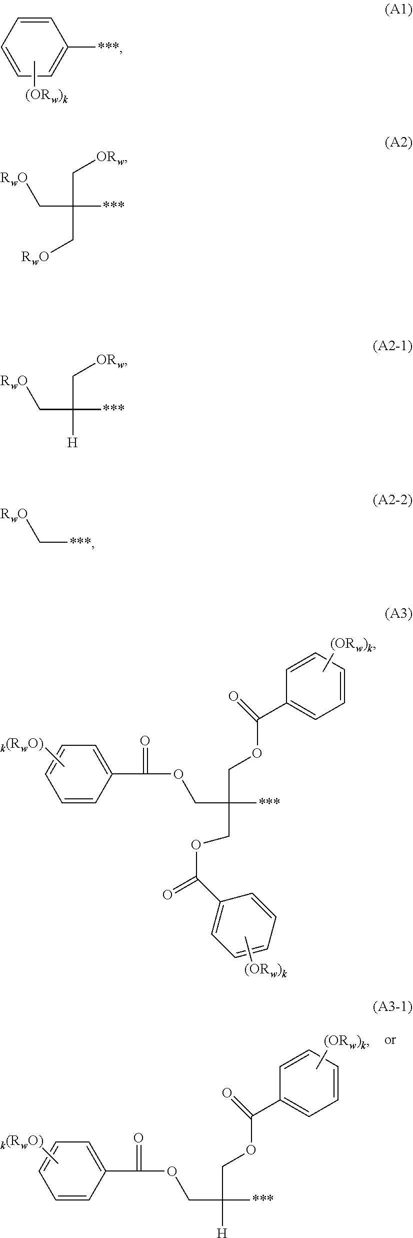

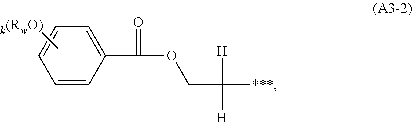

wherein
—*** represents the point where W and V connect;
each R_{w} is independently an aliphatic hydrocarbon group having 10 or more carbon atoms;
k is an integer from 1 to 5;
V is a bond, oxygen, C₁₋₂₀alkylene, C₁₋₆alkynylene, —C(═O)—, ***—C(═O)—O—**, ***—O—C(═O)—**,

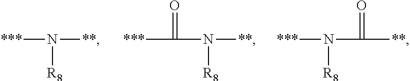

or 5 to 7 member heteroaryl having 1 to 3 heteroatoms selected from oxygen, nitrogen, and sulfur, wherein the heteroaryl is optionally substituted by 1-3 R₈; wherein —** represents the point where V and U connect; and R₈ is H or C₁₋₃₀alkyl; and
U is a bond, oxygen, C₁₋₂₀alkylene, carbonyl, ***—O—C(═O)—** 5 to 7 member heterocyclyl having 1 to 3 heteroatoms selected from oxygen, nitrogen, and sulfur; 5 to 7 member heteroaryl having 1 to 3 heteroatoms selected from oxygen, nitrogen, and sulfur, wherein the heteroaryl is optionally substituted by 1-3 R₈; or a group represented by formula A4, A5, or A6:

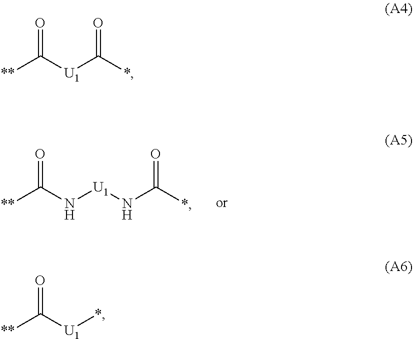

wherein U₁ is C₁₋₆alkylene, C₁₋₆alkyleneoxy, 5 to 7 member heterocyclyl having 1 to 3 heteroatoms selected from oxygen, nitrogen, and sulfur, or 5 to 7 member heteroaryl having 1 to 3 heteroatoms selected from oxygen, nitrogen, and sulfur. The remainder of the variables in Formula I, Ia, B, or Formula I′ are described in any one of the first through ninth embodiments.

In an eleventh embodiment, the present disclosure provides a compound of Formula I′, B, or Formula I or a salt thereof, wherein the TBDAS group is:

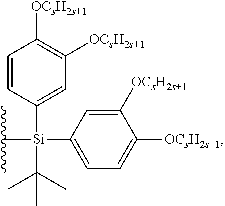

wherein s is an integer from 1 to 30. The remainder of the variables in Formula I, Ia, B, or Formula I′ are described in any one of the seventh through tenth embodiments.

In a twelfth embodiment, the present disclosure provides a compound of Formula I′, B or Formula I or Ia or a salt thereof, wherein P₁ is —O-TBDPS. The remainder of the variables in Formula I, Ia or Formula I′ or B are described in any one of the first through eleventh embodiments.

In a thirteenth embodiment, the present disclosure provides a compound of Formula I, Ia, B, or I′ or a salt thereof, wherein W is represented by Formula A1:

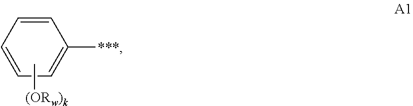

wherein R_{w} is CₙH₂ₙ₊₁; and n is an integer from 1 to 30. The remainder of the variables in Formula I, B, or I′ are described in the tenth embodiment.

In a fourteenth embodiment, the present disclosure provides a compound of Formula I, Ia, B, or I′ or a salt thereof, wherein R_{w} is selected from a group consisting of C₁₂H₂₅, C₁₈H₃₇, C₂₀H₄₁, C₂₂H₄₅, C₂₄H₄₉, C₂₆H₅₃, and C₂₈H₅₇. The remainder of the variables in Formula I, Ia B, or I′ are described in any one of the tenth through thirteenth embodiments.

In a fifteenth embodiment, the present disclosure provides a compound of Formula I, Ia, B, or I′ or a salt thereof, wherein V is a bond, CH₂, CH₂CH₂, C(═O), ***—C(═O)—O—**, or

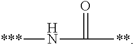

The remainder of the variables in Formula I, Ia, B, or I′ are described in any one of the tenth through fourteenth embodiments.

In a sixteenth embodiment, the present disclosure provides a compound of Formula I, Ia, B, or I′ or a salt thereof, wherein U is a bond, CH₂, CH₂CH₂, carbonyl, triazolylene, piperazinylene,

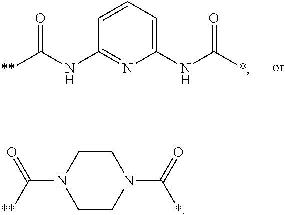

The remainder of the variables in Formula I, Ia, B, or I′ are described in any one of the tenth through fifteenth embodiments.

In a seventeenth embodiment, the present disclosure provides a compound of Formula I, Ia, B, or I′ or a salt thereof, wherein U—V is selected from the group consisting of:

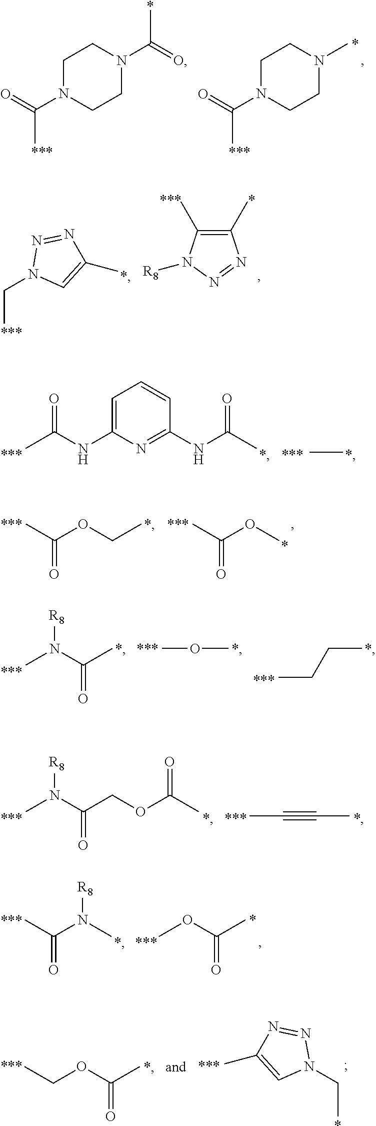

wherein R₈ is H or C₁₋₆alkyl. The remainder of the variables in Formula I, B, or I′ are described in any one of the tenth through sixteenth embodiments.

In an eighteenth embodiment, the present disclosure provides a compound of Formula I or Ia or Formula I′ or B or a salt thereof, wherein Y is selected from the groups consisting of

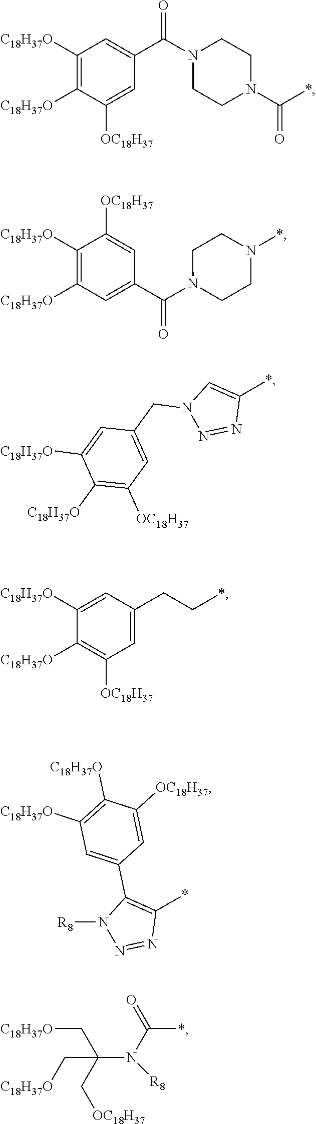

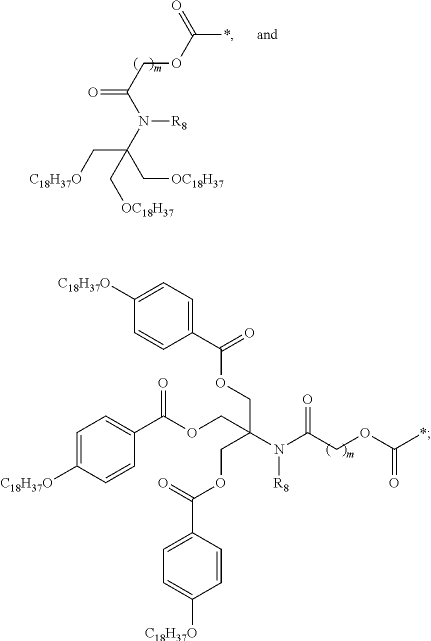

wherein
R₈ is H or C₁₋₆alkyl; and
m is an integer from 1 to 5. The remainder of the variables in Formula I′, B, or Formula I or Ia are described in any one of the first through twelfth embodiments.

In a nineteenth embodiment, the present disclosure provides a compound of Formula I or Ia or Formula I′ or a salt thereof, wherein R₁ and R₂ are independently H or CH₃. The remainder of the variables in Formula I or Ia or Formula I′ are described in the first, second, and/or fifth through eighteenth embodiments. In a specific embodiment, R₁ and R₂ are both H. In another specific embodiment, R₁ and R₂ are both CH₃.

In a twentieth embodiment, the present disclosure provides a compound of Formula I′ or Formula B or a salt thereof, wherein e is 0, 1, or 2; and f is 0, 1, or 2. The remainder of the variables in Formula I′ are described in the first, second, third, and/or fifth through nineteenth embodiments.

In a twenty-first embodiment, the present disclosure provides a compound of Formula I, Ia, I′, or B, or a salt thereof, wherein R₈ is H or C₁₋₄alkyl. The remainder of the variables in Formula I, Ia, I′, or B are described in any one of the tenth through twentieth embodiments.

In a twenty-second embodiment, the present disclosure provides a compound of Formula II or IIa:

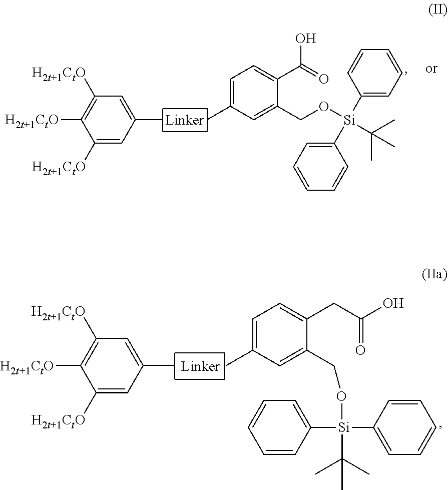

or a salt thereof, wherein:
t is an integer from 10 to 30;
[symbol] is selected from the group consisting of

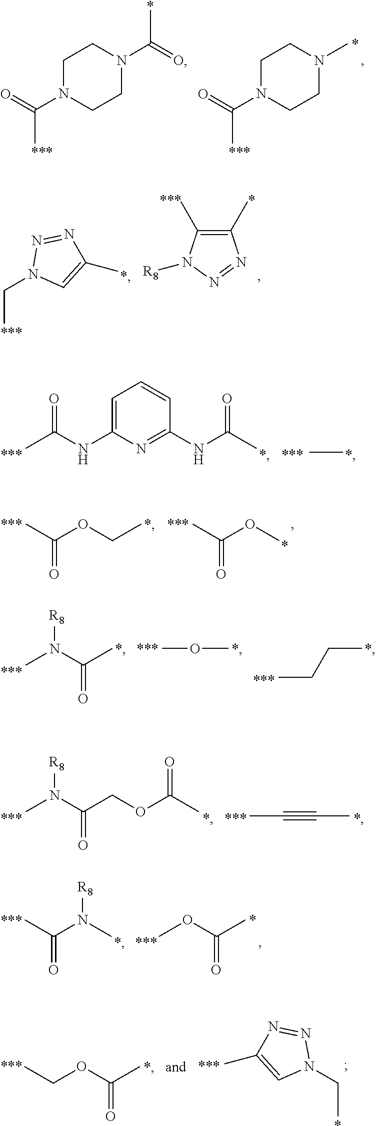

wherein R₈ is H or C₁₋₆alkyl.

In a twenty-third embodiment, the present disclosure provides a compound of Formula II or a salt thereof that is selected from the group consisting of

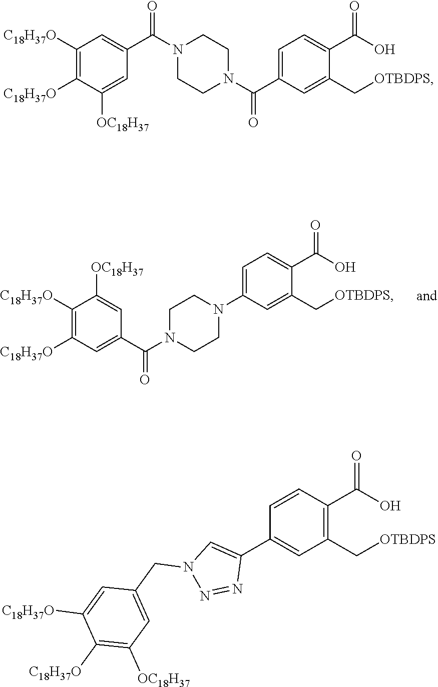

or a salt thereof.

In a twenty-fourth embodiment, the present disclosure provides the following compound:

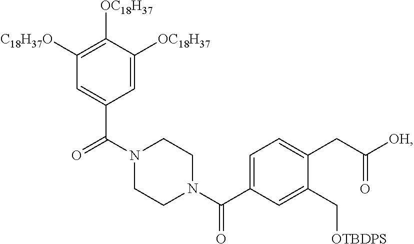

or a salt thereof.

In a twenty-fifth embodiment, the present disclosure provides a compound selected from one of the following formulae:

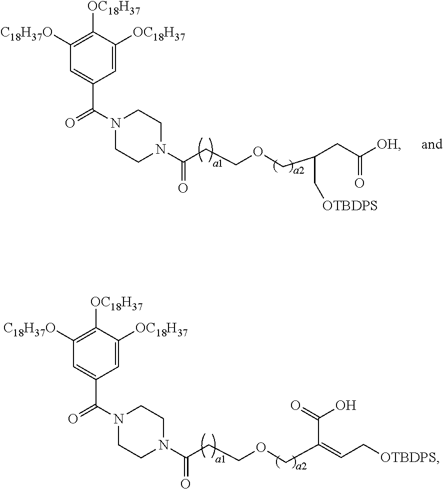

or a salt thereof. The remainder of the variables in the above formulae are described in the first embodiment. In some embodiments, a1 and a2 are each an integer from 1 to 6, 1 to 5, or 1 to 4.

In a twenty-sixth embodiment, the present disclosure provides the compounds depicted in Table 1 and prepared in the Exemplification, both the neutral form and salts thereof.

TABLE 1

| Id | Structure | Chemical Name |
|---|---|---|
| M19 | 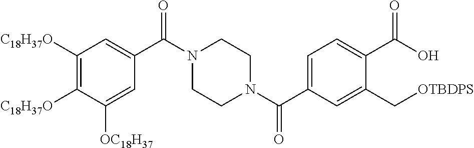 | 2-(((tert-butyldiphenylsilyl)oxy) methyl)-4-(4-(3,4,5- tris(octadecyloxy)benzoyl) piperazine-1-carbonyl)benzoic acid |
| M22 | 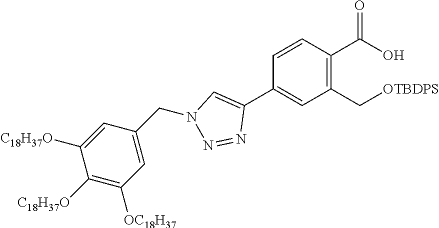 | 2-(((tert- butyldiphenylsilyl)oxy)methyl)-4- (1-(3,4,5-tris(octadecyloxy)benzyl)- 1H-1,2,3-triazol-4-yl)benzoic acid |
| M36 | 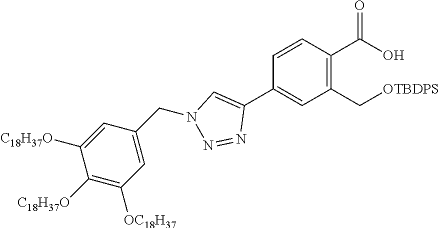 | 2-(((tert-butyldiphenylsilyl)oxy) methyl)-4-(4-(3,4,5- tris(octadecyloxy)benzoyl)piperazin-1- yl)benzoic acid |
| M40 | 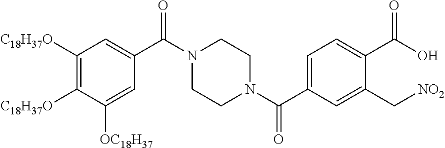 | 2-(nitromethyl)-4-(4-(3,4,5- tris(octadecyloxy)benzoyl) piperazine-1-carbonyl)benzoic acid |
| M50 | 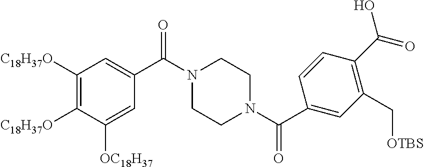 | 2-(((tert- butyldimethylsilyl)oxy)methyl)-4- (4-(3,4,5- tris(octadecyloxy)benzoyl)piperazine- 1-carbonyl)benzoic acid |
| M60 | 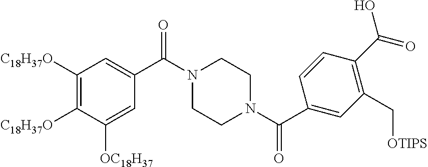 | 2-(((triisopropylsilyl)oxy)methyl)-4- (4-(3,4,5- tris(octadecyloxy)benzoyl)piperazine- 1-carbonyl)benzoic acid |

### 2. 3′-Protected Nucleotides or Oligonucleotides

In a second aspect, the present disclosure describes a nucleotide or an oligonucleotide protected by a 3′-hydroxyl protecting group described herein. In one embodiment, the 3′-hydroxyl protecting group is derived from the Regent described above. In another embodiment, the protected nucleotide or oligonucleotide is separated by selective precipitation. In another embodiment, the protected nucleotide or oligonucleotide is soluble in a non-polar organic solvent such as dichloromethane but precipitate in a polar organic solvent such as acetonitrile.

In a twenty-seventh embodiment, the present disclosure provides a nucleotide or oligonucleotide represented by Formula III or IIIP,

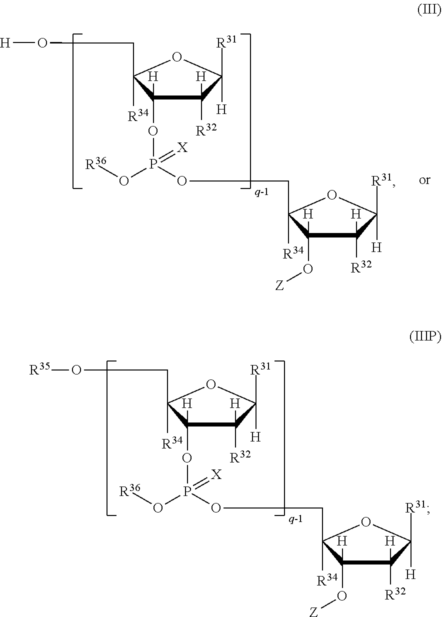

or a salt thereof,
wherein
R³¹, for each occurrence, is independently a nucleobase, wherein the NH₂ of the nucleobase, if present, is optionally protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³².
R³⁵ is a hydroxyl protecting group;
R³⁶, for each occurrence, is independently H, C₁₋₆alkyl group, C₂₋₆alkenyl group, phenyl or benzyl group, each of which is optionally substituted with —CN, —NO₂ or halogen; or
R³⁶ is

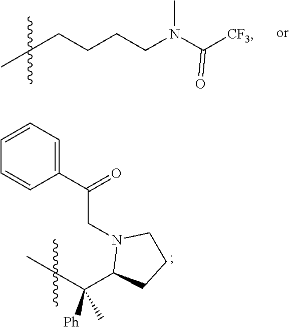 [SMILES: *CCCCN(C)C(=O)C(F)(F)F ; "or" ; *[C@](C)(c1ccccc1)[C@@H]1CCCN1CC(=O)c1ccccc1]

q is an integer from 1 to 20;
X, for each occurrence, is independently O or S;
Z is a group represented by Formula I* or B*,

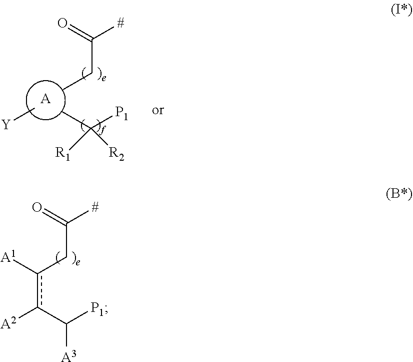

wherein
—#represents the point of attachment for Z;
one of A¹, A² and A³ is Y^{A} and the others are H;
[symbol] is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurance, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

In a twenty-eighth embodiment, the present disclosure provides a nucleotide or oligonucleotide represented by Formula III′ or IIIP′

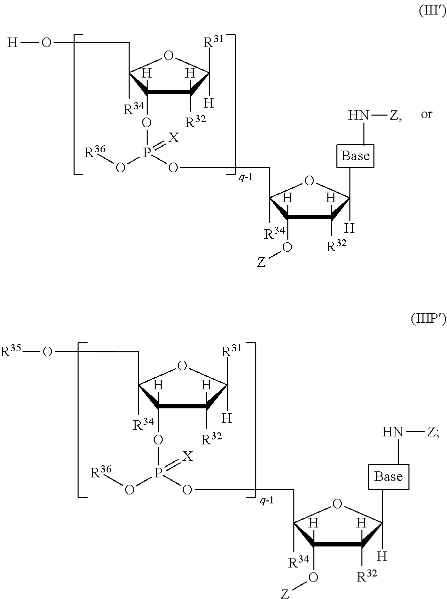

or a salt thereof, wherein:
Q is a hydroxyl protecting group;
[symbol] is a nucleobase comprising a NH₂ group which is modified by Z;
R³¹, for each occurrence, is independently a nucleobase, wherein the NH₂ of the nucleobase, if present, is optionally protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³².
R³⁵ is a hydroxyl protecting group;
R³⁶, for each occurrence, is independently H, C₁₋₆alkyl group, C₂₋₆alkenyl group, phenyl or benzyl group, each of which is optionally substituted with —CN, —NO₂ or halogen; or
R³⁶ is

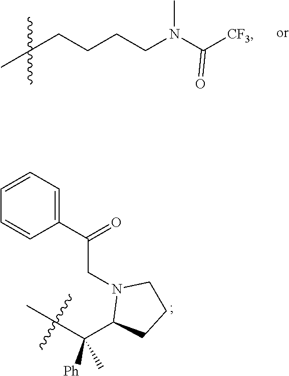 [SMILES: *CCCCN(C)C(=O)C(F)(F)F ; "or" ; *[C@](C)(c1ccccc1)[C@@H]1CCCN1CC(=O)c1ccccc1]

q is an integer from 1 to 20;
X, for each occurrence, is independently O or S;
Z is a group represented by Formula I* or B*,

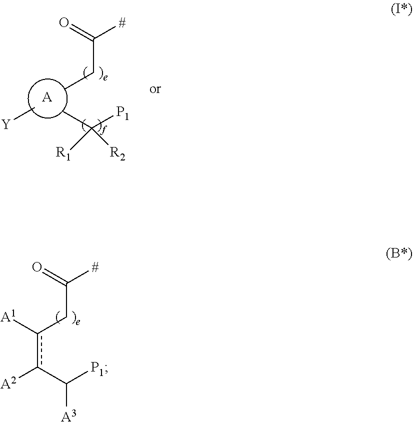

wherein
—#represents the point of attachment for Z;
one of A¹, A² and A³ is Y^{A} and the others are H; [symbol] is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
   f is an integer from 0 to 6.

In certain embodiments, the hydroxyl protecting group of R³² is a silyl protecting group. In certain embodiments, the silyl protecting group is selected from the group consisting of trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl tri(trimethylsilyl)silyl, t-butylmethoxyphenylsilyl, and t-butoxydiphenylsilyl.

In a twenty-ninth embodiment, the present disclosure provides a nucleotide or oligonucleotide represented by Formula III, III′, IIIP, or IIIP′, or a salt thereof, wherein Z is a group represented by Formula I*,

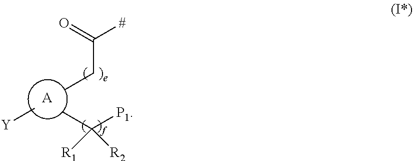

The remainder of the variables in Formula III, III′, IIIP, or IIIP′, are described in the twenty-seventh and/or twenty-eighth embodiments.

In a thirtieth embodiment, the present disclosure provides a nucleotide or oligonucleotide represented by Formula III, III′, IIIP, or IIIP′ or a salt thereof, wherein Z is a group represented by Formula B*,

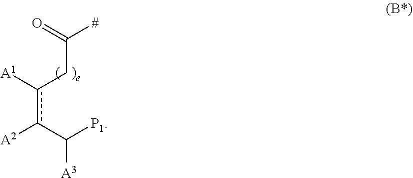

The remainder of the variables in Formula III, III′, IIIP, or IIIP′, are described in the twenty-seventh and/or twenty-eighth embodiments.

In a thirty-first embodiment, the present disclosure provides a nucleotide or oligonucleotide represented by Formula III, III′, IIIP, or IIIP′ or a salt thereof, wherein Z is a group represented by Formula B-1* or B-2*:

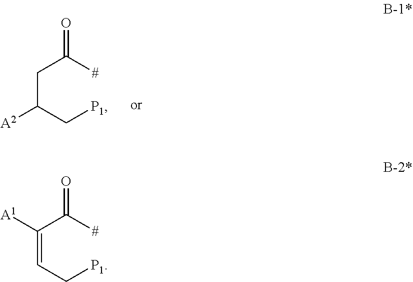

The remainder of the variables in Formula III, III′, IIIP, or IIIP′, are described in the twenty-seventh and/or twenty-eighth embodiments.

In a thirty-second embodiment, the present disclosure provides a nucleotide or oligonucleotide represented by Formula III, III′, IIIP, or IIIP′ or a salt thereof, wherein Y is a hydrophobic group comprising one or more aliphatic hydrocarbon group having 10 or more carbon atoms. The remainder of the variables in Formula III, III′, IIIP, or IIIP′ are described in the twenty-seventh and/or twenty-eighth embodiments.

In a thirty-third embodiment, the present disclosure provides a nucleotide or oligonucleotide represented by Formula III, III′, IIIP, or IIIP′ or a salt thereof, wherein ring A is phenyl or naphthalenyl. The remainder of the variables in Formula III, III′, IIIP, or IIIP′ are described in the twenty-seventh, twenty-eighth, and/or thirty-second embodiments.

In a thirty-fourth embodiment, the present disclosure provides a nucleotide or oligonucleotide represented by Formula III, III′, IIIP, or IIIP′ or a salt thereof, wherein P₁ is a silyl hydroxyl protecting group selected from the following:

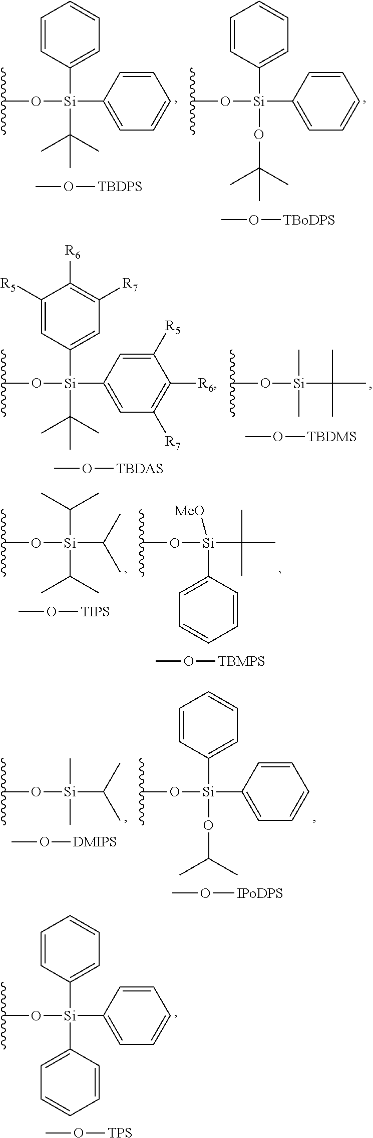

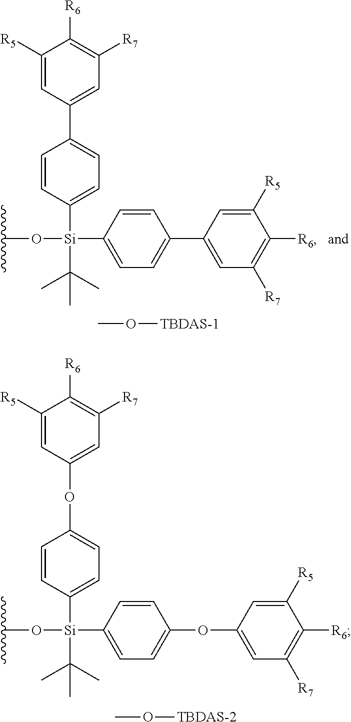

and —OTBDAS-2; wherein

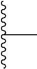

represents the point of attachment for P₁ and R₅, R₆ and R₇ are each independently H, C₁₋₃₀alkyl, or C₁₋₃₀alkoxy. The remainder of the variables in Formula III, III′, IIIP, or IIIP′ are described in any one of the twenty-seventh through thirty-third embodiments.

In a thirty-fifth embodiment, the present disclosure provides a nucleotide or oligonucleotide represented by Formula III, III′, IIIP, or IIIP′ or a salt thereof, wherein P₁ is selected from the group consisting of —O-TBDMS, —O-TIPS, —O-TBDPS, —O-TBoDPS, and —O-TBDAS:

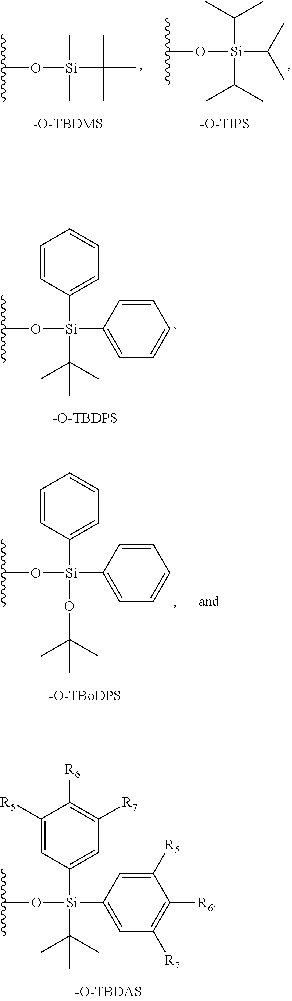

The remainder of the variables in Formula III, III′, IIIP, or IIIP′ are described in any one of the twenty-seventh through thirty-fourth embodiments.

In a thirty-sixth embodiment, the present disclosure provides a nucleotide or oligonucleotide represented by Formula III, III′, IIIP, or IIIP′ or a salt thereof, wherein Z is a group represented by by Formula I** or Ia**:

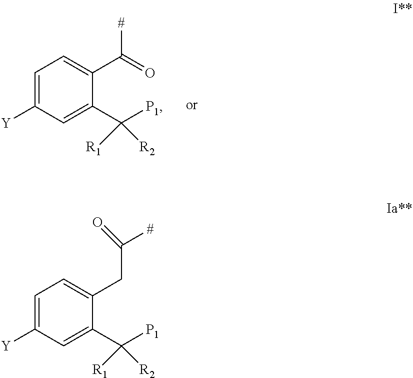

or salt thereof, wherein P₁ is selected from the group consisting of —O-TBDPS, —O-TBoDPS, and —O-TBDAS:

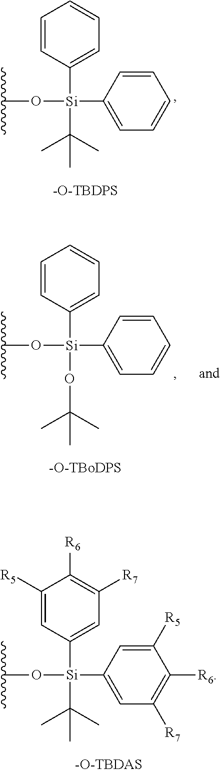

The remainder of the variables in Formula III, III′, IIIP, or IIIP′ are described in any one of the twenty-seventh through thirty-fifth embodiments.

In a thirty-seventh embodiment, the present disclosure provides a nucleotide or oligonucleotide represented by Formula III, III′, IIIP, or IIIP′ or a salt thereof, wherein Y is represented by Formula A:

W—V—U—* (A)

wherein:
—* represents the point of attachment for Y;
W is represented by Formula A1, A2, A2-1, A2-2, A3, A3-1, or A3-2:

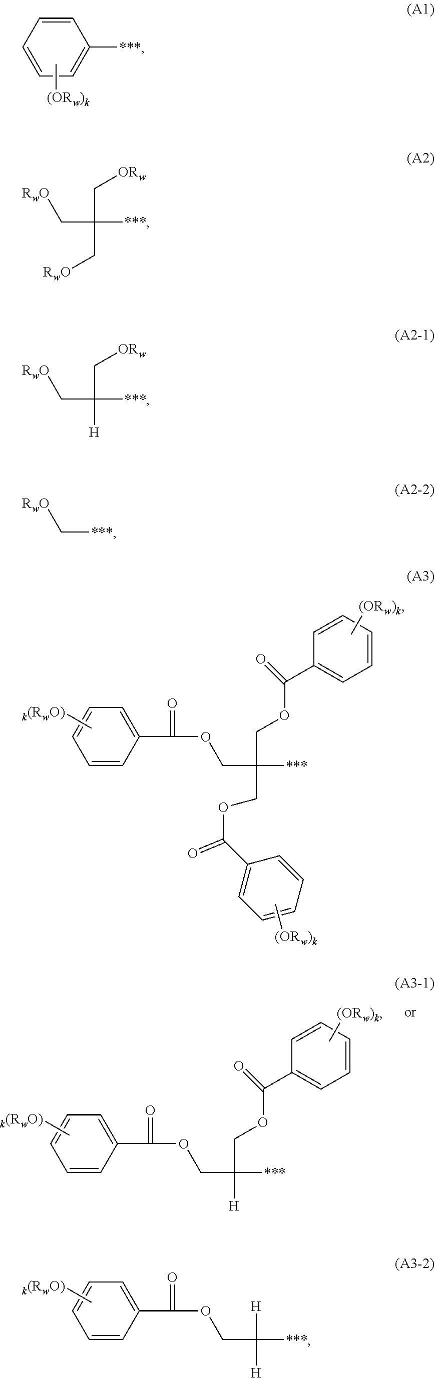

wherein
—*** represents the point where W and V connect;
each R_{w} is independently an aliphatic hydrocarbon group having 10 or more carbon atoms;
k is an integer from 1 to 5;
V is a bond, oxygen, C₁₋₂₀alkylene, C₁₋₆alkynylene, —C(═O)—, ***—C(═O)—O—**, ***—O—C(═O)—**,

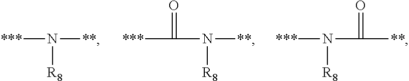

or 5 to 7 member heteroaryl having 1 to 3 heteroatoms selected from oxygen, nitrogen, and sulfur, wherein the heteroaryl is optionally substituted by 1-3 R₈; wherein —** represents the point where V and U connect; and R₈ is H or C₁₋₃₀alkyl; and U is a bond, oxygen, C₁₋₂₀alkylene, carbonyl, ***—O—C(═O)—** 5 to 7 member heterocyclyl having 1 to 3 heteroatoms selected from oxygen, nitrogen, and sulfur; 5 to 7 member heteroaryl having 1 to 3 heteroatoms selected from oxygen, nitrogen, and sulfur, wherein the heteroaryl is optionally substituted by 1-3 R₈; or a group represented by formula A4, A5, or A6:

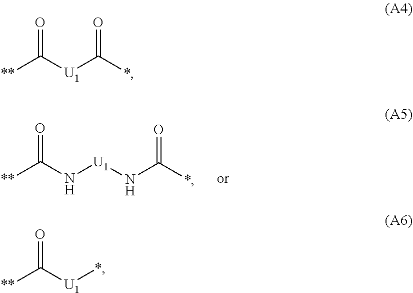

wherein U₁ is C₁₋₆alkylene, C₁₋₆alkyleneoxy, 5 to 7 member heterocyclyl having 1 to 3 heteroatoms selected from oxygen, nitrogen, and sulfur, or 5 to 7 member heteroaryl having 1 to 3 heteroatoms selected from oxygen, nitrogen, and sulfur. The remainder of the variables in Formula III, III′, IIIP, or IIIP′ are described in any one of the twenty-seventh through thirty-sixth embodiments.

In a thirty-eighth embodiment, the present disclosure provides a nucleotide or oligonucleotide represented by Formula III, III′, IIIP, or IIIP′ or a salt thereof, wherein the TBDAS group is:

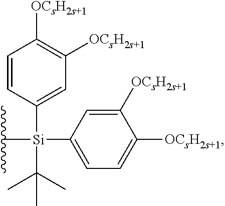

wherein s is an integer from 1 to 30. The remainder of the variables in Formula III, III′, IIIP, or IIIP′ are described in any one of the thirty-fourth through thirty-seventh embodiments.

In a thirty-ninth embodiment, the present disclosure provides a nucleotide or oligonucleotide represented by Formula III, III′, IIIP, or IIIP′ or a salt thereof, wherein P₁ is TBDPS. The remainder of the variables in Formula III, III′, IIIP, or IIIP′ are described in any one of the twenty-seventh through thirty-seventh embodiments.

In a fortieth embodiment, the present disclosure provides a nucleotide or oligonucleotide represented by Formula III, III′, IIIP, or IIIP′ or a salt thereof, wherein W is represented by Formula A1:

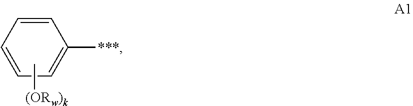

wherein R_{w} is CₙH₂ₙ₊₁; and n is an integer from 1 to 30. The remainder of the variables in Formula III, III′, IIIP, or IIIP′ are described in the thirty-seventh embodiment.

In a forty-first embodiment, the present disclosure provides a nucleotide or oligonucleotide represented by Formula III, III′, IIIP, or IIIP′ or a salt thereof, wherein R_{w} is selected from a group consisting of C₁₂H₂₅, C₁₈H₃₇, C₂₀H₄₁, C₂₂H₄₅, C₂₄H₄₉, C₂₆H₅₃, and C₂₈H₅₇. The remainder of the variables in Formula III, III′, IIIP, or IIIP′ are described in the thirty-seventh and/or fortieth embodiments.

In a forty-second embodiment, the present disclosure provides a nucleotide or oligonucleotide represented by Formula III, III′, IIIP, or IIIP′ or a salt thereof, wherein V is a bond, CH₂, CH₂CH₂, C(═O)—, ***C(═O)—O—**, or

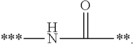

The remainder of the variables in Formula III, III′, IIIP, or IIIP′ are described in any one of the thirty-seventh through forty-first embodiments.

In a forty-third embodiment, the present disclosure provides a nucleotide or oligonucleotide represented by Formula III, III′, IIIP, or IIIP′ or a salt thereof, wherein U is a bond, CH₂, CH₂CH₂, carbonyl, triazolylene, piperazinylene,

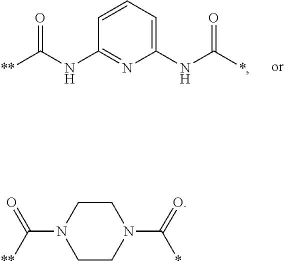

The remainder of the variables in Formula III, III′, IIIP, or IIIP′ are described in any one of the thirty-seventh through forty-second embodiments.

In a forty-four embodiment, the present disclosure provides a nucleotide or oligonucleotide represented by Formula III, III′, IIIP, or IIIP′ or a salt thereof, wherein U—V is selected from the group consisting of

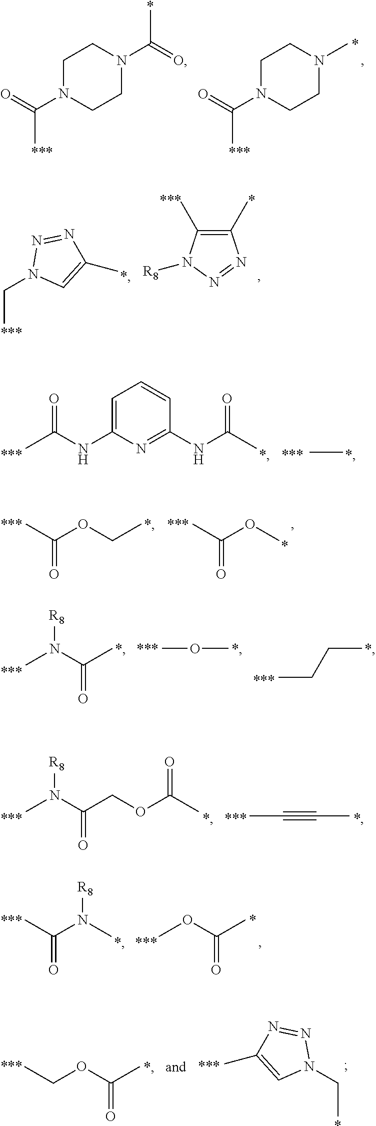

wherein R₈ is H or C₁₋₆alkyl. The remainder of the variables in Formula III, III′, IIIP, or IIIP′ are described in any one of the thirty-seventh through the forty-first embodiments.

In a forty-fifth embodiment, the present disclosure provides a nucleotide or oligonucleotide represented by Formula III, III′, IIIP, or IIIP′ or a salt thereof, wherein Y is selected from the groups consisting of:

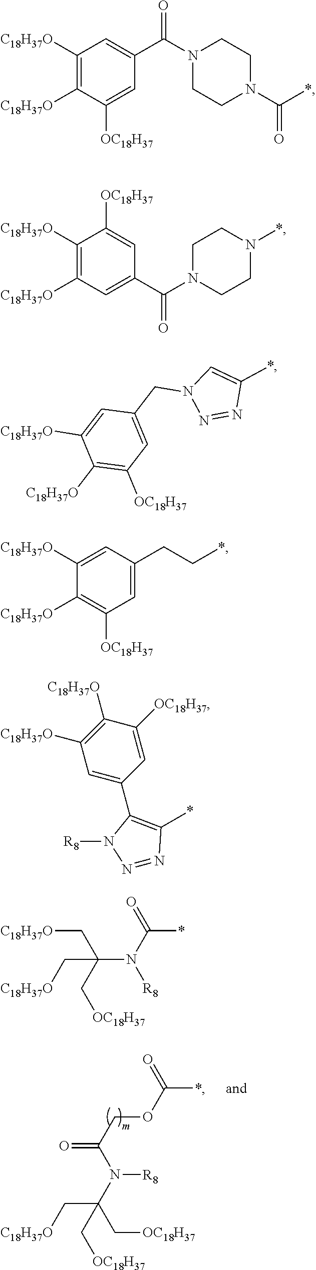

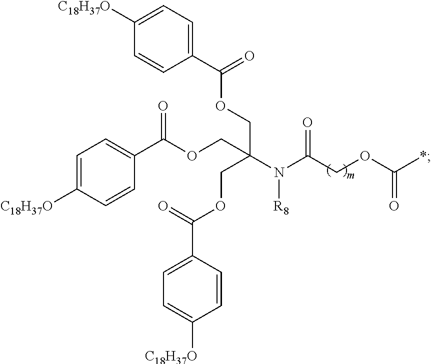

wherein
R₈ is H or C₁₋₆alkyl; and
m is an integer from 1 to 5.
   The remainder of the variables in Formula III, III′, IIIP, or IIIP′ are described in any one of the twenty-seventh through the thirty-ninth embodiments.

In a forty-sixth embodiment, the present disclosure provides a nucleotide or oligonucleotide represented by Formula III, III′, IIIP, or IIIP′ or a salt thereof, wherein R₁ and R₂ are independently H or CH₃. The remainder of the variables in Formula III, III′, IIIP, or IIIP′ are described in any one of the twenty-seventh through forty-fifth embodiments. In a specific embodiment, R₁ and R₂ are both H. In another specific embodiment, R₁ and R₂ are both CH₃.

In a forty-seventh embodiment, the present disclosure provides a nucleotide or oligonucleotide represented by Formula III, III′, IIIP, or IIIP′ or a salt thereof, wherein e is 0, 1, or 2; and f is 0, 1, or 2. The remainder of the variables in Formula III, III′, IIIP, or IIIP′ are described in any one of the twenty-seventh through the forty-sixth embodiments.

In a forty-eighth embodiment, the present disclosure provides a nucleotide or oligonucleotide represented by Formula III, III′, IIIP, or IIIP′ or a salt thereof. wherein R₈ is H or C₁₋₄alkyl. The remainder of the variables in Formula III or HIP are described in the thirty-seventh embodiment. In one embodiment, R₈ is H or methyl.

In a forty-ninth embodiment, the present disclosure provides a nucleotide or oligonucleotide represented by Formula III, III′, IIIP, or IIIP′ or a salt thereof, wherein Z is represented by Formula II* or IIa*,

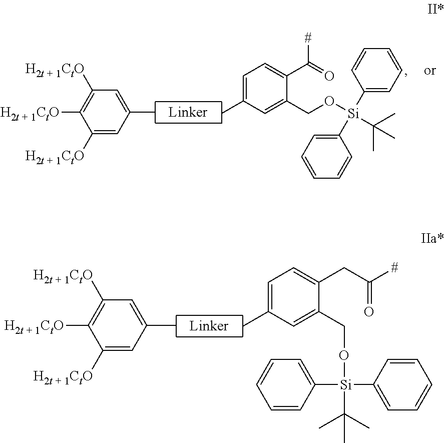

Wherein:
t is an integer from 10 to 30;
[symbol] is selected from the group consisting of

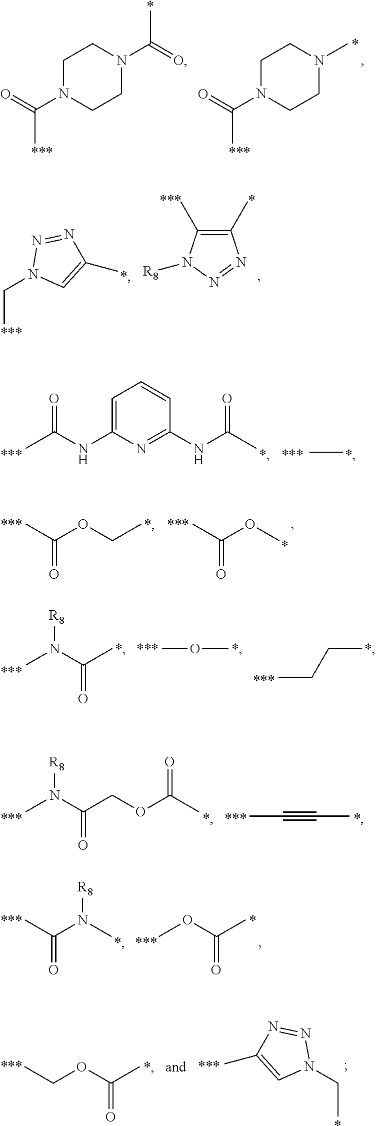

wherein R₈ is H or C₁₋₆alkyl. The remainder of the variables in Formula III, III′, IIIP, or IIIP′ are described in the twenty-seventh and/or twenty-eighth embodiments.

In a fiftieth embodiment, the present disclosure provides a nucleotide or oligonucleotide represented by Formula III, III′, IIIP, or IIIP′ or a salt thereof, wherein Z is

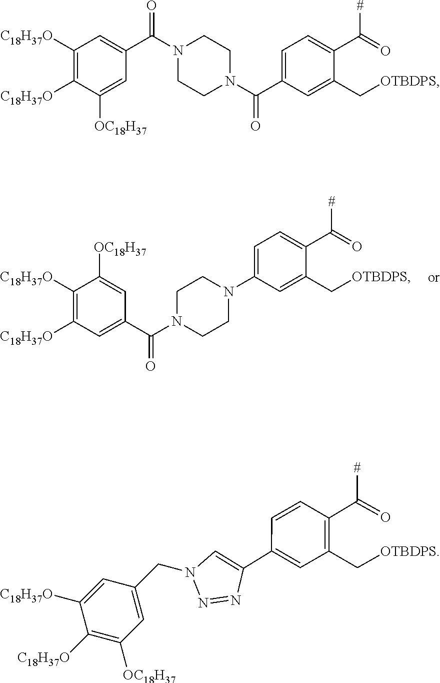

The remainder of the variables in Formula III, III′, IIIP, or IIIP′ are described in the forty-ninth embodiment.

In a fifty-first embodiment, the present disclosure provides a nucleotide or oligonucleotide represented by Formula III, III′, IIIP, or IIIP′ or a salt thereof, wherein Z is

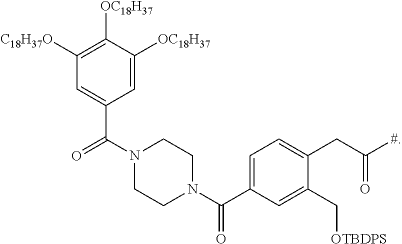

The remainder of the variables in Formula III, III′, IIIP, or IIIP′ are described in the twenty-seventh and/or twenty-eighth embodiments.

In a fifty-second embodiment, the present disclosure provides a nucleotide or oligonucleotide represented by Formula III, III′, IIIP, or IIIP′ or a salt thereof, wherein Z is

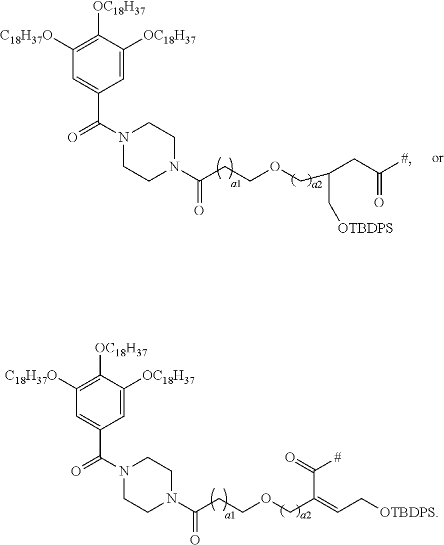

The remainder of the variables in Formula III, III′, IIIP, or IIIP′ are described in the twenty-seventh and/or twenty-eighth embodiments. In some embodiments, a1 and a2 are each an integer from 1 to 6, 1 to 5, or 1 to 4.

In a fifty-third embodiment, the present disclosure provides a nucleotide or oligonucleotide represented by Formula III, III′, IIIP, or IIIP′ or a salt thereof, wherein when X is S, the phosphorothiolate group has S-configuration as shown below:

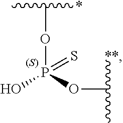

or
R-configuration as shown below:

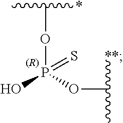

wherein [symbol] indicates the connection point to 3′—OH group and [symbol] indicates the connection point to 5′-OH group. The remainder of the variables in Formula III, III′, IIIP, or IIIP′ are described in any one of the twenty-seventh through the fifty-second embodiments.

In certain embodiments, the present disclosure provides a nucleotide or oligonucleotide represented by Formula III, III′, IIIP, or IIIP′ or a salt thereof, wherein R³¹, for each occurrence, is adenine (A), guanine (G), thymine (T), cytosine (C), or uracil (U). The remainder of the variables in Formula III, III′, IIIP, or IIIP′ are described in any one of the twenty-seventh through the fifty-third embodiments.

In certain embodiments, the present disclosure provides a nucleotide or oligonucleotide represented by Formula III, III′, IIIP, or IIIP′ or a salt thereof, wherein R³², for each occurrence, is independently H, F, Cl, Br, I, or —OCH₂CH₂OMe. The remainder of the variables in Formula III, III′, IIIP, or IIIP′ are described in any one of the twenty-seventh through the fifty-third embodiments. In a specific embodiment, R³², for each occurrence, is independently H or —OCH₂CH₂OMe.

In certain embodiments, the present disclosure provides a nucleotide or oligonucleotide represented by Formula III, III′, IIIP, or IIIP′ or a salt thereof, wherein R³⁴, is H. The remainder of the variables in Formula III, III′, IIIP, or IIIP′ are described in any one of the twenty-seventh through the fifty-third embodiments.

In certain embodiments, the present disclosure provides a nucleotide or oligonucleotide represented by Formula III, III′, IIIP, or IIIP′ or a salt thereof, wherein R³⁵ is 4,4′-dimethoxytrityl. The remainder of the variables in Formula III, III′, IIIP, or IIIP′ are described in any one of the twenty-seventh through the fifty-third embodiments.

In certain embodiments, the present disclosure provides a nucleotide or oligonucleotide represented by Formula III, III′, IIIP, or IIIP′ or a salt thereof, wherein R³⁶ is —CH₂CH₂CN. The remainder of the variables in Formula III, III′, IIIP, or IIIP′ are described in any one of the twenty-seventh through the fifty-third embodiments.

In certain embodiments, the present disclosure provides a nucleotide or oligonucleotide represented by Formula III, III′, IIIP, or IIIP′ or a salt thereof, wherein R³² is —OCH₂CH₂OMe. The remainder of the variables in Formula III, III′, IIIP, or IIIP′ are described in any one of the twenty-seventh through the fifty-third embodiments.

### 3. Process for Preparing Oligonucleotide Fragment

In a third aspect, the present disclosure describes a process of preparing an oligonucleotide fragment bearing a hydroxyl protecting group (e.g., a hydrophobic hydroxyl protecting group) at the 3′-end (when the fragment bears a hydrophobic hydroxyl protecting group, it can be referenced herein as the “3′-fragment”) or an amino protecting group at a nucleobase (when the nucleobase comprises a NH₂ group. It can be referenced herein as the “nucleobase SiLHPG fragment”). It is surprisingly discovered that the methods of the present disclosure for synthesizing the 3′-fragment or the nucleobase SiLHPG fragment can be used to prepare an oligonucleotide fragment having 3 to 20 (e.g., 3 to 10, 3 to 8, 3 to 5 or 4 to 5) nucleotides with high purity without chromatographic purification. In some embodiments, a hydrophobic 3′-hydroxyl protecting group is used, which facilitates the separation of the oligonucleotide fragment product by selective precipitation. In some embodiments, a hydrophobic amino protecting group is used, which facilitates the separation of the oligonucleotide fragment product by selective precipitation. In some embodiments, the liquid phase process comprises (1) 5′-OH deprotection step, (2) coupling step, and (3) oxidation or sulfurization step, wherein the steps (1), (2) and (3) are repeated until the desired number of nucleotides are linked together to form the 3′-oligonucleotide fragment.

In a fifty-fourth embodiment, the present disclosure provides a process for preparing an oligonucleotide fragment of formula (V),

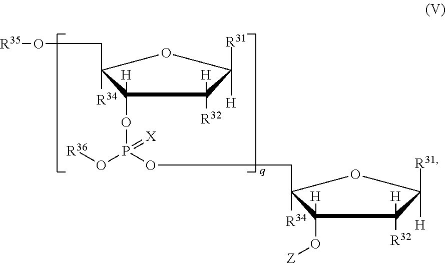

or a salt thereof, comprising the steps of:
1) deprotecting a compound of formula (VA):

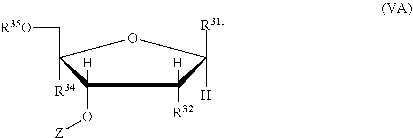

or a salt thereof, to form a compound of formula (VB):

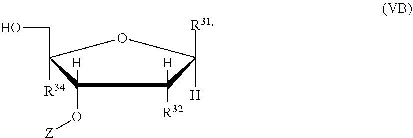

or a salt thereof;
2) reacting the compound of formula (VB), or a salt thereof, with a compound of formula (VC):

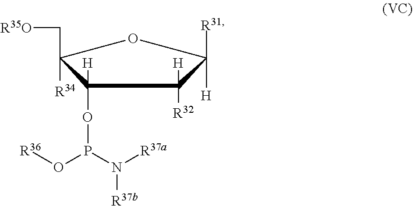

or a salt thereof, to form a compound of formula (VD),

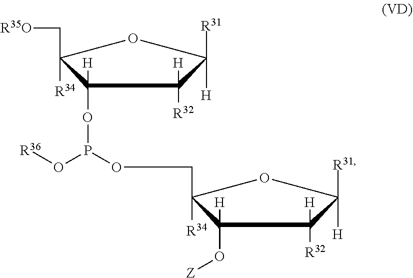

or a salt thereof;
3) sulfurizing or oxidizing the compound of formula (VD), or a salt thereof, with a sulfurization or oxidation agent to form a compound of formula (VE):

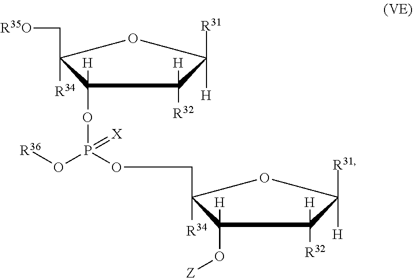

or a salt thereof;
4) deprotecting the compound of formula (VE), or a salt thereof to form a compound of formula (VF):

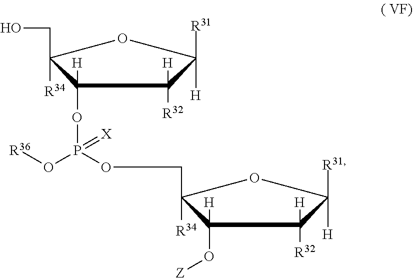

or a salt thereof;
5) when q is equal or greater than 2, starting with the compound of formula (VF), repeating steps 2), 3) and 4) for q-2 times, followed by steps 2) and 3) to yield the fragment of formula (V), or a salt thereof, wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³².
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is

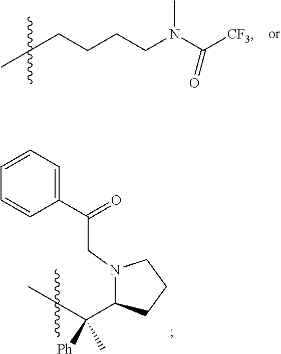 [SMILES: *CCCCN(C)C(=O)C(F)(F)F ; *[C@](C)(c1ccccc1)[C@@H]1CCCN1CC(=O)c1ccccc1]

R^{37a} and R^{37b} are independently C₁₋₆alkyl;
q is an integer from 1 to 20;
X, for each occurrence, is independently O or S;
Z is a group represented by Formula I* or B*,

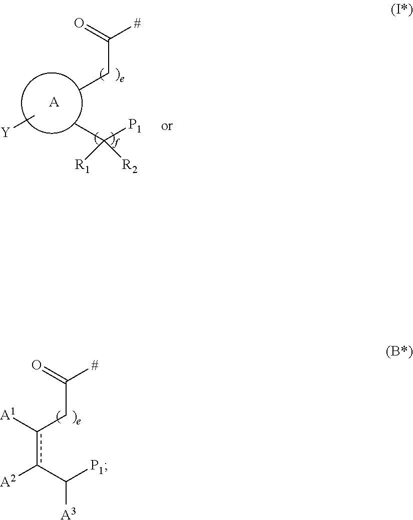

wherein
—#represents the point of attachment for Z;
one of A¹, A² and A³ is Y^{A} and the others are H;
[symbol] is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

In a fifty-fifth embodiment, the present disclosure provides a process for preparing an oligonucleotide fragment of formula (V′),

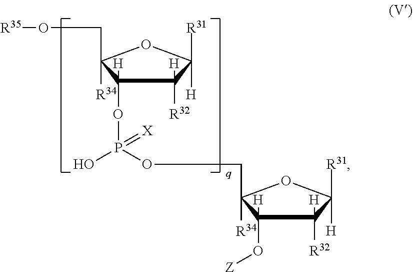

or a salt thereof, comprising the steps of:
1) deprotecting a compound of formula (VA):

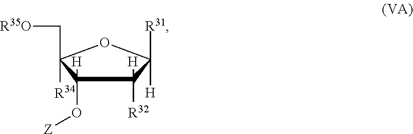

or a salt thereof, to form a compound of formula (VB):

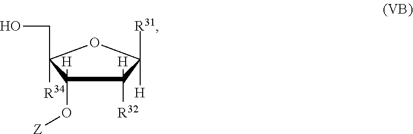

or a salt thereof;
2) reacting the compound of formula (VB), or a salt thereof, with a compound of formula (VC′):

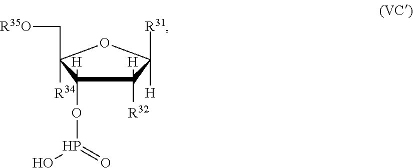

or a salt thereof, to form a compound of formula (VD′),

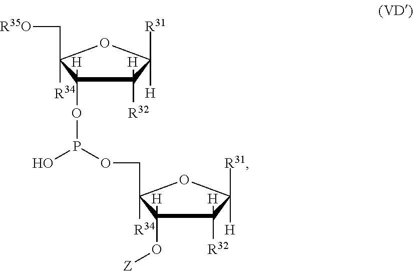

or a salt thereof;
3) sulfurizing or oxidizing the compound of formula (VD′), or a salt thereof, with a sulfurization or oxidation agent to form a compound of formula (VE′):

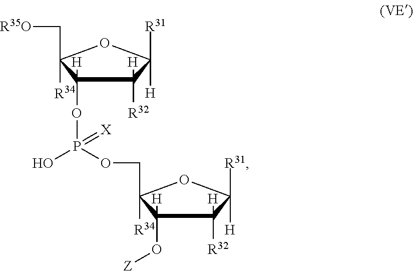

or a salt thereof;
4) deprotecting the compound of formula (VE′), or a salt thereof to form a compound of formula (VF′):

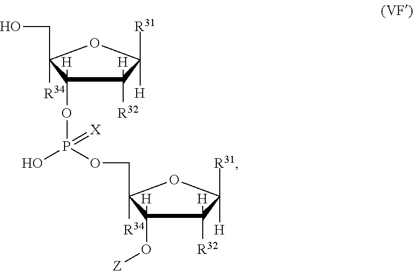

or a salt thereof;
5) when q is equal or greater than 2, starting with the compound of formula (VF′), repeating steps 2), 3) and 4) for q-2 times, followed by steps 2) and 3) to yield the fragment of formula (V′), or a salt thereof, wherein:
R³¹, R³², R³⁴, R³⁵, q, X and Z are as described above for formula (V) in the fifty-fourth embodiment.

In a fifty-sixth embodiment, the present disclosure provides a process for preparing an oligonucleotide fragment of formula (V-C1) or (V-C2),

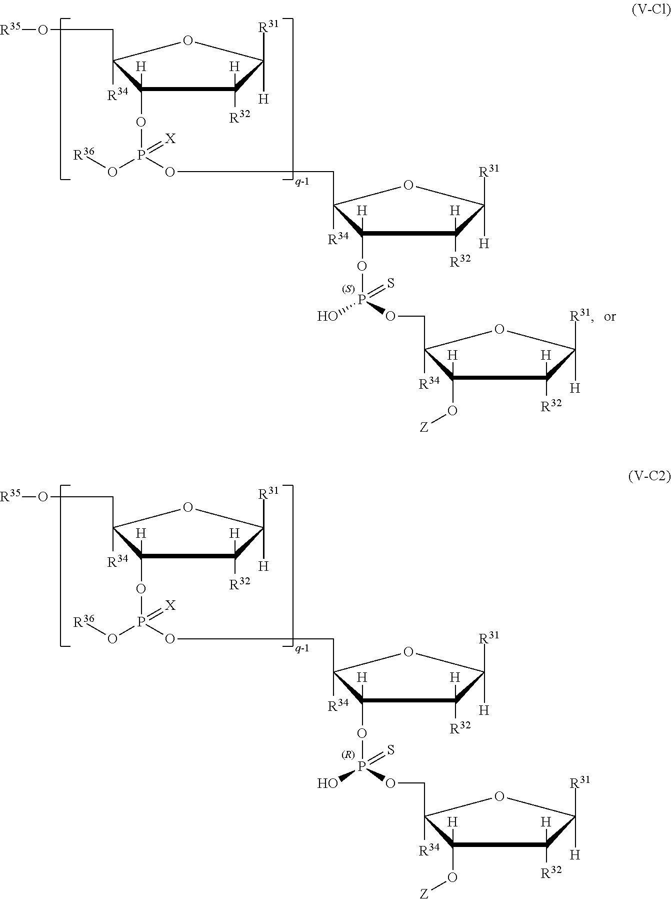

or a salt thereof, comprising the steps of:
1) reacting the compound of formula (VB),

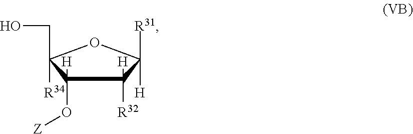

or a salt thereof, with a compound of formula (V-CR1) or (V-CR2),

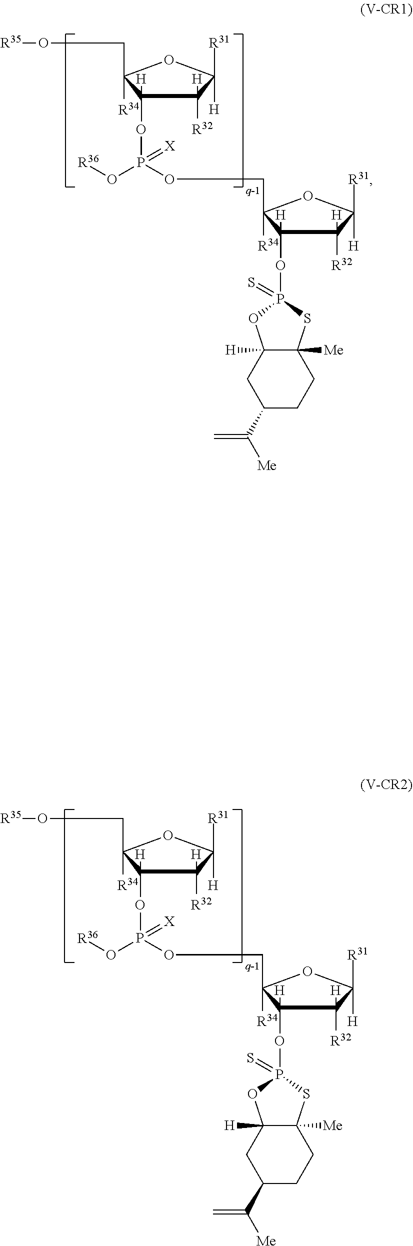

or a salt thereof, and a base, to form the compound of formula (V-C1) or (V-C2), wherein R³¹, R³², R³⁴, R³, R³⁶, q, X and Z are as described above for formula (V) in the fifty-fourth embodiment. The reaction of formula (VB) with (V-CR1) forms the compound of formula (V-C1) and the reaction of formula (VB) with (V-CR2) forms the compound of formula (V-C2).

In a fifty-seventh embodiment, the present disclosure provides a process for preparing an oligonucleotide fragment of formula (V-C1) or (V-C2),

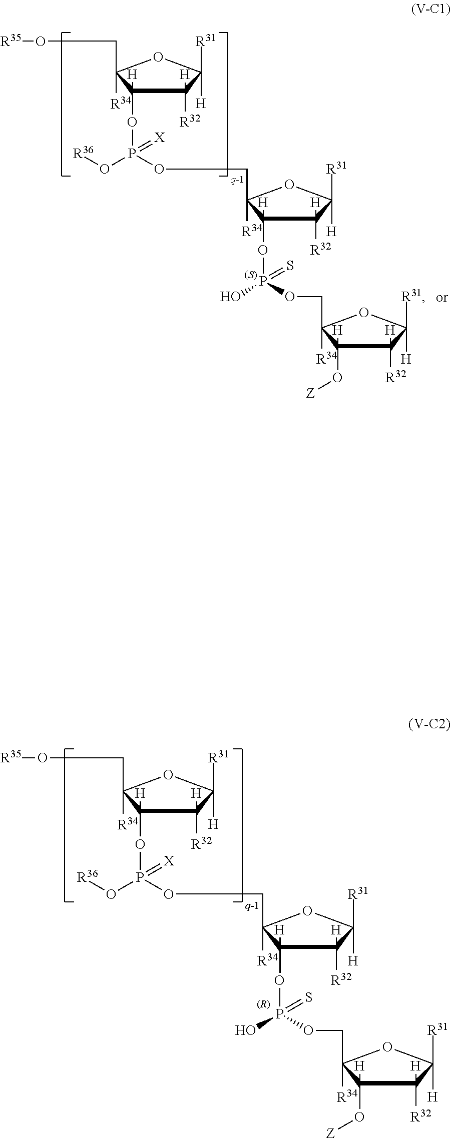

or a salt thereof, comprising the steps of:
1) reacting the compound of formula (VB),

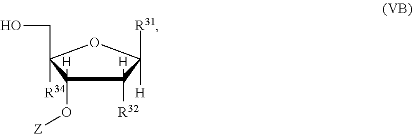

or a salt thereof, with a reagent of formula (VR1) or (VR2),

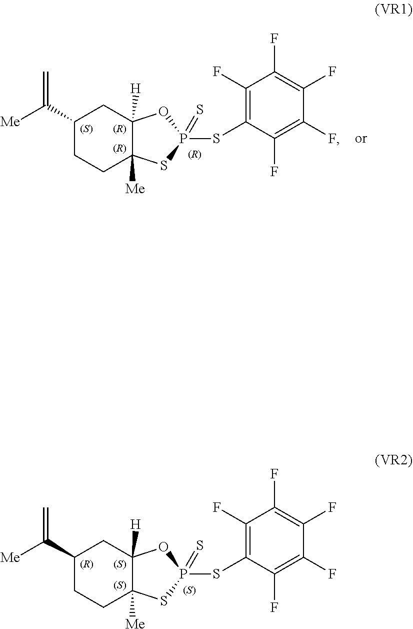

to form a compound of formula (V-CR3) or (V-CR4),

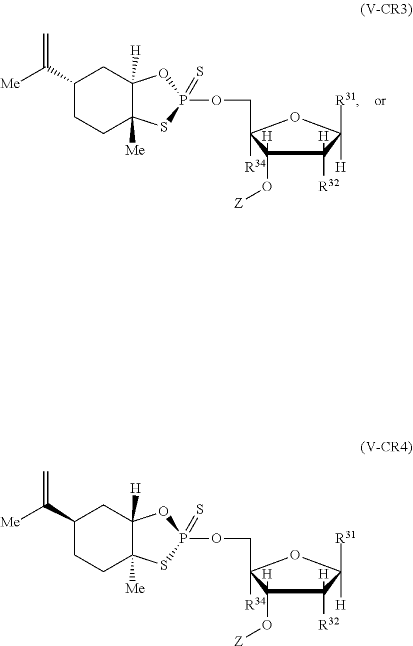

or a salt thereof;
2) reacting the compound of formula (V-CR3) or (V-CR4), or a salt thereof, with a compound of formula (VG):

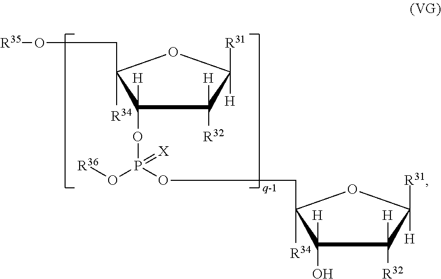

or a salt thereof, and a base, to form a compound of formula (V-C1) or (V-C2), wherein R³¹, R³², R³⁴, R³⁵, R³⁶, q, X and Z are as described above for formula (V) in the fifty-fourth embodiment. The reaction of reagent (VR1) with the compound of formula (VB) forms the compound of formula (V-CR3), which reacts with the compound of formula (VG) to form the compound of formula (V-C1). The reaction of reagent (VR2) with the compound of formula (VB) forms the compound of formula (V-CR4), which reacts with the compound of formula (VG) to form the compound of formula (V-C2).

In a fifty-eighth embodiment, the present disclosure provides a process for preparing an oligonucleotide fragment of formula (VBZ),

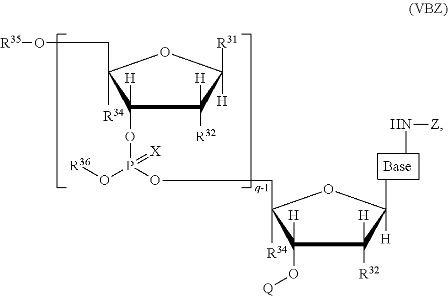

or a salt thereof, comprising the steps of:
1) reacting the compound of formula (VBZ-1),

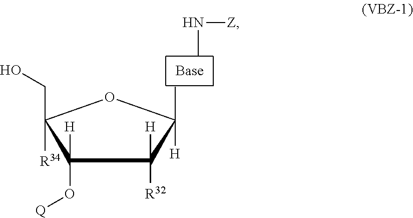

or a salt thereof, with a compound of formula (VBZ-2):

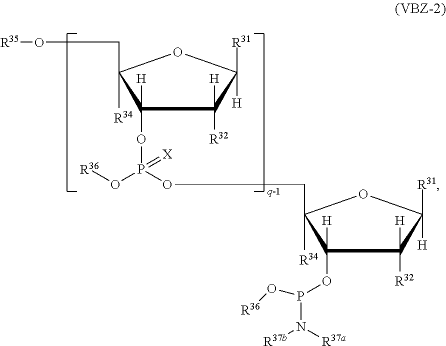

or a salt thereof, to form a compound of formula (VBZ-3),

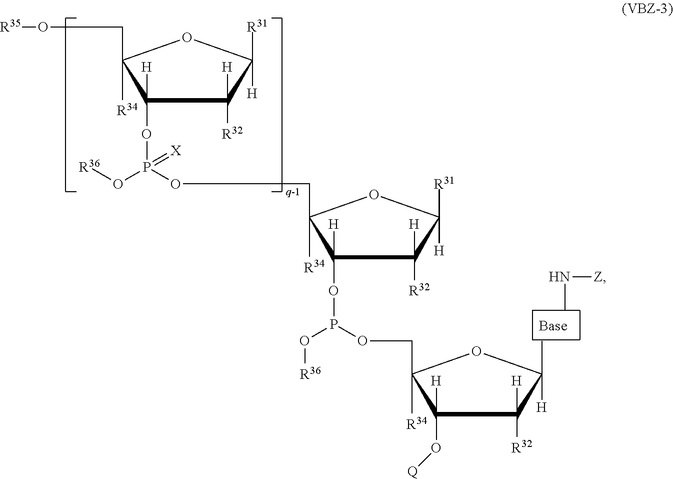

or a salt thereof;
2) sulfurizing or oxidizing the compound of formula (VBZ-3), or a salt thereof, with a sulfurization or oxidation agent to form a compound of formula (VBZ), or a salt thereof;
wherein:
Q is a hydroxyl protecting group; [symbol] is a nucleobase comprising a NH₂ group which is modified by Z; and R³¹, R³², R³⁴, R³⁵, R³⁶, R^{37a}, R^{37b}, q, X and Z are as described above for formula (V) in the fifty-fourth embodiment.

In a fifty-ninth embodiment, the present disclosure provides a process for preparing an oligonucleotide fragment of formula (VBZ) or a salt thereof described in the fifty-eighth embodiment, wherein the compound of formula VBZ-1 is prepared by
1) reacting the compound of formula (VBZ-4),

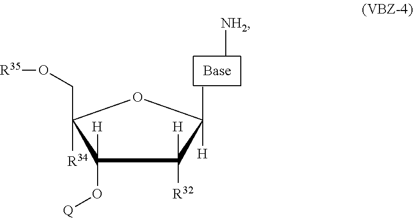

or a salt thereof, with Z—OH to form a compound of formula VBZ-5,

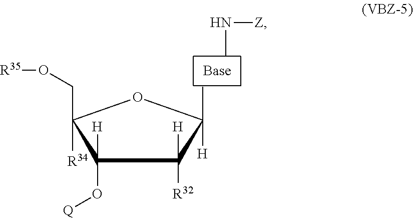

or a salt thereof;
2) deprotecting the compound of formula (VBZ-5) to form the compound of formula (VBZ-1).

In a sixty embodiment, the present disclosure provides a process for preparing an oligonucleotide fragment of formula (V), (V′), (V-C1), (V-C2), or (VBZ) or a salt thereof described in the fifty-fourth through fifty-ninth embodiment, wherein Y is a hydrophobic group comprising one or more aliphatic hydrocarbon group having 10 or more carbon atoms. The remainder of the variables in Formula (V), (V′), (V-C1), (V-C2), or (VBZ) are described in any one of the fifty-fourth through the fifty-ninth embodiments.

In a sixty-first embodiment, the present disclosure provides a process for preparing an oligonucleotide fragment of formula (V), (V′), (V-C1), (V-C2), or (VBZ) or a salt thereof described in the fifty-fourth through fifty-ninth embodiment, wherein no chromatography is used for purifying the reaction product of any one of steps 1), 2), 3) and 4).

In a sixty-second embodiment, the present disclosure provides a process for preparing an oligonucleotide fragment of formula (V), (V′), (V-C1), (V-C2), or (VBZ) or a salt thereof described in the fifty-fourth through fifty-ninth embodiment, wherein the reaction product of any one of steps 1), 2), 3) and 4) is purified by selective precipitation. In certain embodiments, the selective precipitation of the reaction product of any one of steps 1), 2), 3) and 4) or a salt thereof can be achieved by adding acetonitrile to a solution of the crude product in DCM. Alternatively, the solution of the crude product can be added to acetonitrile to precipitate out the desired product.

In certain embodiments, the reaction product of any one of steps 1), 2), 3) and 4) or a salt thereof is purified by extracting a solution comprising the reaction product of any one of steps 1), 2), 3) and 4) or a salt thereof in an organic solvent (MBTE, EtOAc, heptane/MBTE mixture, DCM, etc.) with an aqueous solution (e.g., NaHCO₃/H₂O or NaCl/H₂O) in addition to selective precipitation. In certain embodiments, the extraction is carried out before selective precipitation. Alternatively, the extraction is carried out after selective precipitation. In certain embodiments, the selective precipitation of the reaction product of any one of steps 1), 2), 3) and 4) or a salt thereof can be achieved by adding heptane or a heptane/MBTE mixture to a solution of the crude product in DCM or EtOAc. Alternatively, the solution of the crude product can be added to heptane or a heptane/MBTE mixture to precipitate out the desired product. A heptane/MBTE mixture with a suitale volume ratio (e.g., a volume ratio described herein) can be used.

In a sixty-third embodiment, the present disclosure provides a process for preparing an oligonucleotide fragment of formula (V)

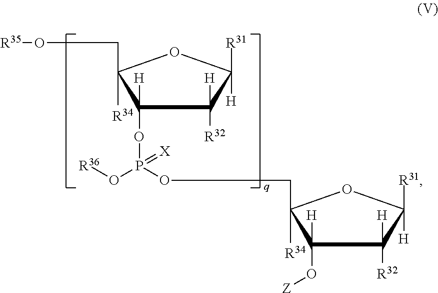

or a salt thereof, comprising the steps of:
a) coupling a nucleotide of formula (V-1):

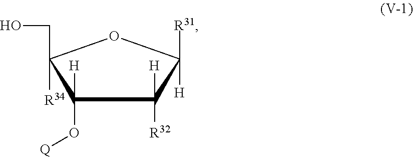

or a salt thereof, with an oligonucleotide fragment of formula (V-2):

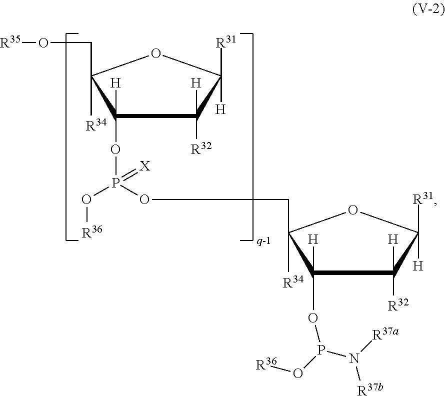

or a salt thereof, in a solution to form an oligonucleotide fragment of formula (V-3),

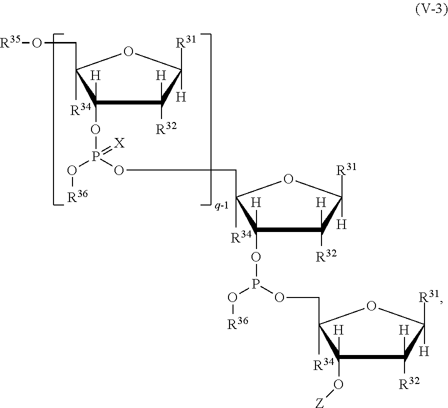

or a salt thereof; and
b) sulfurizing or oxidizing the oligonucleotide of formula (V-3), or a salt thereof, to form an oligonucleotide of formula (V):

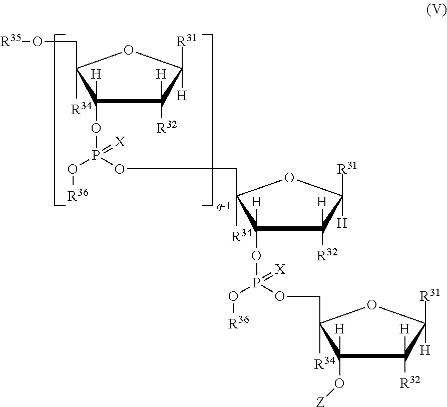

or a salt thereof;
wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³².
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or R³⁶ is

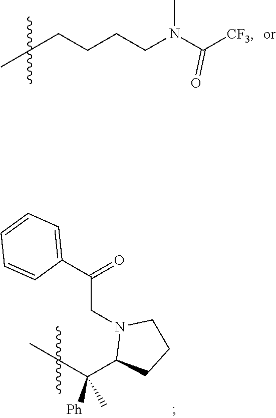 [SMILES: *CCCCN(C)C(=O)C(F)(F)F ; *[C@](C)(c1ccccc1)[C@@H]1CCCN1CC(=O)c1ccccc1]

R^{37a} and R^{37b} are independently C₁₋₆alkyl;
q is an integer from 1 to 20;
X, for each occurrence, is independently O or S;
Z is a group represented by Formula I* or B*,

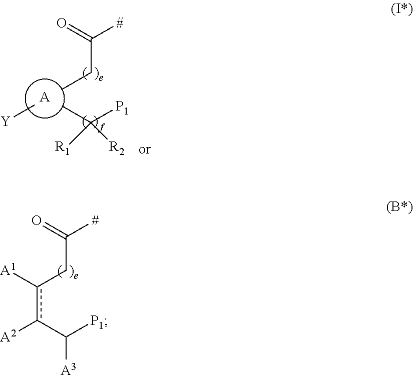

wherein
—#represents the point of attachment for Z;
one of A¹, A² and A³ is Y^{A} and the others are H; [symbol] is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} is independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, and sulfur; wherein R^{8A} and R^{9A}, for each occurance, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

In a sixty-fourth embodiment, the present disclosure provides a process for preparing an oligonucleotide fragment of formula (V*),

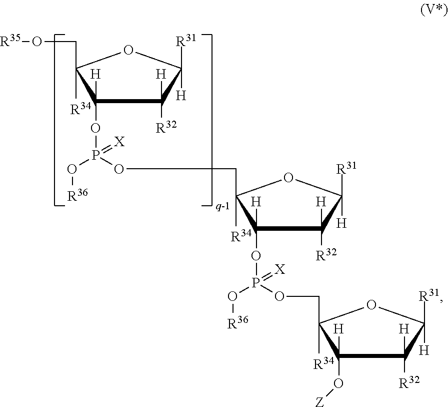

or a salt thereof, comprising the steps of:
a) coupling a nucleotide of formula (V-1):

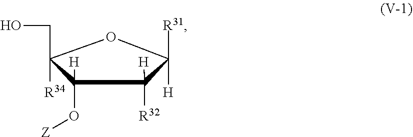

or a salt thereof, with an oligonucleotide fragment of formula (V-2′):

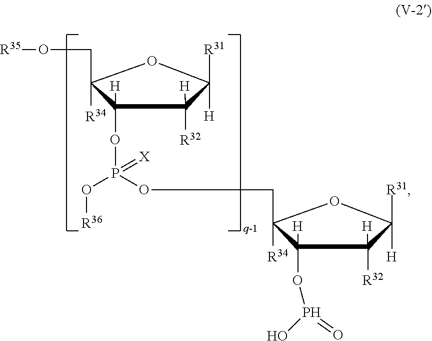

or a salt thereof, in a solution to form an oligonucleotide fragment of formula (V-3′),

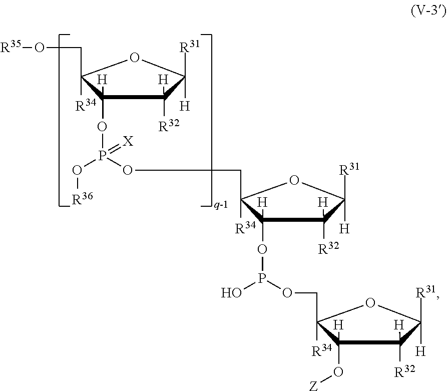

or a salt thereof; and
b) sulfurizing or oxidizing the oligonucleotide of formula (V-3′), or a salt thereof, to form the oligonucleotide of formula (V*) or a salt thereof; wherein R³¹, R³², R³⁴, R³⁵, R³⁶, R^{37a}, R^{37b}, q, X and Z are as described above for formula (V) in the sixty-third embodiment.

In a sixty-fifth embodiment, the present disclosure provides a process for preparing an oligonucleotide fragment of formula (V) or (V*) or a salt thereof described in the sixty-third or sixty-fourth embodiment, wherein Y is a hydrophobic group comprising one or more aliphatic hydrocarbon group having 10 or more carbon atoms.

In a sixty-sixth embodiment, the present disclosure provides a process for preparing an oligonucleotide fragment of formula (V) or a salt thereof described in the fifty-fourth or the sixty-third embodiments, further comprising deprotecting the fragment of formula (V) to form deprotected fragment of formula (VH):

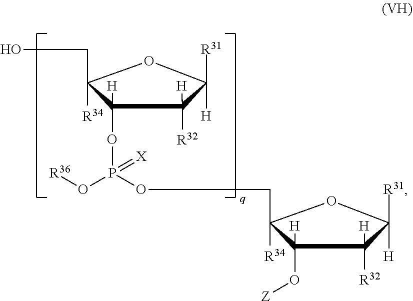

or a salt thereof.

In a sixty-seventh embodiment, the present disclosure provides a process for preparing an oligonucleotide fragment of formula (V′) or a salt thereof described in the fifty-fifth embodiment, further comprising deprotecting the fragment of formula (V′) to form deprotected fragment of formula (VH′):

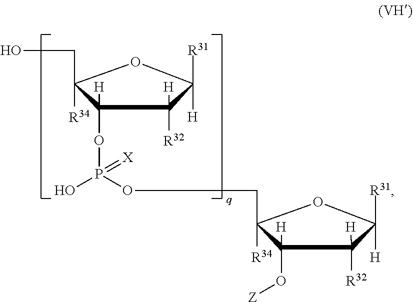

or a salt thereof.

In a sixty-eighth embodiment, the present disclosure provides a process for preparing an oligonucleotide fragment of formula (V-C1) or (V-C2), or a salt thereof described in the fifty-sixth or the fifty-seventh embodiment, further comprising deprotecting the fragment of formula (V-C1) or (V-C2) to form a deprotected fragment of formula (V-C3) or (V-C4):

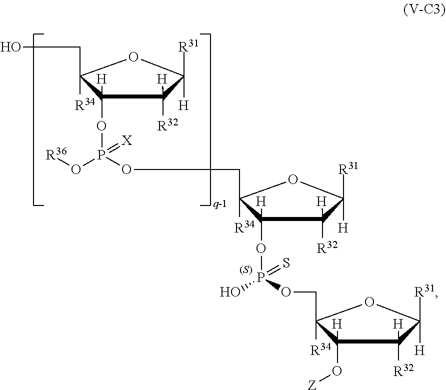

or a salt thereof, or

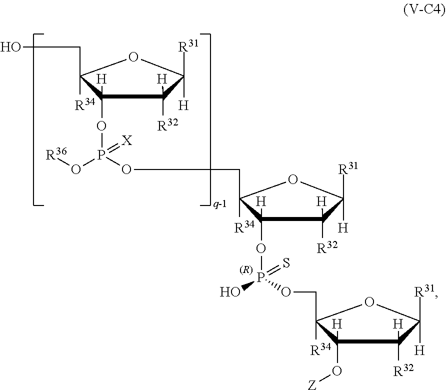

or a salt thereof.

In a sixty-ninth embodiment, the present disclosure provides a process for preparing an oligonucleotide fragment of formula (VBZ), or a salt thereof described in the fifty-eighth embodiment, further comprising deprotecting the fragment of formula (VBZ) to form deprotected fragment of formula (VBZ-6):

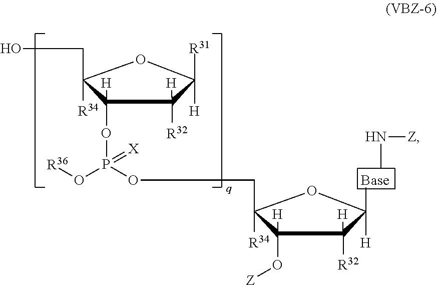

or a salt thereof.

In a seventieth embodiment, the present disclosure provides a process for preparing an oligonucleotide fragment of formula (V*), or a salt thereof described in the sixty-fourth embodiment, further comprising deprotecting the fragment of formula (V*) to form a deprotected fragment of formula (V*-1):

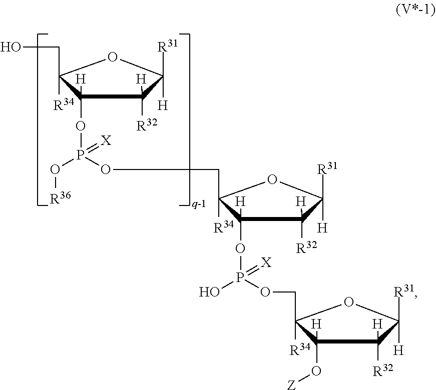

or a salt thereof.

In a seventy-first embodiment, the present disclosure provides a process for preparing an oligonucleotide fragment of formula (V), (V′), (V-C1), (V-C2), (VBZ), or (V*), or a salt thereof described in the fifty-fourth through sixty-fourth embodiment, further comprising desilylation of the fragment of formula (V), (V′), (V-C1), (V-C2), (VBZ), or (V*) to form the fragment of formula (VJ), (VJ′), (V-C5), (V-C6), (VBZ-7), or (V*-2), respectively:

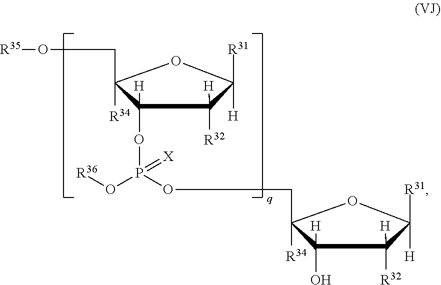

or a salt thereof,

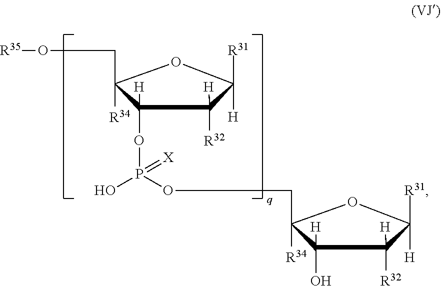

or a salt thereof,

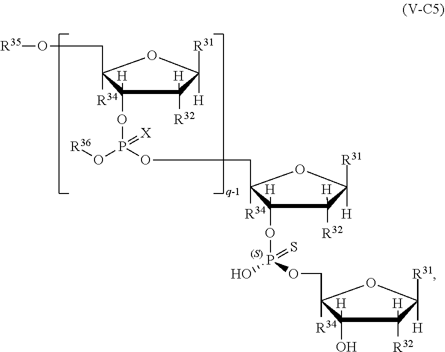

or a salt thereof,

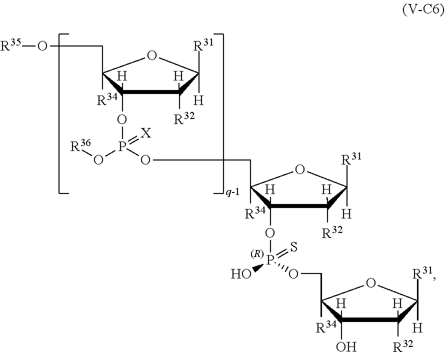

or a salt thereof,

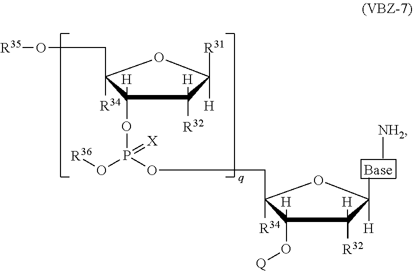

or a salt thereof, or

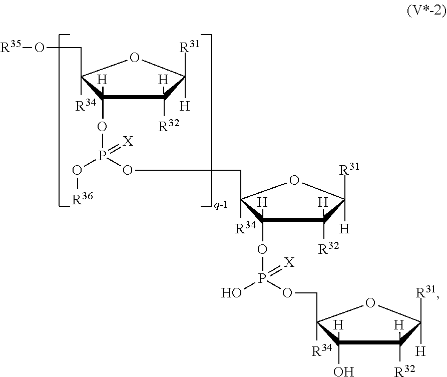

or a salt thereof. In one embodiment, when Q and P₁ in formula (VBZ) are the same, the desilylation reaction forms a compound of formula (VBZ-7′):

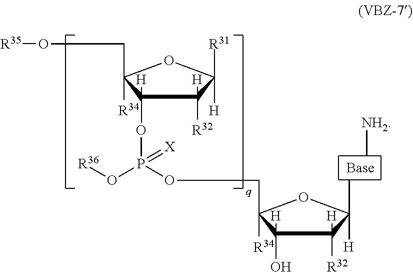

In a seventy-second embodiment, the present disclosure provides a process for preparing the fragment of formula (VJ), (VJ′), (V-C5), (V-C6), (VBZ-7), or (V*-2) described in any one of the seventy-first embodiment, wherein the desilylation reaction is carried out by reacting the compound of formula (V), (V′), (V-C1), (V-C2), (VBZ), or (V*) with HF in the presence of a base.

In a seventy-third embodiment, the present disclosure provides a process described in the seventy-second embodiment, wherein the base is imidazole or pyridine, wherein the imidazole or pyridine are optionally substituted. In certain embodiments, the pyridine and/or imidazole is each independently substituted with one to three substituents selected from halogen, C₁₋₆alkyl, C₁₋₆alkoxy, —OH, and C₁₋₆haloalkyl.

In a seventy-fourth embodiment, the present disclosure provides a process described in the seventy-third embodiment, wherein the desilylation reaction is carried out by reacting the compound of formula (V), (V′), (V-C1), (V-C2), (VBZ), or (V*) with HF in the presence of pyridine and imidazole.

In a seventy-fifth embodiment, the present disclosure provides a process described in the seventy-fourth embodiment, wherein the molar ratio of imidazole to HF is in the range of 0.5:1 to 10:1.

In a seventy-sixth embodiment, the present disclosure provides a process described in the seventy-fifth embodiment, wherein the molar ratio of imidazole to HF is in the range of 1.1:1 to 5:1.

In a seventy-seventh embodiment, the present disclosure provides a process described in the seventy-sixth embodiment, wherein the molar ratio of imidazole to HF is in the range of 2:1.

In a seventy-eighth embodiment, the present disclosure provides a process described in the seventy-fourth through seventy-seventh embodiments, wherein the molar ratio of pyridine to HF is in the range of 100:1 to 1:1.

In a seventy-ninth embodiment, the present disclosure provides a process described in the seventy-fourth through seventy-seventh embodiments, wherein the molar ratio of pyridine to HF is in the range of 1:1.

In an eightieth embodiment, the present disclosure provides a process described in any one of the fifty-fourth through seventy-first embodiments, wherein the fragment for formula (V), (V′), (V-C1), (V-C2), (VBZ), (V*), (VH), (VH′), (V-C3), (V-C4), (VBZ-6), (V*-1), (VJ), (VJ′), (V-C5), (V-C6), (VBZ-7), (VBZ-7′) or (V*-2) is not purified by chromatography.

In an eighty-first embodiment, the present disclosure provides a process described in the eightieth embodiments, wherein the fragment for formula (V), (V′), (V-C1), (V-C2), (VBZ), (V*), (VH), (VH′), (V-C3), (V-C4), (VBZ-6), (V*-1), (VJ), (VJ′), (V-C5), (V-C6), (VBZ-7), (VBZ-7′) or (V*-2) is purified by selective precipitation and/or extraction.

In an eighty-second embodiment, the present disclosure provides a process described in any one of the fifty-fourth through eighty-first embodiments, wherein q is 2 to 5.

In an eighty-third embodiment, the present disclosure provides a process described in any one the eighty-second embodiment, wherein q is 4.

In certain embodiments, for the process described in the third aspect or any embodiments describe therein (e.g., the sixty-fifth to seventy-fifth embodiments), the variables R³, R³², R³⁴, R³⁵, R³⁶, q, and/or Z are described in the second aspect or any one of embodiments described therein (e.g., the twenty-seventh to thirty-third embodiments).

In certain embodiments, for the process described in the third aspect or any one of embodiments describe therein (e.g., the fifty-fourth to eighty-third embodiments), the 5′-OH deprotection step is a detritylation method for removing a 5′-trityl group. It is discovered that when the detritylation reaction is carried out under anhydrous or substantially anhydrous conditions, significant reduction of side reactions (e.g., deamination of nucleobase cytosine or 5-methylcytosine or their derivatives commonly used for oligonucleotide synthesis) can be achieved. The present detritylation method also involves the addition of a cation scavenger to facilitate the completion of the reaction. As a result, the product with high purity can be obtained without the need of chromatography (e.g., column chromatography). Water level of the detritylation reaction can be controlled by the use of drying agent (e.g., molecular sieves), azeoptropic distillation or other suitable methods known in the art. Alternatively, solvents, acids, and other reagents used in the detritylation reaction, substrates to be subjected to detritylation reaction, and the reaction vessels can be dried to meet the residual water levels prior to use for the detritylation reaction.

In certain embodiments, for the process described in the third aspect or any one of embodiments describe therein (e.g., the fifty-fourth to eighty-third embodiments), R³⁶ is one of the following:

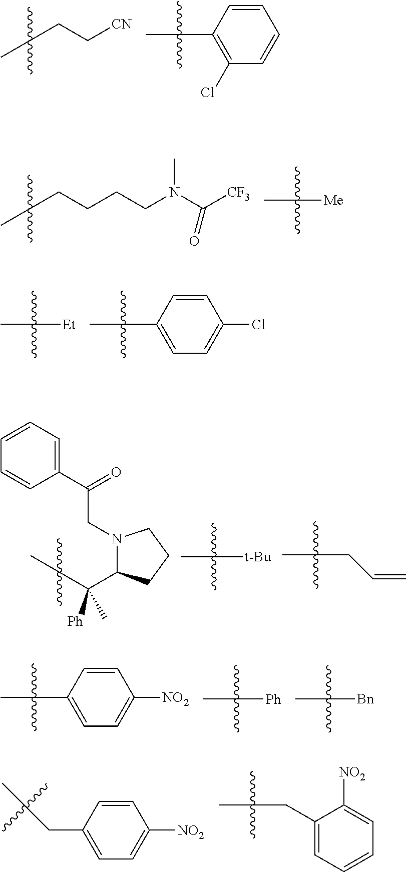

See Nat Biotechnol. 2017 September; 35(9):845-851; J. Org. Chem. 1999, 64, 7515-7522; Biopolymers (Peptide Science), 2001, 60, 3, each of which is incorporated herein by reference.

In certain embodiments, for the process described in the third aspect or any one of embodiments describe therein (e.g., the fifty-fourth to eighty-third embodiments), the 5′-OH deprotection (or detritylation) reaction is carried out in the presence of a drying agent. Any suitable drying agents can be used in the deprotection reaction. In some embodiments, the drying agent is selected from calcium chloride, potassium chloride, sodium sulfate, calcium sulfate, magnesium sulfate and molecular sieves.

In certain embodiments, for the process described in the third aspect or any one of embodiments describe therein (e.g., the fifty-fourth to eighty-third embodiments), the drying agent is molecular sieves.

In certain embodiments, for the process described in the third aspect or any one of embodiments describe therein (e.g., the fifty-fourth to eighty-third embodiments), the size of molecular sieves is 3 Å or 4 Å. In one embodiment, the size of molecular sieves is 3 Å.

In certain embodiments, for the process described in the third aspect or any one of embodiments describe therein (e.g., the fifty-fourth to eighty-third embodiments), the anhydrous or substantially anhydrous solution for the deprotection reaction is obtained by removing water using azeotropic distillation prior to the deprotection reaction.

Alternatively, solvents, acids or acid solutions, and other reagents or solutions comprising the reagents to be used in the detritylation reaction, substrates or substrate solutions to be subjected to detritylation reaction, and the reaction vessels can be dried individually or combined prior to the detritylation reaction.

In certain embodiments, for the process described in the third aspect or any one of embodiments describe therein (e.g., the fifty-fourth to eighty-third embodiments), the deprotection reaction is carried out in the presence of a scavenger selected from a cation scavenger comprising a —SH group, a silane scanveger (such as HSiPh₃, HSiBu₃, triisopropylsilane etc.), siloxane, polystyrene, furan, pyrrole and indole.

In certain embodiments, the deprotection reaction is carried out in the presence of a scavenger selected from 1-dodecanethiol, cyclohexanethiol, 1-octanethiol, triisopropylsilane, indole, 2,3-dimethylfuran, diphenylsilane, 2-mercaptoimidazole, diphenylmethylsilane, phenylsilane, 5-methoxyindole, methylphenylsilane, chlorodimethylsilane, 1,1,3,3-tetramethyldisiloxane, 1-thioglycerol, triphenylsilane, tert-butyldimethylsilane, butylsilane, methyldiethoxysilane, 1,1,3,3,5,5-hexamethyltrisiloxane, hexylsilane, (mercaptomethyl)polystyrene, or dimethylphenylsilane.

In certain embodiments, the cation scavenger is a compound of formula RSH, wherein R is an alkyl, a cycloalkyl, a heterocycloalkyl, an aryl or a heteroaryl group, each of which is optionally substituted.

In certain embodiments, the cation scavenger is CH₃(CH₂)₅SH, CH₃(CH₂)₁₁SH, cyclohexanethiol (CySH), or CH₃CH₂OC(═O)CH₂CH₂SH.

In certain embodiments, for the process described in the third aspect or any one of embodiments describe therein (e.g., the fifty-fourth to eighty-third embodiments), R³⁵ is a 4,4′-dimethoxytrityl (DMT) group.

In certain embodiments, for the process described in the third aspect or any one of embodiments describe therein (e.g., the fifty-fourth to eighty-third embodiments), the deprotection reaction is carried out by reacting the compound of formula (VA) with a detritylation reagent. Any suitable detritylation reagent can be used.

In certain embodiments, the detritylation reagent is a strong organic acid.

In certain embodiments, the detritylation reagent is selected from CF₃COOH, CCl₃COOH, CHCl₂COOH, CH₂ClCOOH, H₃PO₄, methanesulfonic acid (MSA), benzenesulfonic acid (BSA), CClF₂COOH, CHF₂COOH, PhSO₂H (phenylsulfinic acid) etc. In a preferred embodiment, the detritylation reagent is CH₂ClCOOH. In another specific embodiment, the detritylation reagent is CF₃COOH. In yet another specific embodiment, the detritylation reagent is CHCl₂COOH.

In certain embodiments, the detritylation reagent is citric acid. In certain embodiments, the detritylation reagent is saturated citric acid solution.

In certain embodiments, for the process described in the third aspect or any one of embodiments describe therein (e.g., the fifty-fourth to eighty-third embodiments), the coupling reaction of step 2) can be carried out in the presence of an activator described herein (e.g. activators described in the thirty-ninth embodiment). In certain embodiments, the activator is 4,5-dicyanoimidazole (DCI) or 5-ethylthio-1H-tetrazole (ETT).

In certain embodiments, for the process described in the third aspect or any one of embodiments describe therein (e.g., the fifty-fourth to eighty-third embodiments), the sulfurization reaction of step 3) is carried out using a sulfurizing agent, such as 3-amino-1,2,4-dithiazole-5-thione (xanthane hydride or ADTT), 3-(N,N-dimethylamino-methylidene)amino)-3H-1,2,4-dithiazole (DDTT), phenylacetyl disulfide (PADS), 3H-1,2-benzodithiol-3-one 1,1-dioxide (Beaucage Reagent), or phenyl-3H-1,2,4-dithiazol-3-one (POS). In a specific embodimet, the sulfurizing agent is DDTT. In a specific embodiment, the sulfurizing agent is xanthane hydride. In certain embodiments, the sulfurization reaction is carried out in the presence of a base as described herein. In certain embodiments, the base is pyridine or imidazole. In certain embodiments, the sulfurization reaction of step 3) is carried out in the presence of DDTT and 4,5-dicyanoimidazole (DCI).

In certain embodiments, for the process described in the third aspect or any one of embodiments describe therein (e.g., the fifty-fourth to eighty-third embodiments), the oxidation reaction of step 3) is carried out by using standard oxidizing agents known in the literature. Exemplary oxidizing agent include, but are not limited to, tert-butylhydroperoxide (t-BuOOH), (1S)-(+)-(10-camphorsulfonyl)oxaziridine (CSO), (1R)-(−)-(10-camphorsulfonyl)oxaziridine (enantiomer of CSO), I₂, and iodine-pyridine-water oxidizer solution. In a specific embodimet, the oxidizing agent is t-BuOOH.

In certain embodiments, for the process described in the third aspect or any one of embodiments describe therein (e.g., the fifty-fourth to eighty-third embodiments), the coupling/oxidation/detritylation steps are carried out in a one pot reaction. In certain embodiments, the oxidation reagents in the one pot reaction is BPO or tBuOOH:

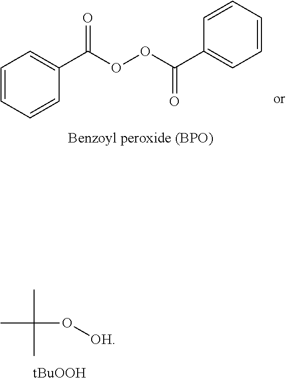

### 4. Process to Prepare Target Oligonucleotides

In a fourth aspect, the present disclosure describes a process for preparing target oligonucleotides, wherein the target oligonucleotide is assembled in the direction of the 3′-terminal to the 5′-terminal (3′-5′ direction). It has been demonstrated that the process of the present disclosure is successfully used to synthesize target oligonucleotides in large quantities. In addition, high purity protected target oligonucleotide can be obtained by the methods of the present disclosure without chromatographic purification.

In certain embodiments, the process described herein involves step by step addition of oligonucleotide fragments in liquid (solution) phase to synthesize the target oligonucleotide. For example, 5-mer and 4-mer fragments are coupled first to synthesize a 9-mer fragment which is further reacted with another 5-mer fragment to synthesize 14-mer oligonucleotide. The 14-mer oligonucleotide can be further coupled with another fragment until the desired length of the target oligonucleotide is obtained. In certain embodiments, a 5-mer fragment having a 3′-hydrophobic hydroxyl protecting group (3′-LHPG) (3′-end fragment) or an amino protecting group at a nucleobase (when the nucleobase comprises a NH₂ group. It can be referenced herein as the “nucleobase LHPG fragment”) is first coupled with 5-mer fragment to form a 10-mer fragment having 3′-LHPG group or nucleobase LHPG group, which is then further reacted with a 4-mer fragment to form a 14-mer fragment, which is in turn coupled with another 4-mer fragment to form the target 18-mer oligonucleotide. In certain embodiments, the 3′-end fragment having n nucleotides (e.g. 5-mer fragment) is synthesized by coupling a single nucleotide having the 3′-LHPG group with a fragment having n−1 nucleotides (e.g., 4-mer fragment). In certain embodiments, the nucleobase LHPG fragment having n nucleotides (e.g. 5-mer fragment) is synthesized by coupling a single nucleotide having the LHPG group at the nucleobase with a fragment having n−1 nucleotides (e.g., 4-mer fragment).

In a eighty-fourth embodiment, the present disclosure provides a process for preparing an oligonucleotide of formula (VI) or (VI-1),

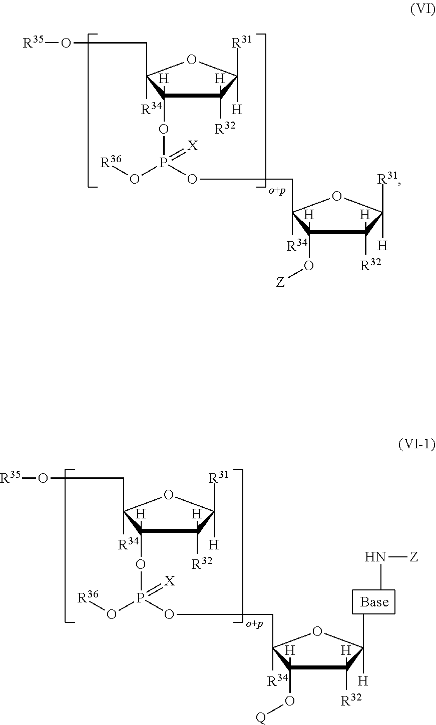

or a salt thereof, comprising
a) coupling an oligonucleotide fragment of formula (F1) or (F1-1):

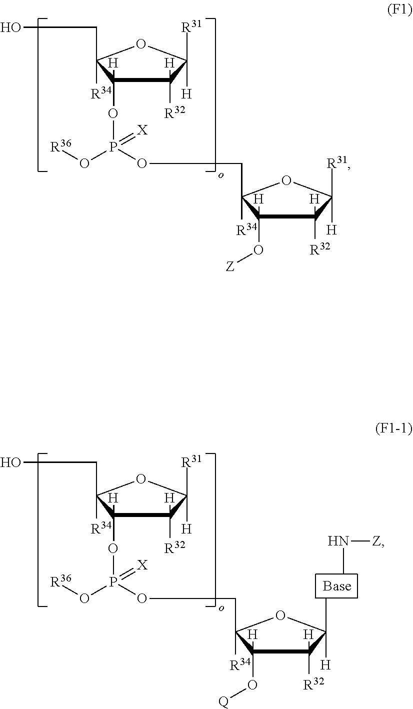

or a salt thereof,
with an oligonucleotide fragment of formula (F2):

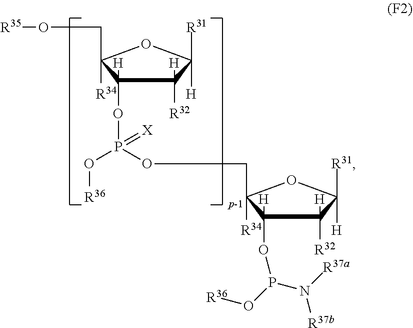

or a salt thereof, in a solution to form an oligonucleotide fragment of formula (F3) or (F3-1),

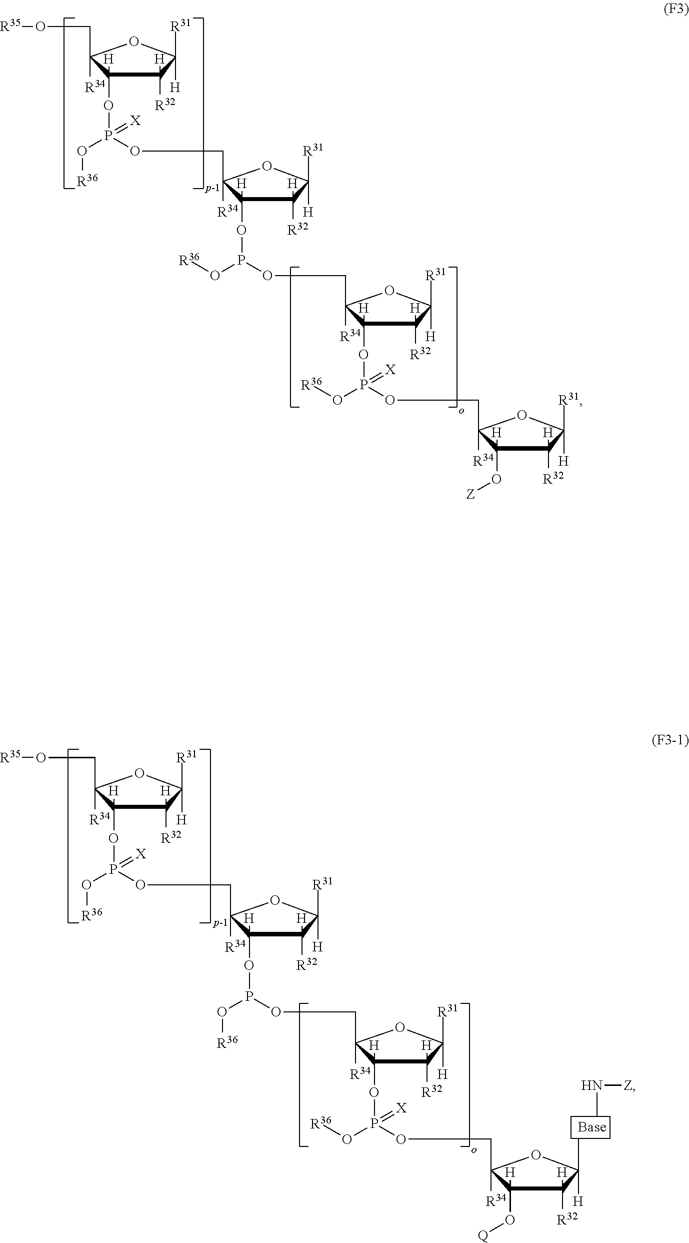

or a salt thereof; and
b) sulfurizing or oxidizing the oligonucleotide fragment of formula (F3) or (F3-1), or a salt thereof, to form the oligonucleotide of formula (VI) or (VI-1) or a salt thereof,
   wherein:
Q is a hydroxyl protecting group;
[symbol] is a nucleobase comprising a NH₂ group which is modified by Z;
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³².
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is

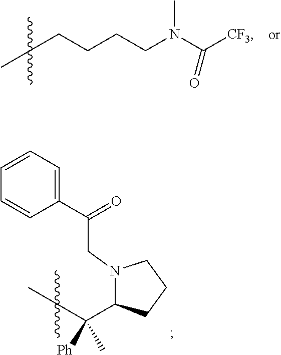 [SMILES: *CCCCN(C)C(=O)C(F)(F)F ; *[C@](C)(c1ccccc1)[C@@H]1CCCN1CC(=O)c1ccccc1]

R^{37a} and R^{37b} are independently C₁₋₆alkyl;
p is an integer from 2 to 20;
is an integer from 1 to 200;
X, for each occurrence, is independently O or S;
Z is a group represented by Formula I* or B*,

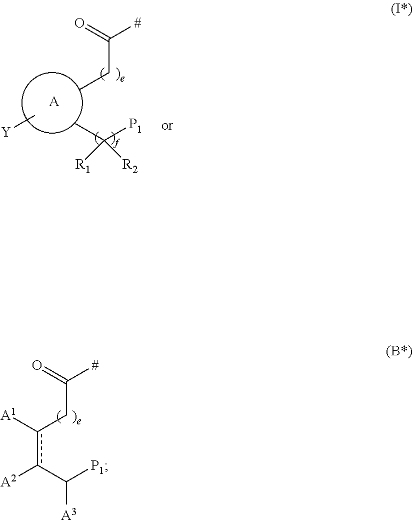

wherein
—#represents the point of attachment for Z;
one of A¹, A² and A³ is Y^{A} and the others are H;
[symbol] is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

In a eighty-fifth embodiment, the present disclosure provides a process for preparing an oligonucleotide of formula (VI′) or (VI′-1),

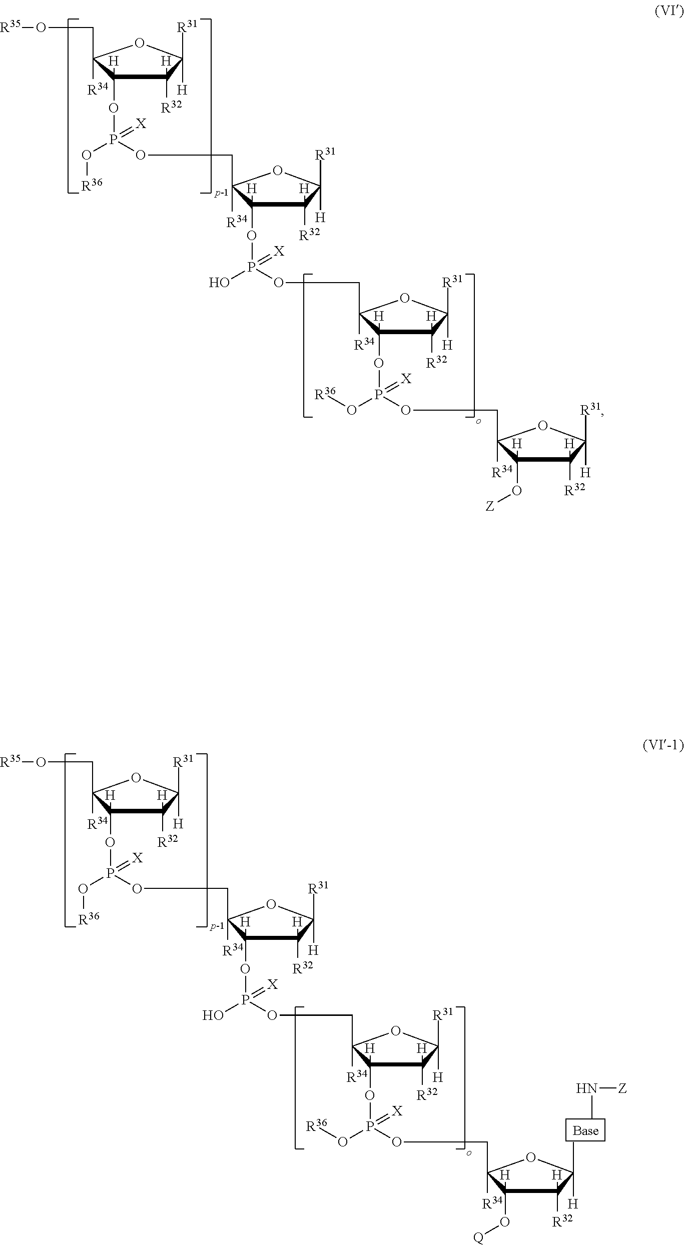

or a salt thereof, comprising
a) coupling an oligonucleotide fragment of formula (F1) or (F1-1):

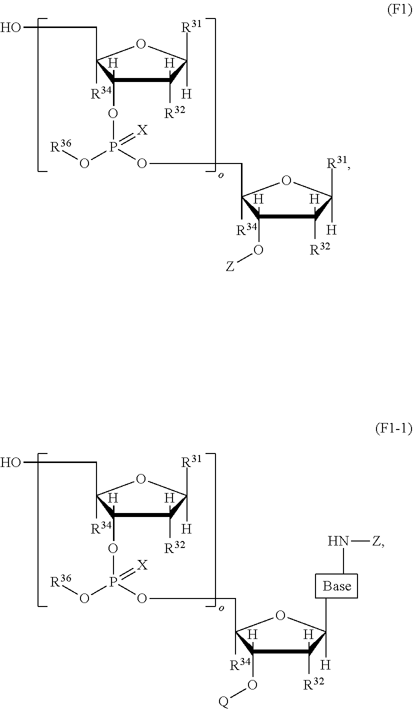

or a salt thereof, with an oligonucleotide fragment of formula (F2′):

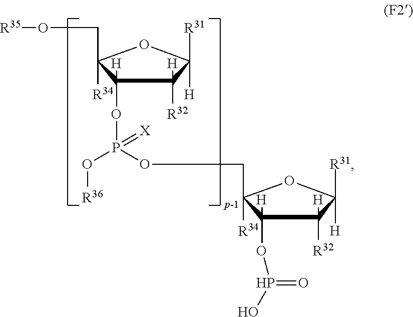

or a salt thereof, in a solution to form an oligonucleotide fragment of formula (F3′) or (F3′-1),

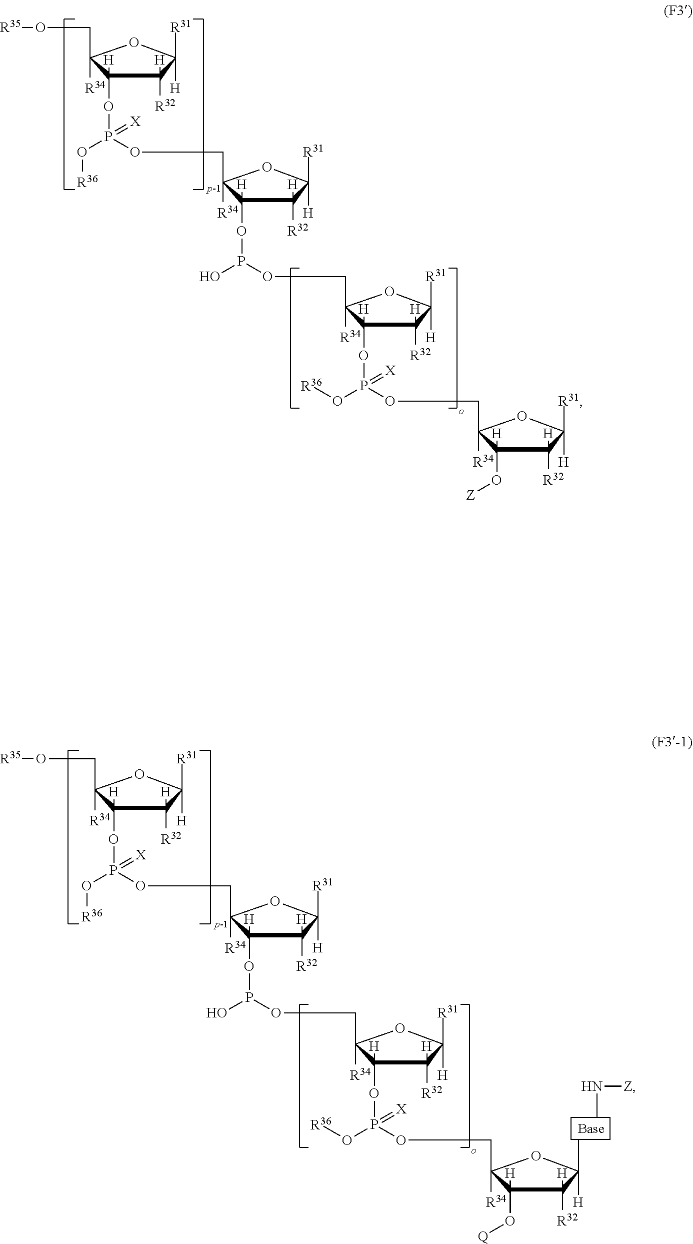

or a salt thereof; and
b) sulfurizing or oxidizing the oligonucleotide fragment of formula (F3′) or (F3′-1), or a salt thereof, to form the oligonucleotide of formula (VI′) or (VI′-1) or a salt thereof, wherein Q, [symbol] , R³¹, R³², R³⁴, R³⁵, R³⁶, R^{37a} and R^{37b}, p, o, X and Z are as described above for Formula (VI) or (VI-1) in the eighty-fourth embodiment.

In a eighty-sixth embodiment, the present disclosure provides a process for preparing an oligonucleotide of formula (VI), (VI′), (VI-1), or (VI′-1) described in the eighty-fourth or eighty-fifth embodiment, wherein Y is a hydrophobic group comprising one or more aliphatic hydrocarbon group having 10 or more carbon atoms.

In a eighty-seventh embodiment, the present disclosure provides a process for preparing an oligonucleotide of formula (VI), (VI′), (VI-1), or (VI′-1) described in the eighty-fourth or eighty-fifth embodiment, further comprising step c) deprotecting the oligonucleotide of formula (VI), (VI′), (VI-1), or (VI′-1) to form an oligonucleotide of formula (VII), (VII-1), (VII′), or (VII′-1)

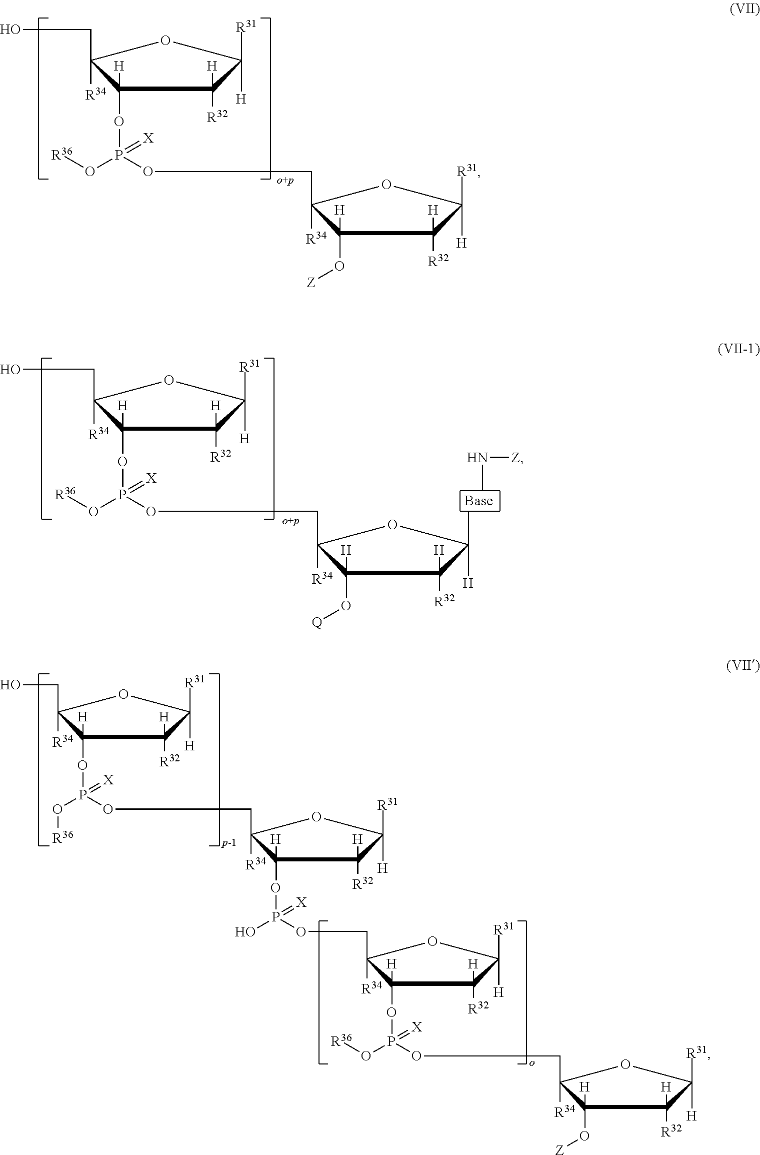

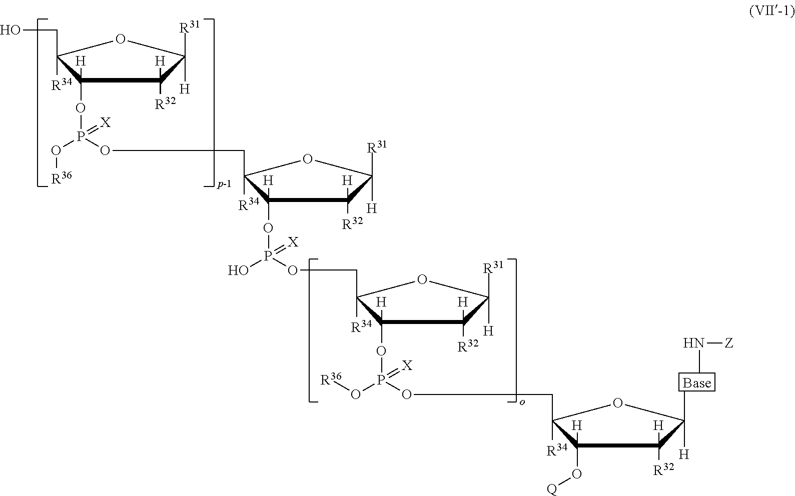

or a salt thereof.

In an eighty-eighth embodiment, the present disclosure provides a process for preparing an oligonucleotide of formula (VII), (VII-1), (VII′), or (VII′-1) described in the eighty-seventh embodiment, wherein starting from oligonucleotide of formula (VII), (VII-1), (VII′), or (VII′-1), the process further comprises repeating steps a), b) and c) for 1 to 10 times, followed by steps a) and b) to form the target oligonucleotide with desired length.

In an eighty-ninth embodiment, the present disclosure provides a process described in the eighty-eighth embodiment, wherein the process further comprises repeating steps a), b) and c) for 1 to 3 times followed by steps a) and b) to form the target oligonucleotide with desired length.

In a ninetieth embodiment, the present disclosure provides a process described in the eighty-fourth through eighty-ninth embodiments, wherein o is an integer from 2 to 20.

In a ninety-first embodiment, the present disclosure provides a process described in the ninetieth embodiment, wherein o is an integer from 2 to 5.

In a ninety-second embodiment, the present disclosure provides a process described in the ninety-first embodiment, wherein o is 4.

In a ninety-third embodiment, the present disclosure provides a process described in the third or fourth aspect (e.g., any one of the fifty-fourth through ninety-second embodiments), wherein Z is a group represented by Formula I*,

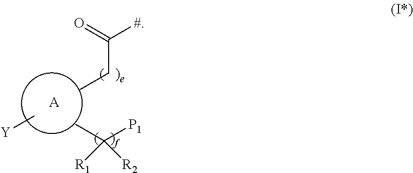

In a ninety-fourth embodiment, the present disclosure provides a process described in the third or fourth aspect (e.g., any one of the fifty-fourth through ninety-second embodiments), wherein Z is a group represented by Formula B*,

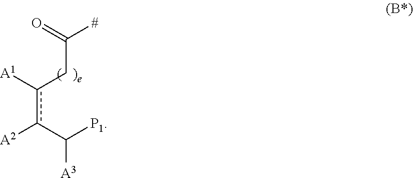

In a ninety-fifth embodiment, the present disclosure provides a process described in the third or fourth aspect (e.g., any one of the fifty-fourth through ninety-second embodiments), wherein Z is a group represented by Formula B-1* or B-2*:

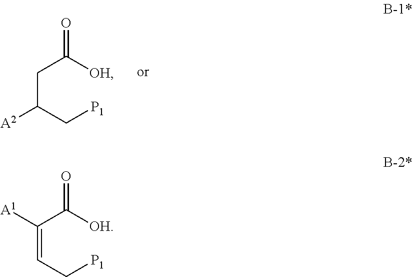

In a ninety-sixth embodiment, the present disclosure provides a process described in the third or fourth aspect (e.g., any one of the fifty-fourth through ninety-second embodiments), wherein ring A is phenyl or naphthalenyl.

In a ninety-seventh embodiment, the present disclosure provides a process described in the third or fourth aspect (e.g., any one of the fifty-fourth through ninety-six embodiments), wherein P₁ is a silyl hydroxyl protecting group selected from the following:

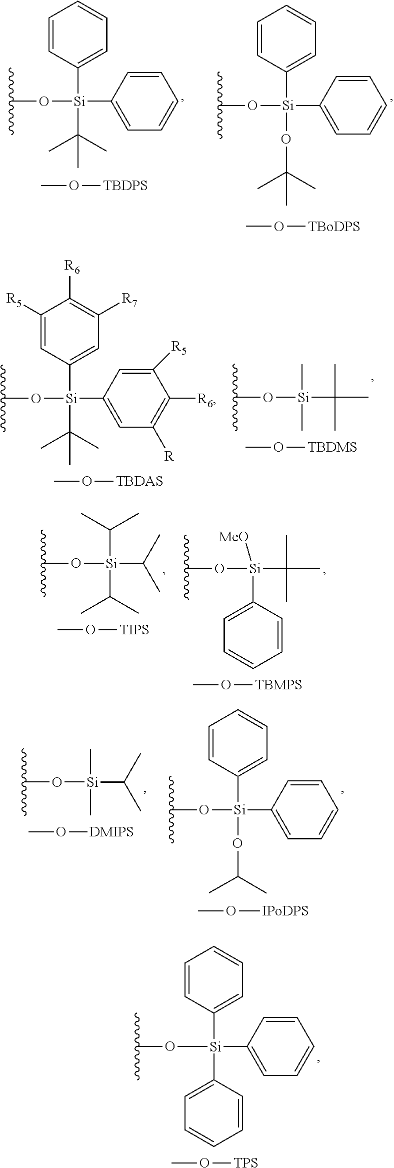

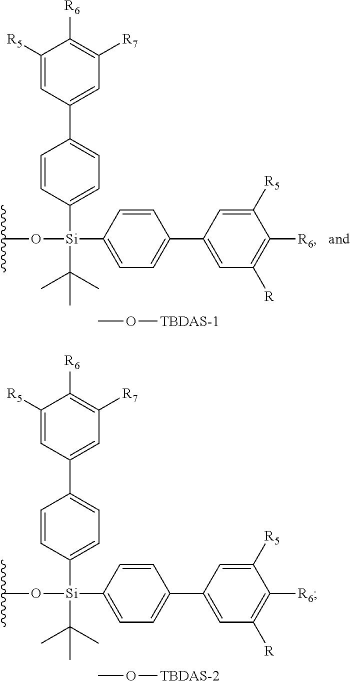

wherein represents the point of attachment for P₁ and R₅, R₆ and R₇ are each independently H, C₁₋₃₀alkyl, or C₁₋₃₀alkoxy.

In a ninety-eighth embodiment, the present disclosure provides a process described in the third or fourth aspect (e.g., any one of the fifty-fourth through ninety-seventh embodiments), wherein P₁ is selected from the group consisting of —O-TBDMS, —O-TIPS, —O-TBDPS, —O-TBoDPS, and —O-TBDAS:

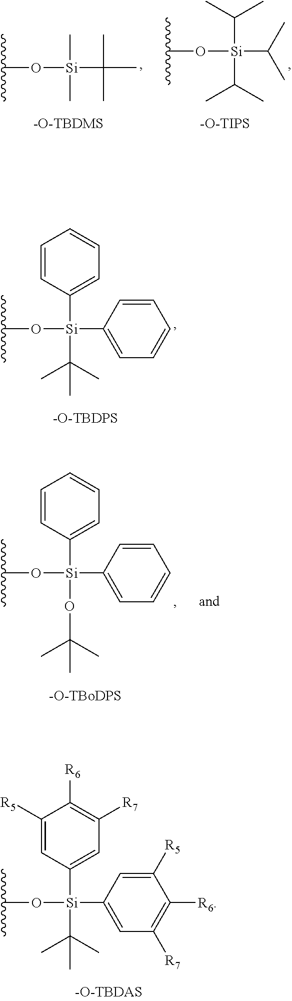

In a ninety-nineth embodiment, the present disclosure provides a process described in the third or fourth aspect (e.g., any one of the fifty-fourth through ninety-third embodiments), wherein Z is a group represented by Formula I** or Ia**:

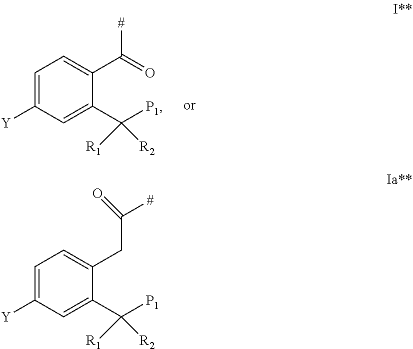

or a salt thereof;
wherein P₁ is selected from the group consisting of —O-TBDPS, —O-TBoDPS, and —O-TBDAS:

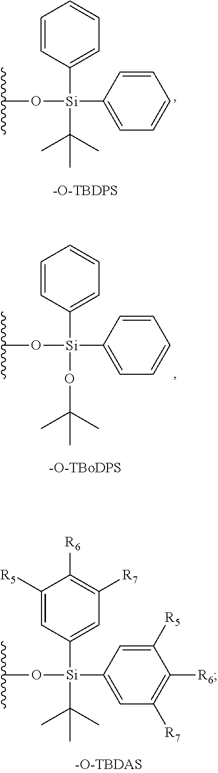

and R₅, R₆ and R₇ are each independently H, C₁₋₃₀alkyl, or C₁₋₃₀alkoxy.

In a hundredth embodiment, the present disclosure provides a process described in the third or fourth aspect (e.g., any one of the fifty-fourth through ninety-ninth embodiments), wherein Y is represented by Formula A:

W—V—U—* (A)

wherein:
—* represents the point of attachment for Y;
W is represented by Formula A1, A2, A2-1, A2-2, A3, A3-1, or A3-2:

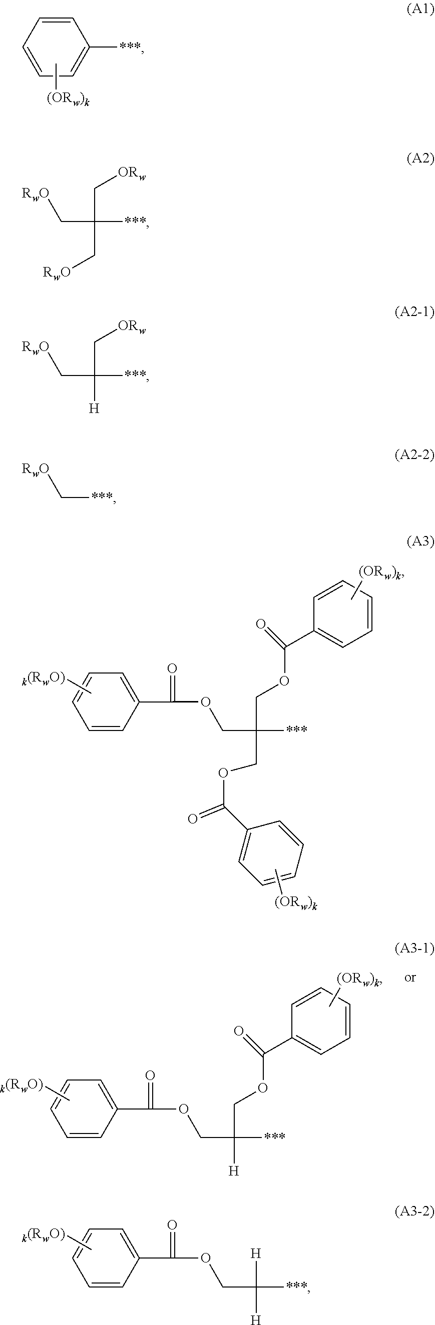

wherein
—*** represents the point where W and V connect;
each R_{w} is independently an aliphatic hydrocarbon group having 10 or more carbon atoms;
k is an integer from 1 to 5;
V is a bond, oxygen, C₁₋₂₀alkylene, C₁₋₆alkynylene, —C(═O)—, ***—C(═O)—O—**, ***—O—C(═O)—**,

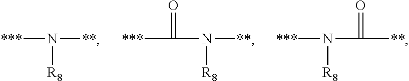

5 to 7 member heteroaryl having 1 to 3 heteroatoms selected from oxygen, nitrogen, and sulfur, wherein the heteroaryl is optionally substituted by 1-3 R₈; wherein —** represents the point where V and U connect; and R₈ is H or C₁₋₃₀alkyl; and
U is a bond, oxygen, C₁₋₂₀alkylene, carbonyl, ***—O—C(═O)—**, 5 to 7 member heterocyclyl having 1 to 3 heteroatoms selected from oxygen, nitrogen, and sulfur; 5 to 7 member heteroaryl having 1 to 3 heteroatoms selected from oxygen, nitrogen, and sulfur, wherein the heteroaryl is optionally substituted by 1-3 R₈; or a group represented by formula A4, A5, or A6:

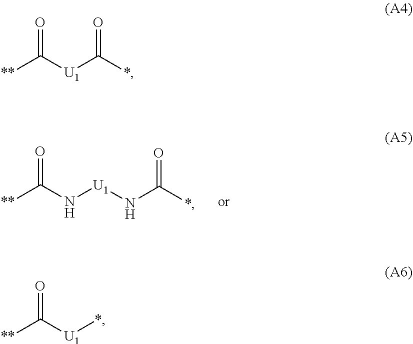

wherein U₁ is C₁₋₆alkylene, C₁₋₆alkyleneoxy, 5 to 7 member heterocyclyl having 1 to 3 heteroatoms selected from oxygen, nitrogen, and sulfur, or 5 to 7 member heteroaryl having 1 to 3 heteroatoms selected from oxygen, nitrogen, and sulfur.

In a one hundred-first embodiment, the present disclosure provides a process described in any one of the ninety-seventh through hundredth embodiments, wherein the TBDAS group is:

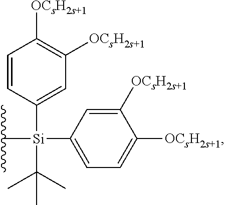

wherein s is an integer from 1 to 30.

In a one hundred-second embodiment, the present disclosure provides a process described in the fifty-fourth through hundredth embodiments, wherein P₁ is TBDPS.

In a one hundred-third embodiment, the present disclosure provides a process described in the hundredth through one hundred-second embodiments, wherein W is represented by Formula A1:

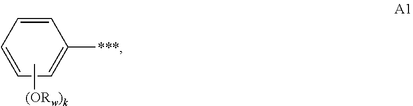

wherein R_{w} is CₙH₂ₙ₊₁;
n is an integer from 1 to 30.

In a one hundred-fourth embodiment, the present disclosure provides a process described in the one hundredth through one hundred-third embodiments, wherein R_{w} is selected from a group consisting of C₁₂H₂₅, C₁₈H₃₇, C₂₀H₄₁, C₂₂H₄₅, C₂₄H₄₉, C₂₆H₅₃, and C₂₈H₅₇.

In a one hundred-fifth embodiment, the present disclosure provides a process described in the hundredth through one hundred-fourth embodiments, wherein V is a bond, CH₂, CH₂CH₂, C(═O)—, ***—C(═O)—O—**, or

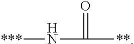

In a one hundred-sixth embodiment, the present disclosure provides a process described in the fifty-fourth through hundredth embodiments, wherein Y is selected from the groups consisting of

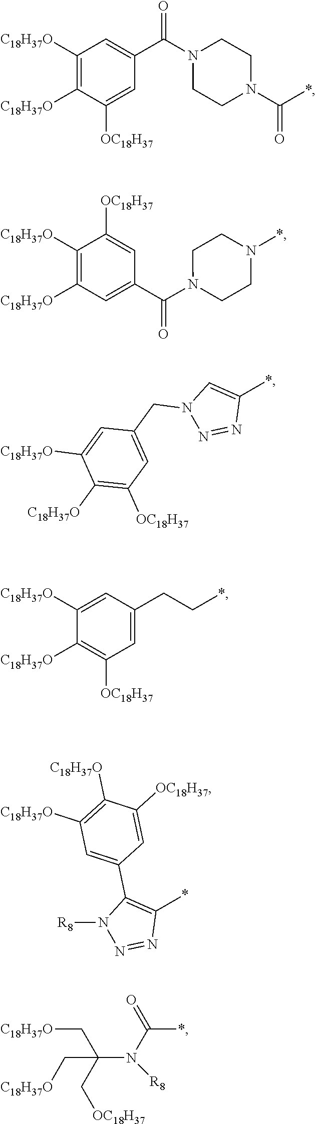

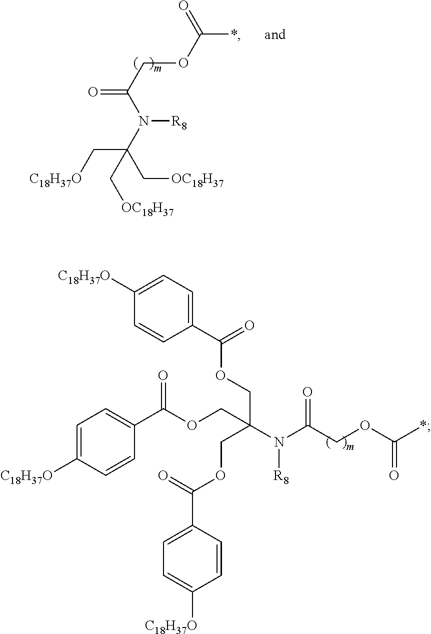

wherein
R₈ is H or C₁₋₆alkyl; and
m is an integer from 1 to 5.

In a one hundred-seventh embodiment, the present disclosure provides a process described in the third or fourth aspect (e.g., the fifty-fourth through one hundred-sixth embodiments), wherein R₁ and R₂ are independently H or CH₃. In a specific embodiment, R₁ and R₂ are both H. In another specific embodiment, R₁ and R₂ are both CH₃.

In a one hundred-eighth embodiment, the present disclosure provides a process described in the third or fourth aspect (e.g., the fifty-fourth through one hundred-seventh embodiments), wherein e is 0, 1, or 2; and f is 0, 1, or 2.

In a one hundred-ninth embodiment, the present disclosure provides a process described in the third or fourth aspect (e.g., the fifty-fourth through one hundred-eighth embodiments), wherein e is 1; and f is 1.

In a one hundred-tenth embodiment, the present disclosure provides a process described in the third or fourth aspect (e.g., the fifty-fourth through one hundred-eighth embodiments), wherein e is 0; and f is 1 or e is 1; and f is 0.

In a one hundred-eleventh embodiment, the present disclosure provides a process described in the third or fourth aspect (e.g., the fifty-fourth through one hundred-tenth embodiments), wherein R₈ is H or C₁₋₄alkyl.

In a one hundred-twelfth embodiment, the present disclosure provides a process described in the third or fourth aspect (e.g., the fifty-fourth through one hundred-eleventh embodiments), wherein Z is represented by Formula II* or IIa*,

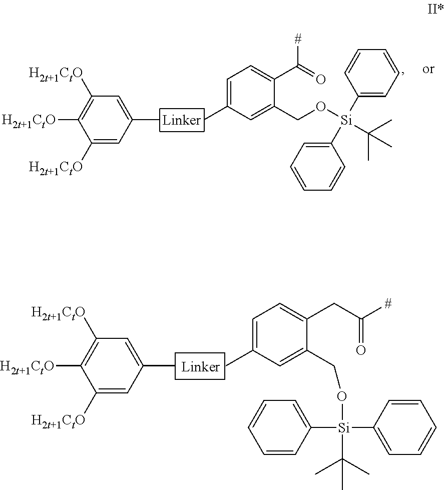

wherein
t is an integer from 10 to 30;
[symbol] is selected from the group consisting of

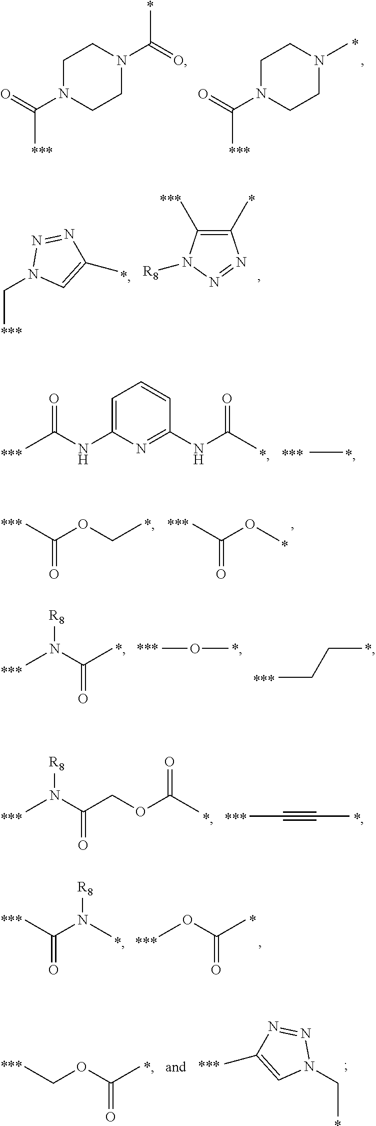

wherein R₈ is H or C₁₋₆alkyl.

In a one hundred-thirteenth embodiment, the present disclosure provides a process described in the third or fourth aspect (e.g., the fifty-fourth through one hundred-twelfth embodiments), wherein Z is:

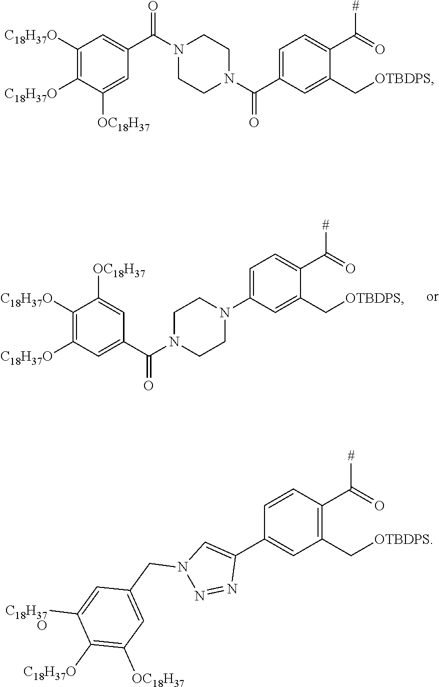

In a one hundred-fourteenth embodiment, the present disclosure a process described in the third or fourth aspect (e.g., the fifty-fourth through ninety-third embodiments), wherein Z is

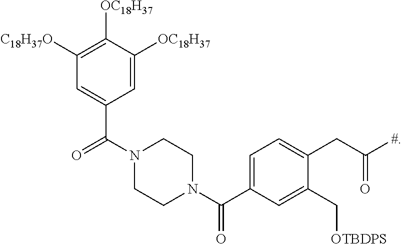

In a one hundred-fifteenth embodiment, the present disclosure a process described in the third or fourth aspect (e.g., the fifty-fourth through ninety-third embodiments), wherein Z is

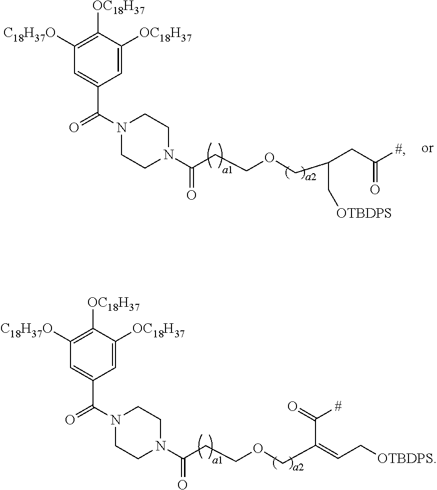

In a one hundred-sixteenth embodiment, the present disclosure provides a nucleotide or oligonucleotide described in the twenty-seventh through fifty-third embodiments or a process described in the fifty-fourth through one hundred-fifteenth embodiments, wherein all of the P═X groups in the nucleotide or oligonucleotide are P═S.

In a one hundred-seventeenth embodiment, the present disclosure provides a nucleotide or oligonucleotide described in the twenty-seventh through fifty-third embodiments or a process described in the fifty-fourth through one hundred-fifteenth embodiments, wherein all of the P═X groups in the nucleotide or oligonucleotide are P═O.

In a one hundred-eighteenth embodiment, the present disclosure provides a nucleotide or oligonucleotide described in the twenty-seventh through fifty-third embodiments or a process described in the fifty-fourth through one hundred-fifteenth embodiments, wherein greater than 10%, 20%, 30%, 40%, 50%, 60%, 70%, 80% or 90% of the P═X groups in the compound or oligonucleotide are P═S.

In a one hundred-nineteenth embodiment, the present disclosure provides a nucleotide or oligonucleotide described in the twenty-seventh through fifty-third embodiments or a process described in the fifty-fourth through one hundred-fifteenth embodiments, wherein 10-90%, 20-80%, 30-70% or 40-60% of the P═X groups in the compound or oligonucleotide are P═S.

In a one hundred-twenty embodiment, the present disclosure provides a nucleotide or oligonucleotide described in the twenty-seventh through fifty-third embodiments or a process described in the fifty-fourth through one hundred-sixteenth embodiments, wherein the nucleobase is selected from the group consisting of cytosine, guanine, adenine, thymine, uracil, hypoxanthine, xanthine, 7-methylguanine, 5,6-dihydrouracil, 5-methylcytosine, and 5-hydroxymethylcytosine, wherein the NH₂ group of the nucleobase, if present, is protected by PhCO—, CH₃CO—, iPrCO—, Me₂N—CH═, or Me₂N—CMe═.

In a one hundred-twenty first embodiment, the present disclosure provides a nucleotide or oligonucleotide described in the twenty-seventh through fifty-third embodiments or a process described in the fifty-fourth through one hundred-fifteenth embodiments, wherein the nucleobase is selected from the group consisting of cytosine, guanine, adenine, thymine, uracil, and 5-methylcytosine, wherein the NH₂ group of the nucleobase, if present, is protected by PhCO—, CH₃CO—, iPrCO—, Me₂N—CH═, or Me₂N—CMe═.

In a one hundred-twenty second embodiment, the present disclosure provides a nucleotide or oligonucleotide described in the twenty-seventh through fifty-third embodiments or a process described in the fifty-fourth through one hundred-twenty first embodiments, wherein
each R³² is independently selected from the group consisting of H, F, and C₁₋₄alkoxy optionally substituted with C₁₋₄alkoxy;
each R³⁴ is independently H or forms a ring with the alkoxy group of R², wherein the ring is a 5 or 6-membered ring optionally substituted with 1 to 3 C₁₋₄ alkyl groups;
each R³⁵ is a 4,4′-dimethoxytirtyl group;
R³⁶ is —CH₂CH₂CN; and
R^{37a} and R^{37b} are independently C₁₋₄alkyl.

In a one hundred-twenty third embodiment, the present disclosure provides a nucleotide or oligonucleotide described in the twenty-seventh through fifty-third embodiments or a process described in the fifty-fourth through one hundred-twenty first embodiments, wherein
each R³² is independently selected from the group consisting of H, F, —OCH₃, —OCH₂CH₂OCH₃, and —OTBDMS; and
each R³⁴ is independently H or forms a ring with the alkoxy group of R³², wherein the ring is a 5-membered ring.

In a one hundred-twenty fourth embodiment, the present disclosure provides a nucleotide or oligonucleotide described in the twenty-seventh through fifty-third embodiments or a process described in the fifty-fourth through one hundred-twenty first embodiments, wherein each R³⁴ is independently H or together with the alkoxy group of R³² form —CH₂—O—.

In a one hundred-twenty fifth embodiment, the present disclosure provides a nucleotide or oligonucleotide described in the twenty-seventh through fifty-third embodiments or a process described in the fifty-fourth through one hundred-twenty first embodiments, wherein
each R³² is independently selected from H or —OCH₂CH₂OMe;
each R³⁴ is H;
each R³⁵ is a 4,4′-dimethoxytirtyl group;
R³⁶ is —CH₂CH₂CN; and
R^{37a} and R^{37b} are both —CH(CH₃)₂.

In a one hundred-twenty sixth embodiment, the present disclosure provides a process described in the fifty-fifth, sixty-fourth, or eighty-fifth embodiment, wherein the salt of the compound of formula (VD′), (V-2′), or (F2′) is selected from trimethyl amine salt, triethyl amine salt, and triisopropyl amine salt.

In a one hundred-twenty seventh embodiment, the present disclosure provides a process described in one hundred-twenty sixth the embodiment, wherein the salt of the compound of formula (VD′), (V-2′), or (F2′) is triethyl amine salt.

In a one hundred-twenty eighth embodiment, the present disclosure provides a nucleotide or oligonucleotide described in the second aspect (e.g., the twenty-eighth embodiment) or a process described in the third or fourth aspect (e.g., any one of the fifty-eighth, fifty-ninth, sixty-ninth, and seventy-first through ninety-second embodiments), wherein the [symbol] is adenine, cytosine, or guanine.

In a one hundred-twenty ninth embodiment, the present disclosure provides a nucleotide or oligonucleotide described in the second aspect (e.g., the twenty-eighth embodiment) or a process described in the third or fourth aspect (e.g., any one of the fifty-eighth, fifty-ninth, sixty-ninth, and seventy-first through ninety-second embodiments), wherein the Q is a silyl protecting group.

In a one hundred-thirtieth embodiment, the present disclosure provides a nucleotide or oligonucleotide described in the second aspect (e.g., the twenty-eighth embodiment) or a process described in the third or fourth aspect (e.g., any one of the fifty-eighth, fifty-ninth, sixty-ninth, and seventy-first through ninety-second embodiments), wherein the Q is selected from the group consisting of trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl tri(trimethylsilyl)silyl, t-butylmethoxyphenylsilyl, and t-butoxydiphenylsilyl.

In a one hundred thirty-first embodiment, the present disclosure provides a nucleotide or oligonucleotide described in the second aspect (e.g. twenty-eighth embodiment) or a process described in the third or fourth aspect (e.g., any one of the fifty-eighth, fifty-ninth, sixty-ninth, and seventy-first through ninety-second embodiments), wherein the Q is t-butyldiphenylsilyl.

In certain embodiments, for the process described in the fourth aspect or any embodiments describe therein (e.g., the eighty-fourth to one hundred-fifteenth embodiments), the variables R³¹, R³², R³⁴, R³⁵, R³⁶, q, and/or Z are described in the second aspect or any one of embodiments described therein (e.g., the twenty-seventh to fifty-third embodiments).

In certain embodiments, for the process described in the fourth aspect or any embodiments describe therein (e.g., the eighty-fourth to one hundred-fifteenth embodiments), the 5′-OH deprotection (or detritylation) step, the coupling step, and the oxidation or sulfurization step are carried out under conditions described in the third aspect or any one of embodiments described therein (e.g., the fifty-fourth to eighty-third embodiments).

In certain embodiments, for a nucleotide or oligonucleotide described in the second aspect or any embodiments described therein or a process described in the third or fourth aspect or any embodiments described therein, when X is S, the phosphorothiolate group can have S-configuration, R-configuration or a mixture thereof (e.g., a racemic mixture).

### EXEMPLIFICATION

### Abbreviation

ACN=acetonitrile
Calcd=calculated
DBU=8-diazabicyclo[5.4.0]undec-7-ene
DCA=CHCl₂COOH or dichloroacetic acid
DCM=dichloromethane
DDTT=3-(N,N-dimethylamino-methylidene)amino)-3H-1,2,4-dithiazole
DCI=4,5-dicyanoimidazole
DIEA=N,N-diisopropylethylamine
DMT or DMTr=4,4′-dimethoxytrityl or bis-(4-methoxyphenyl)phenylmethyl
DMSO=dimethyl sulfoxide
EtOAc or EA=ethyl acetate
ETT=5-ethylthio-1H-tetrazole
h or hr=hour
HBTU=3-[bis(dimethylamino)methyliumyl]-3H-benzotriazol-1-oxide hexafluorophosphate
HOBt=hydroxybenzotriazole
imid=imidazole
IPAC=isopropyl acetate
iPrOH=isopropyl alcohol
MOE=methoxyethyl
MS=molecular sieve
MTBE or TBME=methyl tert-butyl ether
NMI=N-methylimidazole
TBS=tert-Butyldimethylsilyl
Py=pyridine
RBF=round bottom flask
RT=retention time
TBAF=tetra-n-butylammonium fluoride
TBuAA=tributylamine acetate
TBDPSCl=tert-butyl(chloro)diphenylsilane
TCA=trichloroacetic acid
TEA=triethylamine
TEAB=tetraethylammonium bromide
TFA=trifluoroacetic acid
THF=tetrahydrofuran
TLC=thin layer chromatography
Tol=toluene

### Example 1. Synthesis of Compound M19

a. Scheme for Synthesis of Compound M19

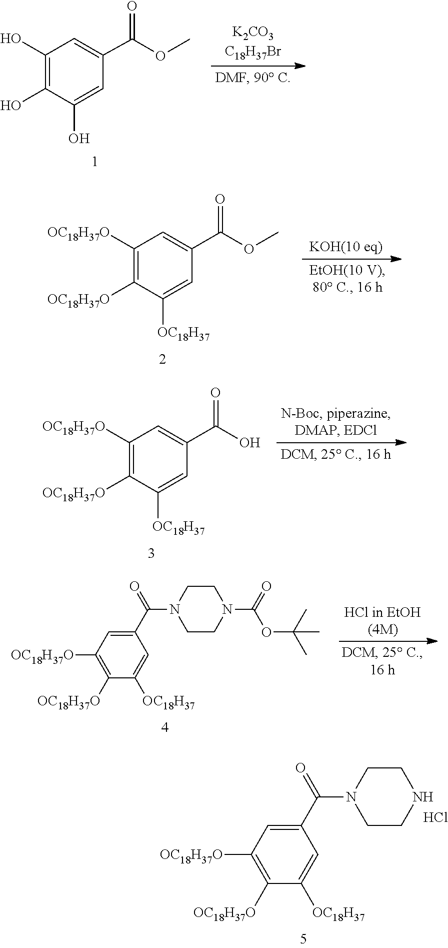

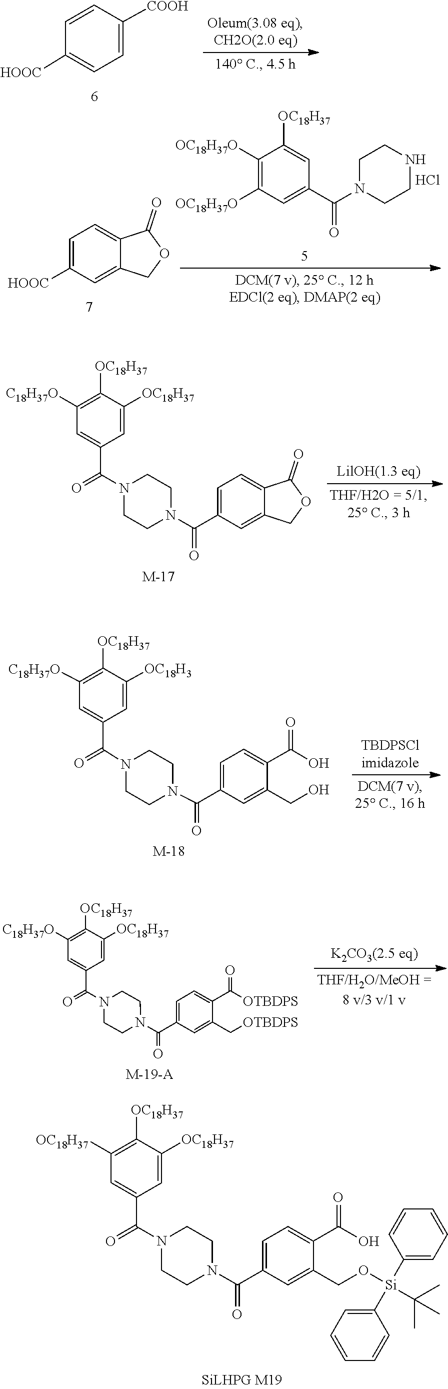

b. Procedures for Synthesis of Compound M19

General Procedure for Preparation of Compound 2

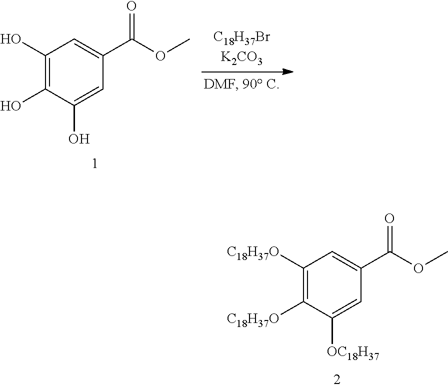

To a mixture of compound 1 (1.20 kg, 6.52 mol, 1.00 eq) and K₂CO₃ (5.40 kg, 39.1 mol, 6.00 eq) in DMF (12 L) was added 1-bromooctadecane (8.69 kg, 26.1 mol, 4.00 eq) in one portion at 25° C. under N₂. The mixture was stirred at 90° C. and stirred for 16 h. TLC (dichloromethane/methanol=5/1, Start material, R_{f}=0.52, product, R_{f}=0.88) indicated no start material was detected. Added 18 L H₂O, cool down to 25° C., filtered and washed with 7 L H₂O and 10 L acetone. The solid was recrystallized with 30 L n-heptanes at 55° C. for 1 h. Cool down to 25° C., filtered and washed the solid with 5 L n-heptanes. Compound 2 (8.60 kg, crude) was obtained as a white solid.

General Procedure for Preparation of Compound 3

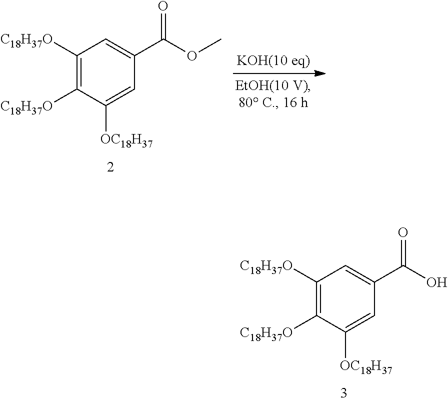

To a mixture of compound 2 (3.00 kg, 3.19 mol, 1.00 eq) in EtOH (15 L) was added solution of KOH (268 g, 4.78 mol, 1.50 eq) in H₂O (3 L) in one portion at 25° C. under N₂. The mixture was stirred at 80° C. and stirred for 16 h. TLC (petroleum ether/ethyl acetate=5/1, start material R_{f}=0.33, product R_{f}=0.86) indicated no start material was detected. Adjust pH to 2-3 with 2N HCl (6 L), cool down to 25° C., poured into 75 L H₂O. Filtered and washed the solid with 20 L H₂O, and 10 L acetone. Dried under oven at 50° C. for 24 h. Dissolved in 28 L DCM, triturated at 40° C. for 1 h. Cool down to 25° C. Filtered and washed with 40 L MeOH. Dried under oven at 50° C. for 48 h. Compound 3 (4.20 kg, 4.53 mol, 71.1% yield) was obtained as a white solid. ¹H NMR: 400 MHz CDCl₃ 7.33 (s, 2H), 4.06-4.01 (m, 6H), 1.84-1.76 (m, 6H), 1.50-1.26 (m, 6H), 1.26 (m, 86H), 0.90-0.87 (t, J=6.8 Hz, 9H).

General Procedure for Preparation of Compound 4

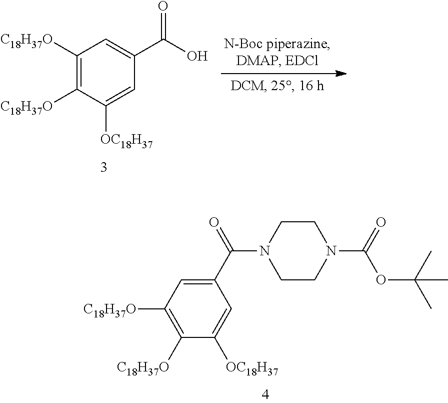

To a mixture of compound 3 (4.00 kg, 4.31 mol, 1.00 eq), EDCI (1.65 kg, 8.62 mol, 2.00 eq) and DMAP (105 g, 862 mmol, 0.200 eq) in DCM (28 L) was added tert-butyl piperazine-1-carboxylate (1.04 kg, 5.61 mol, 1.30 eq) at 25° C. Stirred for 16 h at 25° C. under N₂. TLC (dichloromethane/methanol=20/1, start material R_{f}=0.32, product R_{f}=0.53) indicated no start material was detected. Poured into 50 L MeOH, filtered and washed the cake with 30 L MeOH. Compound 4 (4.62 kg, 4.22 mol, 97.7% yield) was obtained as a white solid. ¹H NMR: 400 MHz CDCl₃ 6.57 (s, 2H), 3.97-3.94 (m, 6H), 3.57-3.29 (m, 8H), 1.82-1.71 (m, 6H), 1.47 (m, 16H), 1.25 (m, 16H), 0.89-0.86 (t, J=6.8 Hz, 9H).

General Procedure for Preparation of Compound 5

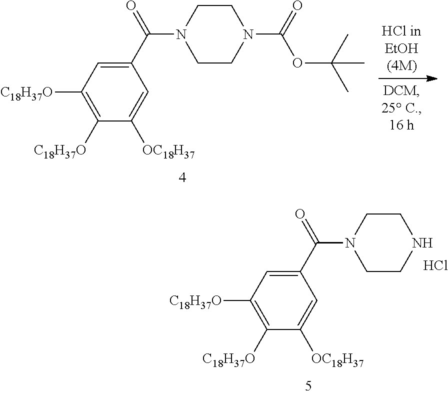

To a mixture of compound 4 (1.50 kg, 1.37 mol, 1.00 eq) in DCM (10 L) was added 4N HCl in EtOH (4 M, 3.42 L, 10.0 eq) in one portion at 25° C. under N₂. The mixture was stirred for 16 h at 25° C. TLC (DCM/MeOH=20/1, product R_{f}=0.74, start material R_{f}=0.18) indicated compound 4 was disappeared. Filtered and washed the solid with 5 L EtOH. Compound 5 (3.80 kg, 3.68 mol, 89.6% yield, HCl salt form) was obtained as a white solid.

General Procedure for Preparation of Compound 7

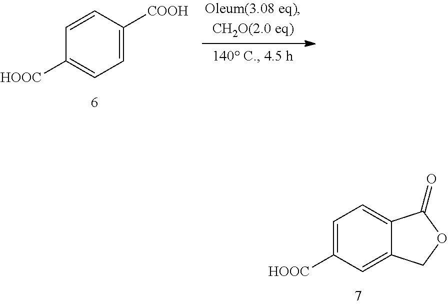 [SMILES: O=C(O)c1ccc(C(=O)O)cc1 ; O=C(O)c1ccc2c(c1)COC2=O]

To a mixture of compound 6 (500 g, 3.01 mol, 1.00 eq) and CH₂O (181 g, 6.02 mol, 2.00 eq) was added Oleum (825 mL) in one portion at 25° C. under N₂. The mixture was stirred at 140° C. for 15 h. Exhaust gas absorption was charged with 10% NaOH aqueous. HPLC (start material: RT=2.73 min; product: RT=2.84 min) showed compound 6 was disappeared. Cooled down to 25° C., quenched with 3300 mL H₂O. Filtered and washed with H₂O until pH was 3-4. Recrystallized with 3200 mL DMF at 80° C. Filtered and washed with 2 L EtOH. Dried under vacuum. Compound 7 (1.60 kg, crude) was obtained as a gray solid. A mixture of compound 7 (1.60 kg, 8.98 mol, 1.00 eq) in DMF (3200 mL) was stirred at 80° C. for 1 h. Cooled down to 25° C. slowly through a period of 16 h. Filtered and washed with 500 mL EtOH. Dried under vacuum. Compound 7 (620 g, 3.48 mol, 38.7% yield) was obtained as a light orange solid.

Alternatively, compound 7 was prepared by the following procedure:

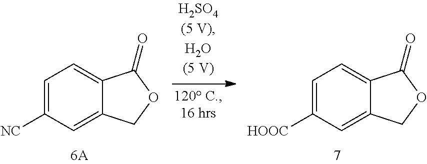 [SMILES: N#Cc1ccc2c(c1)COC2=O ; O=C(O)c1ccc2c(c1)COC2=O]

To a solution of 50% H₂SO₄ aq. (15.0 L) was added compound 6A (1.50 kg, 9.43 mol, 1.00 eq) in one portion at 25° C. under N₂. The mixture stirred at 120° C. for 16 hrs. LCMS (ET49477-3-P1A2, product: RT=0.597 min) showed the starting material was consumed completely. Cooled down to 25° C. Pour into H₂O (ice, 15.0 L), filter and wash the solids with H₂O (2.00 L×5). The filter cake dried under vacuum oven (55° C. for 48 hrs). Compound 7 (2.50 kg, 14.0 mol, 74% yield, 98.8% purity) was obtained as a white solid. ESI⁺: MS: calcd for C₉H₆O₄ (M+H)⁺178.0 found 178.1. ¹H NMR (400 MHz, CDCl₃) δ 8.19 (s, 1H), 8.09-8.07 (d, J=8.0 Hz, 1H), 7.93-7.91 (d, J=8.0 Hz, 1H), 5.45 (s, 2H). ¹³C{¹H}NMR (100 MHz, CDCl₃) δ 169.9, 166.5, 147.6, 135.7, 129.8, 128.5, 125.1, 124.0, 70.1.

General Procedure for Preparation of Compound M-17

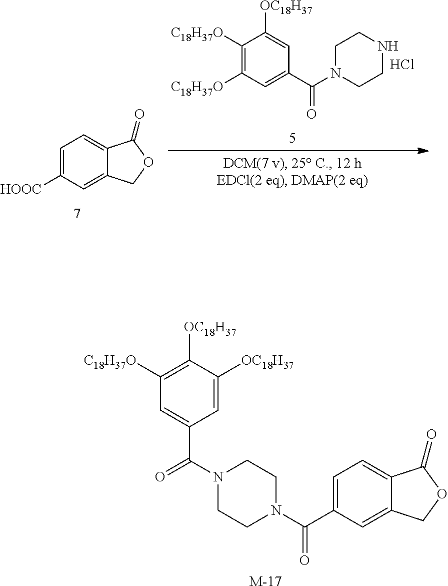

To a mixture of compound 5 (3.60 kg, 3.49 mol, 1.00 eq, HCl) and compound 7 (683 g, 3.84 mol, 1.10 eq) in DCM (24 L) was added DMAP (852 g, 6.98 mol, 2.00 eq), EDCI (1.34 kg, 6.98 mol, 2.00 eq) in one portion at 25° C. under N₂. The mixture was stirred at 25° C. for 2 h. TLC (DCM/MeOH=20/1, start material R_{f}=0.62, product R_{f}=0) indicated the start material was consumed completely. Poured into EtOH (50 L), filtered and washed with EtOH (20 L). Compound M-17 (3.80 kg, 3.29 mol, 94.3% yield) was obtained as a white solid. ¹H NMR: 400 MHz CDCl₃ 7.99-7.97 (d, J=6.8 Hz, 1H), 7.56-7.53 (m, 2H), 6.59 (s, 2H), 5.36 (s, 2H), 3.97-3.94 (t, J=6.4 Hz, 6H), 3.78-3.44 (m, 8H), 1.82-1.71 (m, 7H), 1.46-1.42 (m, 7H), 1.30-1.26 (m, 93H), 0.89-0.86 (t, J=6.8 Hz, 9H)

General Procedure for Preparation of Compound M-18

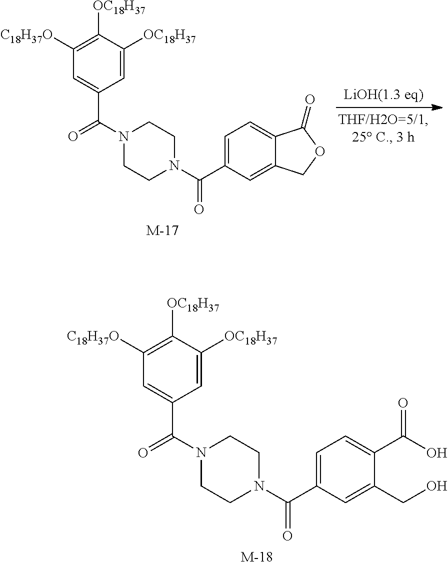

To a mixture of compound M-17 in THF (20 L) was added solution of LiOH·H₂O (175 g, 4.16 mol, 1.30 eq) in H₂O (4000 mL) in one portion at 25° C. under N₂. The mixture was stirred at 25° C. and stirred for 3 h. TLC (DCM/MeOH=20/1, start material R_{f}=0.46, product R_{f}=0.05) indicated the start material was consumed completely. Concentrated and diluted with 40 L H₂O, adjust pH to 4-5 with 1N HCl (10 L). Filtered and washed with 45 L H₂O until the pH was 6-7. Washed with 4 L ACN. Compound M-18 (3.90 kg, crude) was obtained as a white solid. ¹H NMR: ET29928-65-P1A1 400 MHz CDCl₃ 8.05-8.00 (m, 1H), 7.57-7.54 (m, 1H), 7.38-7.36 (m, 1H), 6.59 (s, 2H), 6.6 (s, 2H), 4.79 (s, 2H), 3.97-3.94 (m, 8H), 3.81-3.45 (m, 8H), 1.82-1.77 (m, 4H), 1.45 (m, 7H), 1.29-1.25 (m, 90H), 0.89-0.86 (t, J=7.2 Hz, 9H).

General Procedure for Preparation of Compound M-19-A

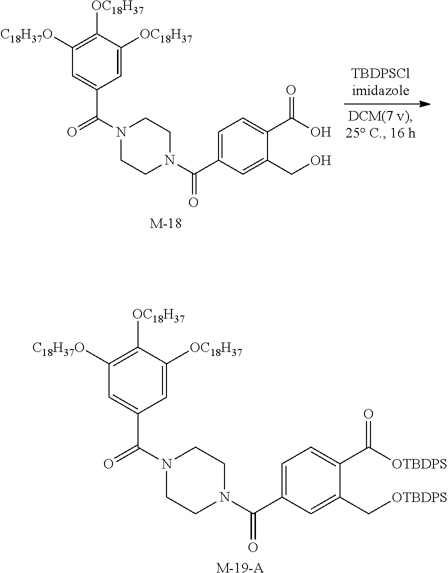

To a mixture of compound M-18 (1.70 kg, 1.45 mol, 1.00 eq) in DCM (20 L) was added imidazole (986 g, 14.5 mol, 10.0 eq) and TBDPSCl (3.98 kg, 14.5 mol, 3.72 L, 10.0 eq) in one portion at 25° C. under N₂. The mixture was stirred at 25° C. and stirred for 2 h. TLC (DCM/MeOH=10/1, start material R_{f}=0.18, product R_{f}=0.92) indicated the start material was consumed completely. The reaction worked up with ET29928-70 together. Washed with H₂O (15 L×2), separated the organic layer and dried over anhydrous Na₂SO₄ and concentrated. Compound M-19-A (5.80 kg, crude) was obtained as a white solid.

General Procedure for Preparation of Compound SiLHPG M19

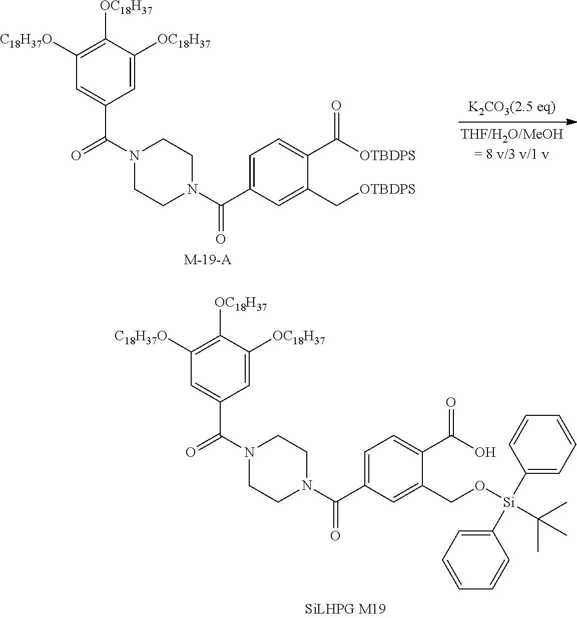

To a mixture of compound M-19-A (2000 g, 485 mmol, 1.00 eq) in THF (16 L) was added solution of K₂CO₃ (87.1 g, 630 mmol, 1.30 eq) in H₂O (6000 mL) and MeOH (2000 mL) one portion at 25° C. under N₂. The mixture was stirred for 16 h at 25° C. TLC (PE/EA=2/1, start material R_{f}=0.43, product R_{f}=0) indicated the start material was consumed completely. Concentrated and diluted with 10 L H₂O, adjust pH to 5 with 1 M KHSO₄, extracted with DCM (10 L×2), dried over anhydrous Na₂SO₄. Concentrated to ˜5 L, poured into 10 L MeOH, filtered and washed with MeOH (5 L×4) to remove the TBDPS byproduct. Dissolved in DCM (5 L) and dropwised into MeCN (10 L), filtered and washed with MeCN (2 L×4), dissolved in DCM(12 L), filtered through silica gel pad, and washed with DCM/EtOAc=1/1 (10 L). Compound SiLHPG M19 (835 g, 591 mmol, 48.8% yield) was obtained as a white solid. ¹H NMR: 400 MHz CDCl₃ δ 8.10-8.08 (d, J=8.0 Hz, 1H), 7.99 (s, 1H), 7.66-7.64 (m, 4H), 7.43-7.32 (m, 7H), 6.59 (s, 2H), 5.21 (s, 2H), 3.99-3.93 (m, 6H), 3.78-3.44 (m, 8H), 1.82-1.75 (m, 6H), 1.47-1.42 (m, 7H), 1.31-1.27 (m, 93H), 1.12 (s, 10H), 0.90-0.87 (t, J=6.8 Hz, 9H).

General Procedure for Preparation of Compound M-18A:

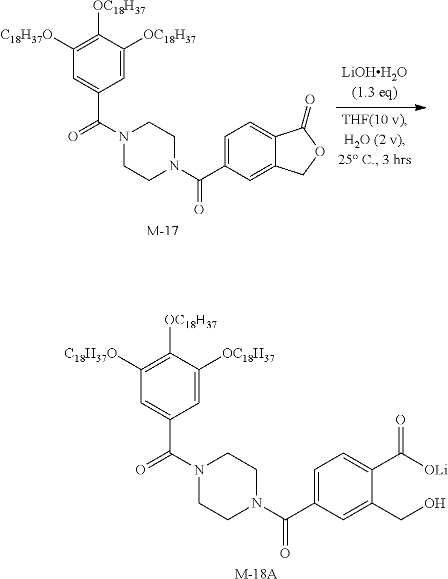

To a mixture of compound M-17 (2.00 kg, 1.73 mol, 1.00 eq) in THF (20.0 L) was added solution of LiOH·H₂O (94.4 g, 2.25 mol, 1.30 eq) in H₂O (4000 mL) in one portion at 25° C. under N₂. The mixture was stirred at 25° C. and stirred for 3 hrs. TLC (dichloromethane/methanol=20/1, start material R_{f}=0.5, product R_{f}=0.1) indicated the start material was consumed completely. Poured into ACN (20.0 L) and filtered. Collect solids combined then dry under vacuum oven (50° C., 10 days). The reactions were carried out with five batches in parallel. Compound M-18A (10.8 kg, 9.15 mol, 106% yield, 87.4% purity) was obtained as a white solid. HRMS: calcd for C₇₄H₁₂₉N₂O₈ (M−Li+2H)⁺1173.9671, found 1173.9755. ¹H NMR: (Li salt was neutralized to free acid for H-NMR). 400 MHz, CDCl₃ δ 8.05-8.00 (m, 1H), 7.57-7.54 (m, 1H), 7.38-7.36 (m, 1H), 6.59 (s, 2H), 6.6 (s, 2H), 4.79 (s, 2H), 3.97-3.94 (m, 8H), 3.81-3.45 (m, 8H), 1.82-1.77 (m, 4H), 1.45 (m, 7H), 1.29-1.25 (m, 90H), 0.89-0.86 (t, J=7.2 Hz, 9H).

Alternatively, compound M19 was prepared by the following procedure:

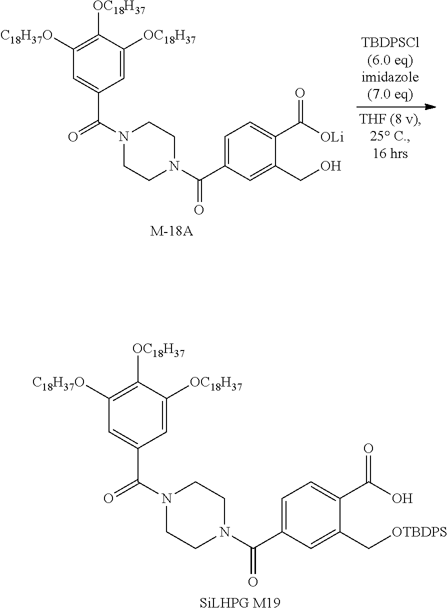

To a mixture of compound M-18A (3.00 kg, 2.54 mol, 1.00 eq) in THF (24.0 L) was added imidazole (1.21 kg, 17.80 mol, 7.0 eq) and TBDPSCl (4.19 kg, 15.3 mol, 3.92 L, 6.0 eq) in 5 portions at 25° C. under N₂. The mixture was stirred at 25° C. and stirred for 16 hrs. HPLC (start material t_{R}=5.92 min, product t_{R}=9.32 min) indicated the start material was ˜5% remained. The reaction diluted with DCM (20.0 L) and wash with H₂O (20.0 L×2). Then the organic layer was dried over anhydrous Na₂SO₄, filtered and concentrated ˜7 L, poured into 15 L MeOH, filtered and washed with MeOH (5 L×4) to remove the TBDPS byproduct. Dissolved in DCM (7 L) and dropwised into MeCN (15 L), filtered and washed with MeCN (5 L×4), the filter cake combined and dried under oven (50° C., 72 hrs) Compound SiLHPG M19 (13.6 kg, crude, 89% purity, 5% M-17, 3% M-18) was obtained as a white solid. The residue was purified by column chromatography (SiO2, Petroleum ether/Ethyl acetate=1/10 to 4/1) (10% DCM was added into PE eluent). The reactions were carried out with three batches in parallel. Concentrate and compound SiLHPG M19 (7.20 kg, 5.09 mol, 66.5% yield, 95.08% purity) was obtained as a white solid. HRMS: calcd for C₉₀H₁₄₇N₂O₈Si (M+H)⁺1412.0848, found 1412.0908. ¹H NMR (400 MHz, CDCl3) δ 8.10-8.08 (d, J=8.0 Hz, 1H), 7.99 (s, 1H), 7.66-7.64 (m, 4H), 7.43-7.32 (m, 7H), 6.59 (s, 2H), 5.21 (s, 2H), 3.99-3.93 (m, 6H), 3.78-3.44 (m, 8H), 1.82-1.75 (m, 6H), 1.47-1.42 (m, 7H), 1.31-1.27 (m, 90H), 1.12 (s, 10H), 0.90-0.87 (t, J=6.8 Hz, 9H). ¹³C{1H} NMR (100 MHz, CDCl₃) δ 170.8, 169.9, 153.3, 144.7, 139.8, 139.4, 135.4, 134.8, 133.1, 132.1, 129.9, 129.6, 127.9, 127.7, 125.6, 125.1, 105.7, 73.6, 69.3, 64.0, 60.4, 47.5, 42.3, 31.9, 30.3, 29.4-29.7,26.9, 26.1, 22.7, 14.1.

### Example 2. Synthesis of Compound M22

a. Schemes for Synthesis of Compound M22

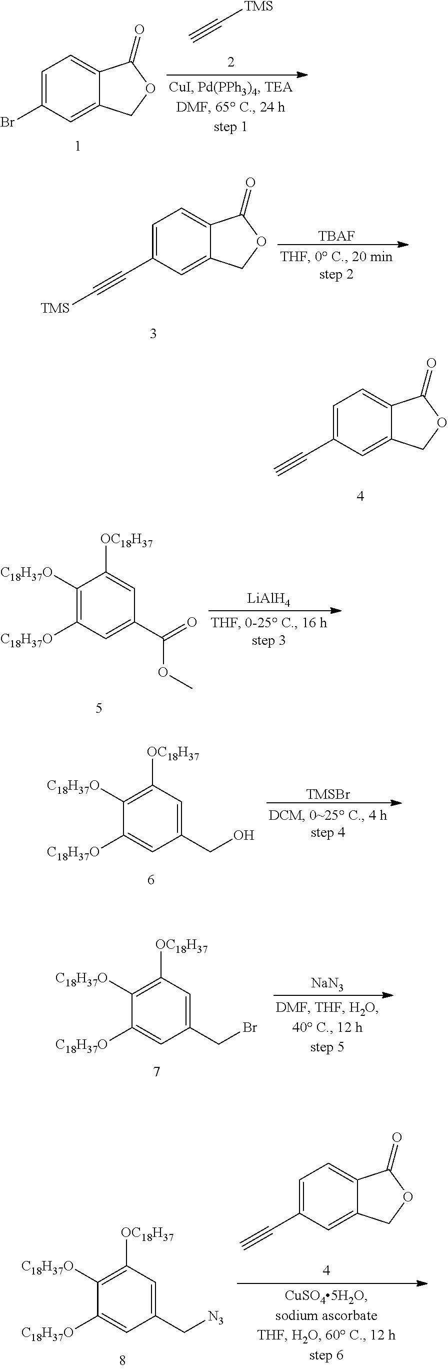

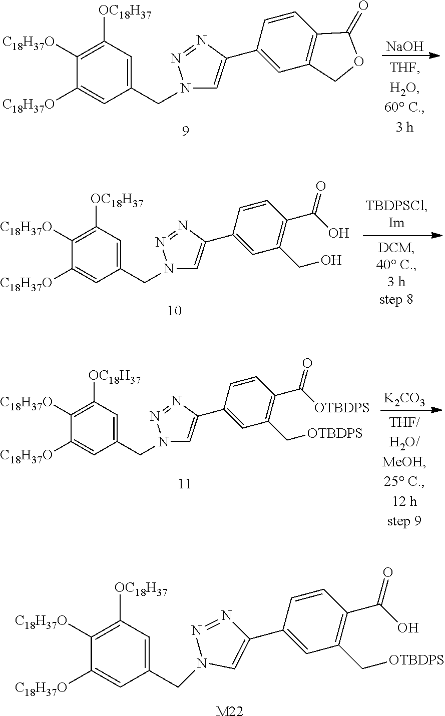

b. Procedures for Synthesis of Compound M22

General Procedure for Preparation of Compound 3

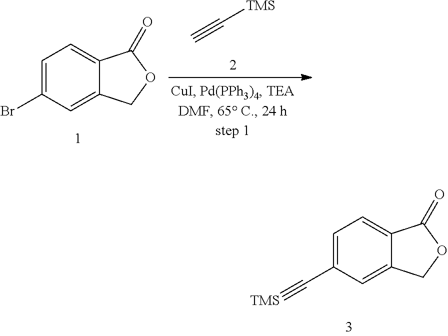

A mixture of compound 1 (24.00 g, 113 mmol), compound 2 (44.2 g, 451 mmol, 62.4 mL), CuI (6.45 g, 33.8 mmol), Pd(PPh3)4 (6.51 g, 5.64 mmol) and TEA (5.70 g, 56.3 mmol, 7.83 mL) in DMF (144 mL) was degassed and purged with N₂ for 3 times, and then the mixture was stirred at 65° C. for 24 h under N₂ atmosphere. The desired product was detected in TLC (Petroleum ether/Ethyl acetate=10/1, product: RT=0.43). The filtrate was diluted with EtOAc (600 mL) and washed with brine (450 mL×3). The organic layer was dried over anhydrous Na₂SO₄ (45.0 g), filtered and concentrated under reduced pressure to give a residue. The residue was purified by column chromatography (SiO2, Petroleum ether/Ethyl acetate=100/1 to 5/1). Compound 3 (15.0 g, 57.8% yield) was obtained as a brown solid.

General Procedure for Preparation of Compound 4

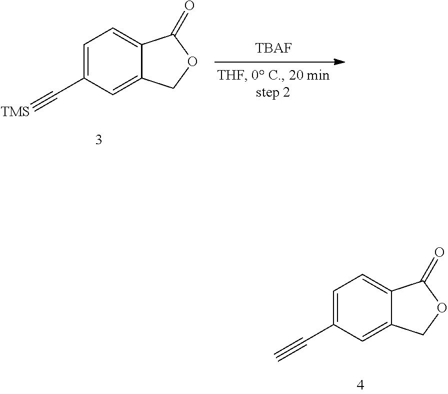

To a solution of compound 3 (15.0 g, 65.1 mmol) in THF (90.0 mL) was added TBAF (1.00 M, 65.1 mmol, 65.1 mL). The mixture was stirred at 0° C. for 20 min. The desired product was detected in LCMS (ET25847-215-P1B, RT=1.446) and TLC (Petroleum ether/Ethyl acetate=5/1, product: Rf=0.43). The reaction mixture was diluted with DCM (300 mL) and washed with brine (300 mL×3). The combined organic layers were dried over anhydrous Na₂SO₄ (30.0 g), filtered and concentrated under reduced pressure to give a residue. The residue was purified by column chromatography (SiO2, Petroleum ether/Ethyl acetate=30/1 to 5/1). Compound 4 (3.20 g, 31.1% yield) was obtained as a yellow solid. ¹H NMR: 400 MHz CDCl₃7.84-7.86 (d, J=8.0 Hz, 1H), 7.53-7.63 (m, 2H), 5.29 (s, 2H), 0.28 (s, 9H).

General Procedure for Preparation of Compound 6

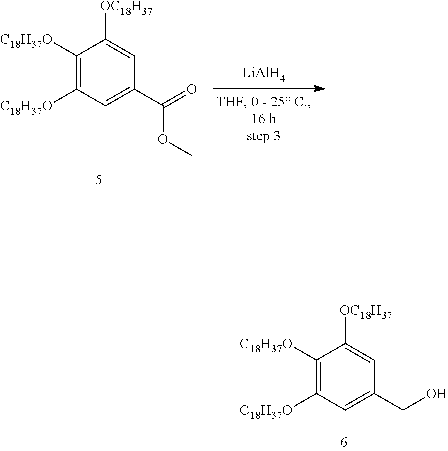

To a solution of compound 5 (48.0 g, 51.0 mmol) in THF (288 mL) was added LiAlH₄ (3.18 g, 83.6 mmol) at 0° C. The mixture was stirred at 25° C. for 16 h. TLC (DCM/MeOH=5/1, product: R_{f}=0.80) indicated compound 5 was consumed completely and one new spot formed. The reaction mixture was quenched by addition Na₂SO₄·10H₂O (90.0 g) and then filtered. The filtrate was concentrated. The residue was diluted with DCM (900 mL) and washed with water (450 mL×2), brine (600 mL) and dried over anhydrous Na₂SO₄ (90.0 g), filtered and concentrated under reduced pressure to give a residue. Compound 6 (40.0 g, crude) was obtained as a white solid. ¹H NMR: 400 MHz CDCl₃ 6.57 (s, 2H), 4.60-4.61 (d, J=4.0 Hz, 2H), 3.93-4.00 (m, 6H), 1.71-1.84 (m, 6H), 1.27-1.31 (m, 90H), 0.87-0.91 (t, J=6.4 Hz, 9H).

General Procedure for Preparation of Compound 7

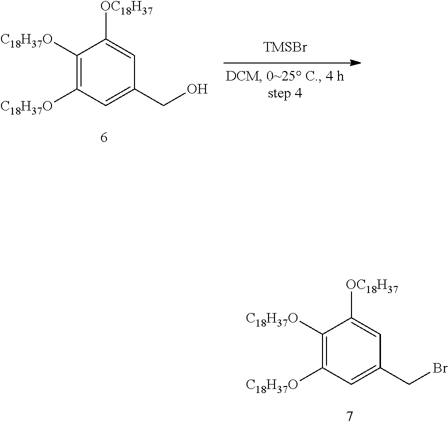

To a solution of compound 6 (40.0 g, 43.8 mmol) in DCM (240 mL) was added TMSBr (8.04 g, 52.5 mmol, 6.82 mL) at 0° C. and stirred for 1 h. Then the mixture was stirred at 25° C. for 3 h. TLC (DCM/MeOH=20/1, product: Rf=0.95) indicated compound 6 was consumed completely and one new spot formed. The reaction mixture was combined and concentrated under reduced pressure to remove solvent. The residue was dissolved in DCM (200 mL) and triturated by ACN (1.00 L). The solid was washed with ACN (200 mL×3) and filtered. Then it was concentrated. Compound 7 (42.0 g, 98.2% yield) was obtained as a light yellow solid. ¹H NMR: 400 MHz CDCl₃ 6.58 (s, 2H), 4.44 (s, 2H), 3.93-3.99 (m, 6H), 1.72-1.84 (m, 6H), 1.27-1.47 (m, 90H), 0.87-0.91 (t, J=6.4 Hz, 9H).

General Procedure for Preparation of Compound 8

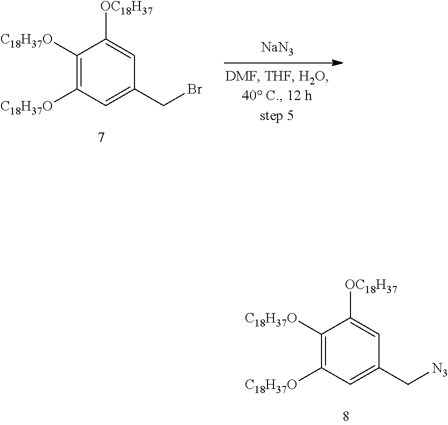

To a solution of compound 7 (42.0 g, 43.0 mmol) in DMF (252 mL) and THF (200 mL) was added NaN3 (4.20 g, 64.5 mmol) in H2O (36.0 mL). The mixture was stirred at 40° C. for 12 h. TLC (Petroleum ether/Ethyl acetate=10/1, product: RT=0.66) indicated compound 7 was consumed completely and one new spot formed. The reaction mixture was diluted with DCM (300 mL) and washed with brine (450 mL×3). The combined organic layers were dried over anhydrous Na₂SO₄ (30.0 g), filtered and concentrated under reduced pressure to give a residue. Compound 8 (40.0 g, crude) was obtained as a white solid. ¹H NMR: 400 MHz CDCl₃ 6.49 (s, 2H), 4.25 (s, 2H), 3.94-4.00 (m, 6H), 1.75-1.84 (m, 6H), 1.27-1.49 (m, 90H), 0.87-0.91 (t, J=6.4 Hz, 9H).

General Procedure for Preparation of Compound 9

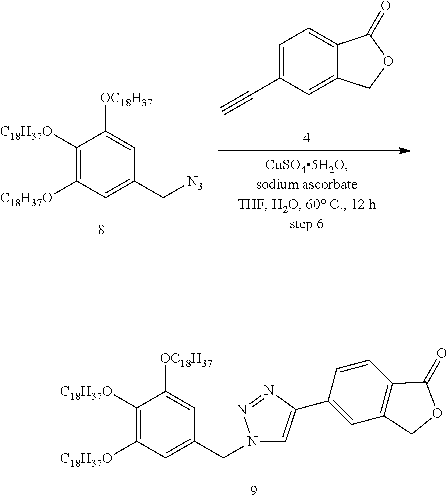

A mixture of compound 9 (2.70 g, 2.88 mmol), compound 4 (682 mg, 4.32 mmol), sodium ascorbate (570 mg, 2.88 mmol), CuSO₄·5H₂O (360 mg, 1.44 mmol) in THF (16.2 mL) and H₂O (5.40 mL) was degassed and purged with N₂ for 3 times, and then the mixture was stirred at 70° C. for 12 h under N₂ atmosphere. The desired product was detected in TLC (Petroleum ether/Ethyl acetate=3/1, R_{f}=0.22). Then it was filtered and concentrated under reduced pressure to remove solvent. The residue was dissolved in DCM (60.0 mL) and triturated with MeOH (600 mL). Compound 9 (3.00 g, crude) was obtained as a yellow solid.

General Procedure for Preparation of Compound 10

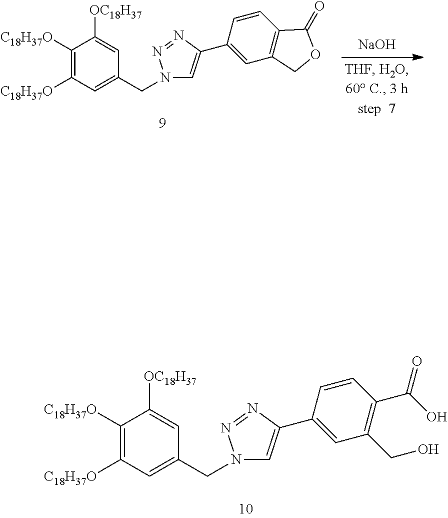

To a solution of compound 9 (3.00 g, 2.74 mmol) in THF (18.0 mL) was added NaOH (438 mg, 11.0 mmol) in H₂O (3.60 mL). The mixture was stirred at 60° C. for 3 h. TLC (Petroleum ether/Ethyl acetate=2/1, product: R_{f}=0.04) indicated compound 9 was consumed completely and one new spot formed. This reaction was combined with the reaction (ET258474-259). The reaction mixture was concentrated under reduced pressure to remove THF. The residue was diluted with H₂O (300 mL). The solution was adjusted to pH ˜2 with HCl (1.00 M) and filtered to pH ˜7 with H₂O and concentrated under reduced pressure to give a residue. Then it was washed by ACN (100 mL). Compound 10 (4.80 g, crude) was obtained as a light yellow solid.

General Procedure for Preparation of Compound 11

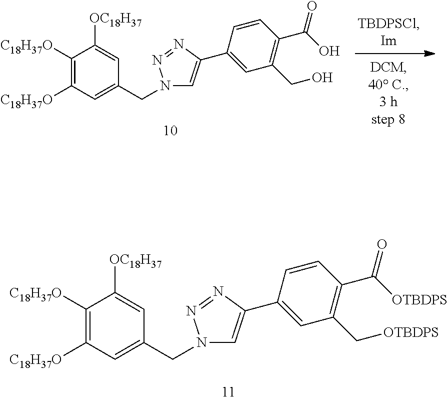

To a solution of compound 10 (4.80 g, 4.32 mmol) in DCM (28.8 mL) was added TBDPSCl (2.96 g, 10.8 mmol, 2.77 mL) and Im (880 mg, 12.9 mmol). The mixture was stirred at 40° C. for 3 h. TLC (Petroleum ether/Ethyl acetate=2/1, product: R_{f}=0.94) indicated compound 10 was consumed completely and one new spot formed. The reaction mixture was quenched by addition NaHCO₃ (150 mL), and extracted with DCM (300 mL). The combined organic layers were washed with brine (300 mL×2), dried over anhydrous Na₂SO₄, filtered and concentrated under reduced pressure to give a residue. The crude product was re-dissolve in DCM (90.0 mL) and dropped into MeOH (600 mL) with vigorous stirring. Desired product was precipitated out, filtered and concentrated under reduced pressure to give a residue. Then it was washed by MeOH (300 mL×3). Compound 11 (6.05 g, crude) was obtained as a white solid.

General Procedure for Preparation of Compound M22

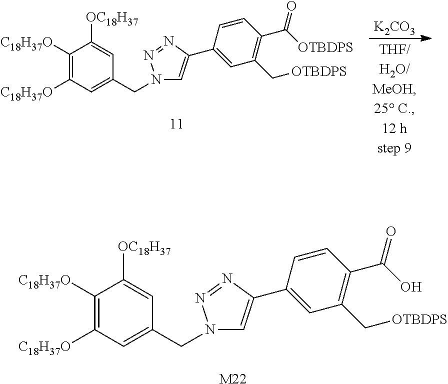

To a solution of compound 11 (6.00 g, 3.76 mmol) in THF (36.0 mL) and MeOH (6.00 mL) was added K₂CO₃ (1.30 g, 9.42 mmol) in H₂O (12 mL). The mixture was stirred at 25° C. for 12 h. TLC (Petroleum ether/Ethyl acetate=2/1, product: R_{f}=0.43) indicated compound 11 was consumed completely and one new spot formed. The reaction was combined and concentrated under reduced pressure. The reaction mixture was quenched by addition brine (200 mL), and adjusted to pH ˜2 with KHSO₄ aqueous solution (200 mL, 1.00 M). Then it was extracted with DCM (300 mL) and washed with brine (100 mL×2), dried over anhydrous Na₂SO₄, filtered and concentrated under reduced pressure to give a residue. The crude product was re-dissolve in DCM (50.0 mL) and dropped into MeOH (300 mL) with vigorous stirring. Desired product was precipitated out and filtered. Compound SiLHPG M22 (4.5 g, 88.2% yield) was obtained as a white solid. ¹H NMR: 400 MHz CDCl₃ 8.17 (s, 1H), 8.12-8.10 (d, J=8.0 Hz, 1H), 7.94-7.92 (d, J=8.0 Hz, 1H), 7.72-7.64 (m, 5H), 7.39-7.30 (m, 6H), 6.52 (s, 2H), 5.47 (s, 2H), 5.18 (s, 2H), 3.97-3.92 (m, 6H), 1.82-1.74 (m, 6H), 1.47-1.42 (m, 6H), 1.31-1.26 (m, 84H), 1.11 (s, 9H), 0.90-0.87 (t, J=6.8 Hz, 9H).

### Example 3. Synthesis of Compound M36

a. Schemes for Synthesis of Compound M36

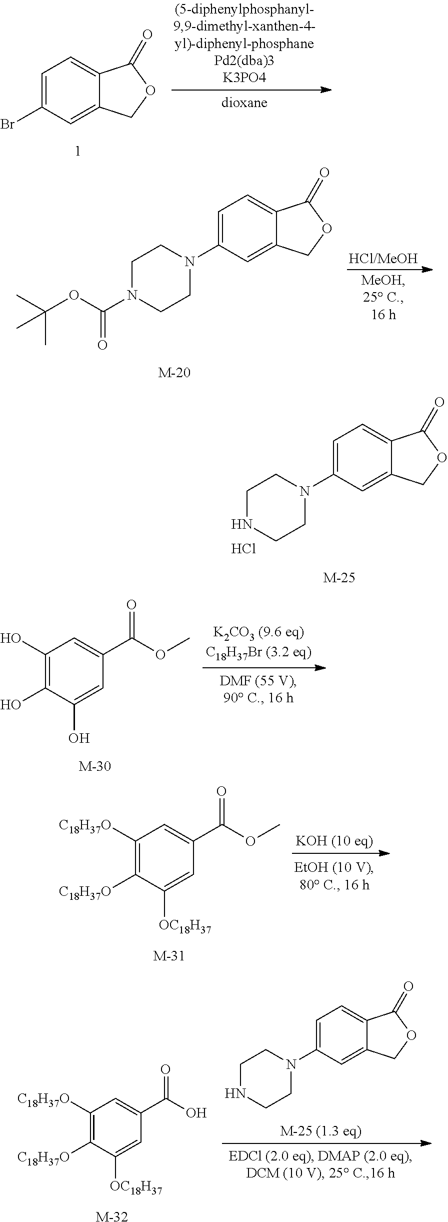

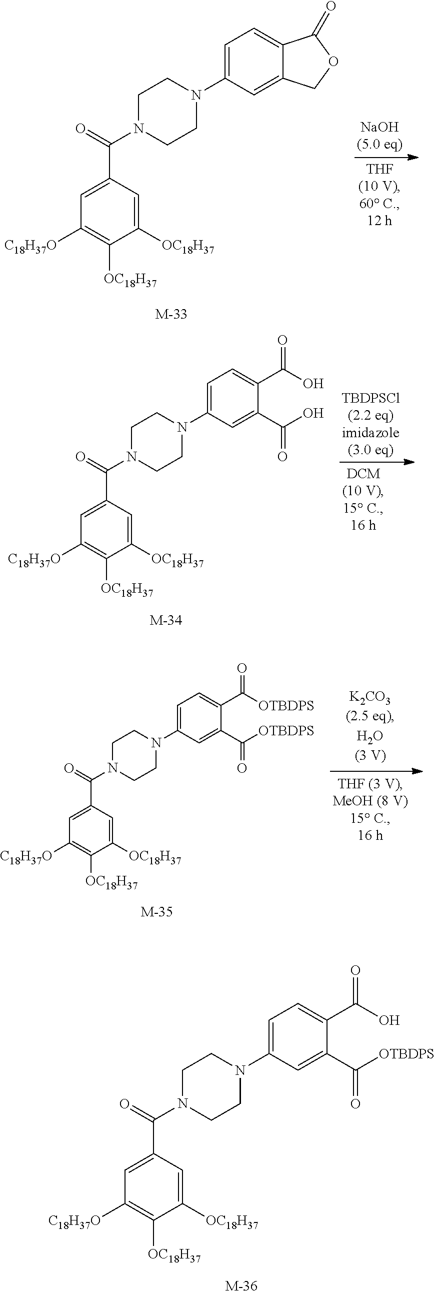

b. Procedures for Synthesis of Compound M36

General Procedure for Preparation of Compound M20

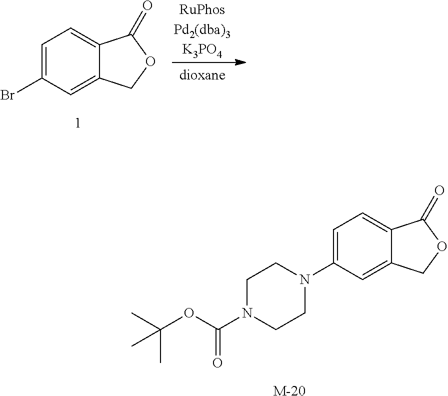 [SMILES: O=C1OCc2cc(Br)ccc21 ; CC(C)(C)OC(=O)N1CCN(c2ccc3c(c2)COC3=O)CC1 ; "M-20"]

To the three-necked round bottom flask charged with N₂ was added compound 1 (70 g, 328 mmol, 1.00 eq), tert-butyl piperazine-1-carboxylate (61.2 g, 328. mmol, 1 eq), K₃PO₄ (139 g, 657.19 mmol, 2 eq) and Tol. (700 mL). The mixture was purged and degassed with N₂ for 3 times. Then to the solution was added RuPhos (15.3 g, 32.8 mmol, 0.1 eq) and Pd₂(dba)₃ (10.71 g, 16.43 mmol, 0.05 eq). The reaction solution was purged and degassed with N2 for 3 times and warmed to 100° C. It was stired at 100° C. for 16h. The reaction solution turned to black. TLC (Petroleum ether/Ethyl acetate=2/1, starting material Rf=0.70, product Rf=0.30) indicated the starting material was consumed and the a new point was formed. The reaction was cooled to 20° C. Then it was filtered to remove solid and wash with ethyl acetate twice (1000 mL and 500 mL). The organic layers were combined and concentrated to give a crude solid. The solid was stired with a solution of MTBE:DCM (800 mL, V/V=10/1) for 16 hrs. Then it was filtered to give the solid which was dried under oil pump. M20 was obtained (64 g, 201.03 mmol, 61.18% yield) as off-white solid. ¹H NMR: 400 MHz CDCl₃ 7.76 (d, J=8.8 Hz 1H), 7.01 (dd, J=2.0 Hz, J=8.8 Hz, 1H), 6.80 (s, 1H), 5.21 (s, 2H), 3.59-3.62 (m, 4H), 3.35-3.38 (m, 4H), 1.49 (s, 9H).

General Procedure for Preparation of Compound M25

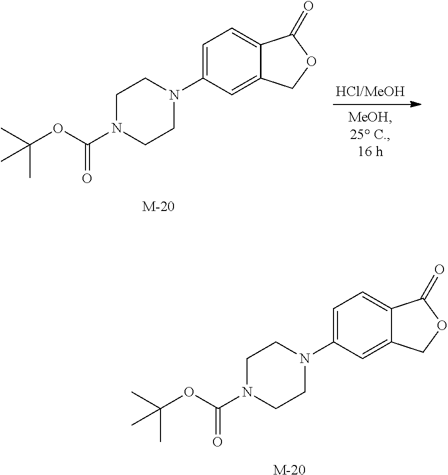 [SMILES: CC(C)(C)OC(=O)N1CCN(c2ccc3c(c2)COC3=O)CC1 ; "M-20" ; CC(C)(C)OC(=O)N1CCN(c2ccc3c(c2)COC3=O)CC1 ; "M-20"]

To a solution of M-20 (30.0 g, 1.0 eq) in DCM (60 mL) was added HCl/MeOH(150 ml, 6.37 eq, 4M). The mixture was stirred at 25° C. for 16 hr. TLC (Dichloromethane:Methanol=20:1, Rf=0.0) indicated Reactant M20 was consumed completely. The reaction mixture was concentrated under reduced pressure to remove MeOH and DCM. The crude product was washed with DCM (200 ml). Then it was filtered and concentrated under reduced pressure to give compound M-35 (27 g, crude) as a white solid. ¹H NMR: 400 MHz DMSO-d₆ 9.43 (s, 1H), 7.66 (d, J=8.4 Hz 1H), 7.13-7.18 (m, 2H), 5.28 (s, 2H), 3.60-3.63 (m, 4H), 3.16-3.19 (m, 4H).

General Procedure for Preparation of Compound M31

Compound M31 was prepared based on the same procedure for making compound 2 in Example 1.

General Procedure for Preparation of Compound M32

Compound M32 was prepared based on the same procedure for making compound 3 in Example 1.

General Procedure for Preparation of Compound M33

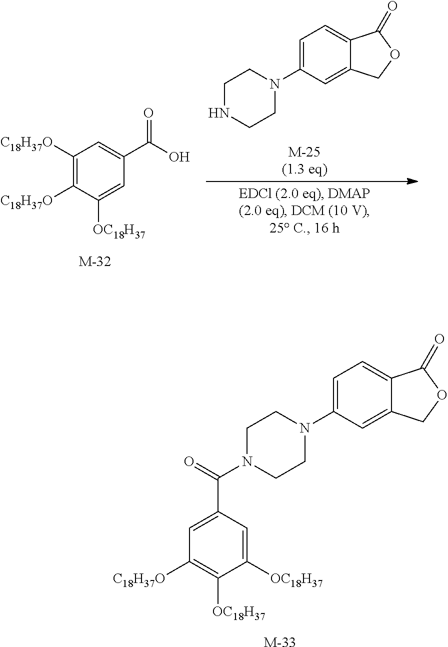

To a solution of compound M-32 (80.0 g, 1.0 eq) in DCM (50 mL) was added DMAP (21.1 g, 2.0 eq), M-25 (28.4 g, 1.3 eq) and EDCI (33.1 g, 2.0 eq). The mixture was stirred at 25° C. for 16 hr. TLC (Dichloromethane:Methanol=20:1, Rf=0.60) indicated Reactant M-32 was consumed completely. The reaction mixture was quenched by adding NaHCO3 (800 mL), and extracted with DCM (800 mL×3). The combined organic layers were washed with brine (800 mL), dried over anhydrous Na₂SO₄, filtered and concentrated under reduced pressure to give a crude product which was re-dissolve in DCM (160 mL) and dropped into ACN (4800 ml) with vigorous stirring. The solid was collected by filtration and dried under reduced pressure to give compound M-33 (96.0 g, 85.1 mmol, 98.69% yield) as a white solid. ¹H NMR: 400 MHz CDCl₃7.78 (d, J=8.4 Hz 1H), 7.78 (d, J=8.4 Hz 1H), 6.82 (s, 1H), 6.63 (s, 2H), 5.22 (s, 2H), 3.97(t, J=6.4 Hz 1H), 3.41-3.98 (m, 8H), 1.75-1.84 (m, 6H), 1.26-1.48 (m, 96H), 0.88(t, J=6.4 Hz 9H).

General Procedure for Preparation of Compound M34

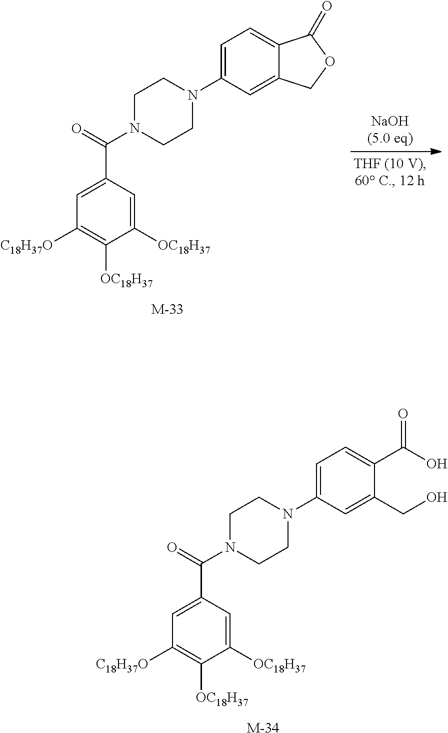

To a solution of M-33 (116 g, 1.0 eq) in THF (1160 mL) was added NaOH(20.56 g, 5.0 eq). The mixture was stirred at 60° C. for 12 hr. TLC (Dichloromethane:Methanol=20:1, Rf=0.50) indicated Reactant 1 was consumed completely. The reaction mixture was concentrated under reduced pressure to remove THF. The residue was diluted with H₂O 1.5 L. The solution was adjusted to pH=5˜6 with HCl (1M) and filtered and concentrated under reduced pressure to give a residue. The crude product was was co-evaporated with DCM, THF and ACN six times. Compound M-34 (87 g, 75.93 mmol, 73.82% yield) was obtained as a light yellow solid. ¹H NMR: 400 MHz CDCl₃ 8.01 (d, J=8.4 Hz 1H), 7.22 (s, 1H), 6.85 (s, 1H), 6.75 (d, J=8.8 Hz, 1H), 6.68 (s, 2H), 4.74 (s, 2H), 3.97,\(t, J=6.4 Hz, 6H), 3.41-3.98 (m, 8H), 1.70-1.81 (m, 6H), 1.26-1.48 (m, 96H), 0.83 (t, J=6.4 Hz, 9H),

General Procedure for Preparation of Compound M35

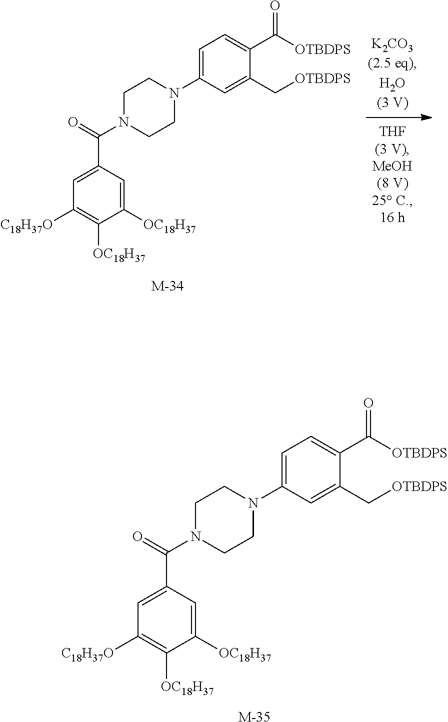

To a solution of compound M-34 (87 g, 1.0 eq) in DCM (870 mL) was added imidazole(14.96 g, 3.0 eq) and TBDPSCl (41.5 mL, 2.2 eq). The mixture was stirred at 25° C. for 16 hr. TLC (Dichloromethane:Methanol=20:1, R_{f}=0.90) indicated Reactant M-34 was consumed completely. The reaction mixture was quenched by addition of NaHCO₃600 mL, and extracted with DCM (700 mL×3). The combined organic layers were washed with brine (500 mL×2), dried over anhydrous Na₂SO₄, filtered and concentrated under reduced pressure to give a crude product which was re-dissolve in DCM (800 mL) and dropped into ACN (2.5 L) with vigorous stirring. The solid was filtered and concentrated under reduced pressure to give compound M-35 (118 g, crude) as a yellow solid. HPLC showed the starting material was consumed completely.

General Procedure for Preparation of Compound M36

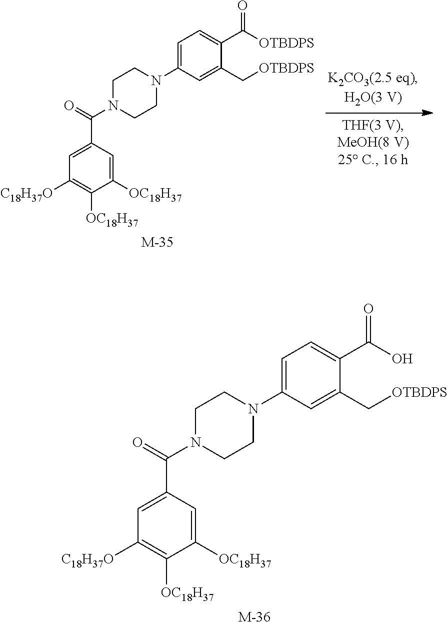

To a solution of compound M-35 (115 g, 1.0 eq) in THF (345 mL) and MeOH (920 mL) was added a solution of K₂CO₃ (24.72 g, 2.5 eq) in H₂O (345 mL). The mixture was stirred at 25° C. for 16 hr. TLC (Dichloromethane: Methanol=20:1, Rf=0.43) indicated compound M-35 was consumed completely. The reaction mixture was concentrated under reduced pressure to remove one quarter solvent. The residue was diluted with brine 450 mL and extracted with DCM (500 mL×4). The combined organic layers were washed with brine (500 mL×3), dried over anhydrous Na₂SO₄, filtered and concentrated under reduced pressure to give a residue. The residue was purified by column chromatography (SiO2, Dichloromethane/Ethyl acetate=1/0 to 10/1). Compound M-36 (25 g, 93% purity) was obtained as a white solid. ¹H NMR: 400 MHz CDCl₃7.99 (d, J=8.4 Hz, 1H), 7.67-7.69 (m, 4H), 7.33-7.40 (m, 7H), 6.75 (dd, J=2.0 Hz, J=8.8 Hz, 1H), 6.63 (s, 2H), 5.18 (s, 2H), 3.97-4.00,(m, 6H), 3.38-3.98 (m, 8H), 1.70-1.81 (m, 6H), 1.26-1.48 (m, 90H), 0.90 (s, 9H), 0.83 (t, J=6.4 Hz, 9H).

### Example 4. Synthesis of Compound M40

a. Scheme of M40 Synthesis

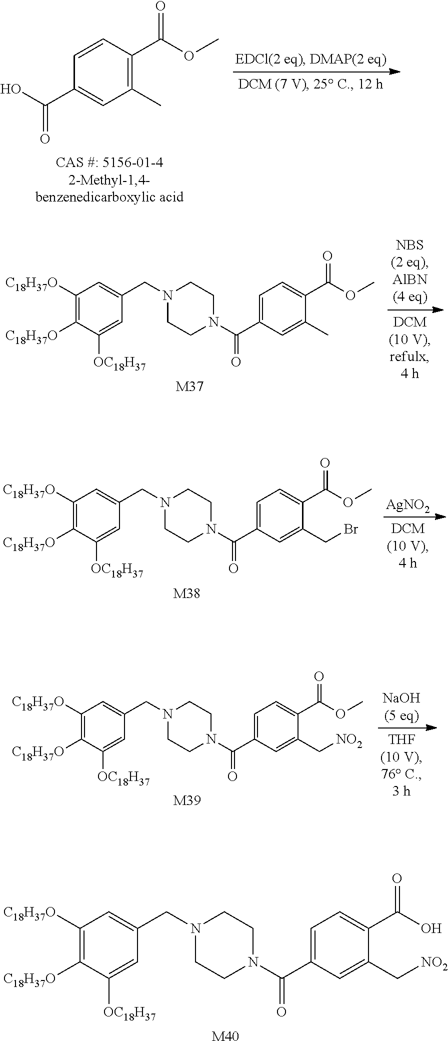

b. Procedures for Synthesis of Compound M40

General Procedure for Preparation of Compound M37

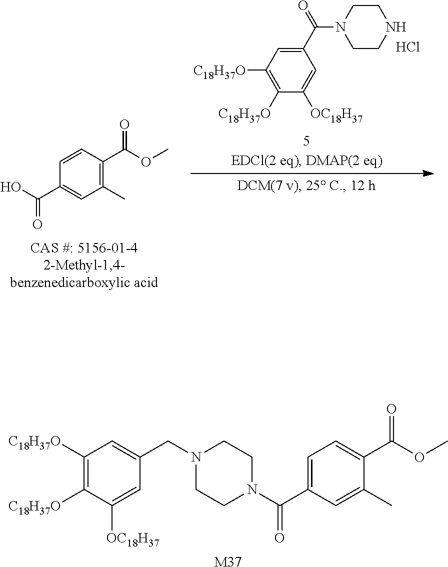

To a mixture of compound 2-Methyl-1,4-benzenedicarboxylic acid (1.1 eq) and compound 5 (1.10 eq) in DCM (7V) was added DMAP (2.00 eq), EDCI (2.00 eq) in one portion at 25° C. under N₂. The mixture was stirred at 25° C. for 2 h. The TLC indicated that the start material was consumed completely. The reaction mixture was poured into EtOH (50 V), filtered and washed with EtOH (10V). Compound M37 was obtained as a white solid.

General Procedure for Preparation of Compound M38

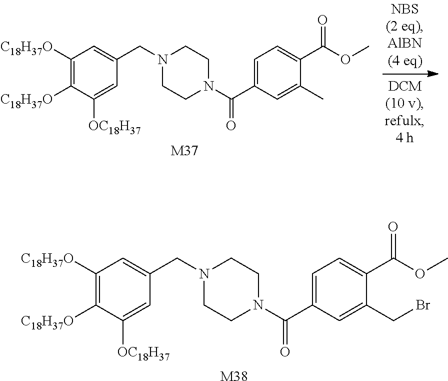

To a mixture of compound M37 (1.0 eq) and AIBN (4.0 eq) in DCM (10 V) was add NBS (2.0 eq) at 25° C. under N₂. The mixture was refluxed for 4 h, The TLC indicated that the start material was consumed completely. After cooling, washed the mixture with saturated aqueous NaHCO₃. Dried the mixture with MgSO₄ and concentrated the filtrate to residue which was precipitated in ACN to yield a white solid M38. See JOC, 2008, 73, 9125-9128.

General Procedure for Preparation of Compound M39

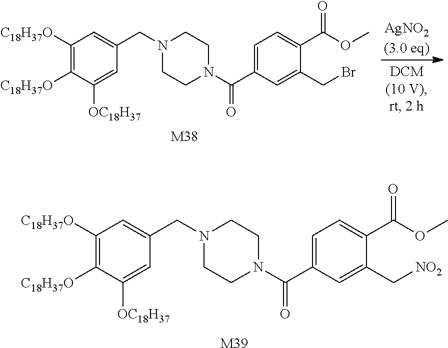

To a solution of compound M38 (1.0 eq) in DCM (10 V) was add AgNO₂ (3.0 eq) at 25° C. under N₂. The mixture was stirred for 2 h. The TLC indicated that the start material was consumed completely. Washed the mixture with saturated aqueous NaHCO₃. Dried the mixture with MgSO₄ and concentrated the filtrate to residue which was precipitated in ACN to yield a white solid M39. See Synthesis 1980, 814-815.

General Procedure for Preparation of Compound M40

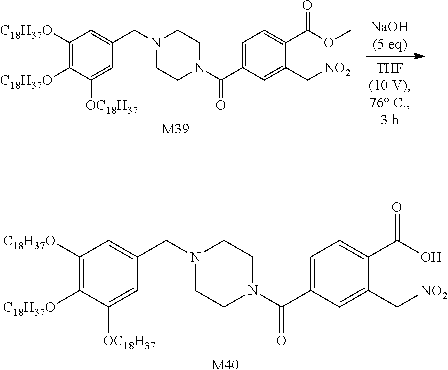

To a solution of compound M39 (1.0 eq) in THF (10 V) was added the solution of 1.0 M NaOH (5.0 eq) slowly in one portion at 25° C. under N₂. The mixture was stirred at 25° C. and stirred for 3 h. The TLC indicated the start material was consumed completely. Concentrated and diluted with H₂O (50 V), adjust pH to 4˜5 with 1N HCl. Filtered and washed with H₂O until the pH was 6-7. Washed with ACN. Compound M40 was obtained in 72% yield and 90% purity as a white solid.

### Example 5. Synthesis of Compound M50

a. Scheme for Synthesis of Compound M50

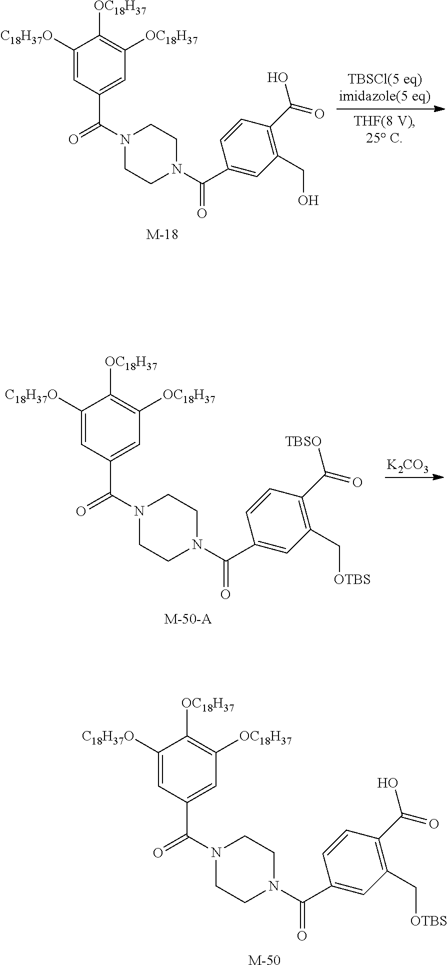

b. Procedures for Synthesis of Compound M50

General Procedure for Preparation of Compound M-50-A

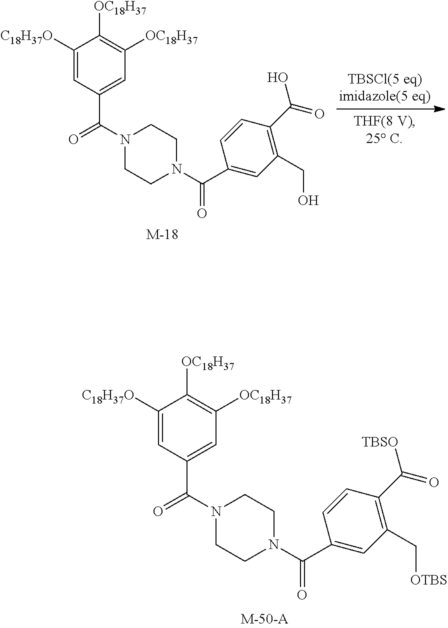

To a solution of compound M-18 (2.0 g, 1.70 mmol, 1.00 eq) in THF (16.0 mL) was added imidazole (579.99 mg, 8.52 mmol, 5.00 eq) and TBSCl (1.28 g, 8.52 mmol, 1.04 mL, 5.00 eq). The mixture was stirred at 25° C. for 2 hrs. HPLC showed the starting material was consumed completely. The residue was diluted with H₂O (50 mL) and extracted with DCM (2×30 mL). The combined organic layers were dried over anhydrous Na₂SO₄, filtered and concentrated under reduced pressure to give a residue. Compound M-50-A (2.39 g, crude) was obtained as a white solid.

General Procedure for Preparation of Compound M-50

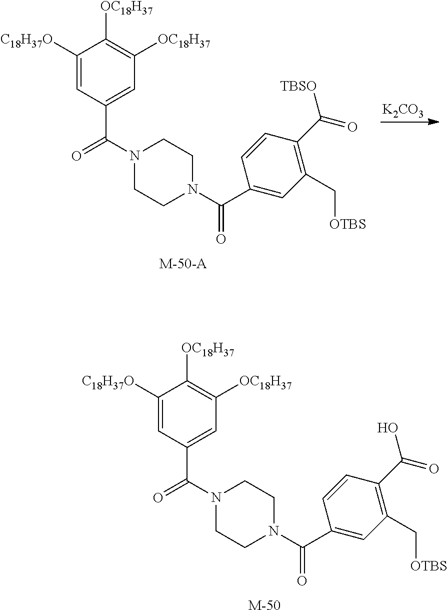

To a solution of compound M-50-A (2.39 g, 1.70 mmol, 1.00 eq) in THF (19.0 mL) and MeOH (2.30 mL) was added solution of K₂CO₃ (305.44 mg, 2.21 mmol, 1.30 eq) in H₂O (7.0 mL). The mixture was stirred at 25° C. for 3 hrs. TLC (DCM/MeOH=20/1, start material Rf=0.50, product Rf=0.30) indicated the start material was consumed completely. Adjust pH to 5 with 1 M KHSO₄ (5 mL). The residue was diluted with H₂O (50 mL) and extracted with DCM (2×30 mL). The combined organic layers were dried over anhydrous Na₂SO₄, filtered and concentrated under reduced pressure to give a residue. The crude product was triturated with ACN (30 V, 70.0 mL) at 25° C. for 30 min. Filtered and concentrated. Compound M-50 (1.00 g, 93.8% purity, 45.67% yield) was obtained as a white solid.

### Example 6. Synthesis of Compound M60

a. Scheme for Synthesis of Compound M60

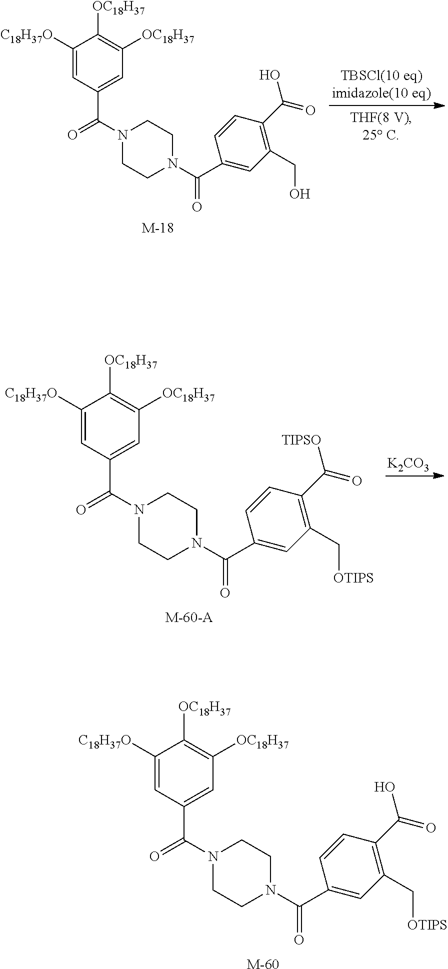

b. Procedures for Synthesis of Compound M60

General Procedure for Preparation of Compound M-60-A

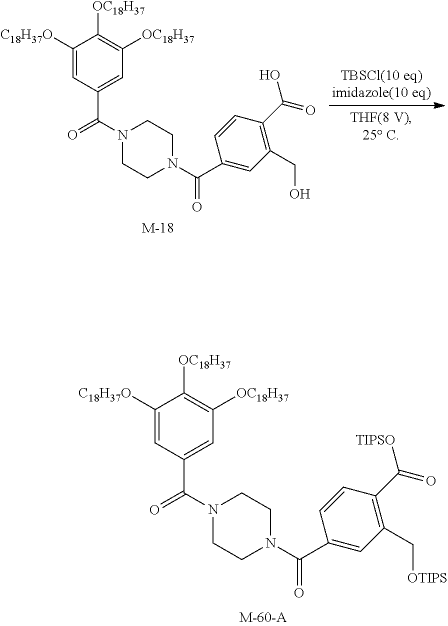

To a solution of compound M-18 (1.0 g, 0.85 mmol, 1.00 eq) in DCM (8.0 mL) was added imidazole (579.99 mg, 8.52 mmol, 10.0 eq) and TIPSCl (1.64 g, 8.52 mmol, 10.00 eq). The mixture was stirred at 25° C. for 8 hrs. HPLC showed the starting material was consumed completely. The residue was diluted with H₂O (50 mL) and extracted with DCM (2×30 mL). The combined organic layers were dried over anhydrous Na₂SO₄, filtered and concentrated under reduced pressure to give a residue. Compound M-60-A (1.27 g, crude) was obtained as a white solid.

General Procedure for Preparation of Compound M-60

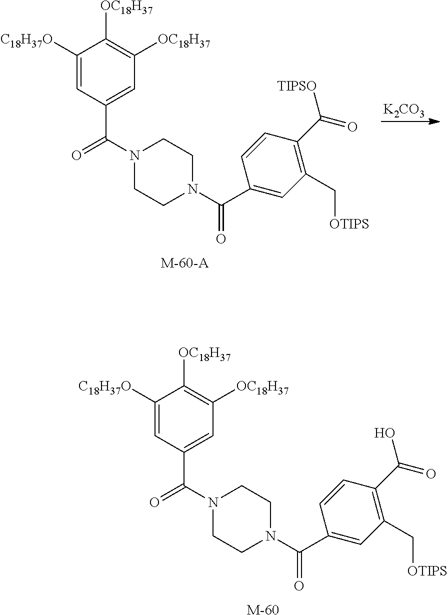

To a solution of compound M-60-A (1.27 g, 0.85 mmol, 1.00 eq) in THF (10.4 mL) and MeOH (1.30 mL) was added solution of K₂CO₃ (153.5 mg, 1.11 mmol, 1.30 eq) in H₂O (4.0 mL). The mixture was stirred at 25° C. for 3 hrs. Adjust pH to 5 with 1 M KHSO₄ (3 mL). The residue was diluted with H₂O (50 mL) and extracted with DCM (2×30 mL). The combined organic layers were dried over anhydrous Na₂SO₄, filtered and concentrated under reduced pressure to give a residue. The crude product was triturated with ACN (30 V, 70.0 mL) at 25° C. for 30 min. Filtered and concentrated to afford compound M-60 (0.90 g, 92% purity, 72.7% yield) as a white solid. Mass Calcd for C₈₃H₁₄₉N₂O₈Si⁺[M+H⁺]: 1330.1, found 1330.4.

### Example 7. Synthesis of Oligonucleotide Fragment A from Reagent M19

a. Scheme for Synthesis of Oligonucleotide Fragment A

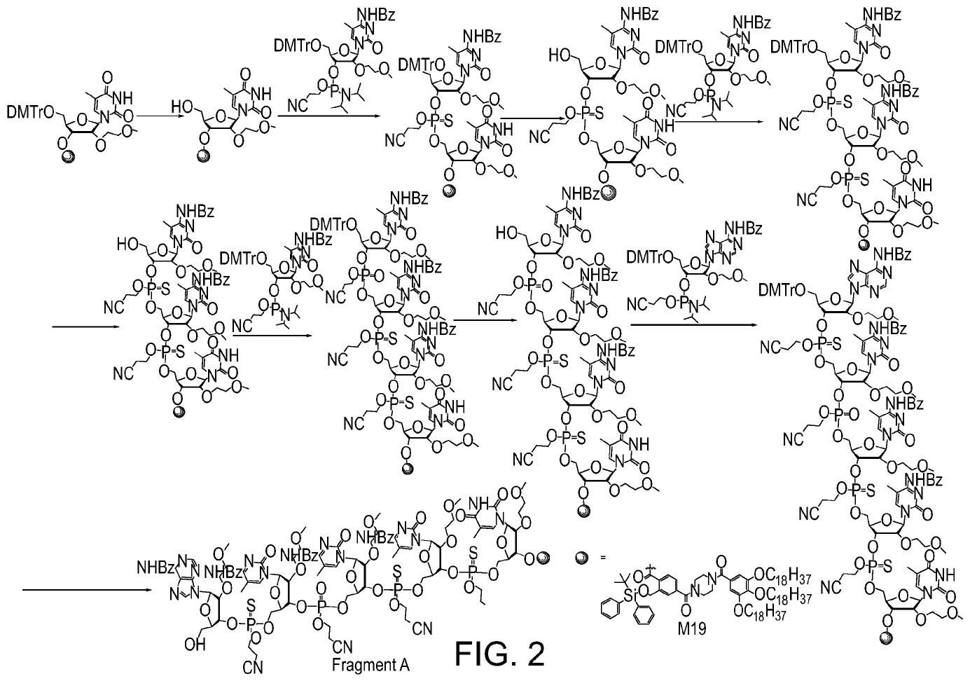

Fragment A was synthesized according to synthetic scheme depicted in FIG. 2.

b. Procedures for Synthesis of Oligonucleotide Fragment A from M19

General Procedure for Preparation of Compound M19-Fragment I-U-DMTr

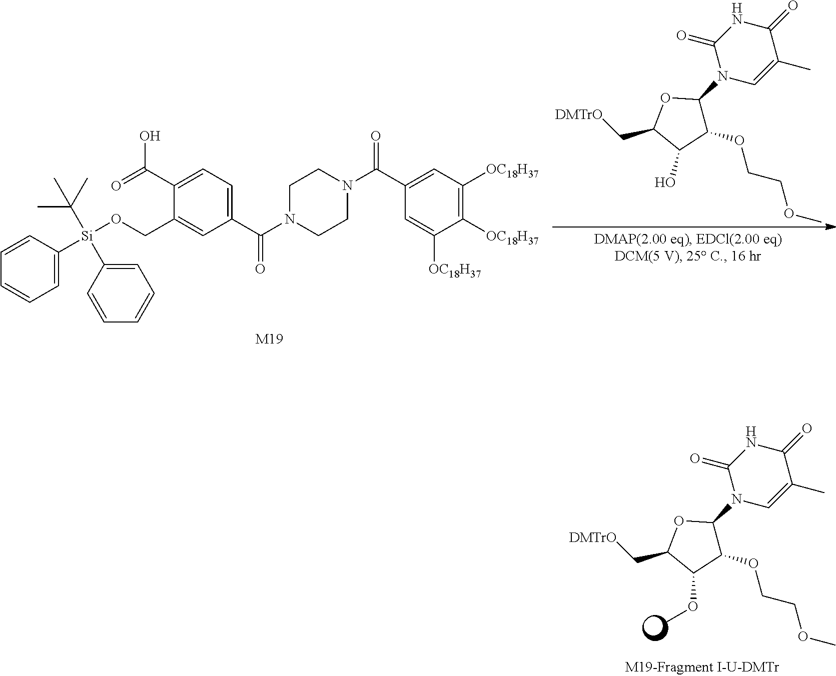

To a solution of M19 (20.00 g, 14.16 mmol, 1.00 eq) and dU (13.14 g, 21.24 mmol, 1.50 eq) in DCM (200 mL) was added DMAP (3.46 g, 28.32 mmol, 2.00 eq) and EDCI (5.43 g, 28.32 mmol, 2.00 eq). The mixture was stirred at 25° C. for 16 hr. TLC (Dichloromethane: Methanol=15:1, product, Rf=0.70) indicated that the reaction was complete and one new spot formed. The reaction was clean according to TLC. The reaction mixture was washed with NaHCO₃ (5% aq, 100 mL), dried over anhydrous Na₂SO₄, filtered and concentrated under reduced pressure to give a residue to 3 V. The crude was dropped into MeOH (600 ml, 30 V) with vigorous stirring. Desire product was precipitated out. Compound M19-Fragment I-U-DMTr (27.00 g, 13.31 mmol, 94.54% yield) was obtained as a white solid.

General Procedure for Preparation of Compound M19-Fragment I-U

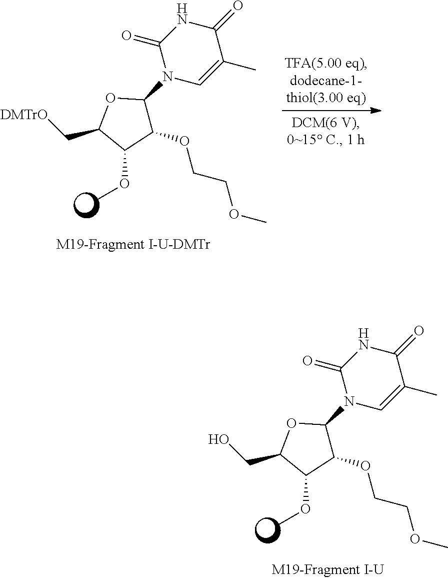

To a solution of M19-Fragment I-U-DMTr (27.0 g, 13.31 mmol, 1.00 eq) in DCM (170 mL) was added dodecane-1-thiol (8.15 g, 39.93 mmol, 9.64 mL, 3.00 eq) and TFA (7.59 g, 66.55 mmol, 4.92 mL, 5.00 eq) at 0° C. The mixture was stirred at 0° C. for 1 hr. TLC (Dichloromethane: Methanol=15:1, product, Rf=0.54) indicated Reactant was consumed completely and one new spot formed. The reaction was clean according to TLC. The reaction mixture was quenched by addition NaHCO₃ (5% aq. 100 mL), and then extracted with DCM (100 mL). The combined organic layers were washed with brine (100 mL×2), dried over anhydrous Na₂SO₄, filtered and concentrated under reduced pressure to give a residue. The crude was re-dissolve in DCM (30 mL) and dropped into MeOH/ACN (3:1, 450 ml) with vigorous stirring. Desire product was precipitated outCompound M19-Fragment I-U (15.00 g, 8.77 mmol, 65.51% yield, and 98.5% purity) was obtained as a white solid.

General Procedure for Preparation of Compound M19-Fragment I-UC

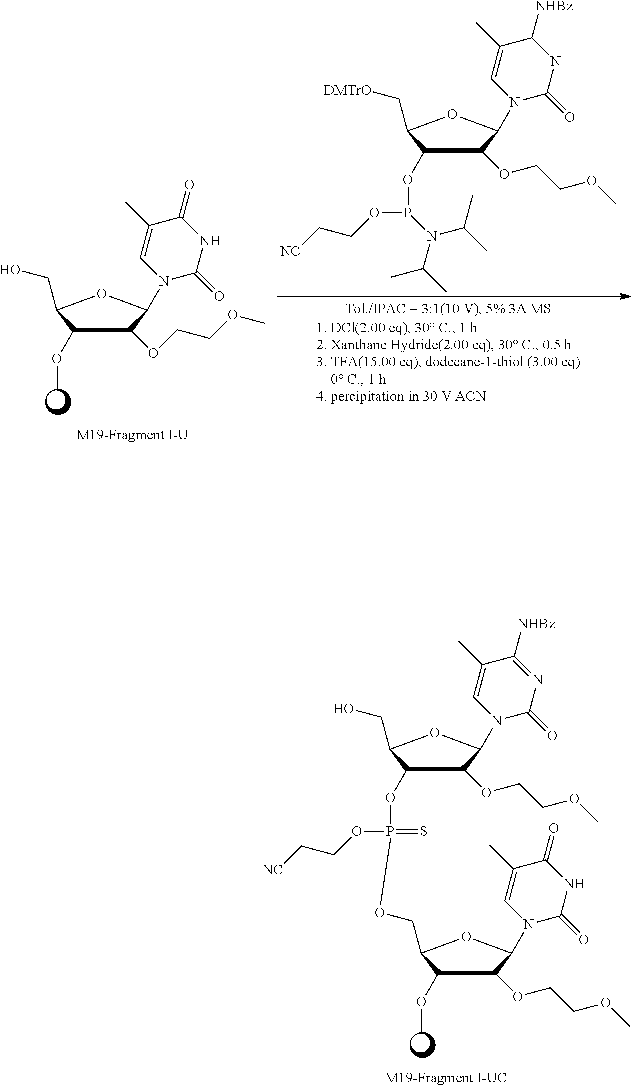

To a solution of M19-Fragment I-U (15.00 g, 8.76 mmol, 1.00 eq) and dC (12.12 g, 13.14 mmol, 1.50 eq) in Tol./IPAC=3:1 (120 mL) was added 3A MS(7.50 g) and stired for 1 hr. Then to the mixture was added DCI (2.07 g, 17.55 mmol, 2.00 eq). The mixture was stirred at 30° C. for 1 hr. TLC (Dichloromethane: Methanol=20:1, product: R_{f}=0.53) indicated the reactant M19-Fragment I-U were consumed completely and one new spot formed. The reaction was clean according to TLC. To the crude was added HX (2.64 g, 17.55 mmol, 2.00 eq). The mixture was stirred at 30° C. for 0.5 hr, and then to the mixture was added dodecane-1-thiol (5.31 g, 26.31 mmol, 6.30 mL, 3.00 eq) and TFA (15.00 g, 134.49 mmol, 9.75 mL, 15.00 eq) at 0° C. The mixture was stirred at 0° C. for 1 hr. TLC (Dichloromethane: Methanol=20:1, product: R_{f}=0.43) indicated the reactant was consumed completely and one new spot formed. The reaction was clean according to TLC. The reaction mixture was diluted with NMI (175.2 mmol, 14 mL, 20.00 eq) to pH 7, filtered and concentrated under reduced pressure to give a residue. The filter liquor was dropped into ACN (800 ml), precipitated for 0.5 h, filtered for 3.5 h with 9 cm Buchner funnel to give 14.3 g (98% yield, 97.7% purity) as a white solid.

General Procedure for Preparation of Compound M19-Fragment I-UCC

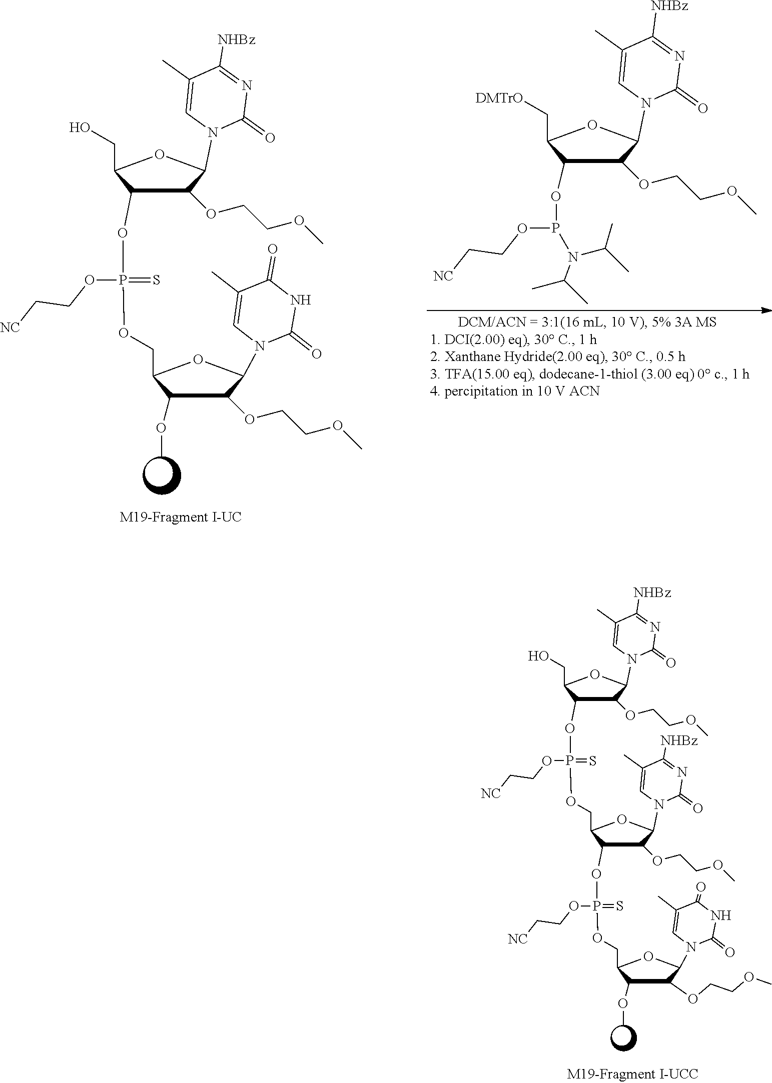

To a solution of M19-Fragment I-UC (19.00 g, 8.40 mmol, 1.00 eq) and dC (11.55 g, 12.60 mmol, 1.50 eq) in Tol./IPAC=3:1 (150 mL) was added 3A molecular sieve(7.5 g). The mixture was stirred for 1 hr, followed by DCI (1.97 g, 16.80 mmol, 2.00 eq). Then it was stirred at 30° C. for 1 hr. TLC (Dichloromethane: Methanol=20:1, product: Rf=0.53) indicated the compound M19-Fragment I-UC was consumed completely and one new spot was formed. The reaction was clean according to TLC. To the mixture was added HX (2.51 g, 16.80 mmol, 2.00 eq). The mixture was stirred at 30° C. for 0.5 hr, followed by dodecane-1-thiol (5.04 g, 25.20 mmol, 5.94 mL, 3.00 eq) and TFA (14.16 g, 126 mmol, 9.21 mL, 15.00 eq) at 0° C. The mixture was stirred at 0° C. for 1 hr. TLC (Dichloromethane: Methanol=15:1, product: Rf=0.43) indicated the reaction was complete and one new spot was formed. The reaction was clean according to TLC. To the reaction mixture was added NMI (168 mmol, 14.5 mL, 20.00 eq). The mixture was dropped into ACN (800 mL). The solid was precipitated for 0.5 h and filtered for 2.0 h with 15 cm Buchner funnel to afford 15.4 g (94% yield, 95.0% purity) as a white solid.

General Procedure for Preparation of Compound M19-Fragment I-UCCC

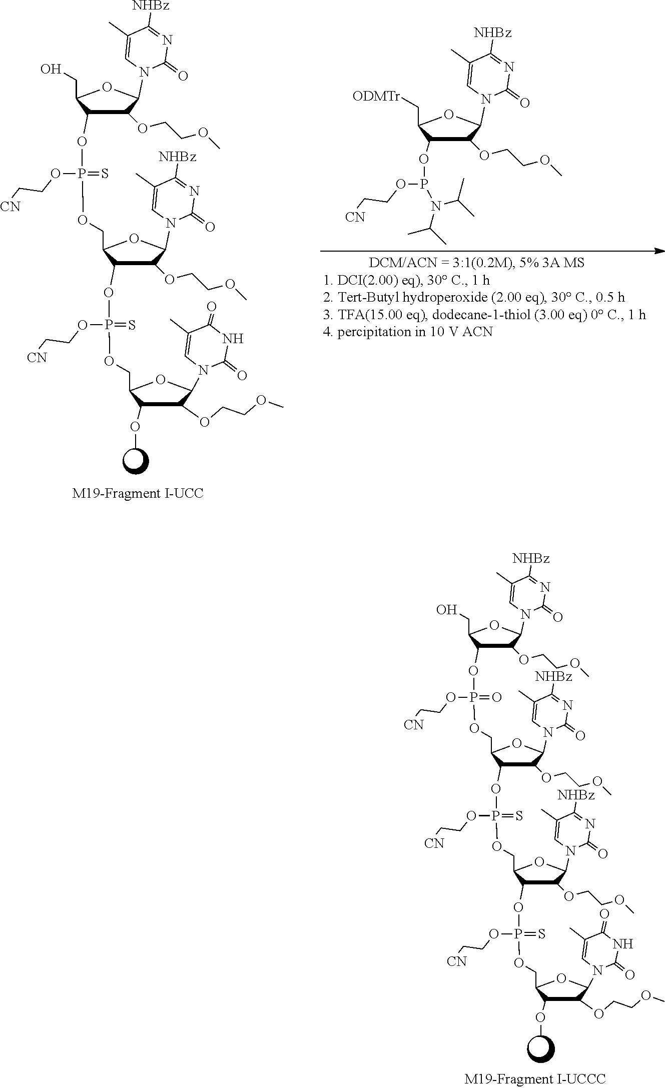

To a solution of M19-Fragment I-UCC (5.00 g, 1.78 mmol, 1.00 eq) and dC (2.46 g, 2.67 mmol, 1.50 eq) in Tol./ACN=3:1 (40 mL) was added 3A MS(2.0 g). The mixture was stirred at 30° C. for 1 hr, followed by DCI (420.05 mg, 3.56 mmol, 2.00 eq). The mixture was stirred at 30° C. for 1 hr. TLC (Dichloromethane: Methanol=15:1, Product: R_{f}=0.53) indicated that the reaction was complete and one new spot was formed. The reaction was clean according to TLC. To the mixture was added tert-butyl hydroperoxide (320.5 mg, 3.56 mmol, 2.00 eq). The mixture was stirred at 30° C. for 0.5 hr, followed by dodecane-1-thiol (1.08 g, 5.34 mmol, 1.28 mL, 3.00 eq) and TFA (3.04 g, 26.68 mmol, 1.98 mL, 15.00 eq) at 0° C. The mixture was stirred at 0° C. for 1 hr. TLC (Dichloromethane: Methanol=20:1, product: R_{f}=0.43) indicated that the reaction was complete and one new spot formed. The reaction mixture was quenched by addition NaHCO₃ (2%) 100 mL and Na₂SO₃ (2 eq) at 0° C., filtered and extracted with DCM (80 mL×3). The combined organic layers were washed with brine (100 mL×2), dried over anhydrous Na₂SO₄, filtered and concentrated under reduced pressure to give a residue, which was dropped into ACN (200 mL) with vigorous stirring. Desire product was precipitated out. The cake was washed with ACN (30 mL*2), filtered for 1.5 h with 7 cm Buchner funnel to afford 5.00 g (84% yield, 90.9% purity) as a white solid.

General Procedure for Preparation of Oligonucleotide Fragment A

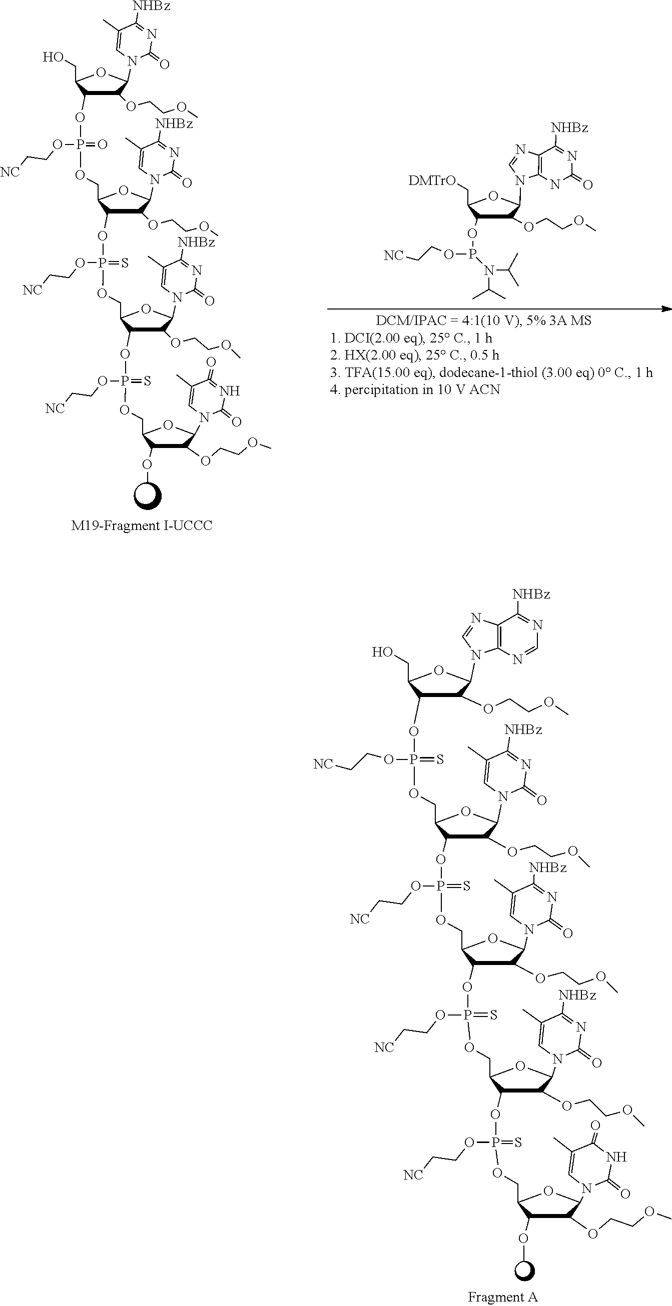

To a solution of M19-Fragment I-UCCC (4.00 g, 1.20 mmol, 1.00 eq) and dA (1.67 g, 1.79 mmol, 1.50 eq) in Tol./ACN=3:1 (40 mL) was added 3A MS(2.0 g). The mixture was stirred at 30° C. for 1 hr, folloed by DCI (282.36 mg, 2.39 mmol, 2.00 eq). The mixture was stirred at 30° C. for 1 hr. TLC (Dichloromethane: Methanol=20:1, Product: R_{f}=0.53) indicated that the reaction was complete and one new spot was formed. The reaction was clean according to TLC, and then added glucose (0.5 eq). To the mixture was added HX (358.99 mg, 2.39 mmol, 2.00 eq). The mixture was stirred at 30° C. for 0.5 hr, and then was added dodecane-1-thiol (725.66 mg, 3.59 mmol, 0.86 mL, 3.00 eq) and TFA (2.04 g, 17.93 mmol, 1.33 mL, 15.00 eq) at 0° C. The mixture was stirred at 0° C. for 1 hr. TLC (Dichloromethane: Methanol=15:1, product: R_{f}=0.43) indicated the reaction was complete and one new spot was formed. The reaction mixture was quenched by addition of NaHCO₃ (2%) 80 mL at 0° C., filtered and extracted with DCM (50 mL×3). The combined organic layers were washed with brine (80 mL×2), dried over anhydrous Na₂SO₄, filtered and concentrated under reduced pressure to give a residue, which was dropped into ACN (200 mL) with vigorous stirring. Desire product was precipitated out. The cake was washed with ACN (30 mL*2), filtered for 1.5 h with 7 cm Buchner funnel, to afford 4.20 g (85% yield, 86.4% purity) of fragment A as a white solid.

### Example 8. Synthesis of Oligonucleotide Fragment B from Reagent M19

a. Scheme for Synthesis of Oligonucleotide Fragment B

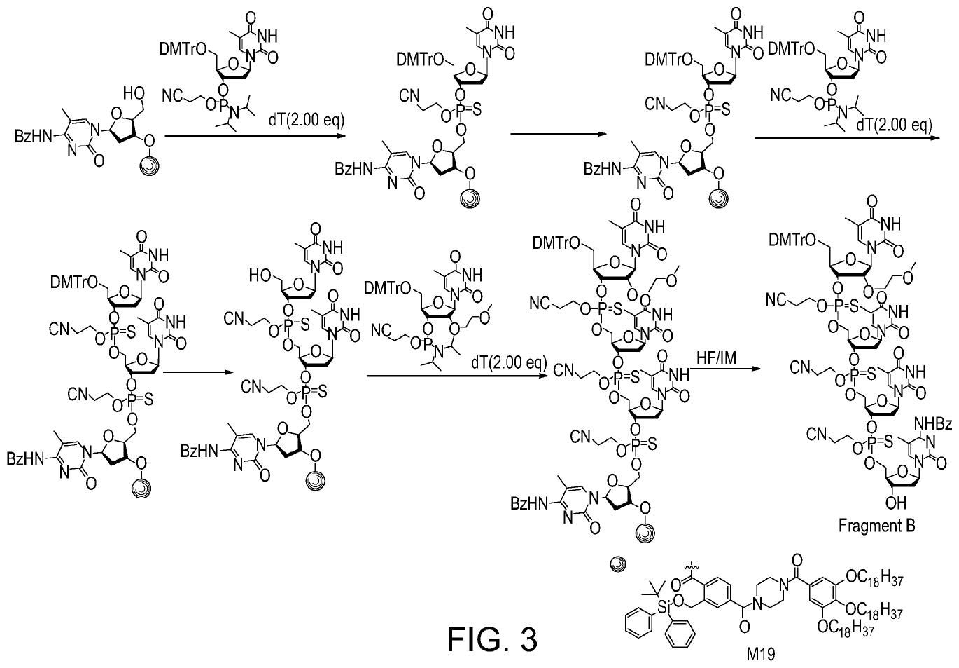

Fragment B was synthesized according to synthetic scheme depicted in FIG. 3.

b. Procedures for Synthesis of Oligonucleotide Fragment B from M19

General Procedure for Preparation of Compound M19-Fragment III-C-DMTr

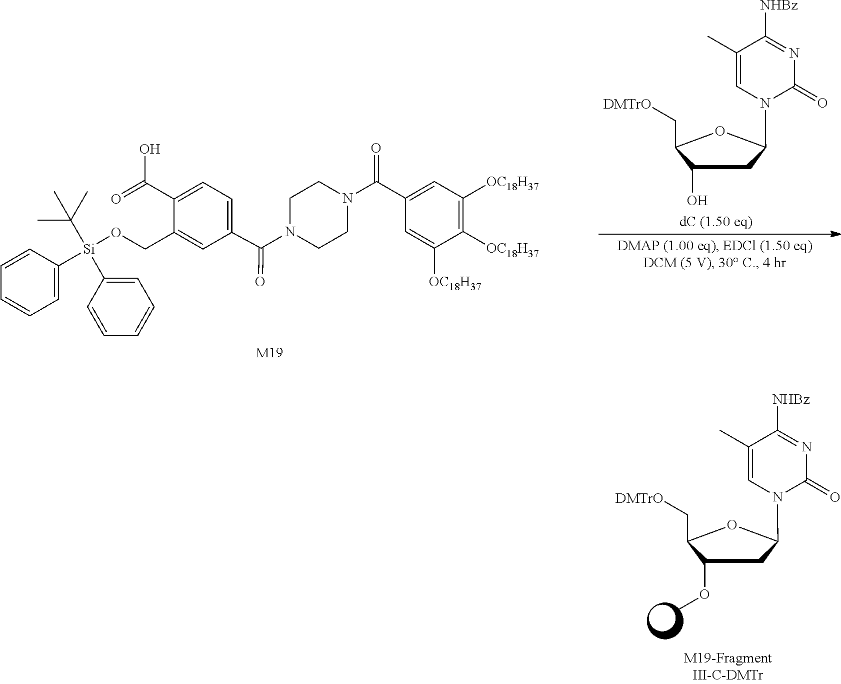

To a solution of M19 (20.00 g, 14.16 mmol, 1.00 eq) and dC (13.76 g, 21.24 mmol, 1.50 eq) in DCM (200 mL) was added DMAP (1.73 g, 14.16 mmol, 1.00 eq) and EDCI (4.08 g, 21.24 mmol, 1.50 eq). The mixture was stirred at 30° C. for 12 hr. TLC (Dichloromethane: Methanol=10:1, product, Rf=0.70) indicated that the reaction was complete and one new spot was formed. The reaction was clean according to TLC. The reaction mixture was washed with NaHCO₃ (300 mL), dried over anhydrous Na₂SO₄, filtered and concentrated under reduced pressure to give a residue to 5 V, which was dropped into ACN/MeOH (3:1, 1000 ml, 50 V) with vigorous stirring. Desired product was precipitated out. Compound M19-Fragment III-C-DMTr (28.90 g, 14.15 mmol, 99.94% yield) was obtained as a white solid.

General Procedure for Preparation of Compound M19-Fragment III-C

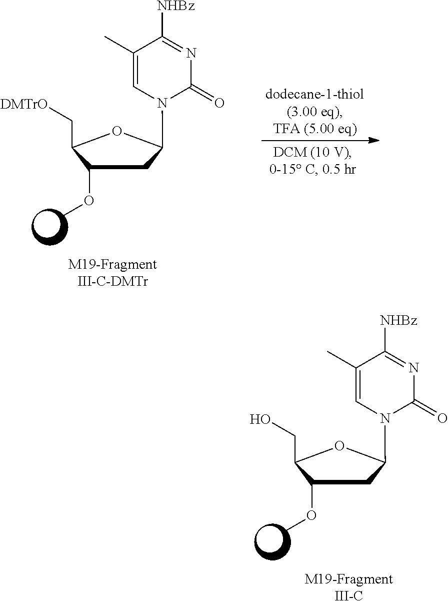

To a solution of M19-Fragment III-C-DMTr (28.0 g, 13.72 mmol, 1.00 eq) in DCM (250 mL) was added dodecane-1-thiol (8.32 g, 41.12 mmol, 9.84 mL, 3.00 eq) and TFA (7.80 g, 68.56 mmol, 5.08 mL, 5.00 eq). The mixture was stirred at 0° C. for 1 hr. TLC (Dichloromethane: Methanol=10:1, product, Rf=0.54) indicated that the reaction was complete and one new spot was formed. The reaction was clean according to TLC. The reaction mixture was diluted with NMI (9.00 g, 109.72 mmol, 8.76 mL, 8.00 eq). The crude was dropped into ACN (900 ml, 30 V) with vigorous stirring. Desired product was precipitated out. Compound M19-Fragment III-C(23.00 g, 13.22 mmol, 96.42% yield) was obtained as a white solid.

General Procedure for Preparation of Compound M19-Fragment III-CT

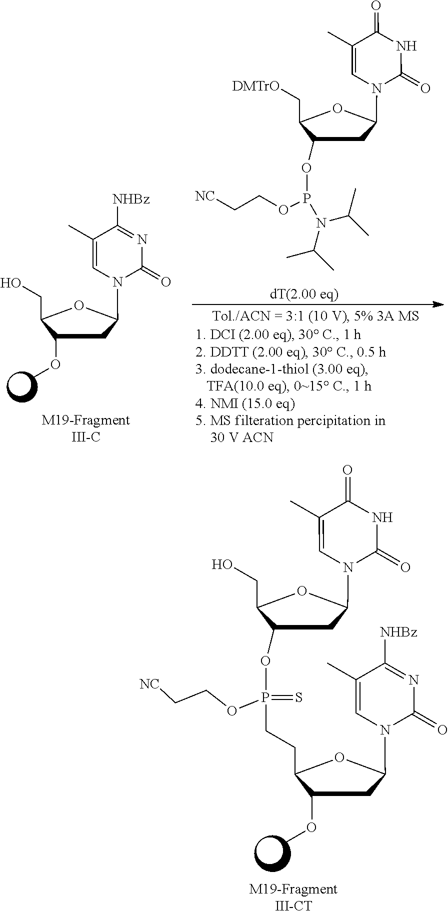

To a solution of M19-Fragment III-C(16.00 g, 9.20 mmol, 1.00 eq) and dT (10.28 g, 13.80 mmol, 1.50 eq) in Tol./ACN=3:1 (140 mL) was added 3A MS(7.00 g) and stired for 1 hr. The mixture was added DCI (2.17 g, 18.40 mmol, 2.00 eq). The mixture was stirred at 25° C. for 1 hr. TLC (Dichloromethane: Methanol=20:1, product: Rf=0.53) indicated that the reaction was complete and one new spot was formed. The reaction was clean according to TLC. To the reaction mixture was added HX (2.77 g, 18.42 mmol, 2.00 eq). The mixture was stirred at 30° C. for 0.5 hr, followed by dodecane-1-thiol (5.61 g, 27.70 mmol, 6.63 mL, 3.00 eq) and TFA (10.53 g, 92.33 mmol, 6.84 mL, 10.00 eq) at 0° C. The mixture was stirred at 0° C. for 0.5 hr. TLC (Dichloromethane: Methanol=20:1, product: Rf=0.45) indicated that the reaction was complete and one new spot was formed. The reaction was clean according to TLC. The reaction mixture was diluted with NMI (11.37 g, 138.50 mmol, 11.04 mL, 15.00 eq). The crude was dropped into ACN (500 ml, 30 V), precipitated for 0.5 h, filtered for 1.5 h with 15 cm Buchner funnel to give 14.0 g (93.30% yield, 97.28% purity) M19-Fragment III-CT as a white solid.

General Procedure for Preparation of Compound M19-Fragment III-CTT

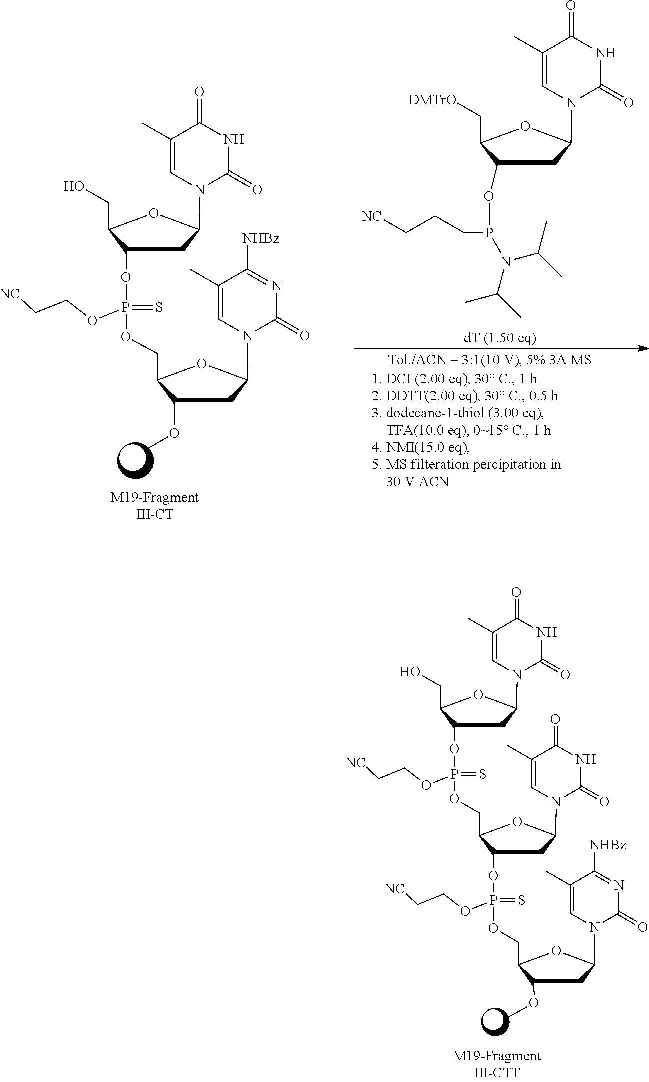

To a solution of M19-Fragment III-CT (15.00 g, 7.10 mmol, 1.00 eq) and dT (7.93 g, 10.65 mmol, 1.50 eq) in Tol./ACN=3:1 (120 mL) was added 3A molecular sieve(2.00 g). The mixture was stirred for 1 hr, followed by DCI (1.68 g, 14.20 mmol, 2.00 eq). The mixture was stirred at 30° C. for 1 hr. TLC (Dichloromethane: Methanol=20:1, product: Rf=0.42) indicated that the reaction was complete and one new spot was formed. The reaction was clean according to TLC. The crude was added HX (2.16 g, 14.37 mmol, 2.00 eq). The mixture was stirred at 30° C. for 0.5 hr, and then was added dodecane-1-thiol (4.37 g, 21.57 mmol, 5.17 mL, 3.00 eq) and TFA (8.20 g, 71.90 mmol, 5.32 mL, 10.00 eq) at 0° C. The mixture was stirred at 0° C. for 0.5 hr. TLC (Dichloromethane: Methanol=10:1, product: Rf=0.54) indicated the reaction was complete and one new spot was formed. The reaction was clean according to TLC. The reaction mixture was added NMI (8.86 g, 107.85 mmol, 8.60 mL, 15.00 eq). The mixture was dropped into ACN (200 mL, 30 V), precipitated for 0.5 h, filtered for 1.0 h with 7 cm Buchner funnel, to afford 5.76 g (96.60% yield, 93.99% purity) of M19-Fragment III-CTT as a white solid.

General Procedure for Preparation of Compound M19-Fragment III-CTTU-DMTr

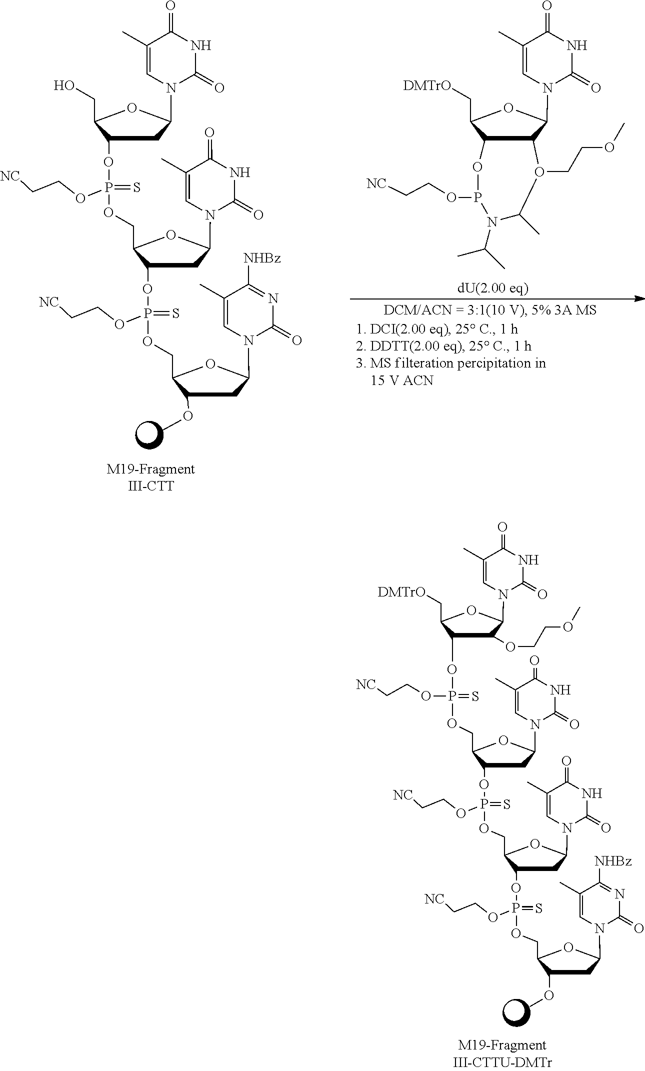

To a solution of M19-Fragment III-CTT (10.5 g, 4.22 mmol, 1.00 eq) and dU (5.19 g, 6.34 mmol, 1.50 eq) in Tol./ACN=3:1 (80 mL) was added 3A MS (0.5 g). The mixture was stirred at 25° C. for 1 hr, followed by DCI (997.52 mg, 8.45 mmol, 2.00 eq). The mixture was stirred at 25° C. for 1 hr. TLC (Dichloromethane: Methanol=10:1, Product: Rf=0.50) indicated that the reaction was complete and one new spot was formed. The reaction was clean according to TLC. The crude was added HX (1.74 g, 8.46 mmol, 2.00 eq). The mixture was stirred at 25° C. for 0.5 hr. The reaction mixture was filtered and concentrated under reduced pressure to give a residue to 5V, which was dropped into ACN (150 mL, 30 V), precipitated for 0.5 h, filtered for 1.0 h with 7 cm Buchner funnel, to afford 4.22 g of M19-Fragment III-CTTU-DMTr (92.5% yield, 91.7% purity) as a white solid.

General Procedure for Preparation of Oligonucleotide Fragment B

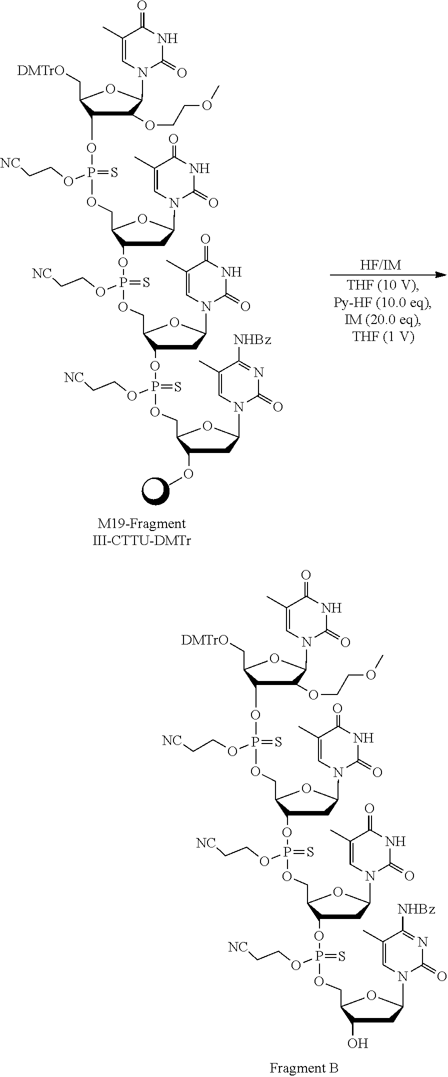

To a solution of imidazole (631.13 mg, 9.27 mmol, 20.00 eq) in THF (2.00 mL) was dropwise added pyridine hydrofluoride (132.51 mg, 4.64 mmol, 120.46 uL, 70% purity, 10.00 eq) at 0° C. The mixture was added into the solution of M19-Fragment III-CTTU-DMTr (1.50 g, 463.54 umol, 1.00 eq) in THF (15.00 mL). The mixture was stirred at 0-20° C. for 1 hr. TLC (Dichloromethane: Ethyl acetate: Methanol=20:10:1, Product: Rf=0.05) indicated that the reaction was complete and one new spot was formed. The reaction was clean according to TLC. The reaction mixture was washed with NaHCO₃ (30 mL), DI water to PH=7, dried over anhydrous Na₂SO₄, filtered and concentrated under reduced pressure. The crude was dissolved with DCM (3 ml, 2V) and dropped into TBME (50 ml) with vigorous stirring. Desired product was precipitated out. Oligonucleotide II (800 mg, 434.37 umol, 93.71% yield) was obtained as a white solid.

### Example 9. Synthesis of Oligonucleotide Fragment C

a. Scheme for Synthesis of Oligonucleotide Fragment C from M22

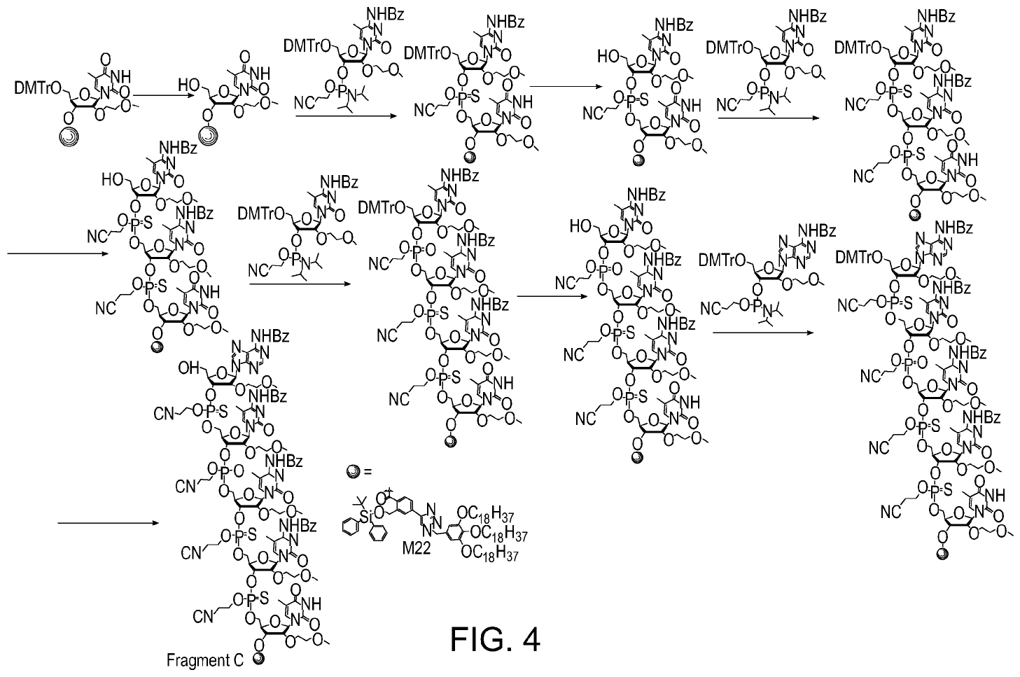

Fragment C was synthesized according to synthetic scheme depicted in FIG. 4.

b. Procedures for Synthesis of Oligonucleotide Fragment C from M22

General Procedure for Preparation of Compound M22-Fragment I-U-DMTr

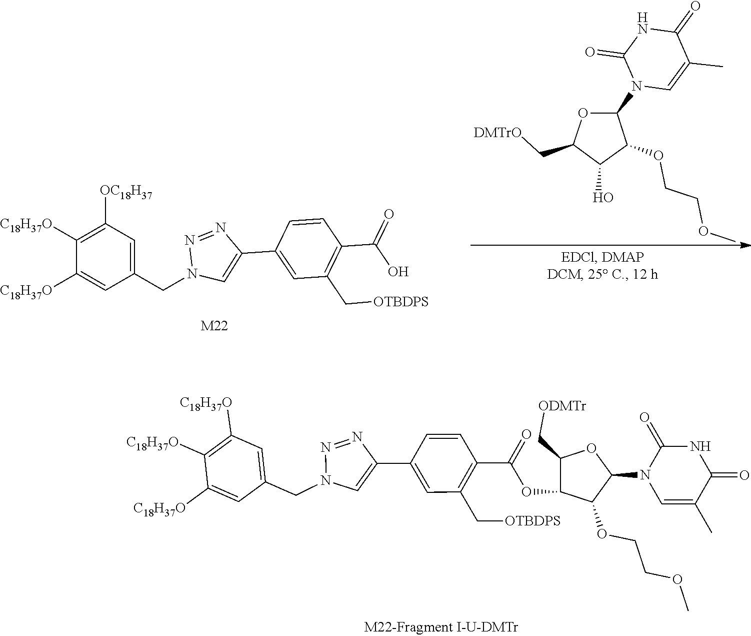

To a solution of M22 (3.10 g, 2.30 mmol) and compound U-DMTr (2.84 g, 4.58 mmol) in DCM (18.0 mL) was added DMAP (560 mg, 4.58 mmol) and EDCI (879 mg, 4.58 mmol). The mixture was stirred at 25° C. for 12 h. TLC (Petroleum ether/Ethyl acetate=1/1, product: Rf=0.34) indicated M22 was consumed, and one major new spot was detected. The reaction mixture was concentrated under reduced pressure. The residue was diluted with DCM (100 mL) and washed with saturated NaHCO₃ solution (40.0 mL×3). The combined organic layers were washed with brine (100 mL×3), dried over anhydrous Na₂SO₄, filtered and concentrated under reduced pressure to give a residue. The crude product was re-dissolve in DCM (60.0 mL) and dropped into MeOH/ACN (3/1, 300 mL) with vigorous stirring. Desired product was precipitated out, filtered and concentrated under reduced pressure to give a residue. M22-Fragment I-U-DMTr (5.20 g, crude) was obtained as a white solid.

General Procedure for Preparation of Compound M22-Fragment I-U

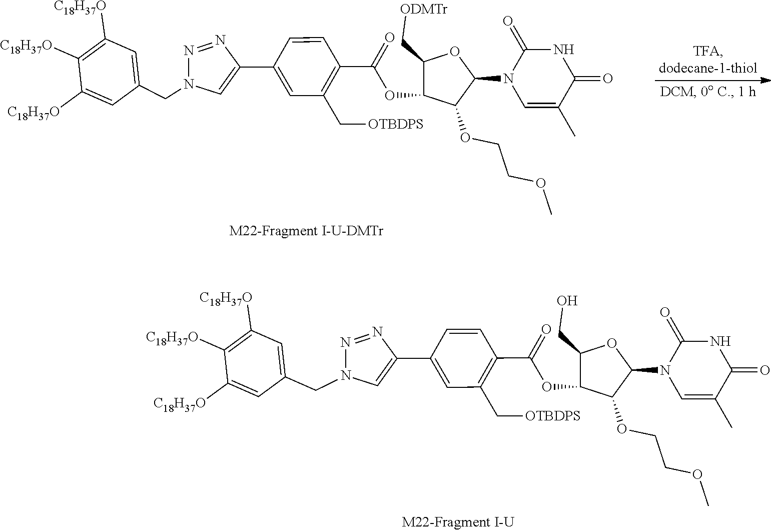

To a solution of M22-Fragment I-U-DMTr (5.10 g, 2.61 mmol) in DCM (30.0 mL) was added dodecane-1-thiol (1.58 g, 7.83 mmol, 1.88 mL) and TFA (2.38 g, 20.9 mmol, 1.55 mL). The mixture was stirred at 0° C. for 1 h. TLC (Petroleum ether/Ethyl acetate=1/1, product: R_{f}=0.28) indicated M22-Fragment I-U-DMTr was consumed completely and two new spots formed. The reaction mixture was quenched by addition Py (26.0 eq) at 0° C., and then diluted with DCM (150 mL) and washed with saturated NaHCO₃ aqueous solution (150 mL×4). The combined organic layers were washed with brine (150 mL×3), dried over anhydrous Na₂SO₄ Na₂SO₄ (15.0 g), filtered and concentrated under reduced pressure to give a residue. The residue was purified by column chromatography (SiO₂, DCM/MeOH=150/1 to 100/1). M22-Fragment I-U (2.90 g, 64.6% yield, 96.0% purity) was obtained as a white solid.

General Procedure for Preparation of Compound M22-Fragment I-UC

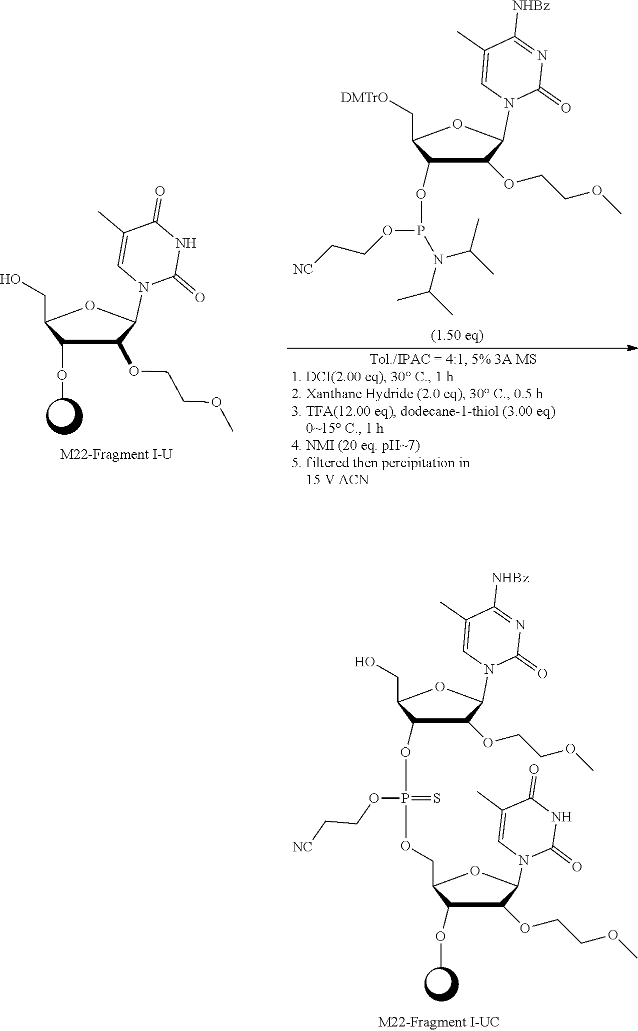

To a solution of M22-Fragment I-U (2.00 g, 1.21 mmol) and C-DMTr (1.67 g, 1.82 mmol) in Tol. (9.60 mL) and IPAC (2.40 mL) was added DCI (286 mg, 2.42 mmol) and Molecular sieve 3A (2.00 g). The mixture was stirred at 30° C. for 1 h. TLC (Dichloromethane/Methanol=20/1, product: R_{f}=0.43) indicated M22-Fragment I-U was consumed completely and many new spots formed. Then Xanthane Hydride (365 mg, 2.42 mmol) was added into the reaction mixture. Then mixture was stirred at 30° C. for 0.5 h. Then dodecane-1-thiol (727 mg, 3.60 mmol, 860 uL) and TFA (2.04 g, 18.0 mmol, 1.33 mL). The mixture was stirred at 0° C. for 1 h. TLC (Dichloromethane/Methanol=20/1, product: R_{f}=0.34) indicated M22-Fragment I-UC-DMTr was consumed completely and many new spots formed. The reaction mixture was quenched by addition NMI (20.0 eq) and then filtered. Then it was triturated by ACN (300 mL) and filtered. M22-Fragment I-UC (2.64 g, 95.2% yield, 95.1% purity) was obtained as a white solid.

General Procedure for Preparation of Compound M22-Fragment I-UCC

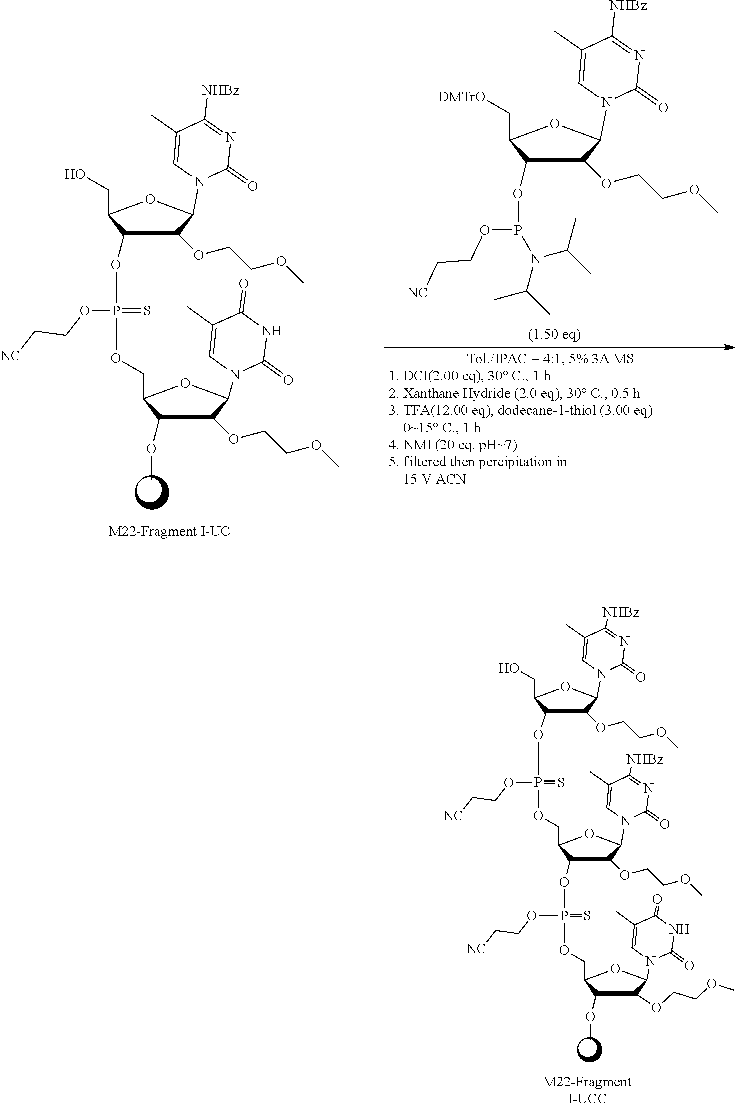

To a solution of M22-Fragment I-UC (2.60 g, 1.18 mmol) and C-DMTr (1.64 g, 1.77 mmol) in Tol. (12.6 mL) and IPAC (3.00 mL) was added DCI (279 mg, 2.36 mmol) and Molecular sieve 3A (2.00 g). The mixture was stirred at 30° C. for 1 h. TLC (Dichloromethane/Methanol=20/1, product: Rf=0.48) indicated M22-Fragment I-UC was consumed completely and many new spots formed. Then Xanthane Hydride (358 mg, 2.38 mmol) was added into the reaction mixture. The mixture was stirred at 30° C. for 0.5 h. Then dodecane-1-thiol (716 mg, 3.54 mmol, 846 uL) and TFA (2.02 g, 17.7 mmol, 1.31 mL). The mixture was stirred at 0° C. for 1 h. TLC (Dichloromethane/Methanol=20/1, product: Rf=0.41) indicated M22-Fragment I-UCC-DMTr was consumed completely and two new spots formed. The reaction mixture was quenched by addition NMI (20.0 eq) and then filtered. Then it was triturated by ACN (300 mL) and filtered. M22-Fragment I-UCC (3.23 g, crude) was obtained as a white solid.

General Procedure for Preparation of Compound M22-Fragment I-UCCC

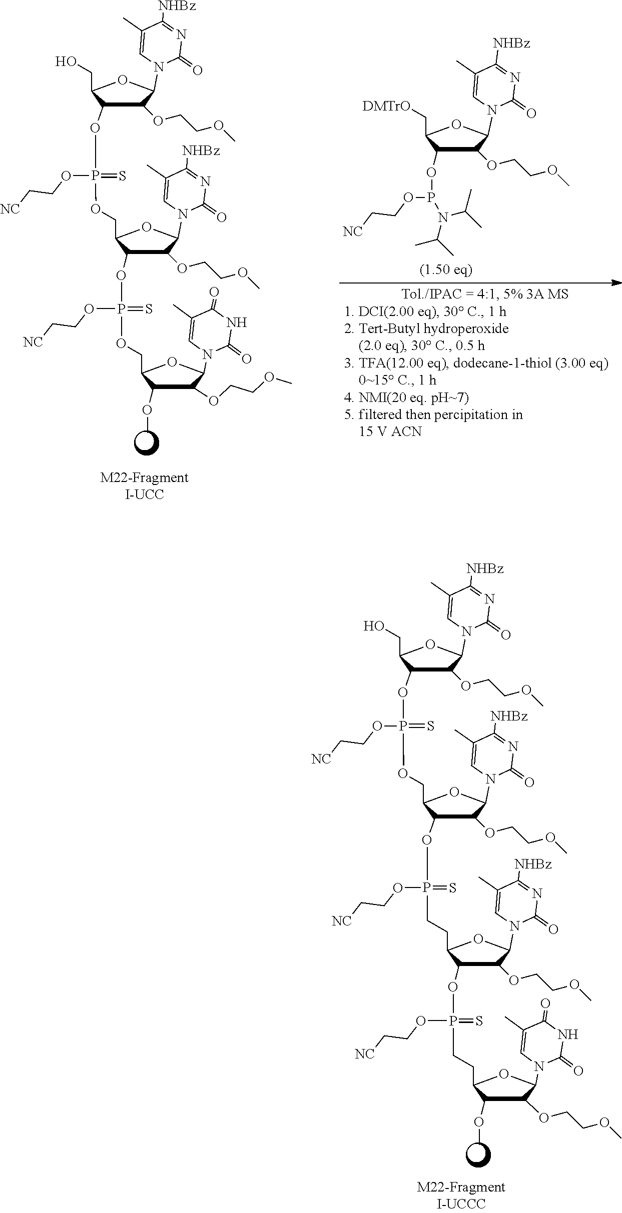

To a solution of M22-Fragment I-UCC (3.20 g, 1.16 mmol) and C-DMTr (1.61 g, 1.74 mmol) in Tol. (15.4 mL) and ACN (3.80 mL) was added DCI (275 mg, 2.32 mmol) and Molecular sieve 3A (1.50 g). The mixture was stirred at 30° C. for 1 h. TLC (Dichloromethane/Methanol=20/1, two times, R_{f}=0.59) indicated M22-Fragment I-UCC was consumed completely and many new spots formed. Then Tert-Butyl hydroperoxide (5.50 M, 1.18 mmol, 106.85 uL) was added into the reaction mixture. The mixture was stirred at 30° C. for 0.5 h. Then dodecane-1-thiol (711 mg, 3.52 mmol, 840 uL) and TFA (2.00 g, 17.6 mmol, 1.30 mL). The mixture was stirred at 0° C. for 1 h. TLC (Dichloromethane/Methanol=20/1, three times, product: R_{f}=0.54) indicated M22-Fragment I-UCCC-DMTr was consumed completely and two new spots formed. The reaction mixture was quenched by addition NMI (20.0 eq) and then filtered. Then it was triturated by ACN (300 mL) and filtered. The white solid was concentrated under reduced pressure to give a residue. M22-Fragment I-UCCC (3.60 g, crude) was obtained as a white solid.

General Procedure for Preparation of Oligonucleotide Fragment C

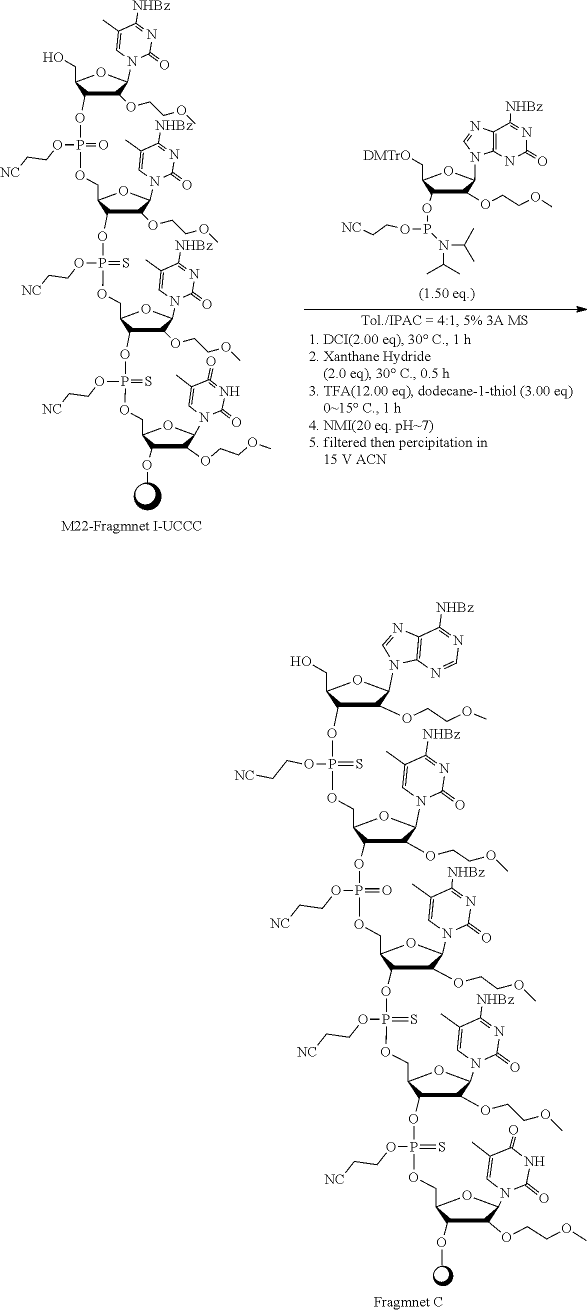

To a solution of M22-Fragment I-UCCC (3.40 g, 1.03 mmol) and A-DMTr (1.45 g, 1.55 mmol) in Tol. (16.0 mL) and ACN (4.00 mL) was added DCI (244 mg, 2.06 mmol) and Molecular sieve 3A (1.00 g). The mixture was stirred at 30° C. for 1 h. TLC (Dichloromethane/Methanol=15/1, three times, R_{f}=0.47) indicated M22-Fragment I-UCCC was consumed completely and many new spots formed. Then Xanthane Hydride (307 mg, 2.04 mmol) was added into the reaction mixture. The mixture was stirred at 30° C. for 0.5 h. Then dodecane-1-thiol (615 mg, 3.04 mmol, 727 uL) and TFA (1.73 g, 15.2 mmol, 1.12 mL) was added into the reaction mixture, and the mixture was stirred at 0° C. for 1 h. TLC (Dichloromethane/Methanol=15/1, three times, product: R_{f}=0.45) indicated M22-Fragment I-UCCCA-DMTr was consumed completely and two new spots formed. The reaction mixture was quenched by addition NMI (20.0 eq) and then filtered. Then it was triturated by ACN (600 mL) and filtered. Oligonucleotide fragment C (3.90 g, 90.3% yield and 84.2% purity) was obtained as a white solid.

### Example 10. Synthesis of Oligonucleotide Fragment D

a. Scheme for Synthesis of Oligonucleotide Fragment D

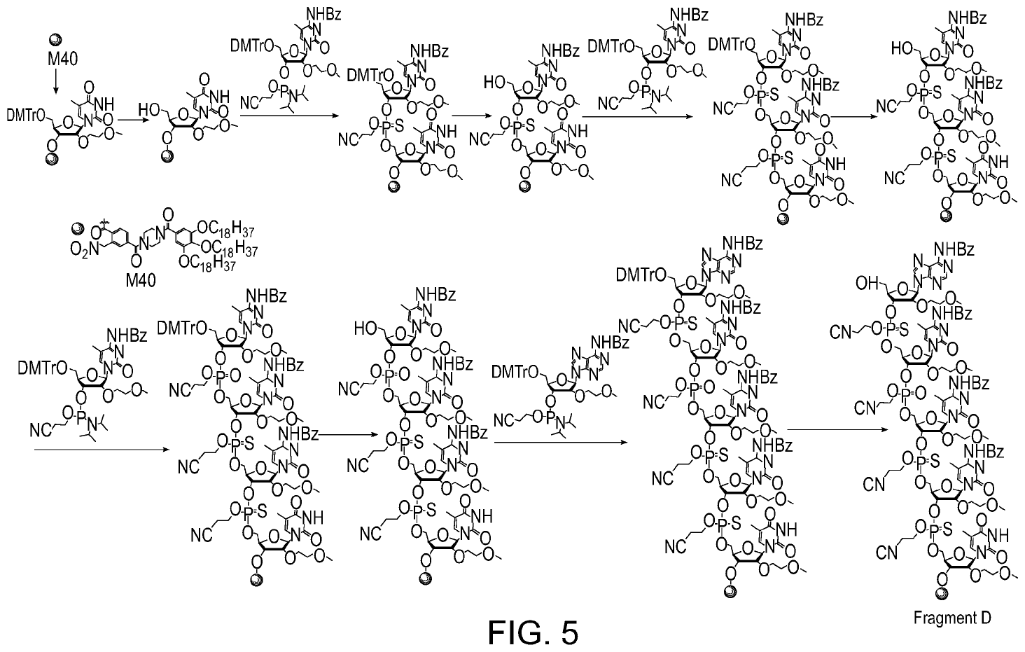

Fragment D was synthesized according to synthetic scheme depicted in FIG. 5.

b. Procedures for Synthesis of Oligonucleotide Fragment D

Procedures for synthesizing oligonucleotide fragment D were similar to those described for the synthesis of oligonucleotide fragment A.

### Example 11. Synthesis of Oligonucleotide Fragment E

a. Scheme for Synthesis of Oligonucleotide Fragment E

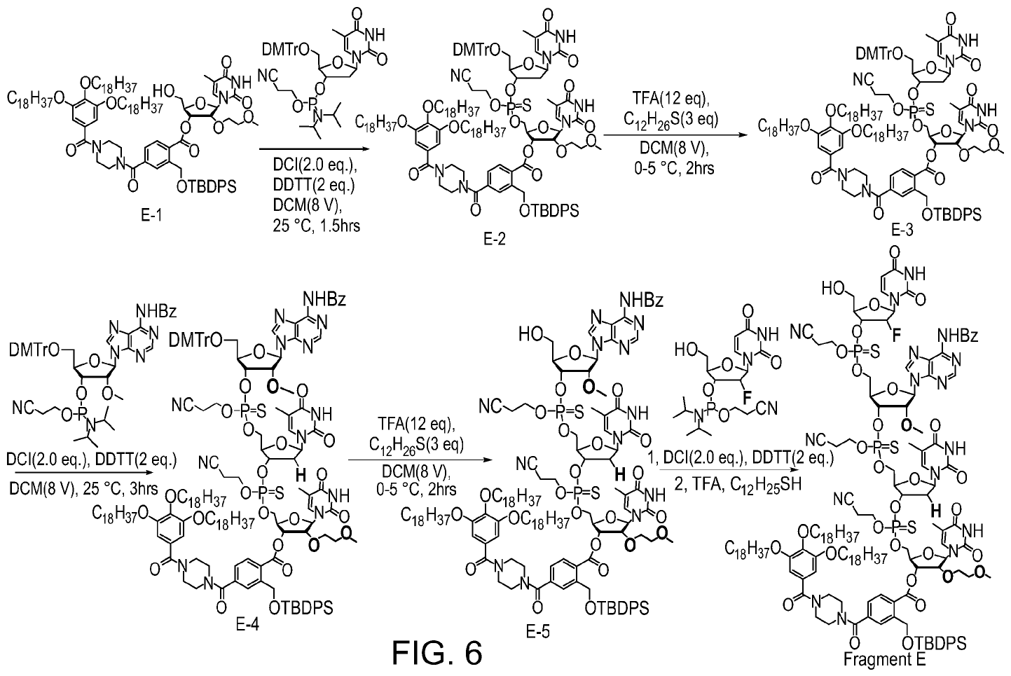

Fragment E was synthesized according to synthetic scheme depicted in FIG. 6.

b. Procedures for Synthesis of Oligonucleotide Fragment E

General Procedure for Preparation of Compound E-2

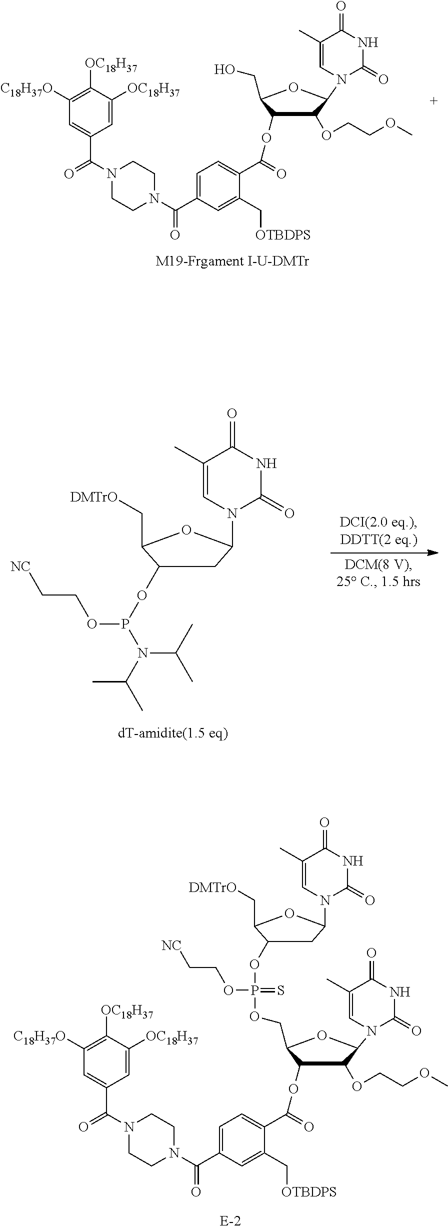

Compound M19-Fragment I-U-DMTr (2.00 g, 1.17 mmol, 1.00 eq) with dry DCM (12.0 ml) and CH₃CN (4.00 mL) were concentrated under reduced pressure to remove water three times. To a solution of compound M19-Fragment I-U-DMTr (2.00 g, 1.17 mmol, 1.00 eq) in DCM (16 mL) was added 3A MS (1.60 g) in one portion at 25° C. under Ar and stir for 0.5 hour. dT-amidite (1.31 g, 1.75 mmol, 1.5 eq) and DCI (276 mg, 2.34 mmol, 2.00 eq) were added, and the mixture was stirred at 25° C. for 1 hour. HPLC showed the starting material was consumed completely. DDTT (480 mg, 2.34 mmol, 2.00 eq) was added into reaction solution. The mixture was stirred at 25° C. for 0.5 hr. HPLC showed the starting material was consumed completely. The crude product was triturated with ACN (160 mL) at 25° C. for 1 hr. The mixture was filtered and the cake was concentrated in vacuum. Compound E-2 (2.3 g, 964 umol, 83.0% yield) was obtained as a white solid. Mass calcd for C₁₃₇H₁₉₈N₇O₂₂PSSiNa⁺[M+Na⁺]: 2408.4, found: 2408.4.

General Procedure for Preparation of Compound E-3

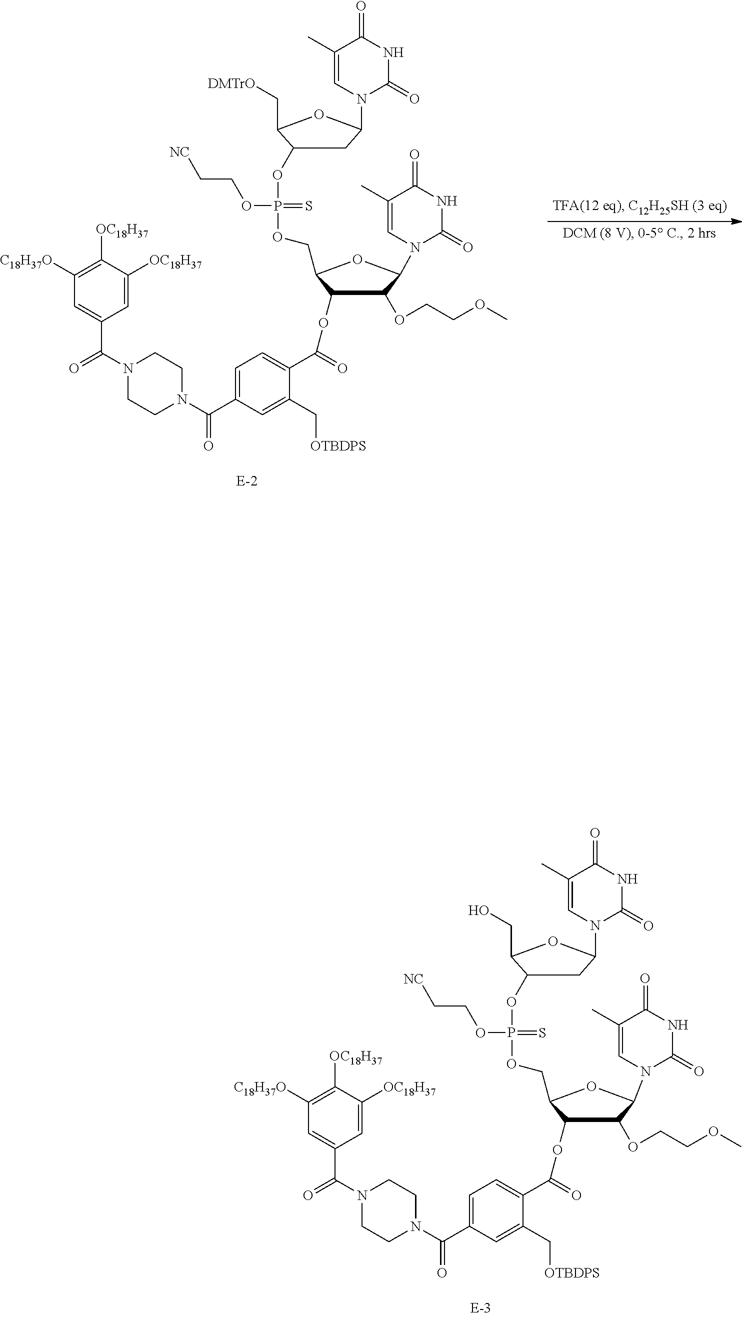

To a solution of compound E-2 (2.30 g, 964 umol, 1.00 eq) in DCM (20.0 mL) was added C₁₂H₂₅SH (585 mg, 2.89 mmol, 693 uL, 3.00 eq) in one portion at 25° C. under N₂. TFA (1.32 g, 11.57 mmol, 856.38 uL, 12 eq) was added into solution, and the mixture was stirred at 0-5° C. and stirred for 2 hours. LCMS showed the starting material was consumed completely. NMI (1.19 g, 14.5 mmol, 1.15 mL, 15.0 eq) was added into the reaction and stir at 0-5° C. for 0.5 hr. The crude product was triturated with ACN (200 mL) at 25° C. for 1 hr. The mixture was filtered and the cake was concentrated in vacuum. Compound E-3 (2.00 g, 960 umol, 99% yield) was obtained as a white solid. Mass calcd for C₁₁₆H₁₈₁N₇O₂₀PSSi⁺[M+H⁺]: 2083.3, found: 2083.3.

General Procedure for Preparation of Compound E-4

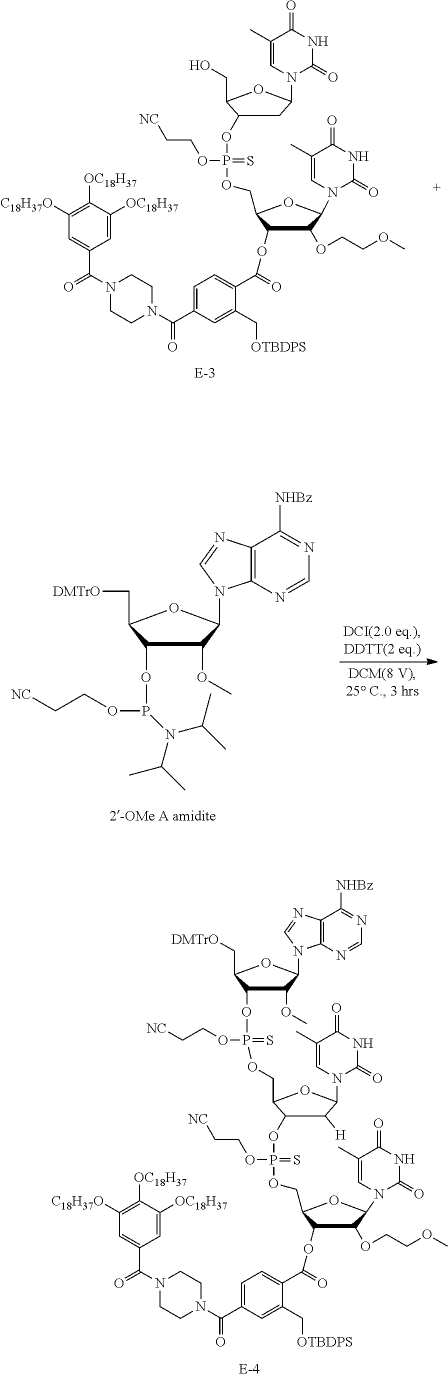

Compound E-3 (2.00 g, 959 umol, 1.00 eq) with dry DCM (12.0 ml) and ACN (4.00 mL) were concentrated under reduced pressure to remove water three times. To a solution of compound E-3 (2.00 g, 959 umol, 1.00 eq) in DCM (16 mL) was added 3A MS (1.60 g) in one portion at 25° C. under Ar and stir for 0.5 hr. 2′-OMe A amidite (1.28 g, 1.44 mmol, 1.50 eq) and DCI (227 mg, 1.92 mmol, 2.00 eq) were added, and the mixture was stirred at 25° C. for 1 hr. LCMS showed the starting material was consumed completely. DDTT (395 mg, 1.92 mmol, 2.00 eq) was added into reaction solution, and the mixture was stirred at 25° C. for 0.5 hr. LCMS showed the starting material was consumed completely. The crude product was triturated with ACN (160 mL) at 25° C. for 1 hr. The mixture was filtered and the cake was concentrated in vacuum. Compound E-4 (2.11 g, 727 umol, 75.6% yield) was obtained as a white solid. Mass calcd for C158H219N13O28P2S2SiNa+[M+Na⁺]: 2923.5, found: 2924.5.

General Procedure for Preparation of Compound E-5

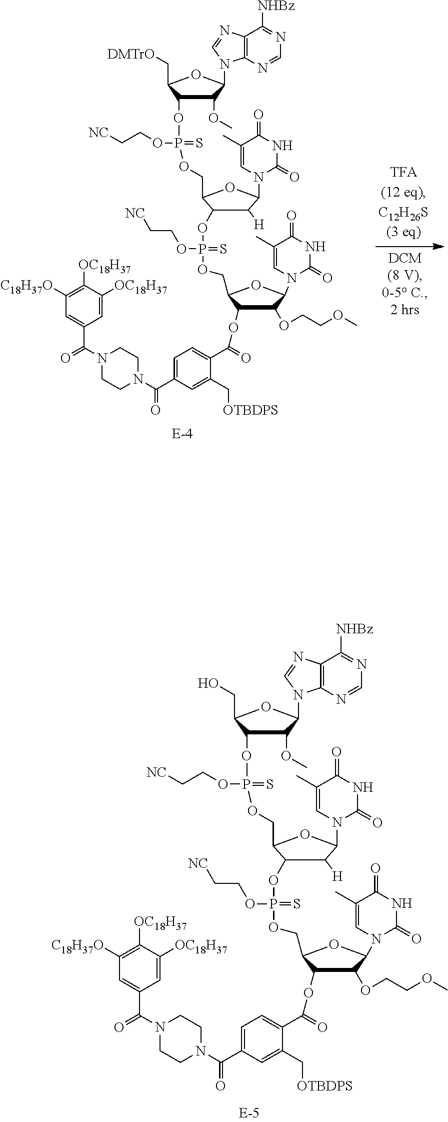

Compound E-4 (2.1 g, 723 umol, 1.00 eq) with dry DCM (12.0 ml) and ACN (4.00 mL) were concentrated under reduced pressure to remove water three times. To a solution of compound E-4 (2.1 g, 723 umol, 1.00 eq) in DCM (20.0 mL) was added C₁₂H₂₅SH (439 mg, 2.17 mmol, 520 uL, 3.00 eq) in one portion at 25° C. under N₂. TFA (990 mg, 8.68 mmol, 643 uL, 12.0 eq) was added into solution, and the mixture was stirred at 0-5° C. and stirred for 2 hrs. LCMS showed the starting material was consumed completely. NMI (891 mg, 10.8 mmol, 865 uL, 15 eq) was added into the reaction and stir at 0-5° C. for 0.5 hr. The crude product was triturated with ACN (200 mL) at 25° C. for 1 hr. The mixture was filtered and the cake was concentrated in vacuum. Compound E-5 (1.7 g, 653.77 umol, 90.37% yield) was obtained as a white solid. Mass calcd for C₁₃₇H₂₀₂N₁₃O₂₆P₂S₂Si⁺[M+H]: 2599.3, found: 2599.4.

General Procedure for Preparation of Compound E-6

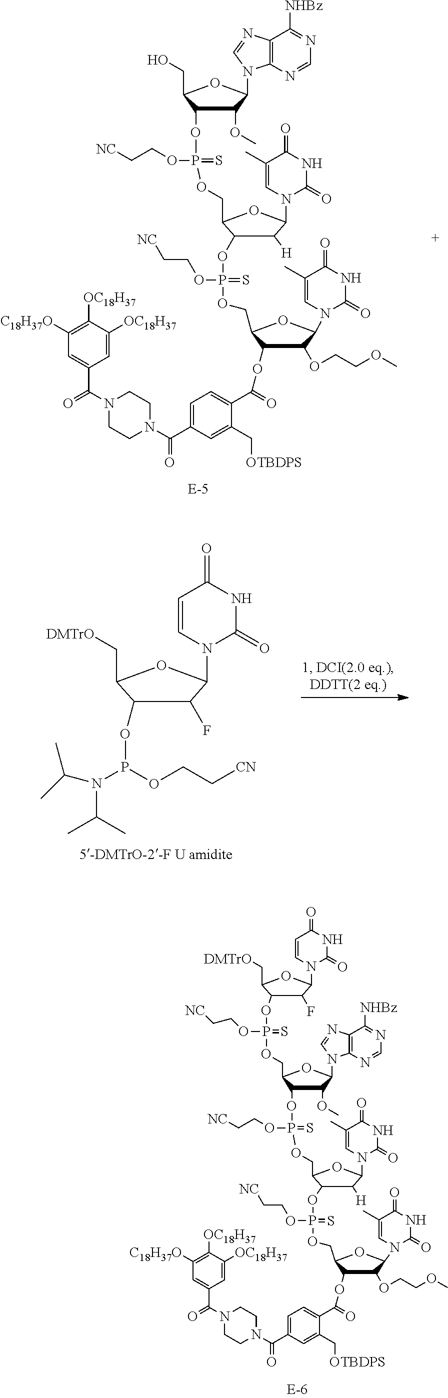

Compound E-5 (1.00 g, 385 umol, 1.00 eq) with dry DCM (6.00 ml) and ACN (2.00 mL) were concentrated under reduced pressure to remove water three times. To a solution of compound E-5 (1.00 g, 385 umol, 1.00 eq) in DCM (8.00 mL) was added 3A MS (1.60 g) in one portion at 25° C. under Ar and stir for 0.5 hr. 5′-DMTrO-2′-F U amidite (440 mg, 577 umol, 1.50 eq) and DCI (90.8 mg, 769 umol, 2.00 eq) were added into the mixture, and the mixture was stirred at 25° C. for 1 hr. LCMS showed the starting material was consumed completely. DDTT (151 mg, 736 umol, 2.00 eq) was added into reaction solution, and the mixture was stirred at 25° C. for 0.5 hr. LCMS showed the starting material was consumed completely. The crude product was triturated with ACN (80 mL) at 25° C. for 1 hr. The mixture was filtered and the cake was concentrated in vacuum. Compound E-6 (0.9 g, 273 umol, 74.3% yield) was obtained as a white solid. Mass calcd for C₁₇₀H₂₃₃FN₁₆O₃₄P₃S₃Si⁺[M+H]: 3278.5118, found: 3278.6255.

General Procedure for Preparation of Fragment E

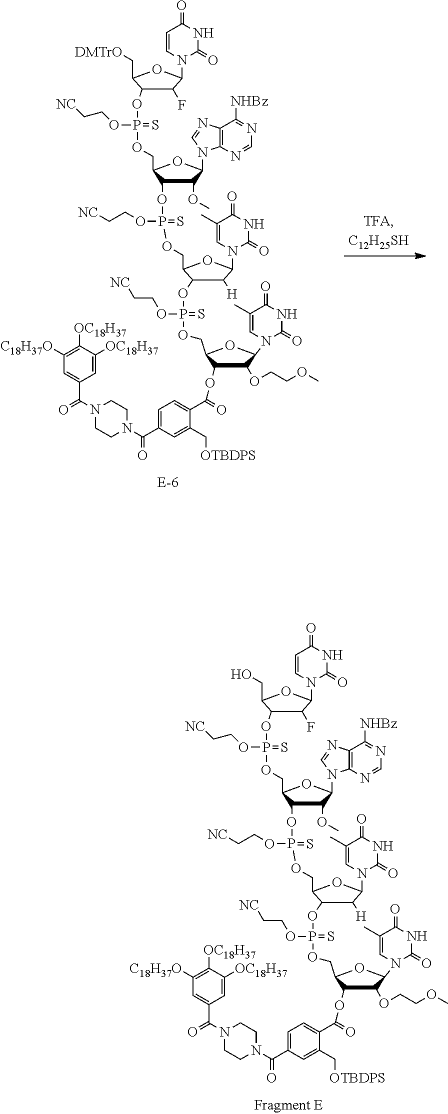

Compound E-6 (800 mg, 244 umol, 1.00 eq) with dry DCM (6.00 ml) and ACN (2.00 mL) were concentrated under reduced pressure to remove water three times. To a solution of Compound E-6 (800 mg, 244 umol, 1.00 eq) in DCM (8.00 mL) was added C₁₂H₂₅SH (148 mg, 732 umol, 176 uL, 3.00 eq) in one portion at 25° C. under N₂. TFA (334 mg, 2.93 mmol, 217 uL, 12.0 eq) was added into solution, and the mixture was stirred at 0-5° C. and stirred for 2 hrs. HRMS showed the starting material was consumed completely. NMI (300 mg, 3.66 mmol, 292 uL 15.0 eq) was added into the reaction and stir at 0-5° C. for 0.5 hr. The crude product was triturated with ACN (80.0 mL) at 25° C. for 1 hr. The mixture was filtered and the cake was concentrated in vacuum. Fragment E (600 mg, 201 umol, 82.6% yield) was obtained as a white solid, HRMS calcd for C₁₄₉H₂₁₄FN₁₆O₃₂P₃S₃Si⁺[M+H]: 2976.3811, found: 2976.3875.

### Example 12. Synthesis of Oligonucleotide Fragment F

a. Scheme for Synthesis of Oligonucleotide Fragment F

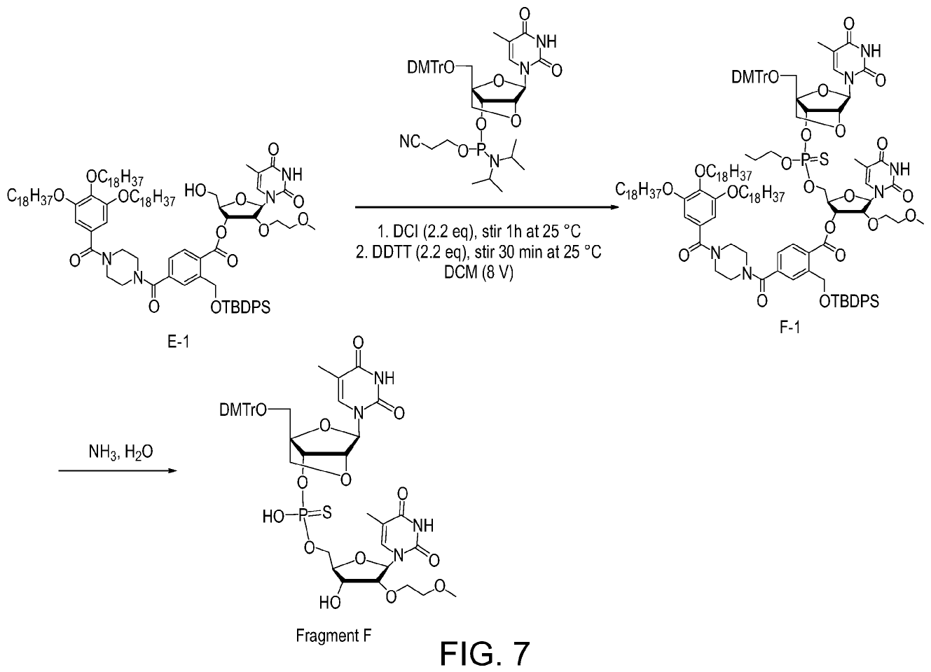

Fragment F was synthesized according to synthetic scheme depicted in FIG. 7.

b. Procedures for Synthesis of Oligonucleotide Fragment F

General Procedure for Preparation of Compound F-1

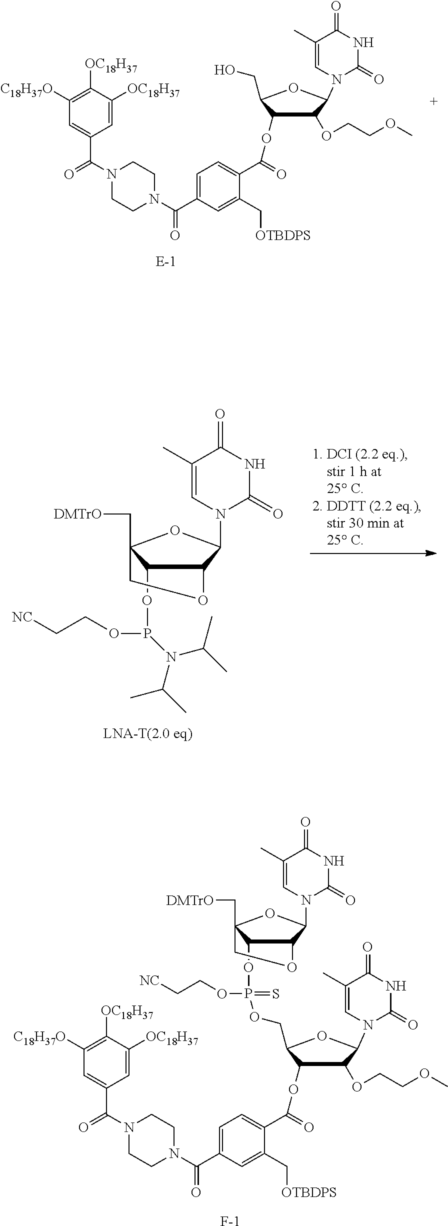

Compound E-1 (0.5 g, 292.31 umol, 1.00 eq) with dry DCM (4.0 ml) and CH₃CN (2.00 mL) were concentrated under reduced pressure to remove water three times. To a solution of compound E-1 (0.5 g, 292.31 umol, 1.00 eq) in DCM (5.00 mL) was added 3A MS (0.50 g) in one portion at 25° C. under Ar and stirred for 0.5 hr. LNA-T amidite (451.81 mg, 584.62 umol, 2.00 eq) and DCI (75.95 mg, 643.08 umol, 2.20 eq) were added, and the mixture was stirred at 25° C. for 1 hr. LCMS showed the starting material was consumed completely. DDTT (480 mg, 2.34 mmol, 2.00 eq) was added into reaction solution, and the mixture was stirred at 25° C. for 0.5 hr. LCMS showed the starting material was consumed completely. The crude product was triturated with ACN (50 mL) at 25° C. for 1 hr. The mixture was filtered and the cake was concentrated in vacuum. Compound F-1 (2.3 g, 964 umol, 83.0% yield) was obtained as a white solid.

General Procedure for Preparation of Fragment F

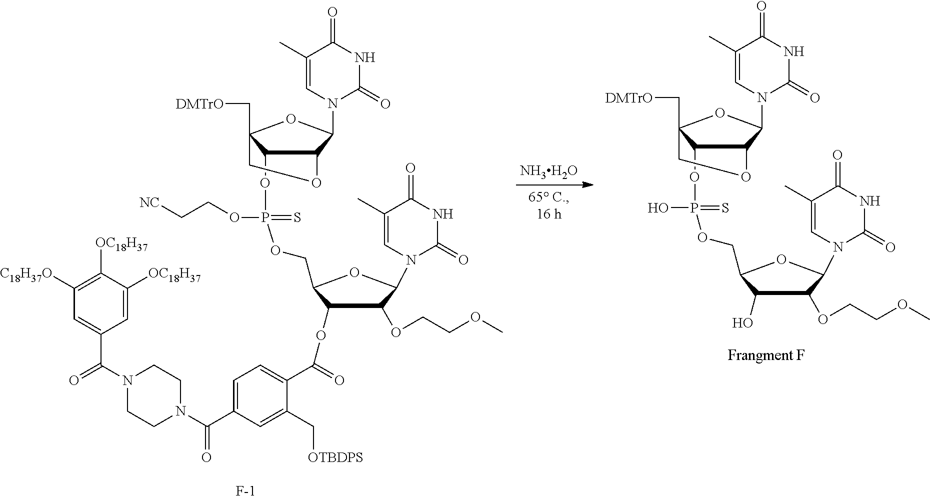

A solution of compound F-1 (50 mg, 19.23 umol, 1.00 eq) saturated with NH₃·H₂O (2.00 mL) was stirred at 70° C. for 16 hours in a 4 mL of sealed tube. Without any purification and the reaction mixture was filtered, the filtrate was provided for LCMS. Fragment F is confirmed by LCMS: HRMS calcd for C₄₅H₅₀N₄O₁₆PS⁻ [M−H⁺]: 965.2686, found: 965.2846.

### Example 13. Synthesis of Oligonucleotide Fragment G

a. Scheme for Synthesis of Oligonucleotide Fragment G

Fragment G was synthesized according to synthetic scheme depicted below.

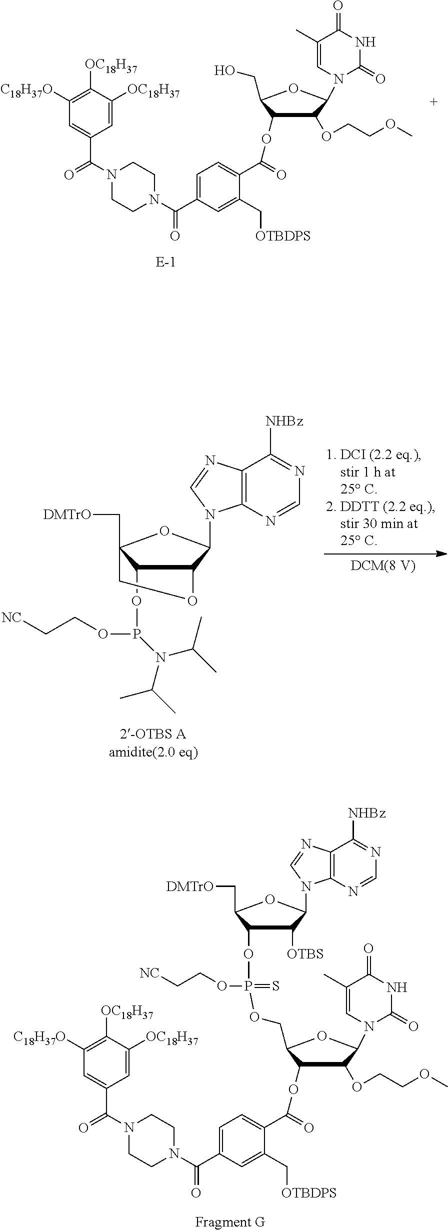

b. Procedures for Synthesis of Oligonucleotide Fragment G

Compound E-1 (500 mg, 292 umol, 1.00 eq) with dry DCM (4.0 ml) and ACN (2.00 mL) were concentrated under reduced pressure to remove water three times. To a solution of compound E-1 (500 mg, 292 umol, 1.00 eq) in DCM (4.00 mL) was added 3A MS (500 mg) in one portion at 25° C. under Ar and stir for 0.5 hr. 2′-OTBS A amidite (578 mg, 585 umol, 2.00 eq) and DCI (75.9 mg, 643 umol, 2.20 eq) were added into above mixture, and the mixture was stirred at 25° C. for 1 hr. LCMS showed the starting material was consumed completely. DDTT (480 mg, 2.34 mmol, 2.00 eq) was added into reaction solution, and the mixture was stirred at 25° C. for 0.5 hr. LCMS showed the starting material was consumed completely. The crude product was triturated with ACN (40 mL) at 25° C. for 1 hr. The mixture was filtered and the cake was concentrated in vacuum. Fragment G (700 mg, 266 umol, 91.1% yield) was obtained as a white solid. Mass calcd for C150H216N10O22PSSi2+[M+H+]: 2628.5043, found: 2628.5959.

### Example 14. Synthesis of Oligonucleotide Fragment H

a. Scheme for Synthesis of Oligonucleotide Fragment H

Fragment H was synthesized according to the synthetic scheme depicted below:

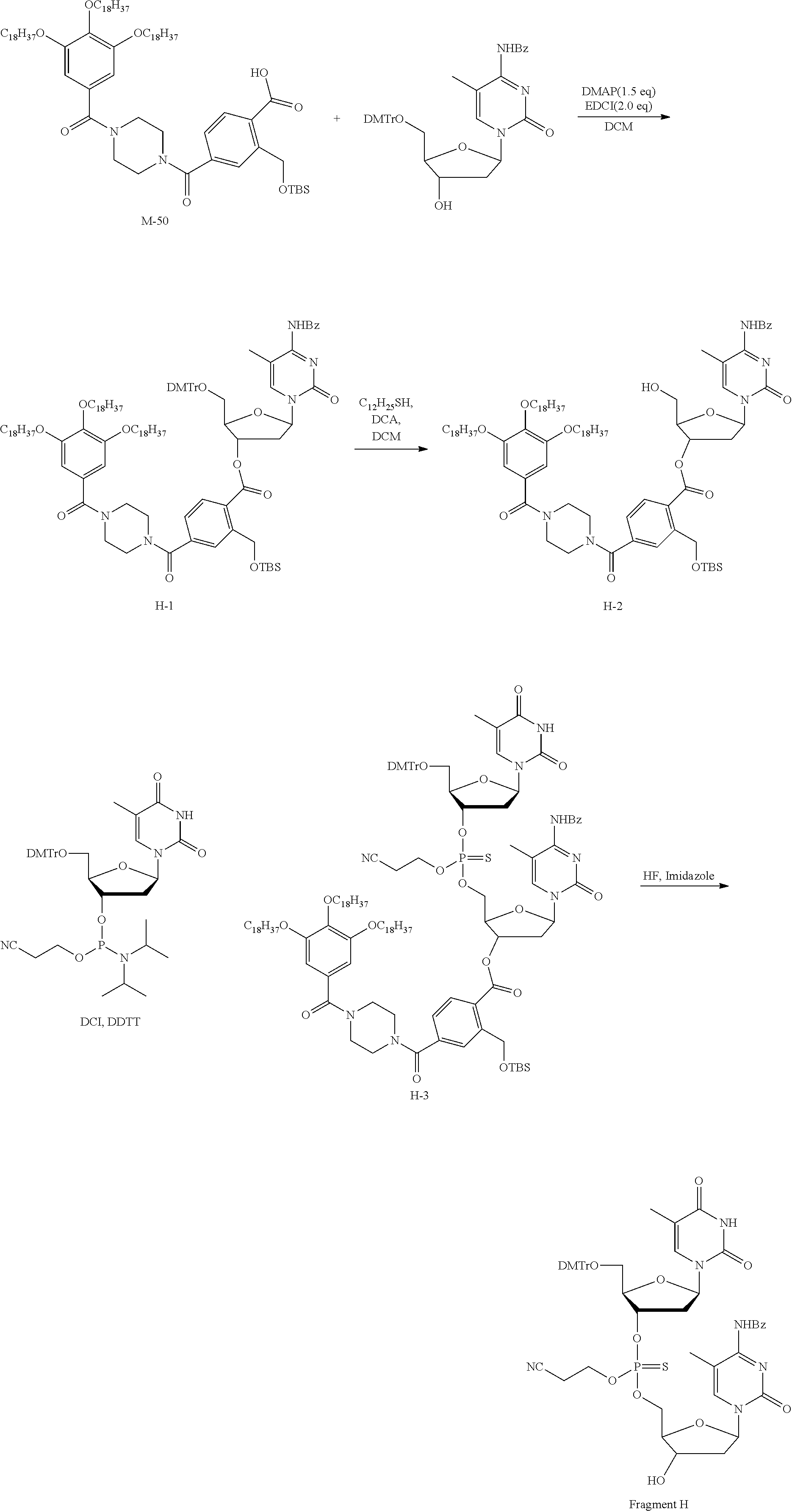

b. Procedures for Synthesis of Oligonucleotide Fragment H

General Procedure for Preparation of Compound H-1

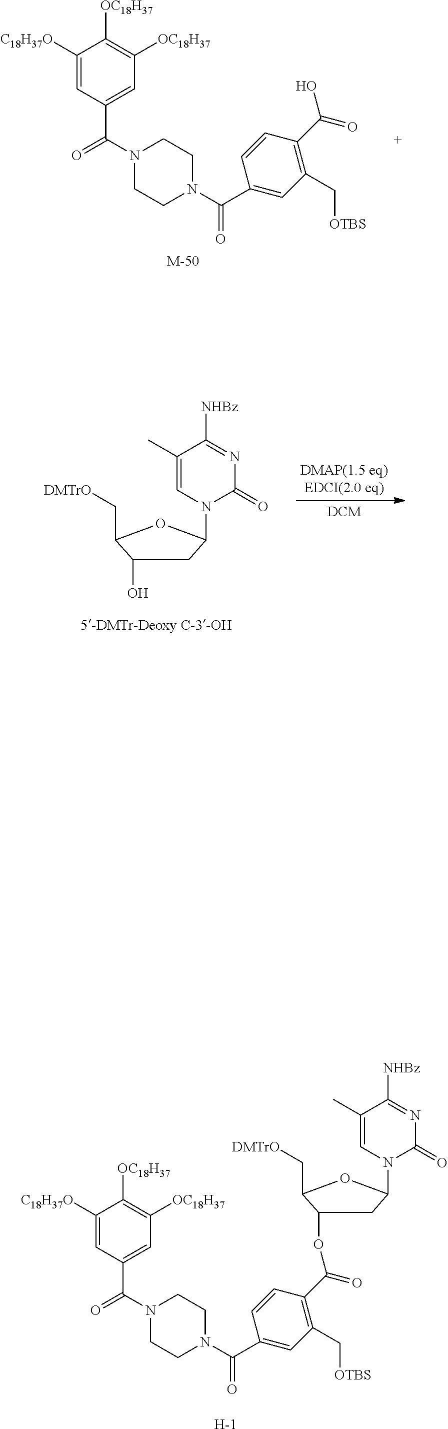

To a solution of compound M-50 (0.8 g, 621.08 umol, 1.00 eq) and 5′-DMTr-Dexoy C-3′-OH (804.57 mg, 1.24 mmol, 2.00 eq) in DCM (6.0 mL) was added DMAP (113.82 mg, 931.62 umol, 1.50 eq). The mixture was stirred at 25° C. for 0.5 h and EDCI (238.12 mg, 1.24 mmol, 2.00 eq) was added. The mixture was stirred at 25° C. for 3 hrs. HPLC showed the starting material was consumed completely. The residue was diluted with H₂O (50 mL) and extracted with DCM (2×30 mL). The combined organic layers were dried over anhydrous Na₂SO₄, filtered and concentrated under reduced pressure to give a residue. The crude product was triturated with EtOH (30 V, 30.0 mL) at 25° C. for 30 min. The crude product was triturated with ACN (30 V, 30.0 mL) at 25° C. for 30 min. Filtered and concentrated. Compound H-1 (0.8 g, 0.41 mmol, 92.0% purity, 67.17% yield) was obtained as a white solid. Mass calcd for C₁₁₈H₁₇₉N₅O₁₄Si₂+[M+2H⁺]/2: 959.1, found: 960.

General Procedure for Preparation of Compound H-2

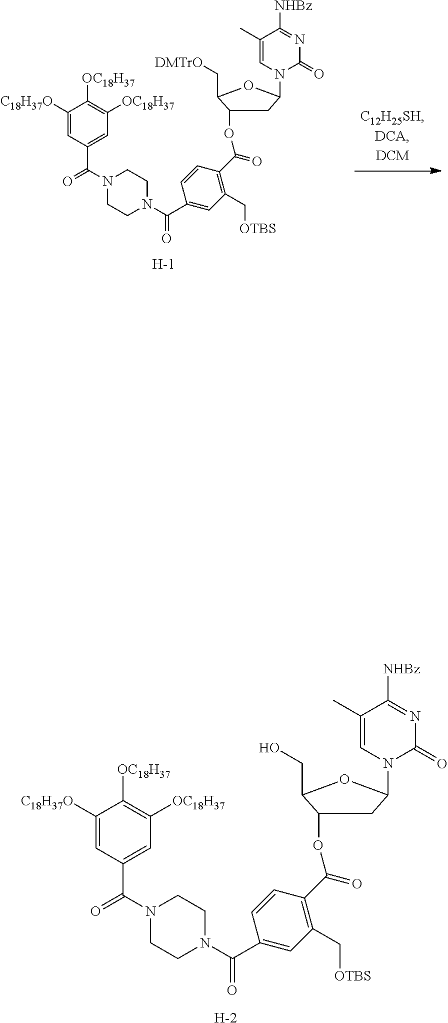

To a solution of compound H-1 (2.0 g, 1.04 mmol, 1.00 eq) in DCM (16.0 mL) was added C₁₂H₂₅SH (274.40 mg, 1.36 mmol, 324.74 uL, 1.30 eq) and DCA (1.08 g, 8.34 mmol, 685.20 uL, 8.00 eq) at 0-5° C. The mixture was stirred at 0-5° C. for 2.5 hrs and NMI (856.20 mg, 10.43 mmol, 831.26 uL, 10.00 eq) was added. The mixture was stirred at 0-5° C. for 0.5 hrs. The residue was diluted with NaHCO₄/H₂O (50 mL) and extracted with DCM (2×30 mL). The combined organic layers were dried over anhydrous Na₂SO₄, filtered and concentrated under reduced pressure to give a residue. The crude product was triturated with ACN (30 V, 60.0 mL) at 25° C. for 30 min. Filtered and concentrated. Compound H-2 (1.4 g, 713.26 umol, 82.3% purity, 68.4% yield) was obtained as a white solid. Mass calcd for C₉₇H₁₆₀N₅O₁₂Si⁺[M+H⁺]: 1615.1, found: 1615.6.

General Procedure for Preparation of Compound H-3

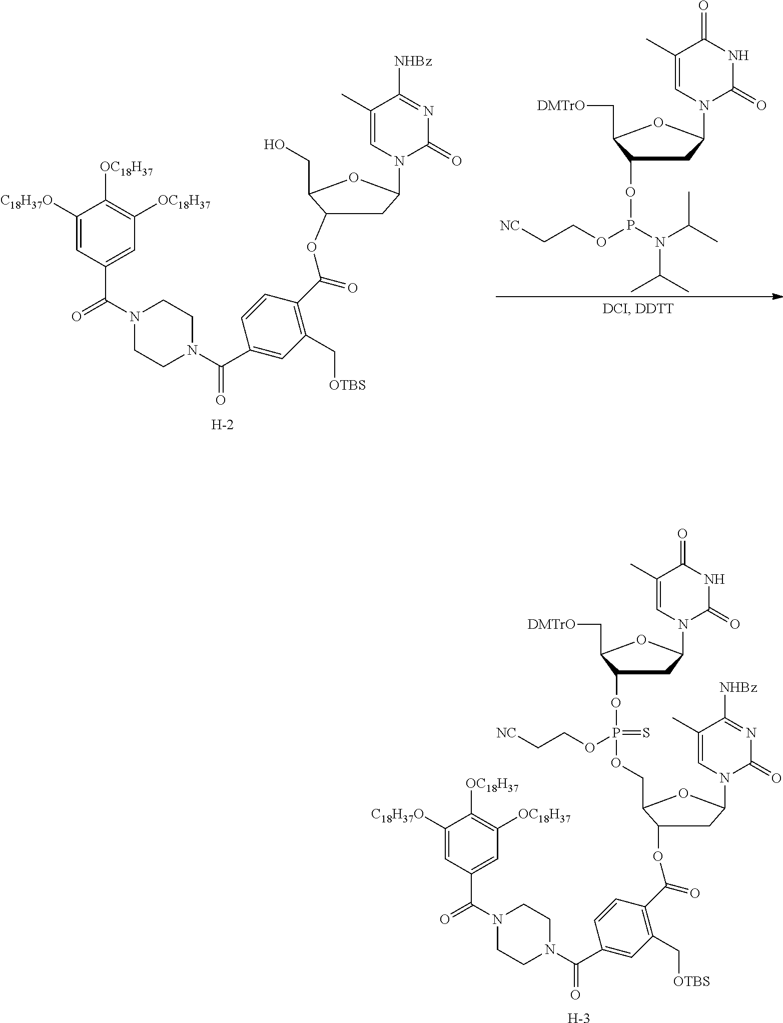

Compound H-2 (900 mg, 557 umol, 1.00 eq) with dry DCM (8.0 ml) and ACN (2.00 mL) were concentrated under reduced pressure to remove water three times. To a solution of compound H-2 (900 mg, 557 umol, 1.00 eq) in DCM (8.00 mL) was added 3A MS (800 mg) in one portion at 25° C. under Ar and stir for 0.5 hr. 5′-DMTr-Dexoy T-3′-phosphoramidite (830 mg, 1.11 mmol, 2.00 eq) and DCI (197.4 mg, 1.67 mmol, 3.0 eq) were added into above mixture. The mixture was stirred at 25° C. for 1 hr. LCMS showed the starting material was consumed completely. DDTT (343 mg, 1.67 mmol, 3.00 eq) was added into reaction solution. The mixture was stirred at 25° C. for 0.5 hr. LCMS showed the starting material was consumed completely. The crude product was triturated with ACN (40 mL) at 25° C. for 1 hr. The mixture was filtered and the cake was concentrated in vacuum. Compound H-3 (600 mg, 145.85 mmol, 47.3% yield) was obtained as a white solid. Mass calcd for C₁₁₀H₁₇₆N₈O₁₈PSSi⁺[M−DMTr+H⁺] 1988.2; found 1989.0.

General Procedure for Preparation of Fragment H

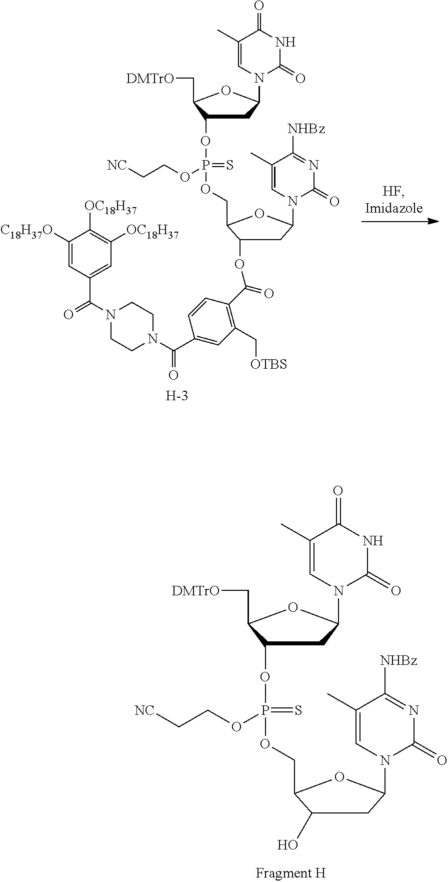

To a solution of compound H-3 (0.2 g, 87.29 umol, 1.00 eq) in THF (1.6 mL) was added solution of imidazole (118.86 mg, 1.75 mmol, 20.0 eq) and pyridine/hydrofluoride (24.94 mg, 872.95 umol, 70% purity, 10.0 eq) in THF (0.6 mL). The mixture was stirred at 0-5° C. for 20 hrs (Compound H-3 was converted to Fragment H with >95% conversion). The structure of Fragment H was confirmed by LCMS. Mass calcd for C₅₁H₅₄N₆O₁₃PS⁺[M+H⁺]: 1021.3, found: 1021.4

### Example 15. Synthesis of Oligonucleotide Fragment J

a. Scheme for Synthesis of Oligonucleotide Fragment J

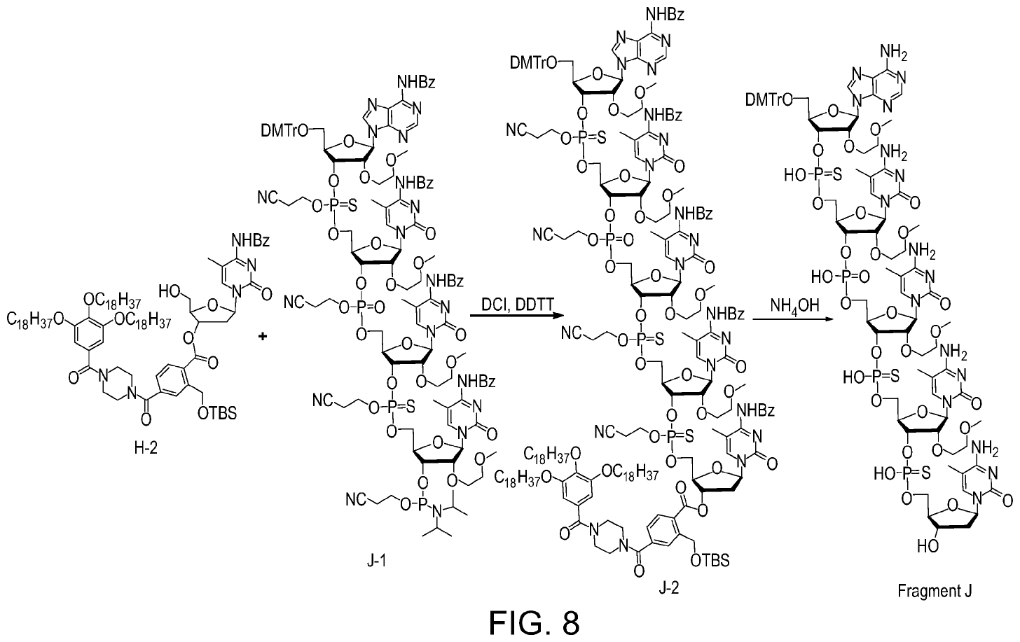

Fragment J was synthesized according to synthetic scheme depicted in FIG. 8.

b. Procedures for Synthesis of Oligonucleotide Fragment J

General Procedure for Preparation of Compound J-2

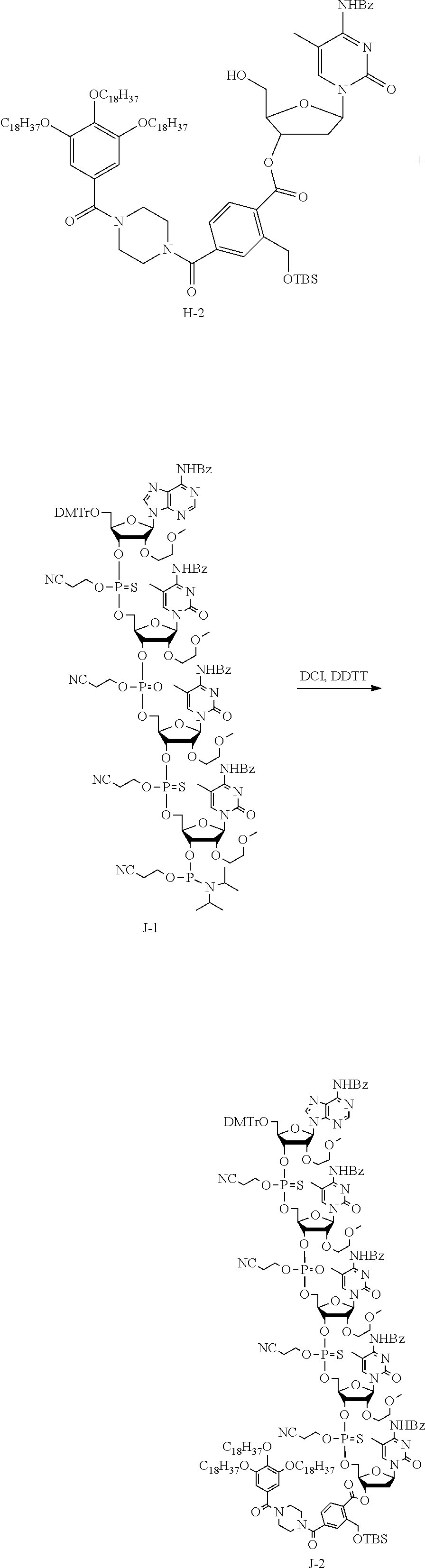

Compound H-2 (500 mg, 0.309 umol, 1.00 eq) with dry DCM (8.0 ml) and ACN (2.00 mL) were concentrated under reduced pressure to remove water three times. To a solution of compound H-2 (500 mg, 0.309 mmol, 1.00 eq) in DCM (5.00 mL) was added 3A MS (500 mg) in one portion at 25° C. under Ar and stir for 0.5 hr. 5′-DMTr-MOE-ACCC-3′-phosphoramidite (Compound J-1 (synthetic procedures are described in WO2020/227618, paragraphs [0332]-[0333], which is incorporated herein by reference), 1590 mg, 0.62 mmol, 2.00 eq) and DCI (110 mg, 0.927 mmol, 3.0 eq) were added into above mixture, and the mixture was stirred at 25° C. for 1 hr. DDTT (254 mg, 1.23 mmol, 4.00 eq) was added into reaction solution, and the mixture was stirred at 25° C. for 0.5 hr. The crude product was triturated with ACN (50 mL) at 25° C. for 1 hr. The mixture was filtered and the cake was concentrated in vacuum. Compound J-2 (770 mg, 61% yield) was obtained as a white solid. Mass Calcd for C₂₁₀H₂₈₅N₂₃O₄₆P₄S₃Si⁺[M+2H⁺]/2: 2057.4, found 2058.0.

General Procedure for Preparation of Fragment J

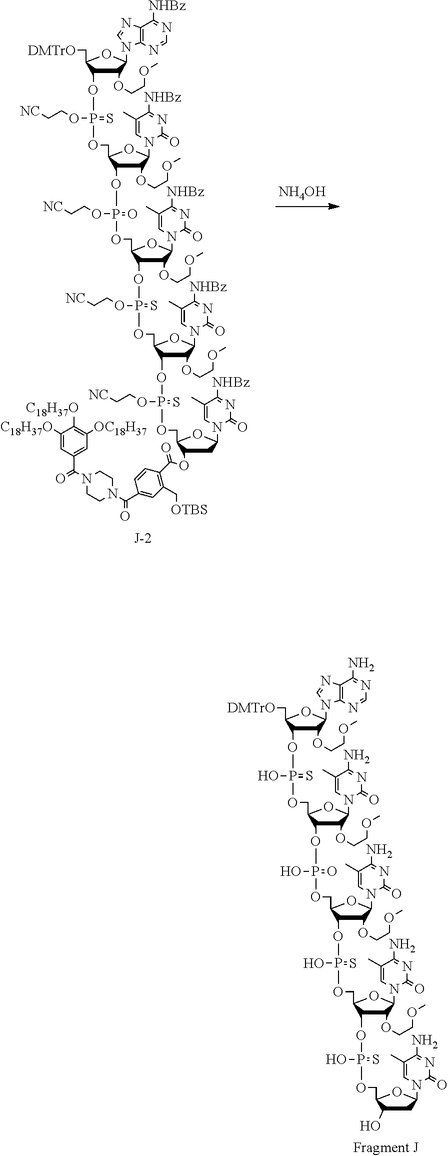

A solution of compound J-2 (50 mg) saturated with NH₃·H₂O (2.00 mL) was stirred at 65° C. for 8 hours in a 4 mL of sealed tube. Without any purification and the reaction mixture was filtered, the filtrate was provided for LCMS. Fragment J: Mass Calcd for C₈₃H₁₀₉N₁₇O₃₄P₄S₃ [M−2H]/2: 1053.8; found 1054.4.

### Example 16. Synthesis of Oligonucleotide Fragment K

a. Scheme for Synthesis of Oligonucleotide Fragment K

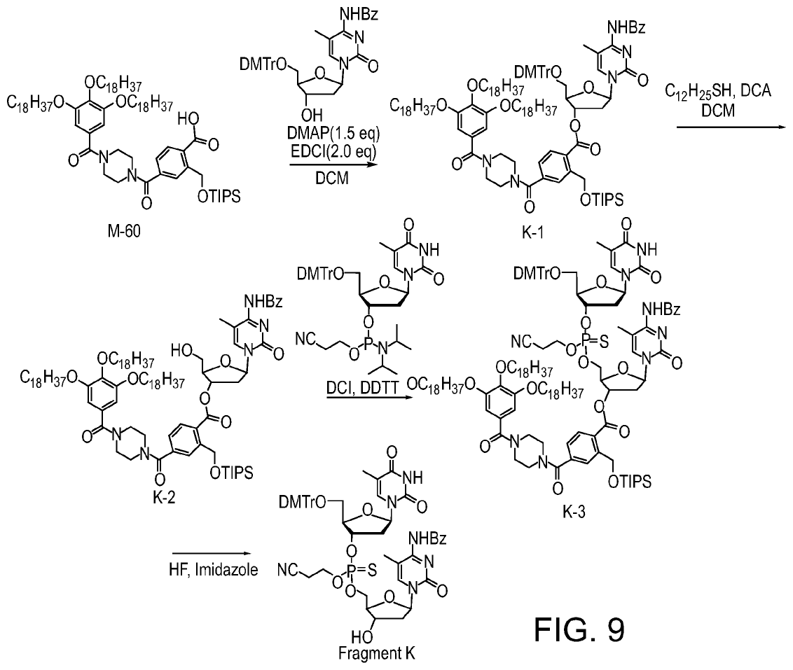

Fragment K was synthesized according to the synthetic scheme depicted in FIG. 9.

b. Procedures for Synthesis of Oligonucleotide Fragment K

General Procedure for Preparation of Compound K-1

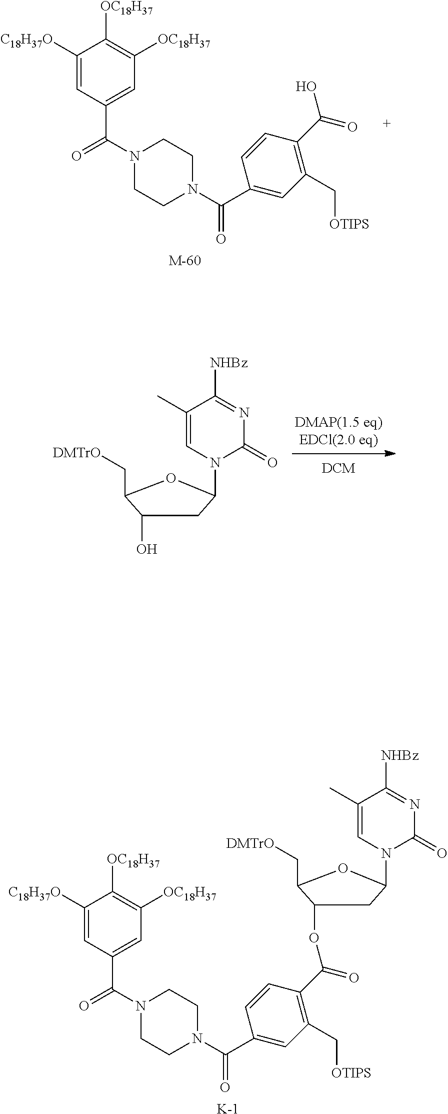

To a solution of compound M-60 (0.9 g, 676 umol, 1.00 eq) and 5′-DMTr-Dexoy C-3′-OH (876.5 mg, 1.35 mmol, 2.00 eq) in DCM (10.0 mL) was added DMAP (124 mg, 1.01 mmol, 1.50 eq). The mixture was stirred at 25° C. for 0.5 h and EDCI (260 mg, 1.35 mmol, 2.00 eq) was added. The mixture was stirred at 25° C. for 3 hrs. HPLC showed the starting material was consumed completely. The residue was diluted with H₂O (50 mL) and extracted with DCM (2×20 mL). The combined organic layers were dried over anhydrous Na₂SO₄, filtered and concentrated under reduced pressure to give a residue. The crude product was triturated with EtOH (30 V, 30.0 mL) at 25° C. for 30 min. The crude product was triturated with ACN (30 V, 30.0 mL) at 25° C. for 30 min. Filtered and concentrated. Compound K-1 (1.2 g, 0.56 mmol, 82.4% yield) was obtained as a white solid. Mass calcd for C₁₂₁H₁₈₄N₅O₁₄Si⁺[M+2H⁺]/2: 980.7, found: 981.1.

General Procedure for Preparation of Compound K-2

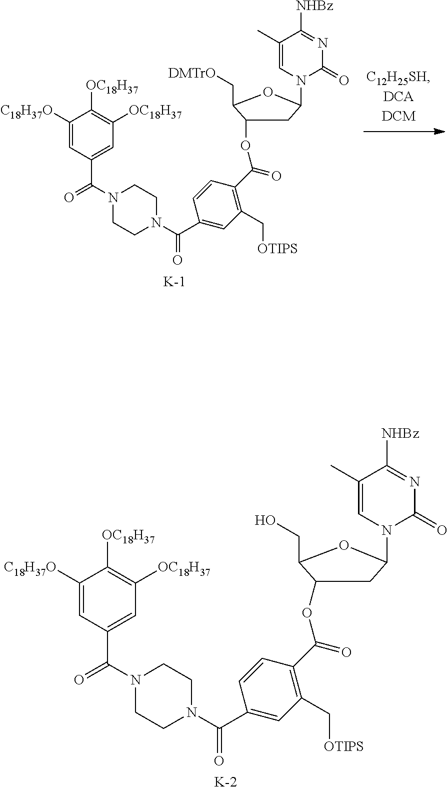

To a solution of compound K-1 (2.0 g, 1.04 mmol, 1.00 eq) in DCM (16.0 mL) was added C₁₂H₂₅SH (274.40 mg, 1.36 mmol, 324.74 uL, 1.30 eq) and DCA (1.08 g, 8.34 mmol, 685.20 uL, 8.00 eq) at 0-5° C. The mixture was stirred at 0-5° C. for 2.5 hrs and NMI (856.20 mg, 10.43 mmol, 831.26 uL, 10.00 eq) was added. The mixture was stirred at 0-5° C. for 0.5 hrs. The residue was diluted with NaHCO₄/H₂O (50 mL) and extracted with DCM (2×30 mL). The combined organic layers were dried over anhydrous Na₂SO₄, filtered and concentrated under reduced pressure to give a residue. The crude product was triturated with ACN (30 V, 60.0 mL) at 25° C. for 30 min. Filtered and concentrated. Compound K-2 (1.4 g, 713.26 umol, 68.4% yield) was obtained as a white solid. Mass Calcd for C₁₀₀H₁₆₆N₅O₁₂Si⁺[M+H⁺]: 1657.2, found: 1657.9.

General Procedure for Preparation of Compound K-3

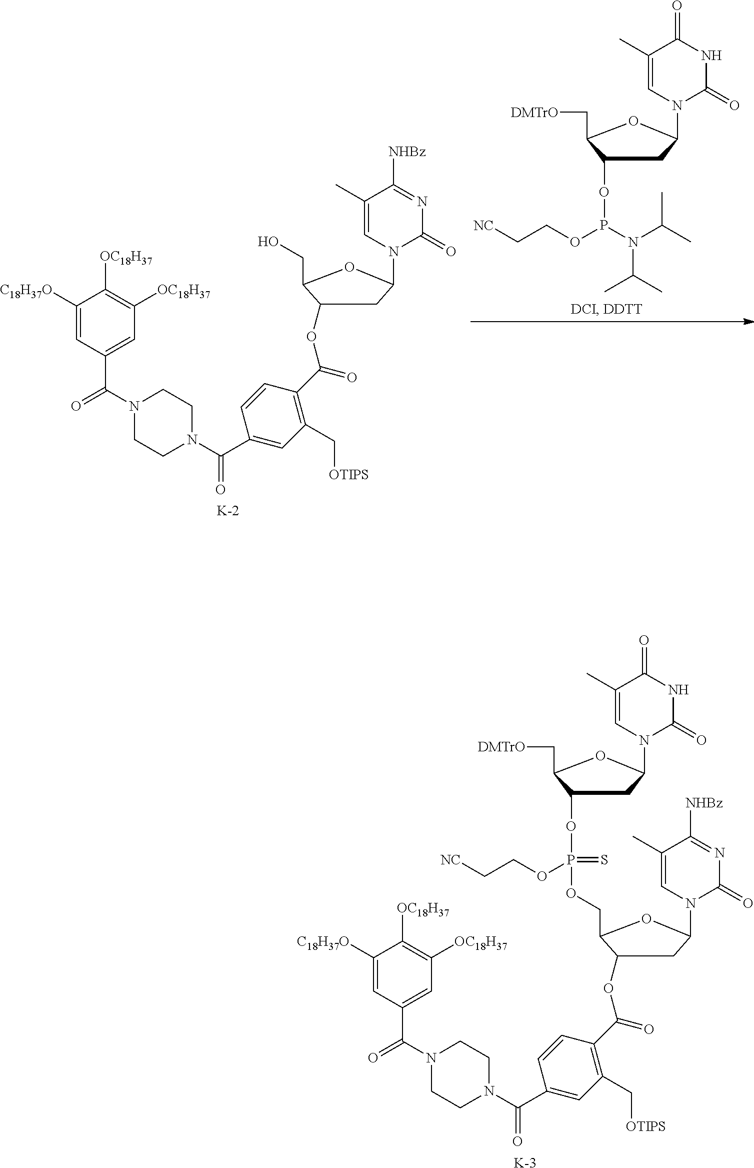

Compound K-2 (1.0 g, 603 umol, 1.00 eq) with dry DCM (8.0 ml) and ACN (2.00 mL) were concentrated under reduced pressure to remove water two times. To a solution of compound K-2 (1.0 g, 603 umol, 1.00 eq) in DCM (8.00 mL) was added 3A MS (800 mg) in one portion at 25° C. under Ar and stir for 0.5 hr. 5′-DMTr-Dexoy T-3′-phosphoramidite (898 mg, 1.21 mmol, 2.00 eq) and DCI (213.7 mg, 1.81 mmol, 3.0 eq) were added into above mixture. The mixture was stirred at 25° C. for 1 hr. LCMS showed the starting material was consumed completely. DDTT (372 mg, 1.81 mmol, 3.00 eq) was added into reaction solution, and the mixture was stirred at 25° C. for 0.5 hr. The crude product was triturated with ACN (40 mL) at 25° C. for 1 hr. The mixture was filtered and the cake: was concentrated in vacuum. Compound K-3 (1.20 g, 77.3% yield) was obtained as a white solid. Mass calcd for C₁₁₃H₁₈₂N₈O₁₈PSSi⁺[M−DMTr+H⁺]: 2031.3, found 2031.0.

General Procedure for Preparation of Fragment K

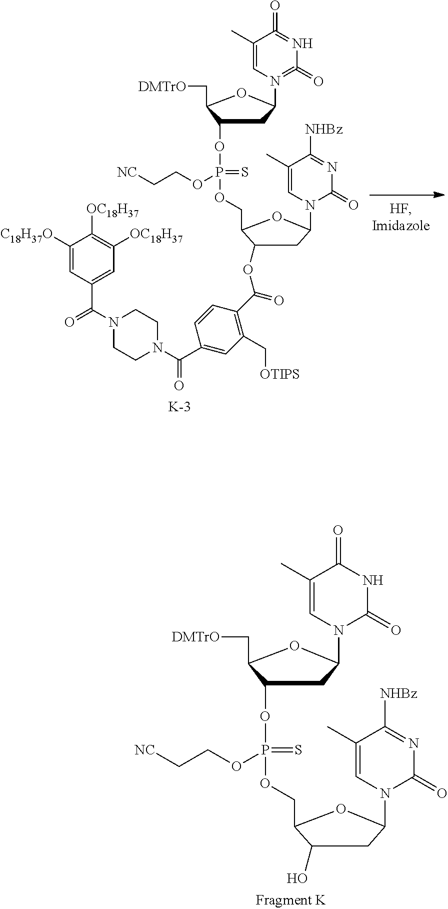

To a solution of compound K-3 (0.2 g, 85.7 umol, 1.00 eq) in THF (1.6 mL) was added solution of imidazole (116.7 mg, 1.71 mmol, 20.0 eq) and pyridine/hydrofluoride (24.5 mg, 857.2 umol, 70% purity, 10.0 eq) in THF (0.6 mL). The mixture was stirred at 25° C. for 20 hrs (Compound K-3 was converted to Fragment K with >95% conversion). The structure of Fragment K was confirmed by LCMS. Mass calcd for C₅₁H₅₃N₆NaO₁₃PS⁺[M+Na⁺]: 1043.3, found: 1043.9.

### Example 17. H Phosphate Chemistry for Preparing Fragment L

a. Scheme for Synthesis of Fragment L

Fragment L was synthesized according to synthetic scheme depicted below:

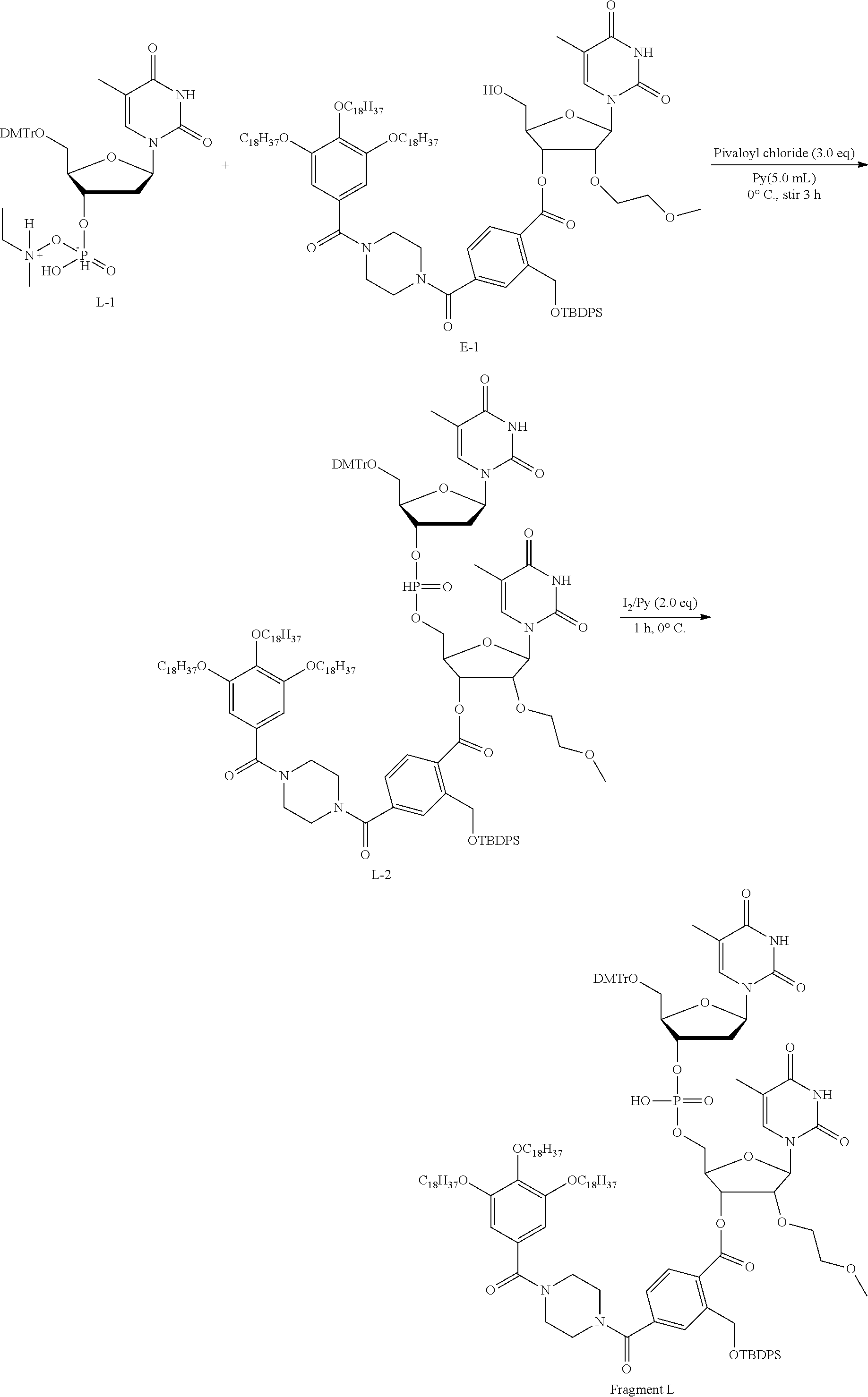

b. Procedures for Synthesis of Oligonucleotide Fragment L

General Procedure for Preparation of Fragment L

A mixture of compound L-1 (0.5 g, 0.70 mmol, 3.00 eq), pivaloyl chloride (85 mg, 0.70 mmol, 3.0 eq) in pyridine (5 mL) was stirred at 0° C. for 1 h, compound E-1 (402 mg, 235.1 mmol, 1.0 eq) was added, and the reaction mixture was stirred 0° C. for 2.0 h. A solution of iodine (120 mg, 0.47 mmol, 2.00 eq) and pyridine (121 mg, 1.53 mmol, 6.5 eq) in THF/H₂O (3 mL, 5:1, v/v) was added to the reaction mixture dropwise at 0-5° C. and stirred at 0-5° C. for 1 h. A 4 wt % aqueous solution of Na₂S₂O₃ (116 mg, 0.47 mmol, 2.00 eq) was added dropwise at 0-5° C. and stirred at 15-25° C. for 10 min. EtOAc (50 mL) was added and stirred vigorously for 30 min. The top organic layer was separated, washed with 5 wt % NaHCO₃ solution (2×30 mL), brine (30 mL), dried over MgSO₄, filtered, and concentrated. The crude product was triturated with ACN (40 mL) at 25° C. for 1 hr. The mixture was filtered and the cake was concentrated in vacuum. Fragment L (400 mg) was obtained as a white solid. Mass calcd for C₁₃₄H₁₉₅N₆O₂₃PSi [M]/2: 1157.6, found: 1157.6.

### Example 18. Synthesis of Chiral Oligonucleotide Fragment M

a. Scheme for Synthesis of Chiral Oligonucleotide Fragment M

Chiral oligonucleotide Fragment M was synthesized according to the synthetic scheme depicted below:

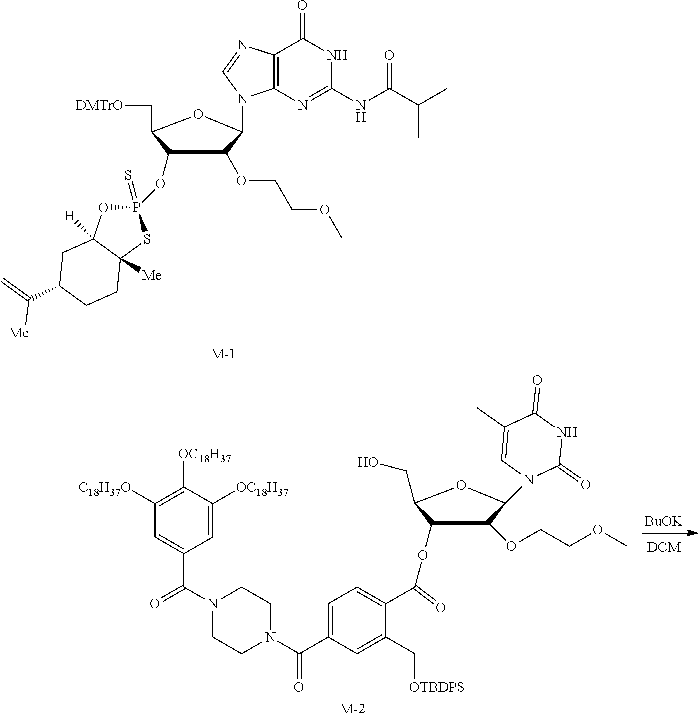

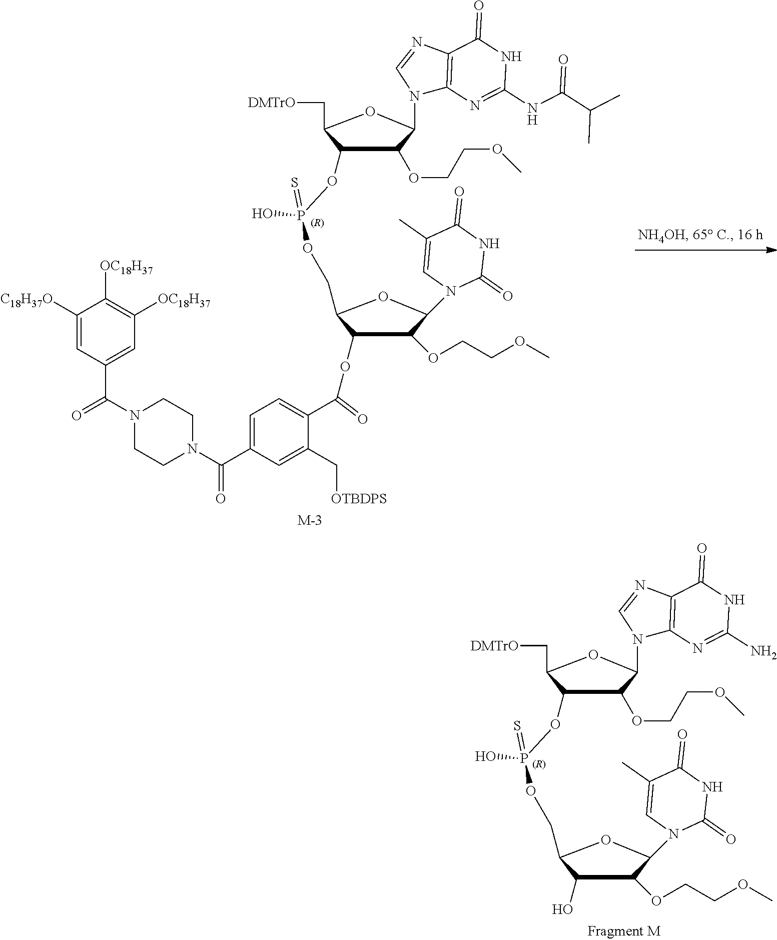

b. Procedures for Synthesis of Oligonucleotide Fragment M

General Procedure for Preparation of Compound M-3

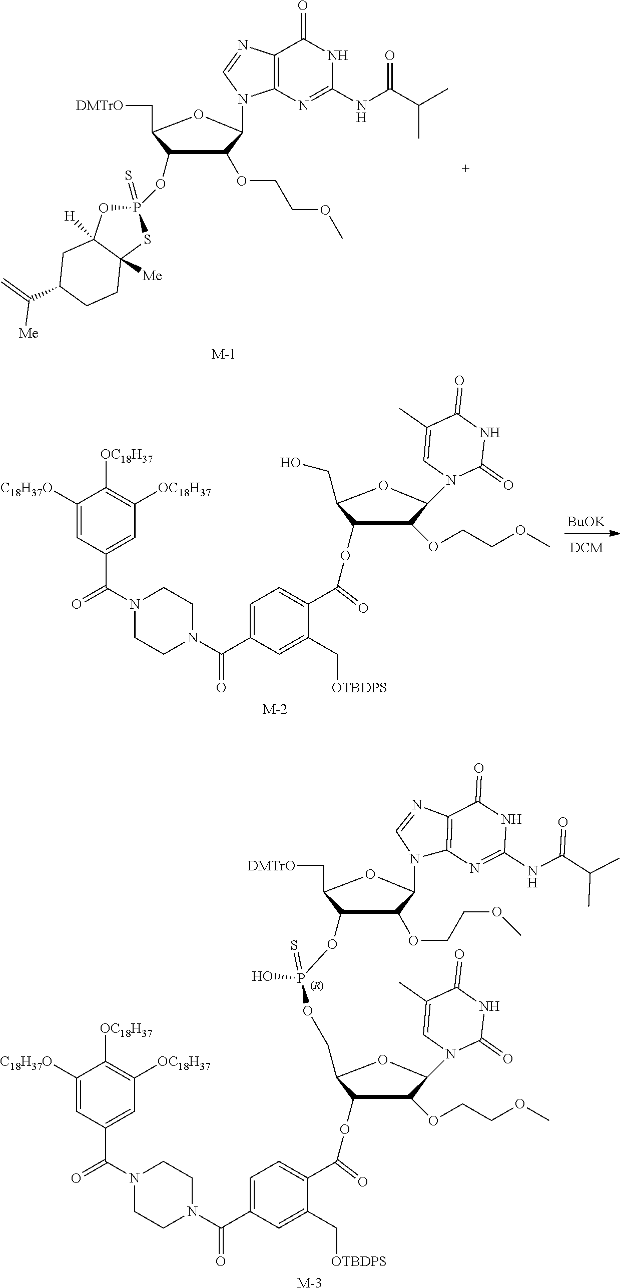

Compound M-1 (134.7 mg, 140.3 umol, 1.00 eq) with dry DCM (5.0 ml) and ACN (10.00 mL) were concentrated under reduced pressure to remove water two times. To a solution of compound M-1 (134.7 mg, 140.3 umol, 1.00 eq) and compound M-2 (160 mg, 93.5 ummol, 1.0 eq) in DCM (2.00 mL) was added 3A MS (200 mg) in one portion at 25° C. under Ar and stir for 0.5 hr. BuOK (1.0 M, 281 uL, 3.00 eq) was added into above mixture. The mixture was stirred at 25° C. for 1 hr. LCMS showed the starting material was consumed completely. The mixture was filtered and washed with DCM (0.5 mL). The crude product was triturated with ACN (20 mL) at 25° C. for 1 hr. The mixture was filtered and the cake was concentrated in vacuum. Chiral Fragment M (175 mg, 69.9 umol, 74.7% yield) was obtained as a white solid. Mass calcd for C₁₂₀H₁₈₉N₉0₂₂PSSi⁺[M−DMTr+H⁺]: 2200.3, found: 2200.1. ³¹P NMR (162 MHz, CDCl₃) δ 58.6 ppm.

General Procedure for Preparation of Fragment M

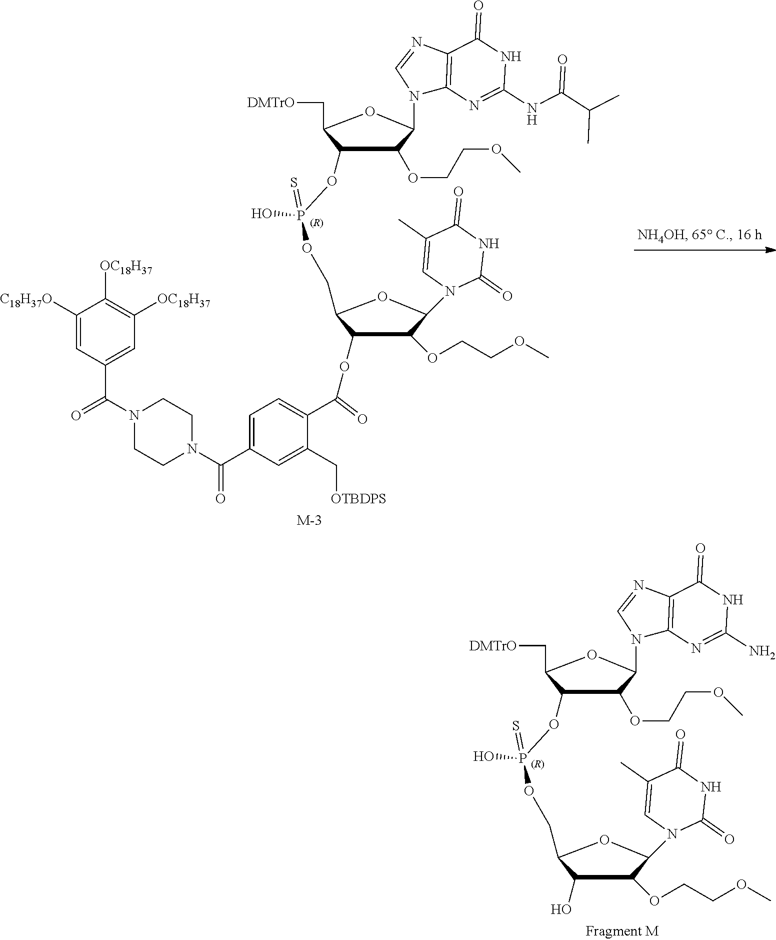

A solution of compound M-3 (30 mg) saturated with NH₃·H₂O (2.00 mL) was stirred at 65° C. for 16 hours in a 4 mL of sealed tube. Without any purification and the reaction mixture was filtered, the filtrate was provided for LCMS. Fragment M was confirmed by LCMS. HRMS Calcd for C₄₇H₅₆N₇O₁₆P⁻ [M−H⁻]: 1036.3169, found: 1036.3502.

### Example 19. Synthesis of Chiral Oligonucleotide Fragment N

a. Scheme for Synthesis of Chiral Oligonucleotide Fragment N

Chiral oligonucleotide Fragment N was synthesized according to the synthetic scheme depicted below:

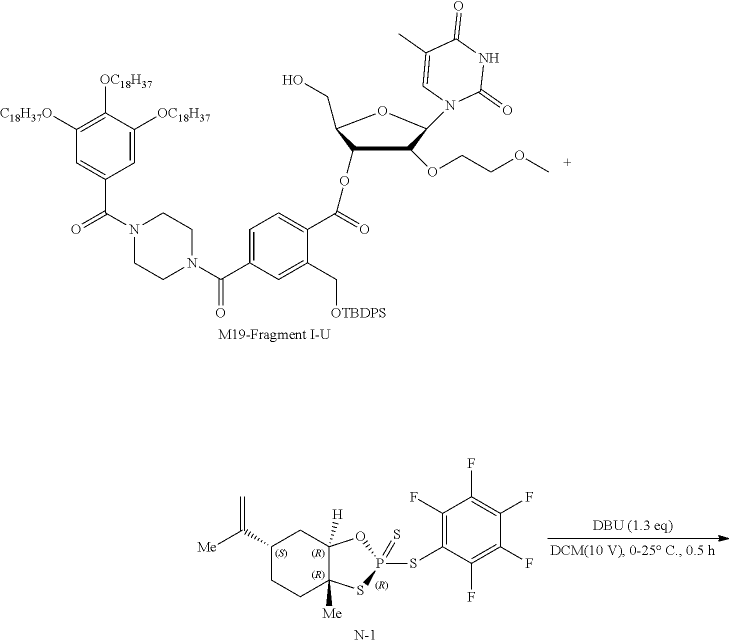

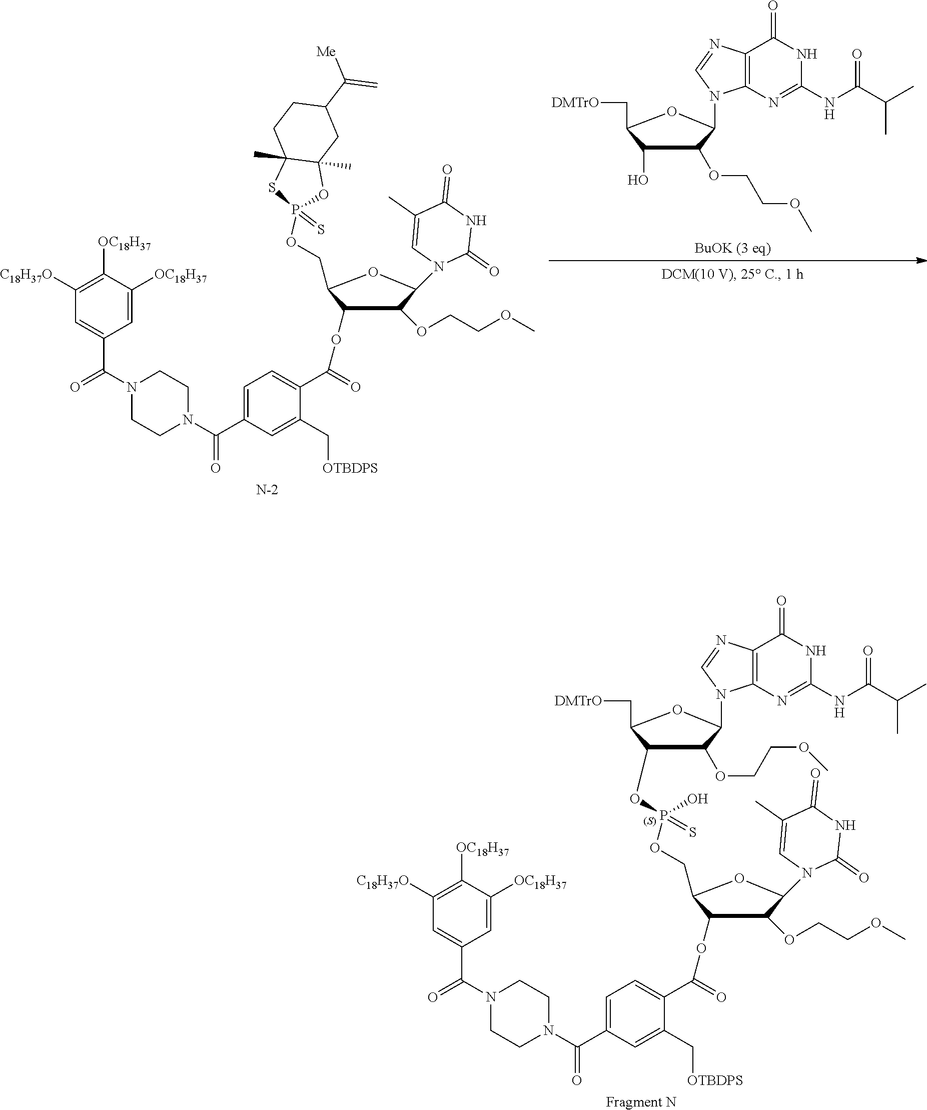

General Procedure for Preparation of Compound N-2

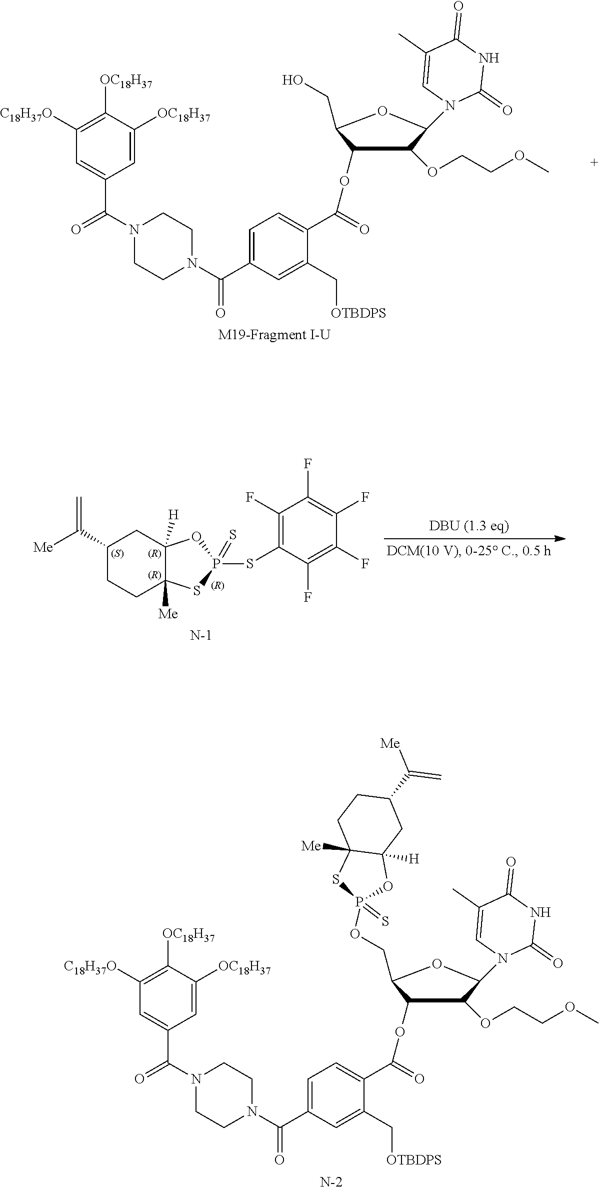

Compound M19-Fragment I-U (200 mg, 117 umol, 1.00 eq) with dry DCM (1.0 ml) and DCM (3.0 mL) were concentrated under reduced pressure to remove water two times. To a solution of compound M19-Fragment I-U (200 mg, 117 umol, 1.00 eq) and compound N-1 (68 mg, 152 ummol, 1.30 eq) in DCM (2.00 mL) was added 3A MS (200 mg) in one portion at 25° C. under Ar and stir for 0.5 hr. DBU (23 mg, 152 umol, 1.30 eq) was added into above mixture, and the mixture was stirred at 25° C. for 1 hr. The mixture was filtered and washed with DCM (1.0 mL). The crude product was triturated with ACN (30 mL) at 25° C. for 1 hr. The mixture was filtered and the cake was concentrated in vacuum. Compound N-2 (215 mg, 84.6 umol, 84.6% yield) was obtained as a white solid.

General Procedure for Preparation of Fragment N

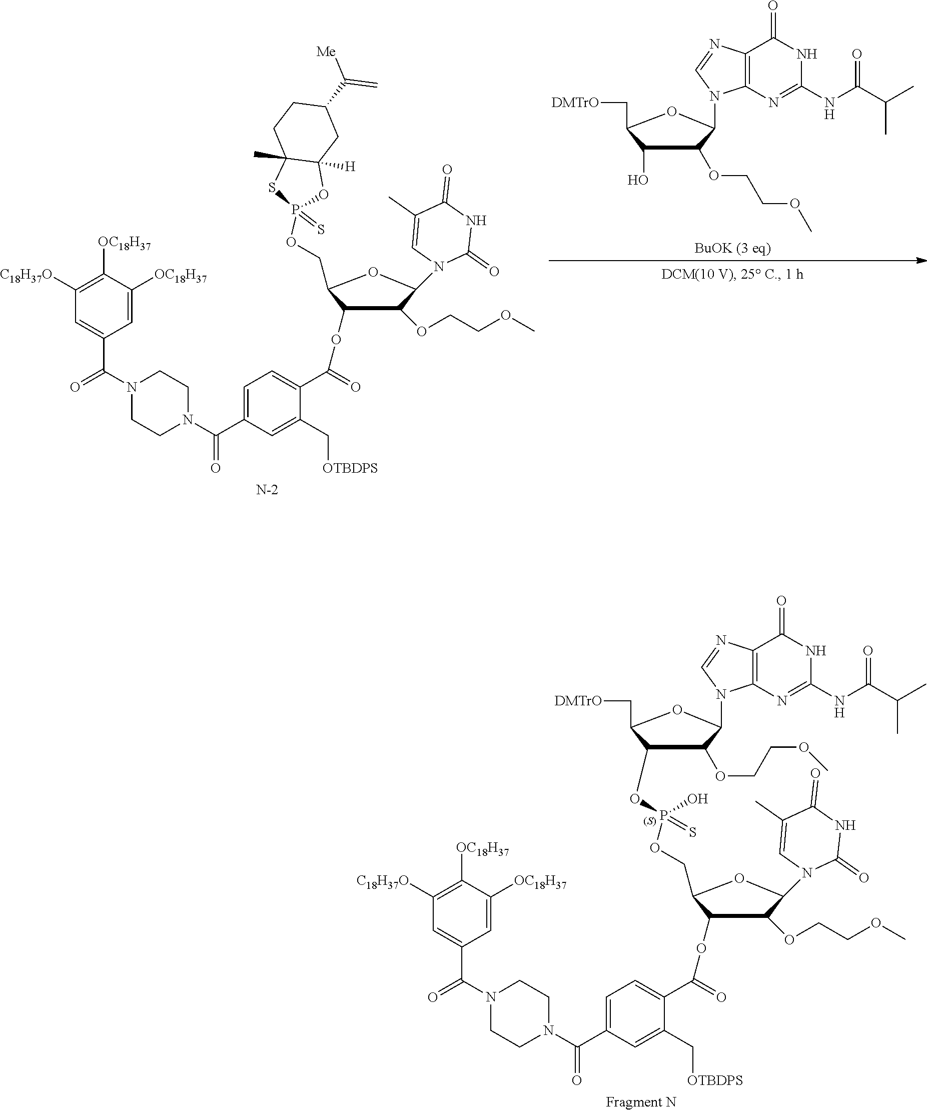

Compound N-2 (109.4 mg, 153 umol, 1.50 eq) with dry CH₃CN (1.0 ml) and DCM (3.0 mL) were concentrated under reduced pressure to remove water two times. To a solution of compound 5 (109.4 mg, 153 umol, 1.50 eq) and 5′-DMTr-MOE G-3′—OH (200 mg, 102.2 umol, 1.0 eq) in DCM (2.00 mL) was added 3A MS (200 mg) in one portion at 25° C. under Ar and stir for 0.5 hr. BuOK (1.0 M, 307 uL, 3.0 eq) was added into above mixture, and the mixture was stirred at 25° C. for 1 hr. The mixture was filtered and washed with DCM (1.0 mL). The crude product was triturated with ACN (25 mL) at 25° C. for 1 hr. The mixture was filtered and the cake was concentrated in vacuum. Chiral Fragment N (175 mg, 54.1 umol, 68.1% yield) was obtained as a white solid. Mass calcd for C₁₂₀H₁₈₉N₉0₂₂PSSi⁺[M−DMTr+H⁺]: 2200.3, found: 2200.1. ³¹P NMR (162 MHz, CDCl₃) δ 58.6 ppm.

### Example 20. Synthesis of Oligonucleotide Fragment O

a. Scheme for Synthesis of Oligonucleotide Fragment O

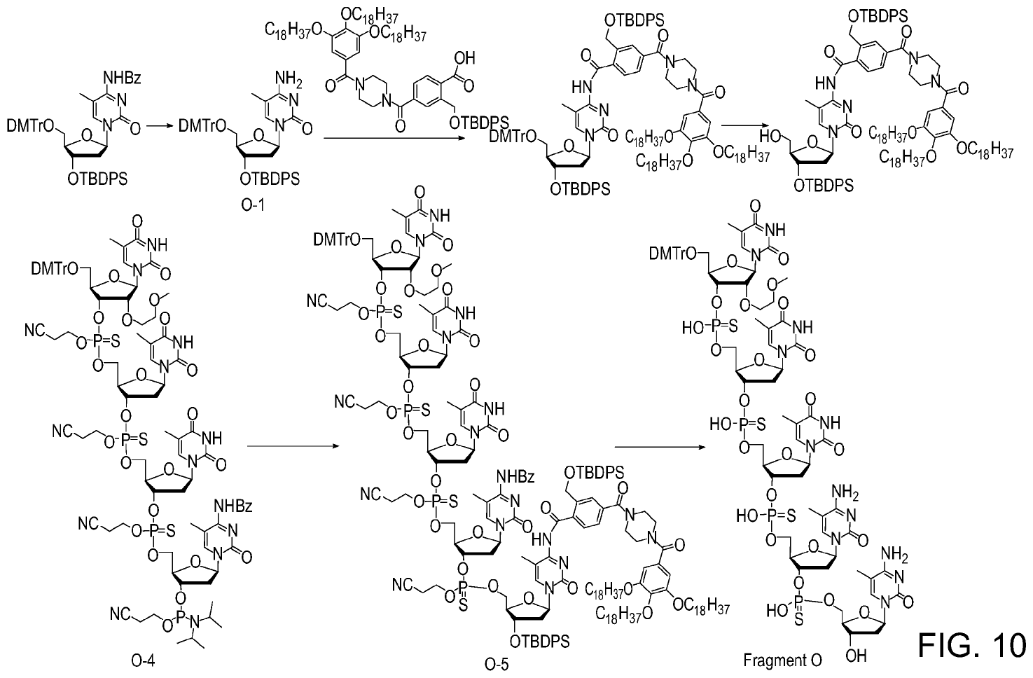

Oligonucleotide Fragment N was synthesized according to the synthetic scheme depicted in FIG. 10.

b. Procedures for Synthesis of Oligonucleotide Fragment O

General procedure for preparation of Compound O-1

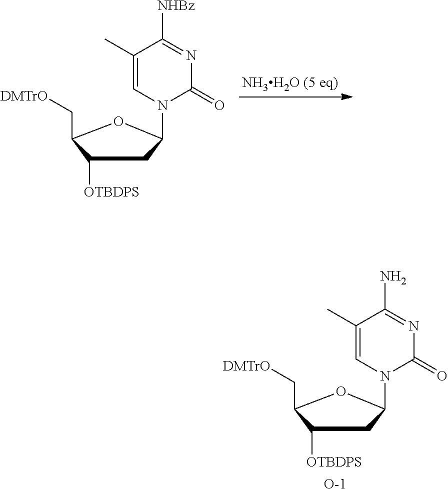

A solution of 5′-DMTrO-C-OTBDPS-3′ (5.0 g, 5.2 mmol, 1.00 equiv), NH3·H2O (25%, 3.6 g, 26.0 mmol, 5.00 equiv) and THF (40 mL) was stirred at 20±5° C. for 1.0 h (HLPC indicated 42.8% conversion of 5′-DMTrO-C-OTBDPS-3′). The mixture was concentrated to give a residue of crude product which was purified by silica gel chromatography (0-10% THF in DCM as eluent). The compound O-1 was obtained as a pale-yellow solid (1.6 g, 36.0% yield). 1H NMR (300 MHz, DMSO-d6) δ 8.02 (s, 1H), 7.90 (d, J=9.0 Hz, 1H), 7.64 (d, J=9.0 Hz, 2H), 7.58-7.09 (m, 22H), 6.93-6.72 (m, 5H), 5.96 (s, 1H), 4.33 (t, J=4.5 Hz, 1H), 4.12 (s, 1H), 3.63 (t, J=3.0 Hz, 1H), 3.39 (s, 2H), 3.34 (t, J=3.0 Hz, 2H), 3.28-3.19 (m, 2H), 3.18 (s, 3H), 3.05-2.93 (m, 1H), 1.39 (s, 3H), 0.94 (s, 9H). 13C{1H} NMR (75 MHz, DMSO-d6) δ 168.4, 165.8, 158.6, 155.3, 114.9, 135.9, 135.7, 135.4, 133.1, 130.2, 128.6, 128.2, 128.1, 127.9, 113.7, 102.0, 87.8, 86.5, 82.5, 81.7, 71.7, 71.4, 69.2, 63.1, 58.6, 55.5, 27.1, 19.3, 13.3. HRMS calcd for C50H58N₃O₈Si⁺[M+H]+: 856.3988, found: 856.4009

General Procedure for Preparation of Compound O-2

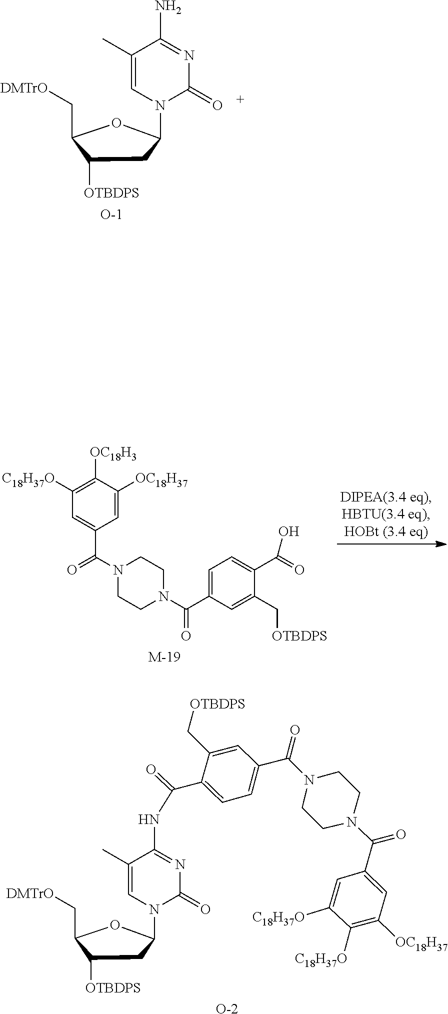

To a solution of compound O-1 (498 mg, 0.64 mmol, 1.50 eq) and compound M19 (600 mg, 0.42 mmol, 1.0 eq) in DCM (5.00 mL) was added DIPEA (186 mg, 3.4 eq), HBTU (549 mg, 3.4 eq), and HOBT (192 mg, 3.4 eq) at 25° C. under and stirred for 4 hours. The crude product was triturated with ACN (40 mL) at 25° C. for 1 hr. The mixture was filtered and the cake was concentrated in vacuum. Compound O-2 (700 mg, 0.32 mmol, 76.7% yield) was obtained as a white solid. Mass calcd for C₁₃₇H₁₉₆N₅0₁₃PSSi₂⁺[M+H⁺]: 2175.4363, found: 2175.4373.

General Procedure for Preparation of Compound O-3

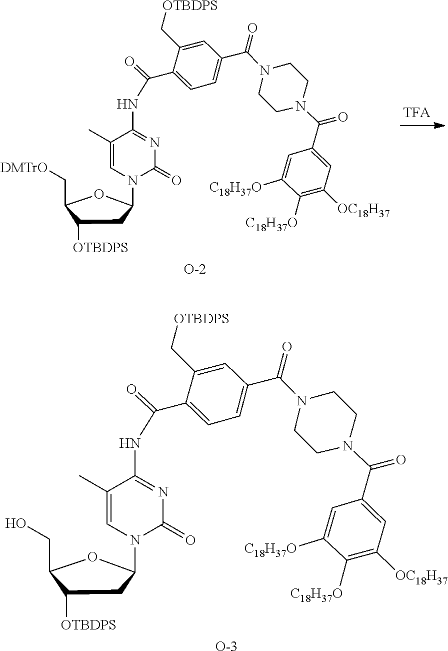

To a solution of compound O-2 (650 mg, 0.298 mmol, 1.00 eq) in DCM (6.0 mL) was added C₁₂H₂₅SH (180 mg, 3.0 eq) and DCA (339 mg, 10.0 eq) at 0-5° C. The mixture was stirred at 0-5° C. for 2.5 hours and NMI (293.5 mg, 12.00 eq) was added. The mixture was stirred at 0-5° C. for 0.5 hrs. The residue was diluted with NaHCO₄/H₂O (50 mL) and extracted with DCM (2×30 mL). The crude product was triturated with ACN (40.0 mL) at 25° C. for 30 min. Filtered and concentrated. Compound O-3 (500 mg, 89% yield) was obtained as a white solid. Mass Calcd for C₁₁₆H₁₇₈N₅O₁₁Si₂⁺[M+H⁺]: 1874.3, found: 1874.1.

General Procedure for Preparation of Compound 0-5

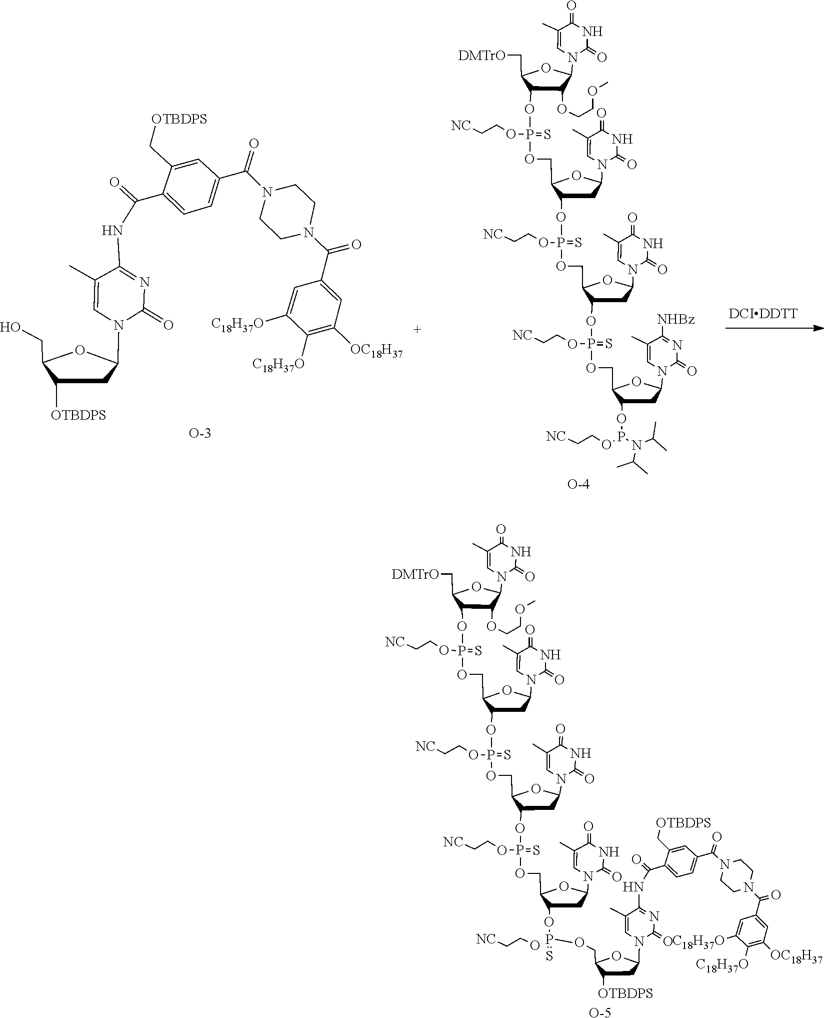

Compound O-3 (0.25 g, 133 umol, 1.00 eq) with dry DCM (4.0 ml) and ACN (4.00 mL) were concentrated under reduced pressure to remove water two times. To a solution of compound O-3 (0.25 g, 133 umol, 1.00 eq) in DCM (4.00 mL) was added 3A MS (200 mg) in one portion at 25° C. under Ar and stir for 0.5 hr. Compound O-5 (816 mg, 0.4 mmol, 3.00 eq) and DCI (50 mg, 426 umol, 3.2 eq) were added into above mixture. The mixture was stirred at 25° C. for 1 hr. DDTT (60 mg, 292 umol, 2.20 eq) was added into reaction solution, and the mixture was stirred at 25° C. for 0.5 hr. The crude product was triturated with ACN (40 mL) at 25° C. for 1 hr. The mixture was filtered and the cake was concentrated in vacuum. Compound O-5 (0.25 g, 50% yield) was obtained as a white solid.

General Procedure for Preparation of Fragment O

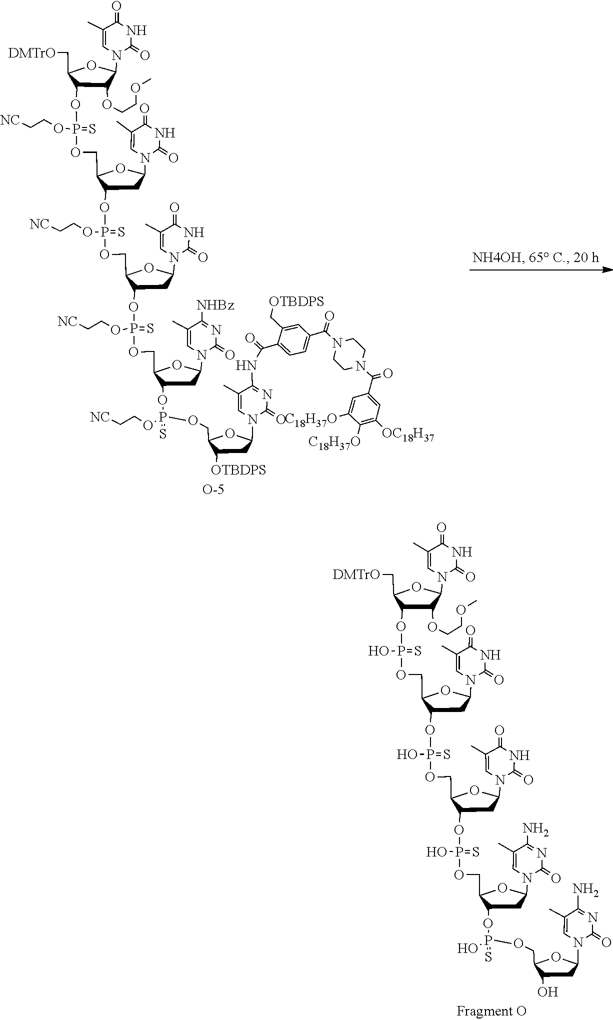

A solution of compound O-5 (20 mg) saturated with NH₃·H₂O (2.00 mL) was stirred at 65° C. for 20 hours in a 4 mL of sealed tube. Without any purification and the reaction mixture was filtered, the filtrate was provided for LCMS. Fragment O was confirmed by LCMS. HRMS Calcd for C₇₄H₉₁N₁₂O₃₁P₄S₄⁻ [M−H⁻]: 1895.3752, found: 1895.3716.

### Example 21. Synthesis of Monomer Fragment P

a. Scheme for Synthesis of Monomer Fragment P

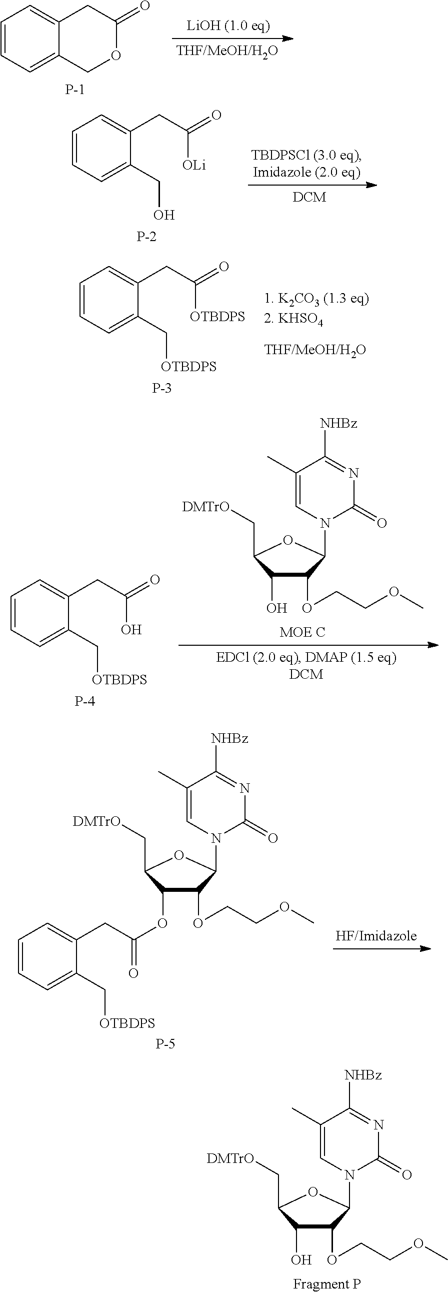

b. Procedures for Synthesis of Fragment P

General Procedure for Preparation of Compound P-5

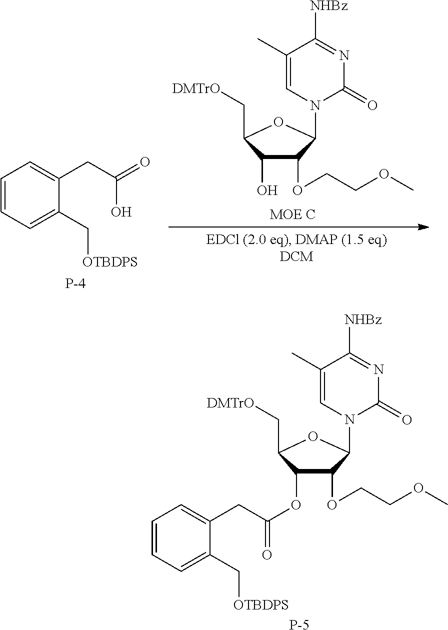

To a solution of compound P-4 ((synthetic procedures are described in European Journal of Organic Chemistry (2003), (12), 2327-2335, which is incorporated herein by reference), 1.0 g, 2.47 mmol, 1.0 eq) and 5′-DMTr-MOE C-3′-OH (1.78 g, 2.47 mmol, 1.0 eq) in DCM (20.0 mL) was added EDCl (947 mg, 2.0 eq) and DMAP (453 mg, 1.5 eq) at 25° C. under and stirred for 24 hours. The mixture was concentrated in vacuum and the residue was purified by silica gel column chromatography (Heptane/EtOAc 5:1 to 3:1). Compound P-5 was obtained as a white solid (1.7 g, 1.48 mmol, 60% yield). Mass calcd for C₆₆H₆₉N₃0₁₁Si⁺[M+H⁺]: 1108.4 found: 1108.4.

General Procedure for Preparation of Fragment P

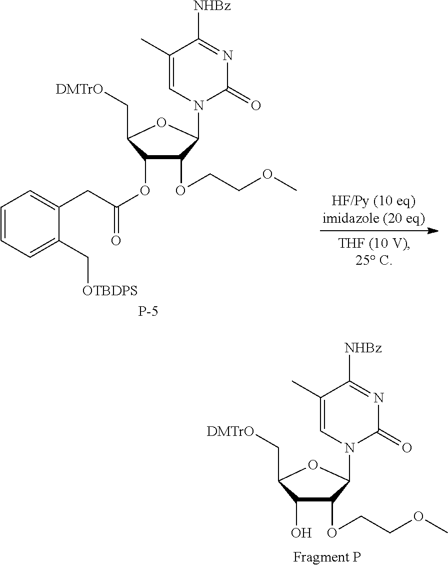

To a solution of compound P-5 (0.5 g, 0.45 mmol, 1.00 eq) in THF (1.0 mL) was added solution of imidazole (614.2 mg, 9.02 mmol, 20.0 eq) and pyridine/hydrofluoride (128.9 mg, 4.5 mmol, 70% purity, 10.0 eq) in THF (2.0 mL). The mixture was stirred at 25° C. for 20 hrs (Compound P-5 was converted to Fragment P with >95% conversion).

c. Alternative Scheme for Synthesis of Monomer Fragment P

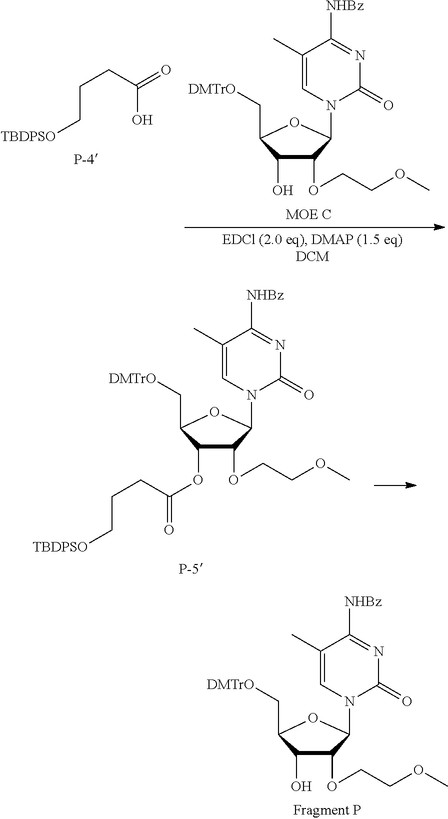

d. Alternative Procedures for Synthesis of Fragment P

General Procedure for Preparation of Compound P-5′

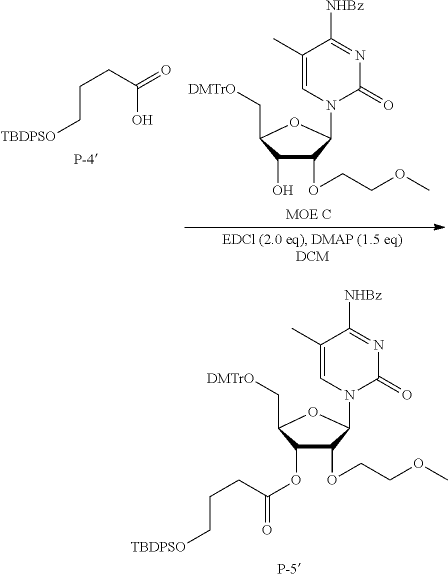

To a solution of compound P-4′ (1.0 g, 2.92 mmol, 1.0 eq) and 5′-DMTr-MOE C-3′-OH (2.10 g, 2.92 mmol, 1.0 eq) in DCM (10.0 mL) was added EDCl (2.80 g, 5.0 eq) and DMAP (0.71 g, 2.0 eq) at 25° C. under and stirred for 24 hours. The mixture was concentrated in vacuum and the residue was purified by silica gel column chromatography (Heptane/EtOAc 5:1 to 3:1). Compound P-5′ was obtained as a white solid (1.97 g, 90% yield). Mass calcd for C61H67N3011Si+[M+H⁺]: 1046.4 found: 1046.4.

General Procedure for Preparation of Fragment P

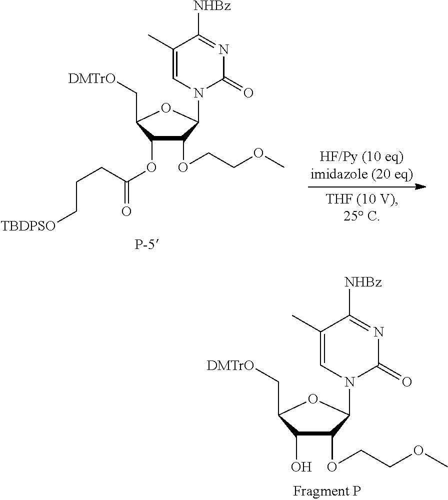

To a solution of compound P-5′ (0.3 g, 0.29 mmol, 1.00 eq) in THF (1.0 mL) was added solution of imidazole (390.2 mg, 5.73 mmol, 20.0 eq) and pyridine/hydrofluoride (81.9 mg, 2.9 mmol, 70% purity, 10.0 eq) in THF (2.0 mL). The mixture was stirred at 25° C. for 20 hours and Compound P-5′ was converted to Fragment P with ˜60% conversion.

e. Alternative Scheme for Synthesis of Monomer Fragment P

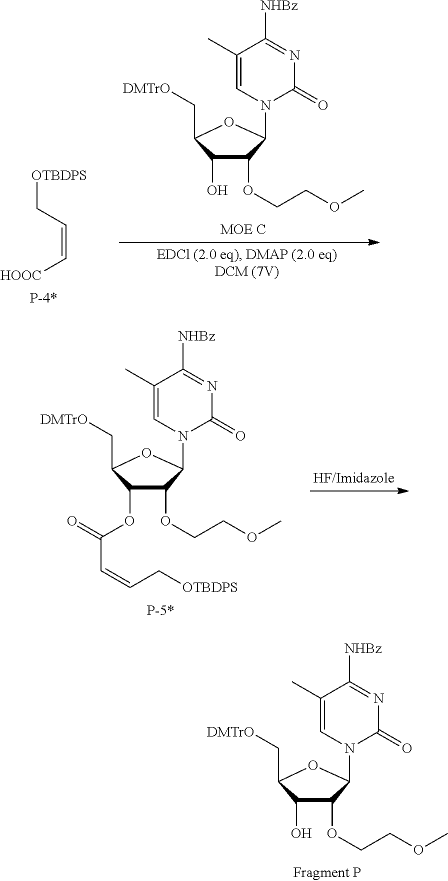

General procedure for preparation of Compound P-5*

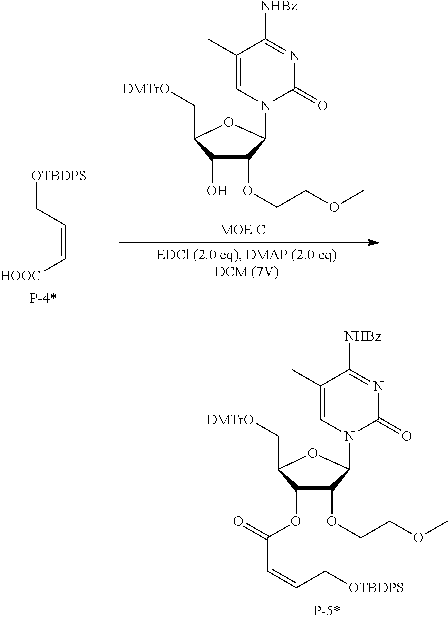

To a solution of compound P-4* (2.12 g, 6.23 mmol, 1.5 eq) and 5′-DMTr-MOE C-3′-OH (3.0 g, 4.16 mmol, 1.0 eq) in DCM (20.0 mL) was added EDCl (1.59 g, 2.0 eq) and DMAP (1.02 g, 2.0 eq) at 25° C. under and stirred for 24 hours. The mixture was concentrated in vacuum and the residue was purified by silica gel column chromatography (Heptane/EtOAc 5:1 to 3:1). Compound P-5* was obtained as a sticky oil (2.5 g, 93% yield). Mass calcd for C₆₁H₆₅N₃0₁₁Si⁺[M+H⁺]: 1046.4 found: 1044.4.

General Procedure for Preparation of Fragment P

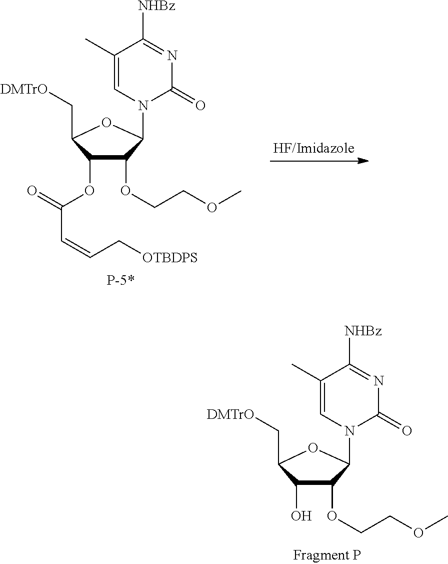

To a solution of compound P-5* (0.15 g, 0.145 mmol, 1.00 eq) in THF (1.0 mL) was added solution of imidazole (195.1 mg, 2.87 mmol, 20.0 eq) and pyridine/hydrofluoride (41 mg, 1.45 mmol, 70% purity, 10.0 eq) in THF (2.0 mL). The mixture was stirred at 50° C. for 6 hrs and Compound P-5* was converted to Fragment P with˜90% conversion.

### Example 22. Synthesis of Oligonucleotide Fragment B from M40

a. Scheme for Synthesis of Oligonucleotide Fragment B from M40

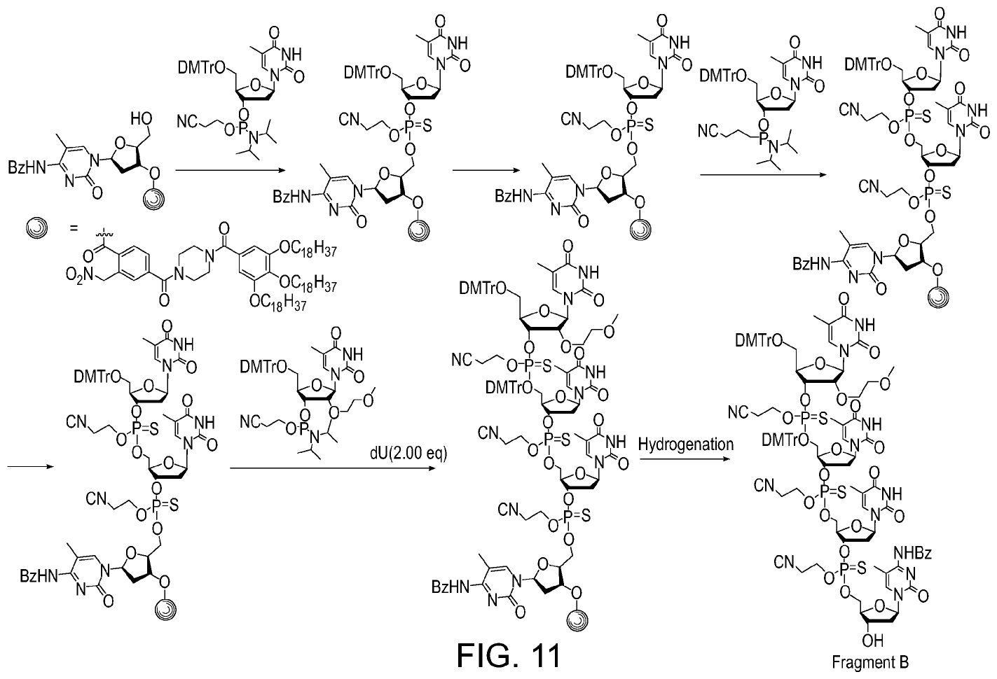

Fragment B was synthesized according to synthetic scheme depicted in FIG. 11.

b. Procedures for Synthesis of Oligonucleotide Fragment B from reagent M40

Procedures for synthesizing oligonucleotide fragment B from reagent M40 were similar those described for the synthesis of oligonucleotide fragment B from M19, except for the last step of selective deprotection of M40. The procedure of selective deprotection of M40 is described as below.

The Procedure of Selective Deprotection of M40

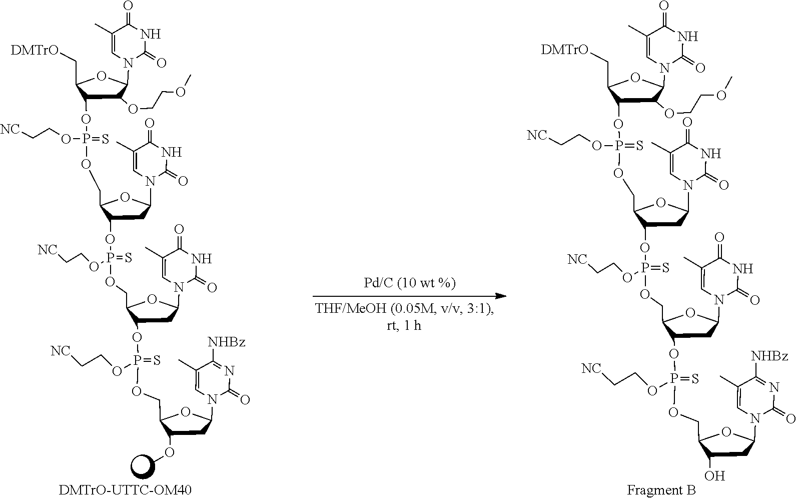

Under a hydrogen atmosphere, stirred a mixture of DMTrO-UTTC-OM40 (1.0 eq) and palladium on carbon (10 wt %) in tetrahydrofuran and methanol (0.05M, v/v, 3:1)) vigorously at room temperature for 1 hour. Filtered and concentrated the reaction mixture to residue which was precipitated in MTBE to yield desire product. Fragment B was obtained as a pale-yellow solid in 70.5% yield and 85.2% purity.

### Example 23. One Pot Procedure for Preparation of P═O Linkage

a. Scheme for One Pot Procedure for Preparation of P═O Linkage

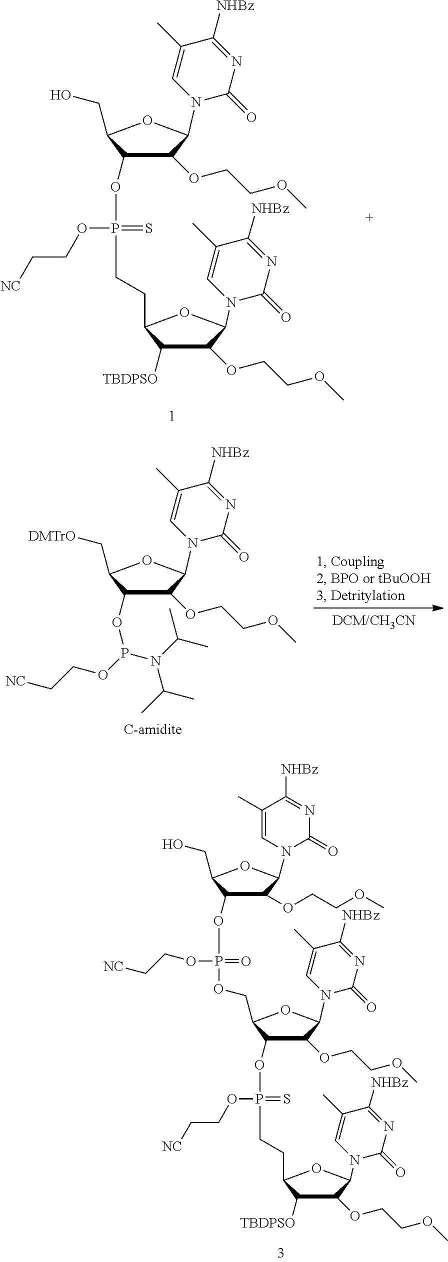

b. Procedure for Preparation of P═O Linkage

A mixture of compound 1 (12 g, 10 mmol, 1.00 eq), MOE C amidite (11.16 g, 12 mmol, 1.20 eq) and 3A MS (12.0 g) in CH₃CN/DCM (100 mL, v/v=1:3) was stirred at 20-30° C. for 1 h, DCI (1.94 g, 15 mmol, 1.50 eq) was added, and the reaction mixture was stirred at 20-30° C. for 1.0 h (HPLC indicated the reaction conversion >99.5%). H₂O (40 mg, 2 mmol, 0.2 eq) was added and the mixture was stirred at 25° C. for 30 min. NMI (1.35 g, 15 mmol, 1.5 eq), BPO (3.89 g, 11 mmol, 1.1 eq) and iodine (278 mg in DCM 6 mL, 1 mmol, 0.1 eq) were added to the reaction mixture at 0-5° C. and stirred at 0-5° C. for 1 h. Piperazine (652 mg, 7 mmol, 0.7 eq) was added and the mixture and stirred at 0-5° C. for 30 min. Dodecane-1-thiol (6.64 g, 3.0 eq) and 3 Å MS (10.0 g) were added and the mixture was stirred at 0-10° C. for 60 min. TFA (13.7 g, 110 mmol, 11.00 eq) was added dropwise at 0-5° C. and stirred at 10-20° C. for 60 min. NMI (9.88 g, 110 mmol, 11.0 eq) was added at 0-5° C. and stirred at 0-5° C. for 10 min. The reaction mixture was filtered to remove 3 Å MS and added into 5% NaHCO₃ solution (120 mL) with vigorous stirring. EtOAc (120 mL) and MTBE (120 mL) were added and stirred vigorously for 10 min. The organic layer was separated, washed with 5% aqueous NaHCO₃ solution (120 mL), H₂O (120 mL), brine (120 mL), dried over MgSO₄ (24 g), filtered, and concentrated in vacuo. The crude product was dissolved in EtOAC (36 mL) slowly added to a mixture of heptane/TBME (216 L, 1:1, v/v). The precipitated product was filtered, washed with heptane/TBME (2×400 mL, 1:1, v/v) and dried under vacuum at 20-30° C. for 16 h to yield compound 3 as a white solid (14.3 g, 80.1% yield). HRMS calcd for C₈₂H₉₇N₁₁O₂₄P₂SSi⁺[M+H]⁺: 1742.5751, found: 1742.5732.

c. Comparison Among Different Oxidation Reagents

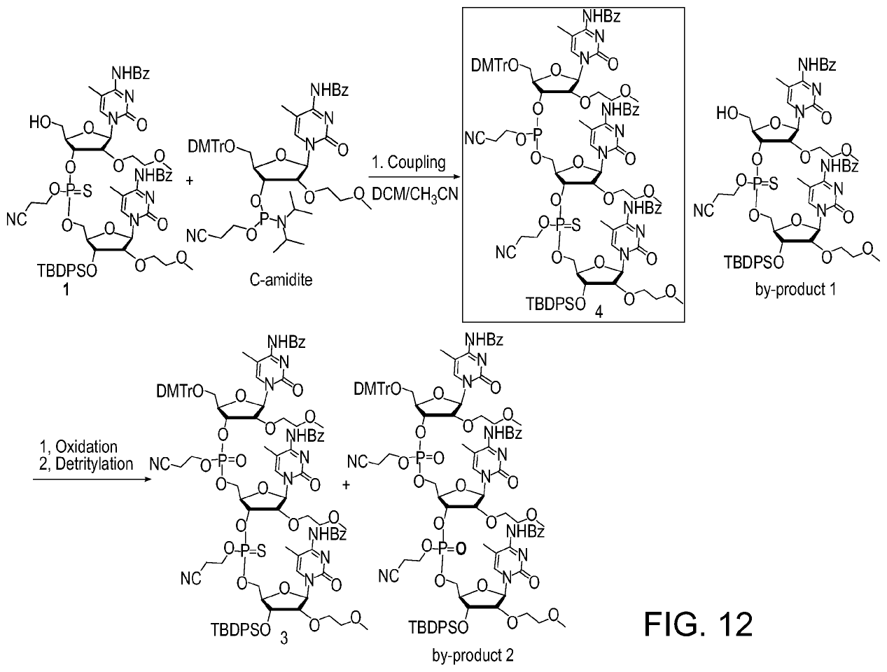

Several oxidation reagents, including iodine/pyridine, mCPBA, BPO, and tBuOOH, have been tested in the one pot procedure (coupling/oxidation/detritylation) for preparing P═O linkage. The scheme, which shows the reaction product 3 and by-products 1 and 2, is depicted in FIG. 12. The performance of each oxidation reagent is summarized in Table 2 below.

| Oxidation | | | |
|---|---|---|---|
| reagent | By-product 1 | By-product 2 | Purification |
| iodine/ | 0.4-1.0% | | Need to remove |
| pyridine | | | pyridine before |
| | | | detritylation |
| mCPBA | | >10% PS was | |
| | | converted to PO | |
| BPO | <0.2% | <0.1% | |
| tBuOOH | <0.2% | <0.1% | |

Oxidation reagents BPO and tBuOOH demonstrated superior oxidation performance than iodine/pyridine and mCPBA in the one-pot procedure for preparation of the P═O linkage in oligonucleotides. When BPO or tBuOOH was used in the one-pot procedure, both of them produced less by-products than iodine/pyridine and mCPBA did. In addition, the one-pot procedure was not successful for iodine/pyridine because it required an additional purification step to remove pyridine before carrying out the detritylation step.

### Example 24. Synthesis of Oligonucleotide I

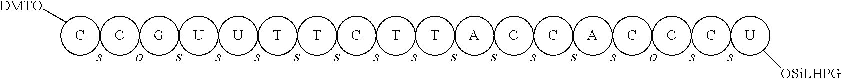

Step 1: Synthesis of Compound I-3

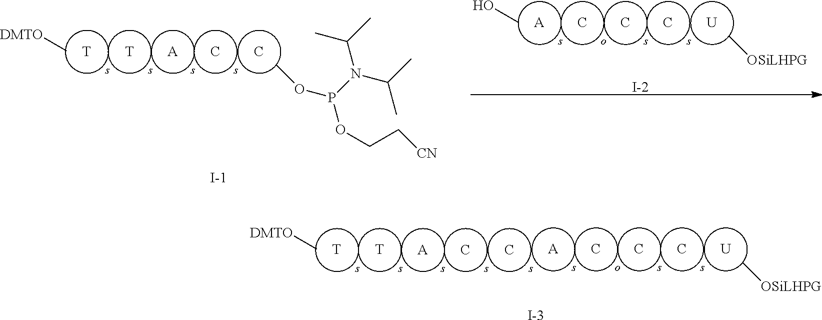

To a first round bottom flask (RBF) was added compound I-2 (1 eq) under Ar. It was dried three times by co-evaporating with (DCM/ACN=3:1,4 v) at 25-30° C. Then DCM/ACN=2:1 (6V) was added to the RBF, followed by 3A MS (5%) at 25-30° C. for 1 h. Compound I-1 (1.5 eq) was then added to a second RBF under Ar and was dried three times by co-evaporating with (ACN 4 v) at 25-30° C. DCM (2V) was added to the second RBF and the resulting solution in the second RBF was added to the first RBF dropwise at 20-25° C., followed by the addition DCI (2 eq). The resulting mixture was stirred at 25-30° C. for 1 h. A sample was taken for analysis. Then to the reaction mixture was added DDTT (2eq). The mixture was stirred at 25-30° C. for 0.5h. A sample was taken for analysis. Then the mixture was filtered to remove 3 Å MS, washed with DCM (2V×2). The resulted solution was slowly added into ACN (50 V) at 20° C. for 0.5 h. Solid was collected by filtration, washed by ACN (5V×2) to afford compound I-3 as a white solid.

Step 2: Detritylation of Compound I-3

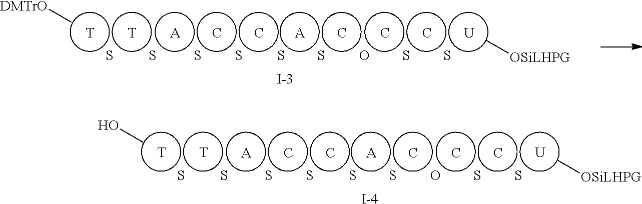

Compound I-3 (1 eq) was added into a RBF under Ar, followed by DCM (7 V) at 0-5° C. under Ar and 3A MS (5%) at 20-25° C. for 1 h. Then to the mixture was added C₁₂H₂₅SH (2 eq), followed by TCA (10 eq) dropwise at 0-5° C. for 2h. A sample was taken for analysis. Py (12 eq) was added at 0-5° C. The mixture was filtered to remove 3 Å MS and washed by DCM (2V×2). The pH value was adjusted to 7˜8 by adding NaHCO₃ (4% wt, 10 V). Then the mixture was extracted with DCM (2V×2). The organic layer was dried over anhydrous MgSO₄, filtered, and washed by DCM (2 V×2). The filtrate was concentrated to ˜5V which was slowly added into ACN (50 V) at 20° C. for 0.5 h. Solid was collected by filtration and washed by ACN (5 V×2) to give compound I-4 as a white solid.

Step 3: Synthesis of Compound I-6

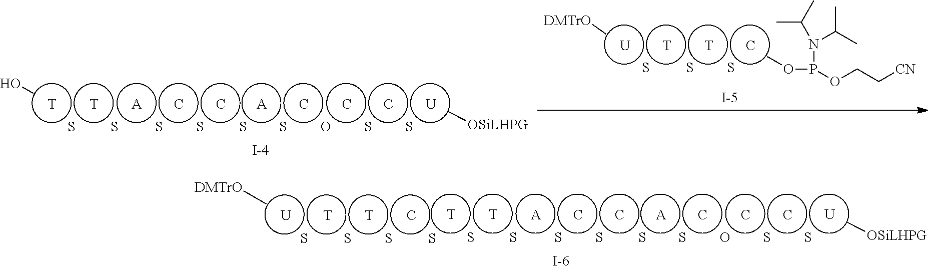

Compound I-4 (1 eq) was added to a first RBF under Ar. It was dried three time by co-evaporating with DCM/ACN(3:1,4 v) at 25-30° C. Then DCM/ACN (2:1, 6V) was added to the RBF, followed by 3A MS (5%) at 25-30 for 1 h. Compound I-5 was added (1.5 eq) to a second RBF under Ar. The mixture was dried three times by co-evaporating with ACN (4 v) at 25-30° C. and then DCM (2V) was added. the resulting solution in the second RBF was transferred to the first RBF dropwise at 20-25° C., followed by the addition of DCI (2 eq). The resulting mixture was stirred at 25-30° C. for 1 h. A sample taken out for analysis. DDTT (2eq) was added to the RBF. The resulting mixture was stirred at 25-30° C. for 0.5h. A sample was taken out for analysis. Then the reaction mixture was filtered to remove 3 Å MS, washed by DCM (2 V×2). The resulting solution was slowly added into ACN (50 V) at 20° C. for 0.5 h. Solid was collected by filtration and washed by ACN (5 V×2) to give compound I-6 as a white solid.

Step 4: Detritylation of Compound I-6

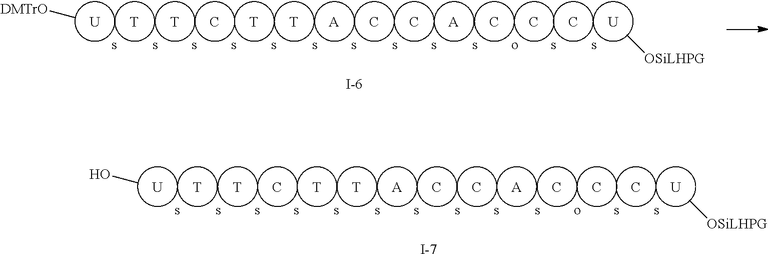

Compound I-6 (1 eq) was added to a round bottom flask (RBF) under Ar, followed by DCM (7 V) at 0-5° C. under Ar and 3A MS (5%) at 20-25° C. for 1 h. Then to the mixture was added C₁₂H₂₅SH (3 eq), followed by TCA (12 eq) dropwise at 0-5° C. for 2h. A sample was taken out for analysis. Then Py (15 eq) was added to the RBF at 0-5° C. The mixture was filtered to remove 3 Å MS and washed with DCM (2 V×2). The pH value was adjusted to 7˜8 by adding NaHCO₃ (4% wt, 10 V). Then it was extracted with DCM (2V×2). The organic layer was dried over anhydrous MgSO₄, filtered, and washed by DCM (2 V×2). The filtrate was concentrated to ˜5V which was slowly added into ACN (50 V) at 20° C., for 0.5 h. Solid was collected by filtration and washed with ACN (5 V×2) to give compound I-7 as a white solid.

Step 5: Synthesis of Oligonucleotide I

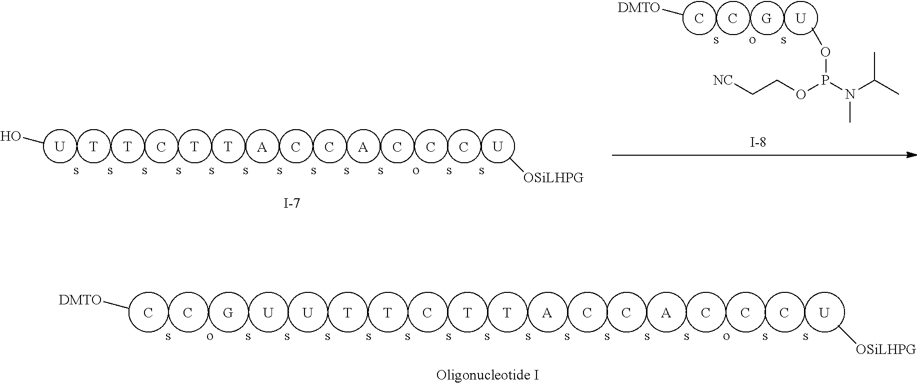

Compound I-7 (1 eq) was added to a first RBF under Ar. It was dried three time by co-evaporating with DCM/ACN(3:1,4 v) at 25-30° C. Then DCM/ACN (3:1, 10 V) was added to the RBF, followed by 3A MS (5%) at 25-30 for 1 h. Compound I-8 was added (1.7 eq) to a second RBF under Ar. The mixture was dried three times by co-evaporating with ACN (4 v) at 25-30° C. and then DCM (2V) was added. the resulting solution in the second RBF was transferred to the first RBF dropwise at 20-25° C., followed by the addition of DCI (2.5 eq). The resulting mixture was stirred at 25-30° C. for 1 h. A sample was taken out for analysis. DDTT (2eq) was added to the RBF. The resulting mixture was stirred at 25-30° C. for 0.5h. A sample was taken out for analysis. Then the reaction mixture was filtered to remove 3 Å MS, washed by DCM (2 V×2). The resulting solution was slowly added into ACN (50 V) at 20° C. for 0.5 h. Solid was collected by filtration and washed by ACN (5 V×2) to give oligonucleotide I in 3.1 g (76.4% yield, 69% UV purity) as a white solid.

### Example 25. Scale up Synthesis of Oligonucleotide I

a. Scheme for Synthesis of Oligonucleotide I

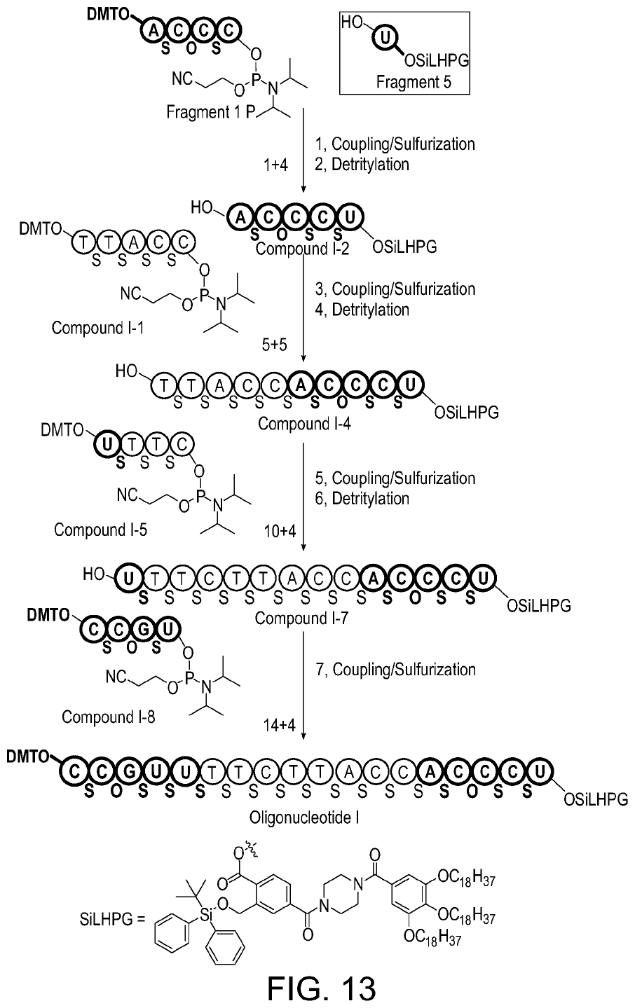

Oligonucleotide I was synthesized at a 50 gram scale according to synthetic scheme depicted in FIG. 13.

b. Procedures for Synthesis of Oligonucleotide I

General Procedure for Preparation of Compound I-2

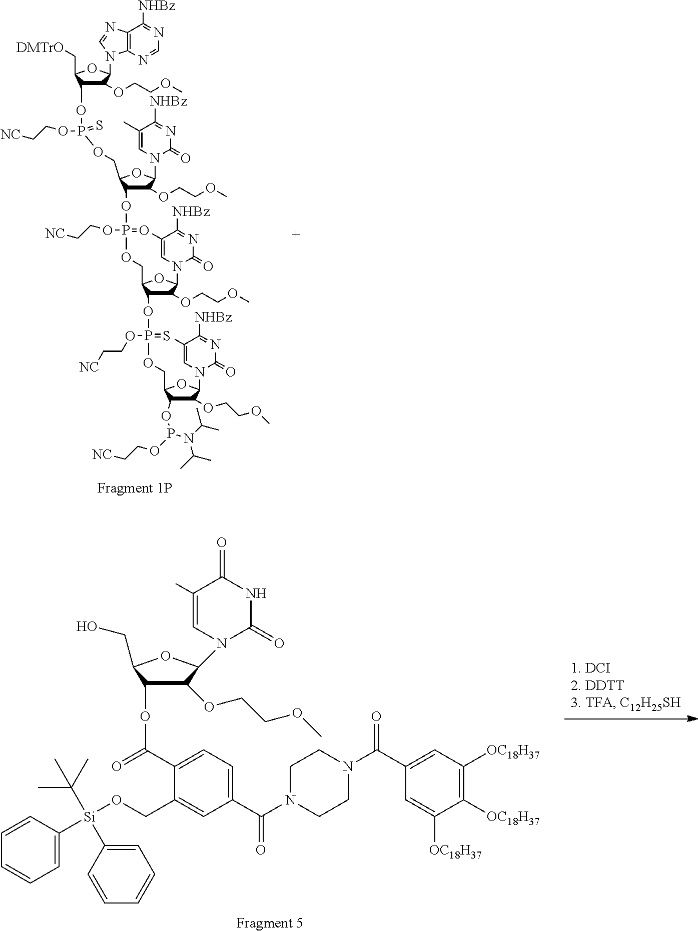

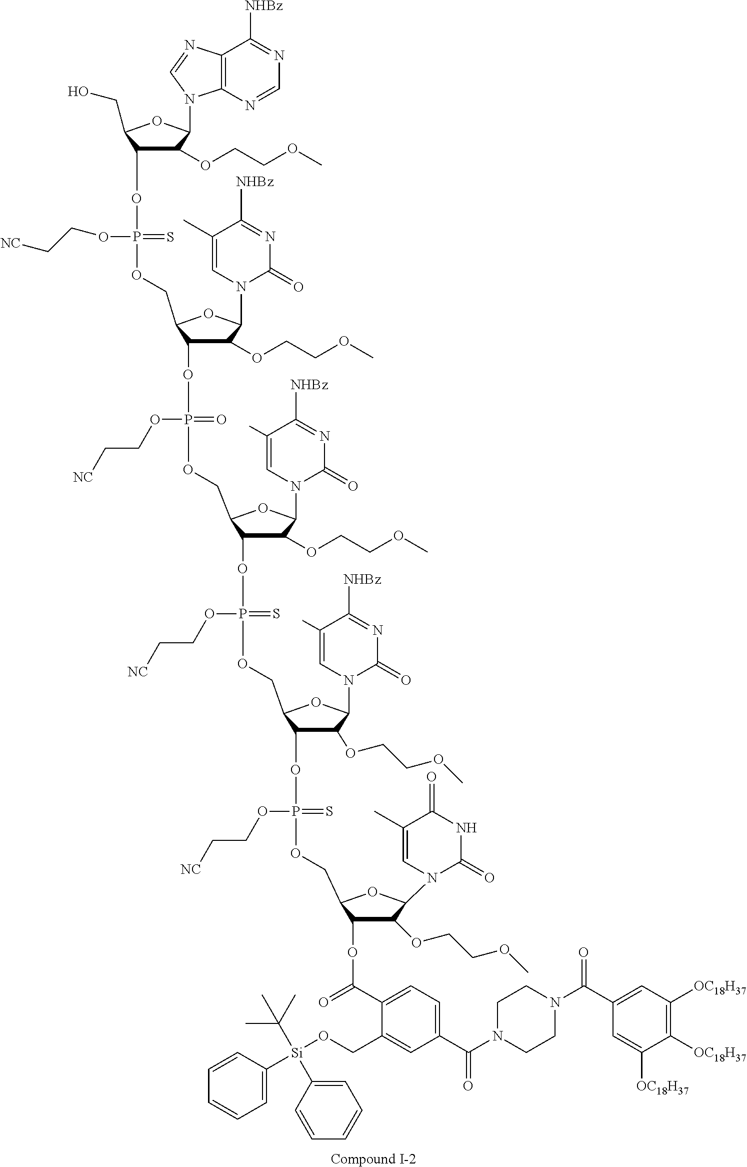

A mixture of Fragment IP (24.0 g, 14.1 mmol), Fragment 5 (44.1 g, 17.2 mmol), 3A MS (5 g/100 mL, 12 g), and DCM (240 mL) under N₂ atmosphere was stirred at 20-25° C. for 1.0 h. DCI (6.6 g, 56.4 mmol) was added and the reaction mixture was stirred for 1.0 h at 20-30° C. DDTT (7.2 g, 35.25 mmol) was added and the reaction mixture was stirred at 20-25° C. for 30 min. Dodecane-1-thiol (9.94 g, 49.3 mmol) was added and the reaction mixture was stirred at 0±5° C. for 10 min. TFA (14.4 g, 126.9 mmol) was added slowly and the reaction mixture was stirred at 0±5° C. for 1.0 h. NMI (12.7 g, 155.1 mmol) was added in 10 min, and the reaction mixture was filtered to remove 3A MS, concentrated to about 100 mL by rotovap and added to CH₃CN (2.6 L) in 30 min with vigorous agitation at 0±5° C. The precipitated product was filtered, washed with ACN (2×100 mL) and dried under vacuum at 20-30° C. for 16 h to yield compound I-2 as a white solid (46.7 g, 85.1% yield).

General Procedure for Preparation of Compound I-3

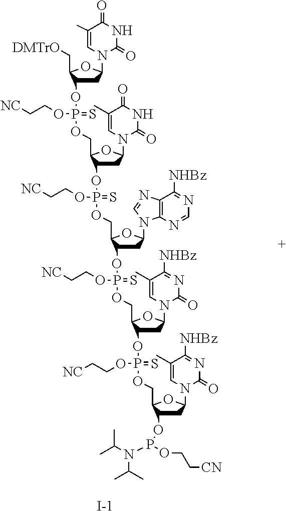

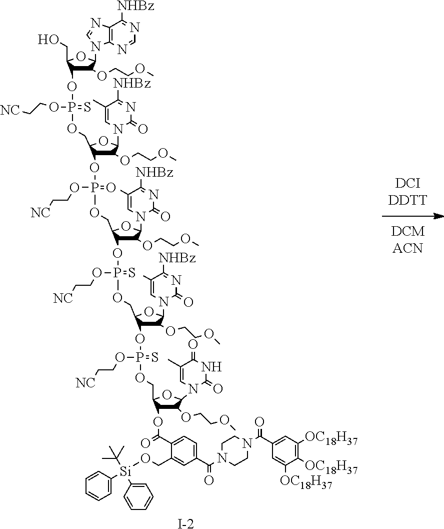

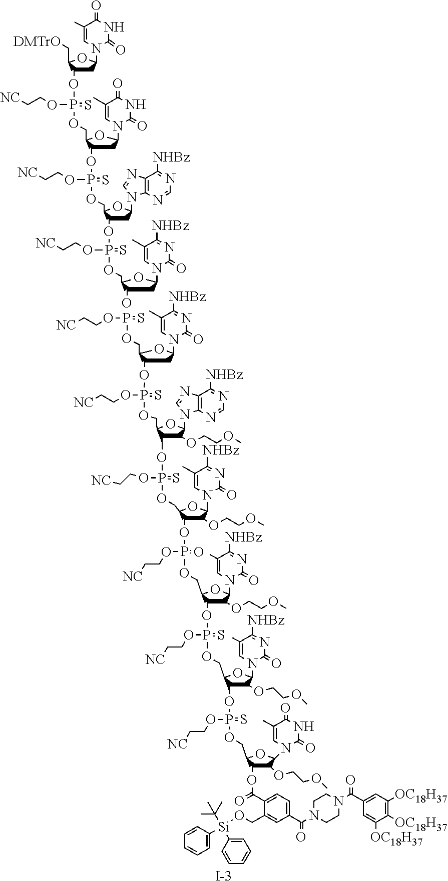

A mixture of compound I-2 (44.0 g, 11.3 mmol), compound I-1 (40.8 g, 15.9 mmol), 3 Å MS (44 g) and ACN/DCM (440 mL, 1:3, v/v) was stirred at 20-25° C. for 1.0 h under N₂ atmosphere. DCI (6.66 g, 56.5 mmol) was added, and the reaction mixture was stirred for 1.0 h. DDTT (5.1 g, 34.9 mmol) was added and the reaction mixture was stirred at 20-25° C. for 30 min. The reaction mixture was filtrated and the 3 Å MS filter cake was washed with DCM (2×50 mL). The combined filtrate was concentrated to about 200 mL on a rotary evaporator and added to ACN (2 L) in 30 min with vigorous agitation at 0±5° C. The precipitated product was filtered, washed with ACN (2×100 mL) and dried under vacuum at 20-30° C. for 12 h to yield compound I-3 as a slightly yellow solid (71.4 g, 98.6% yield).

General Procedure for Preparation of Compound I-4

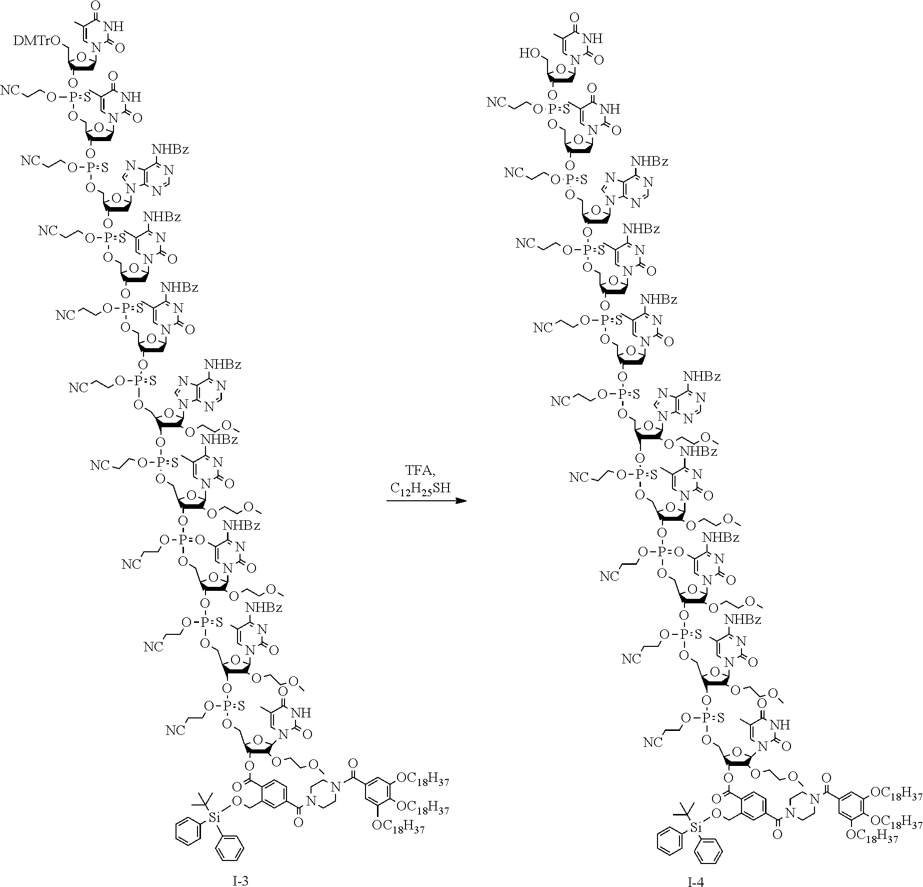

A mixture of compound I-3 (69.0 g, 10.8 mmol), 3 Å MS (71.0 g) and DCM (480 mL) was stirred at 20-25° C. for 1.0 h under N₂ atmosphere and cooled to 0±5° C. Dodecane-1-thiol (5.6 g, 27 mmol) was added and the reaction mixture was stirred at 0° C. for 10 min. TFA (13.6 g, 118.8 mmol) was added slowly and the reaction mixture was stirred at 0±5° C. for 1.5. NMI (11.5 g, 140.4 mmol) was added in 10 min, the reaction mixture was filtered to remove 3 Å MS, concentrated to about 200 mL on rotary evaporator and added to ACN (2.5 L) in 30 min with vigorous agitation at 0±5° C. The precipitated product was filtered, washed with ACN (2×172 mL) and dried under vacuum at 20-30° C. for 14 h to yield compound I-4 as a white solid (55.86 g, 85.0% yield).

General Procedure for Preparation of Compound I-6

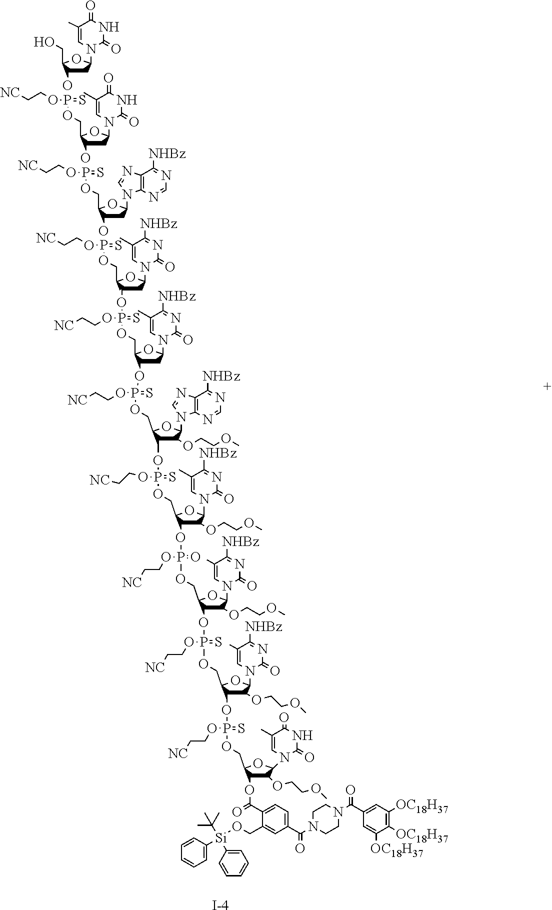

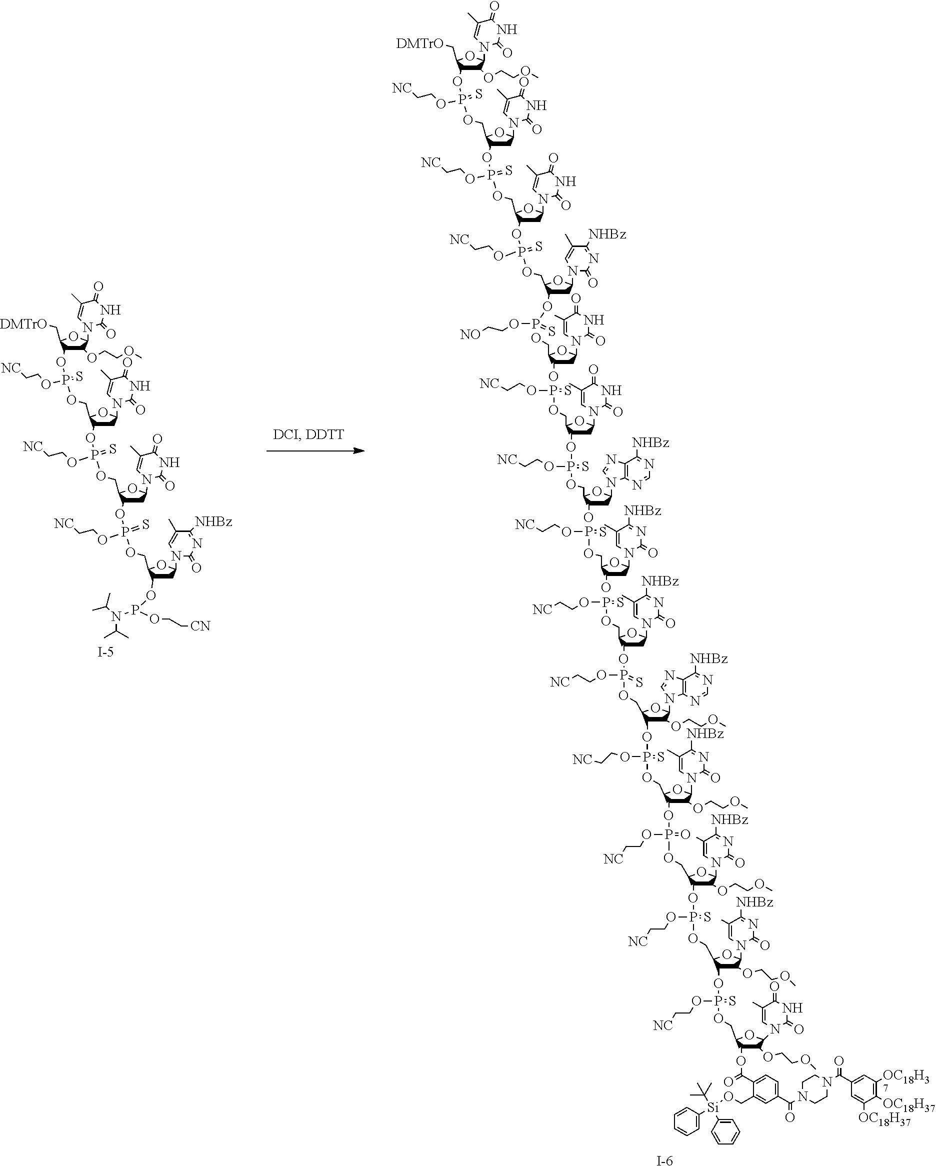

A mixture of compound I-4 (39.9 g, 6.55 mmol), compound I-5 (18.8 g, 9.17 mmol), 3 Å MS (40 g) and ACN/DCM (400 mL, 1:3, v/v) was stirred at 20-25° C. for 1.0 h under N₂ atmosphere. DCI (3.87 g, 32.8 mmol) was added and the reaction mixture was stirred for 1.0 h. DDTT (2.96 g, 14.4 mmol) was added and the reaction mixture was stirred at 20-25° C. for 30 min. DCM (100 mL) was added and the reaction mixture was filtrated and the 3 Å MS filter cake was washed with DCM (2×30 mL). The combined filtrate was concentrated to about 200 mL on a rotary evaporator and slowly added to ACN (1.36 L) in 30 min with vigorous agitation at 0±5° C. The precipitated product was filtered, washed with ACN (3×100 mL) and dried under vacuum at 20-30° C. for 12 h to yield compound I-6 as a light-yellow solid (49.8 g, 94.3% yield).

General Procedure for Preparation of Compound I-7

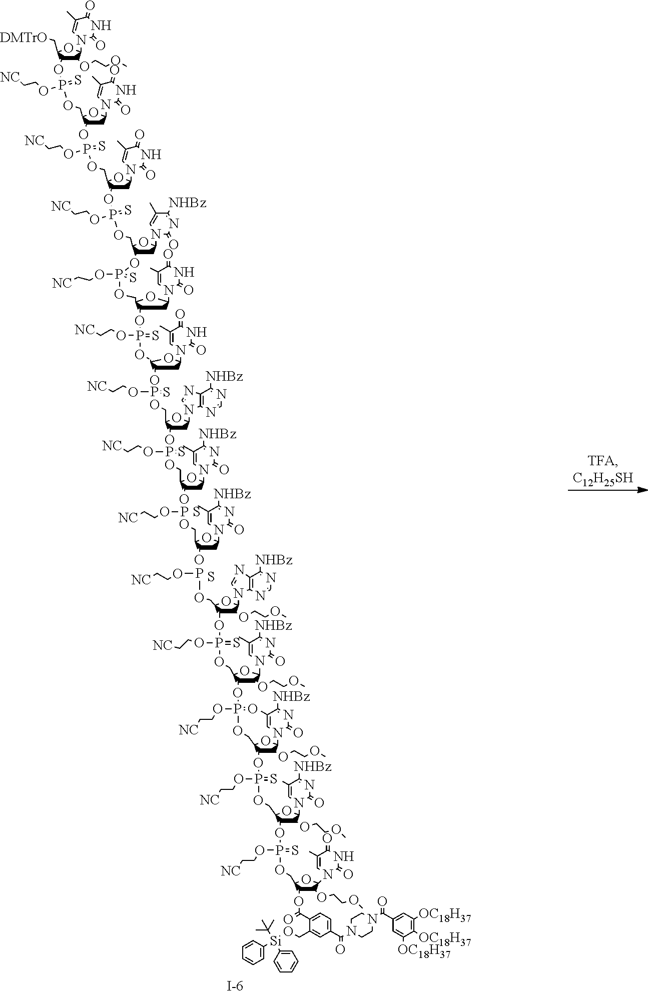

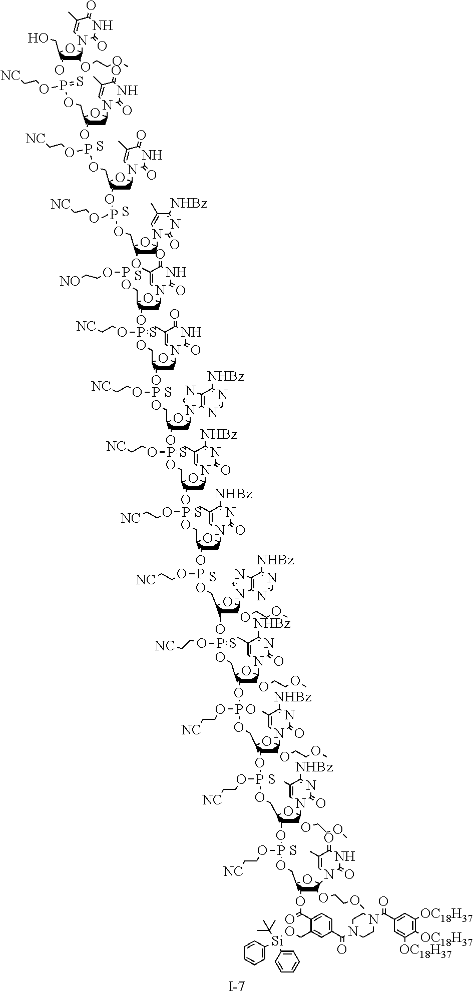

A mixture of compound I-6 (50.0 g, 6.20 mmol), 3 Å MS (18.0 g) and DCM (350 mL) was stirred at 20-25° C. for 1.0 h under N₂ atmosphere and cooled down to 0±5° C. Dodecane-1-thiol (3.77 g, 18.6 mmol) was added, and the reaction mixture was stirred at 0° C. for 10 min. TFA (9.19 g, 80.6 mmol) was added slowly and the reaction mixture was stirred at 0±5° C. for 1.5 h. NMI (8.14 g, 99.2 mmol) was added in 10 min, and the reaction mixture was filtered, and the 3 Å MS filter cake was washed with DCM. The combined filtrate was concentrated to about 200 mL on a rotary evaporator and added to ACN (2.65 L) in 30 min with vigorous agitation at 0±5° C. The precipitated product was filtered, washed with ACN (3×150 mL) and dried under vacuum at 20-30° C. for 14 h to yield compound I-7 as a white solid (45.9 g, 95.3% yield).

General Procedure for Preparation of Oligonucleotide I

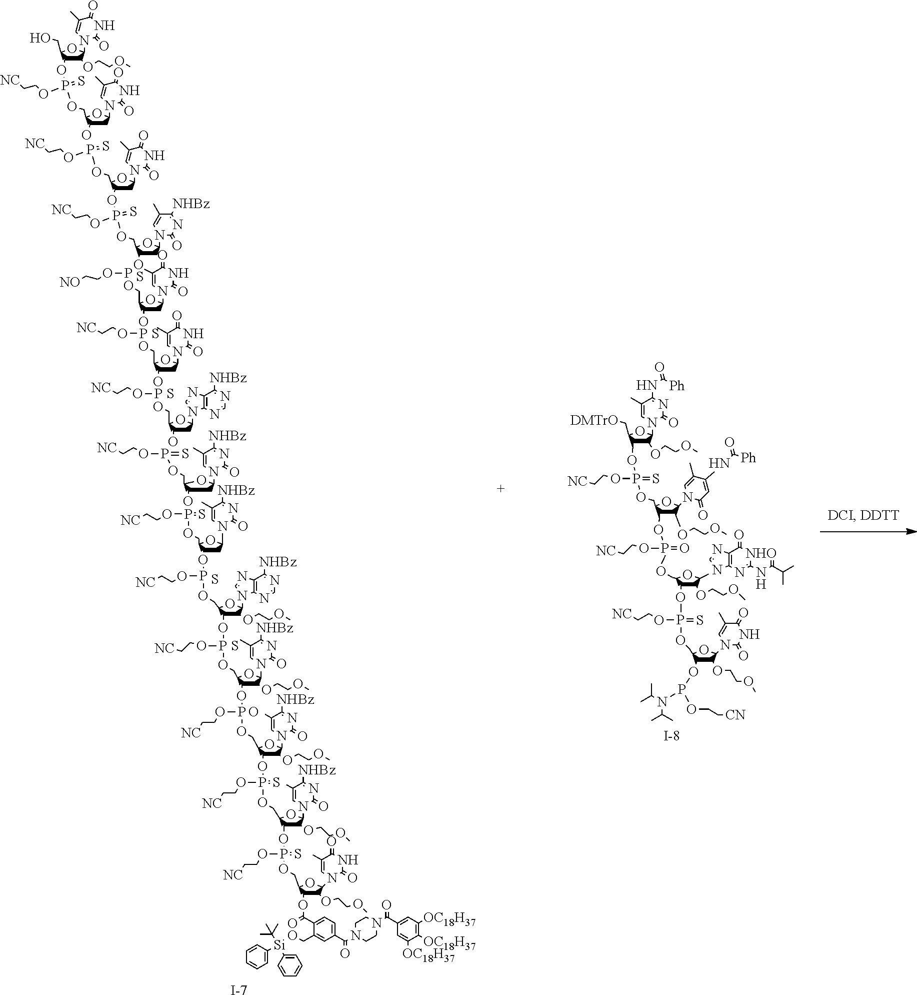

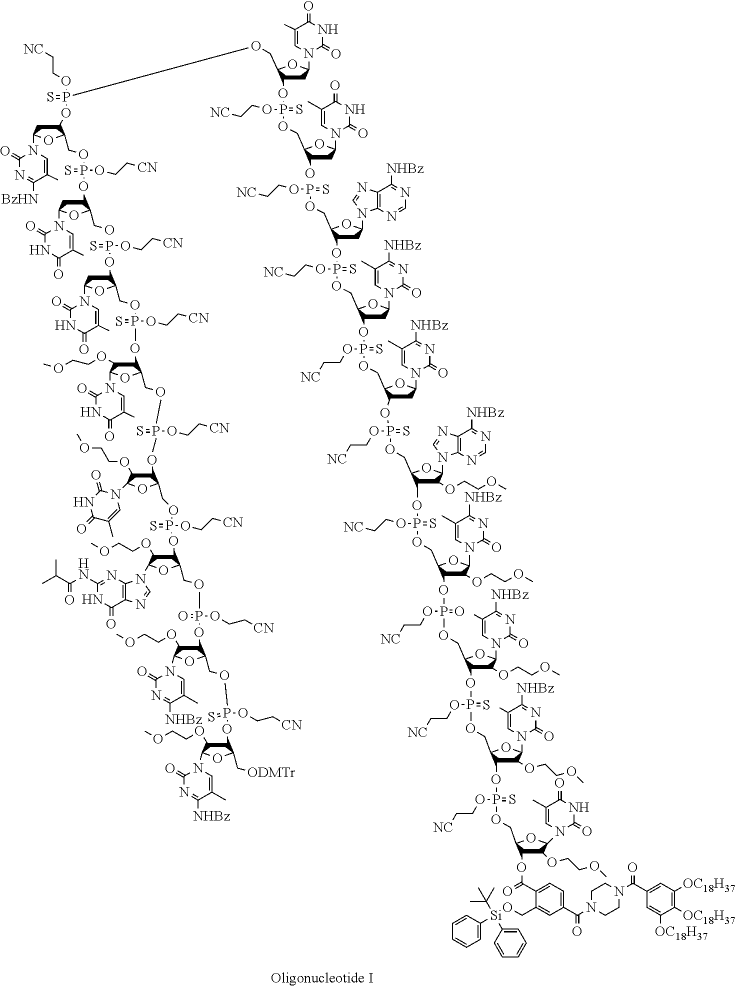

A mixture of compound 1-7 (42.0 g, 5.41 mmol), compound 1-8 (23.9 g, 9.74 mmol), 3 Å MS (42 g) and DCM/CH₃CN (400 mL, 3:1, v/v) was stirred at 20-25° C. for 1.0 h under N₂ atmosphere. DCI (3.2 g, 27.05 mmol) was added, and the reaction mixture was stirred for 1.0 h. DDTT (2.45 g, 11.9 mmol) was added and the reaction mixture was stirred at 20-25° C. for 30 min DCM (200 mL) was added and the reaction mixture was filtrated, and 3 Å MS filter cake was washed with DCM (2×50 mL). The combined reaction mixture was concentrated to about 300 mL on a rotary evaporator and slowly added to ACN (2.50 L) in 30 min with vigorous agitation at 0±5° C. The precipitated product was filtered, washed with ACN (2×150 mL) and dried under vacuum at 20-30° C. for 14 h to yield oligonucleotide I as a light-yellow solid (51.6 g, 94.1% yield).

Characterization of oligonucleotide I: The mixture of oligonucleotide I (100.0 mg) and 30% NH₄OH (2 mL) in a 4 mL pressure flask was stirred at 65° C. for 4 h. The resulting compound was checked in LCMS. The structure of oligonucleotide I was confirmed by LCMS. HRMS calcd for C₂₃₁H₃₁₈N₅₃O₁₁₈P₁₇S₁₅/4⁻ [M]/4: 1682.0, found: 1682.1.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A compound of formula I′ or B: 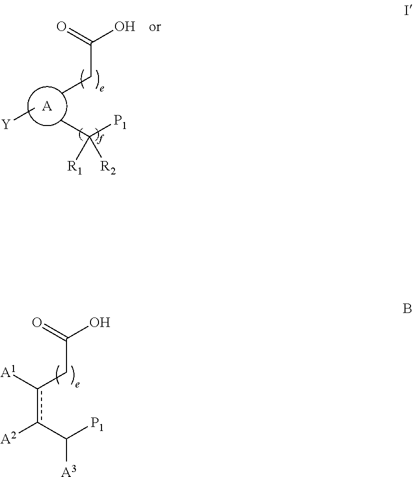
or a salt thereof, wherein:
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}A, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

2. The compound of claim 1, wherein the compound is of formula I′: 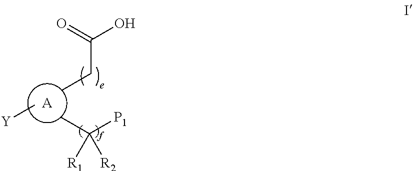
or a salt thereof, wherein:
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

3. The compound of claim 1, wherein the compound is of formula B: 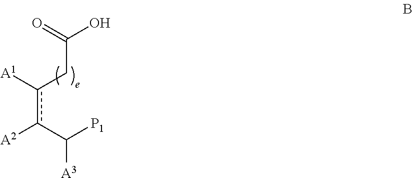
or a salt thereof.

4. The compound of claim 3, wherein the compound is represented by one of the following formula: 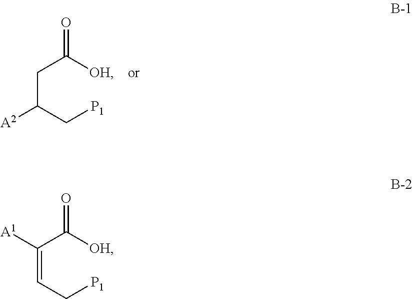
or a salt thereof.

5. The compound of any one of claims 1-4 or a salt thereof, wherein Y is a hydrophobic group comprising one or more aliphatic hydrocarbon group having 10 or more carbon atoms.

6. The compound of any one of claims 1, 2 and 5 or a salt thereof, wherein ring A is phenyl or naphthalenyl.

7. The compound of any one of claims 1 to 6, or a salt thereof, wherein P₁ is a silyl hydroxyl protecting group selected from the following: 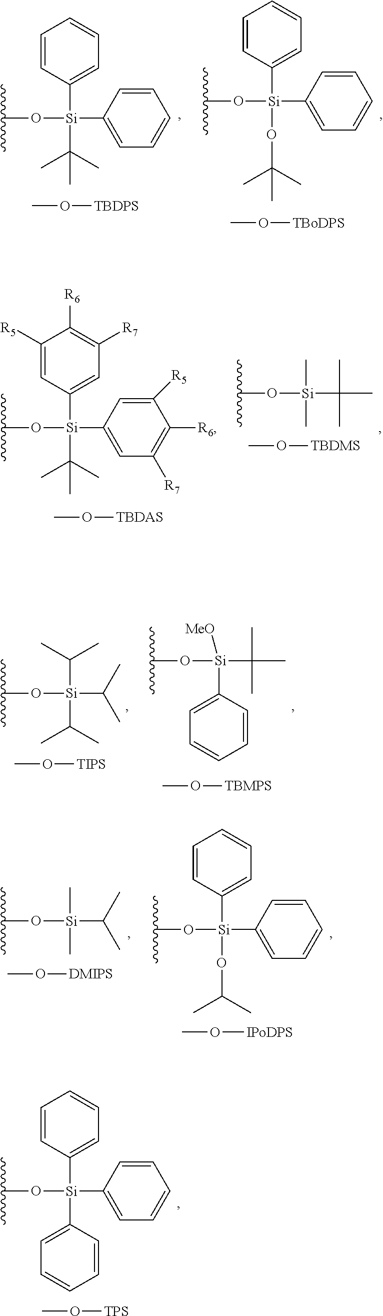 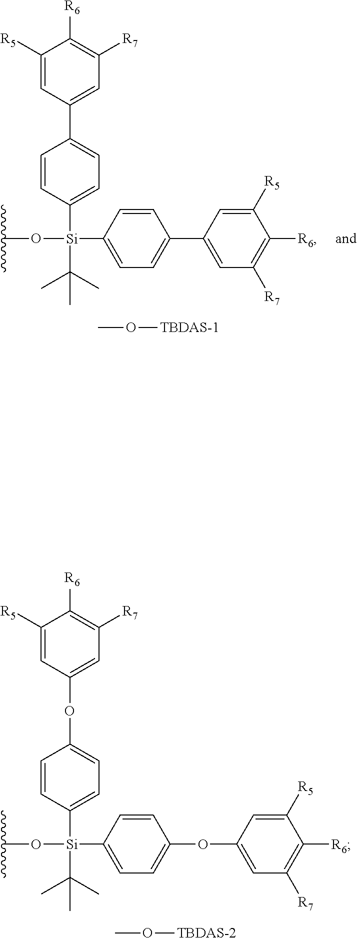
wherein 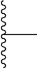
the point of attachment for P₁ and R₅, R₆ and R₇ are each independently H, C₁₋₃₀alkyl, or C₁₋₃₀alkoxy.

8. The compound of any one of claims 1 to 7, or a salt thereof, wherein P₁ is selected from the group consisting of —O-TBDMS, —O-TIPS, —O-TBDPS, —O-TBoDPS, and —O-TBDAS:

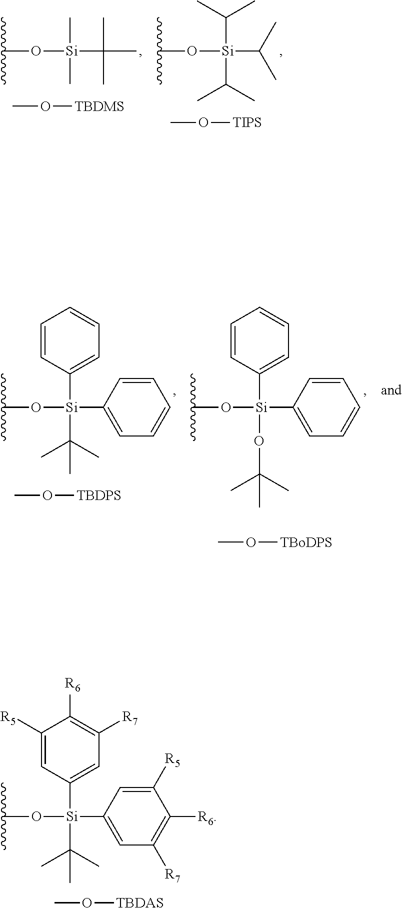

9. The compound of any one of claims 1, 2, and 5 to 8 represented by Formula I or Ia: 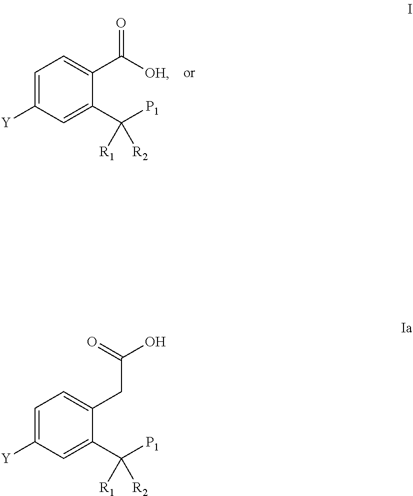
or a salt thereof;
wherein P₁ is selected from the group consisting of —O-TBDPS, —O-TBoDPS, and —O-TBDAS: 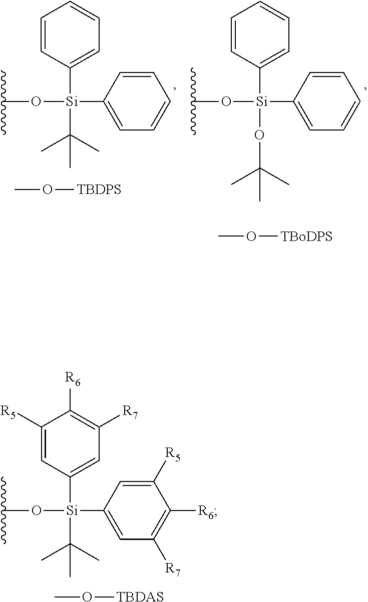
and
R₅, R₆ and R₇ are each independently H, C₁₋₃₀alkyl, or C₁₋₃₀alkoxy.

10. The compound of any one of claims 1 to 9 or a salt thereof, wherein Y is represented by Formula A:
W—V—U—* (A)
wherein:
—* represents the point of attachment for Y;
W is represented by Formula A1, A2, A2-1, A2-2, A3, A3-1, or A3-2: 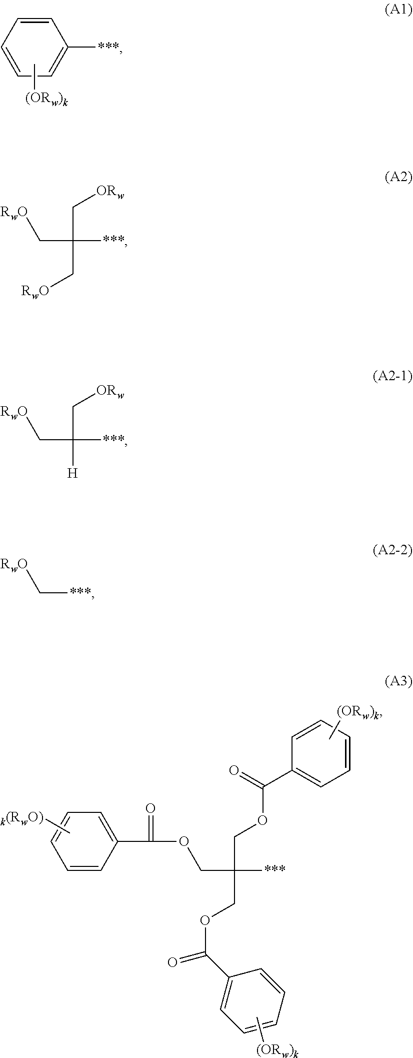 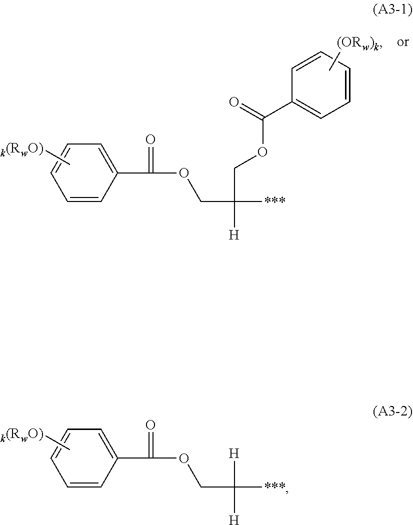
wherein
—*** represents the point where W and V connect;
each R_{w} is independently an aliphatic hydrocarbon group having 10 or more carbon atoms;
k is an integer from 1 to 5;
V is a bond, oxygen, C₁₋₂₀alkylene, C₁₋₆alkynylene, —C(═O)—, ***—C(═O)—O—**, ***—O—C(═O)—**, 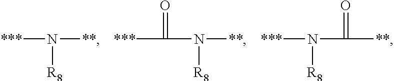
or 5 to 7 member heteroaryl having 1 to 3 heteroatoms selected from oxygen, nitrogen, and sulfur, wherein the heteroaryl is optionally substituted by 1-3 R₈; wherein —** represents the point where V and U connect; and R₈ is H or C₁₋₃₀alkyl; and
U is a bond, oxygen, C₁₋₂₀alkylene, carbonyl, ***—O—C(═O)—**, 5 to 7 member heterocyclyl having 1 to 3 heteroatoms selected from oxygen, nitrogen, and sulfur; 5 to 7 member heteroaryl having 1 to 3 heteroatoms selected from oxygen, nitrogen, and sulfur, wherein the heteroaryl is optionally substituted by 1-3 R₈; or a group represented by formula A4, A5, or A6: 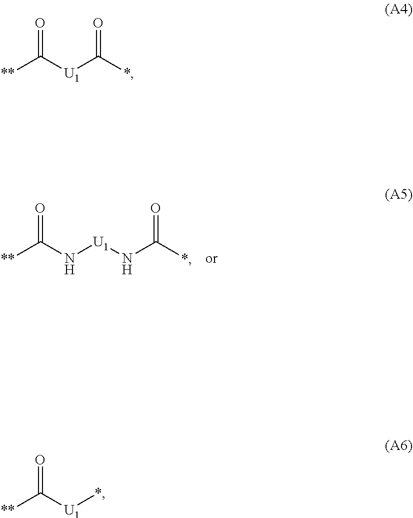
wherein U₁ is C₁₋₆alkylene, C₁₋₆alkyleneoxy, 5 to 7 member heterocyclyl having 1 to 3 heteroatoms selected from oxygen, nitrogen, and sulfur, or 5 to 7 member heteroaryl having 1 to 3 heteroatoms selected from oxygen, nitrogen, and sulfur.

11. The compound of any one of claims 7 to 10 or a salt thereof, wherein the TBDAS group is: 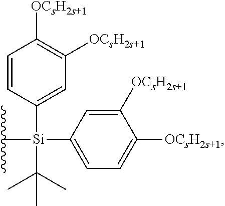
wherein s is an integer from 1 to 30.

12. The compound of any one of claims 1 to 11 or a salt thereof, wherein P₁ is —O-TBDPS.

13. The compound of any one of claims 10-12 or a salt thereof, wherein W is represented by Formula A1: 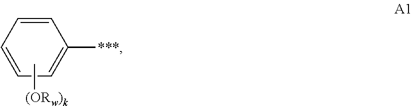
wherein R_{w} is CₙH₂ₙ₊₁;
n is an integer from 1 to 30.

14. The compound of any one of claims 10-13 or a salt thereof, wherein R_{w} is selected from a group consisting of C₁₂H₂₅, C₁₈H₃₇, C₂₀H₄₁, C₂₂H₄₅, C₂₄H₄₉, C₂₆H₅₃, and C₂₈H₅₇.

15. The compound of any one of claims 10-14 or a salt thereof, wherein V is a bond, CH₂, CH₂CH₂, C(═O), ***—C(═O)—O—**, or

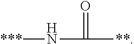

16. The compound of any one of claims 10-15 or a salt thereof, wherein U is a bond, CH₂, CH₂CH₂, carbonyl, triazolylene, piperazinylene,

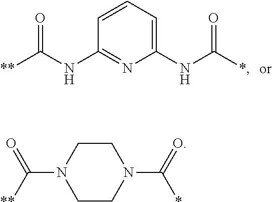

17. The compound of any one of claims 10-14 or a salt thereof, wherein U—V is selected from the group consisting of 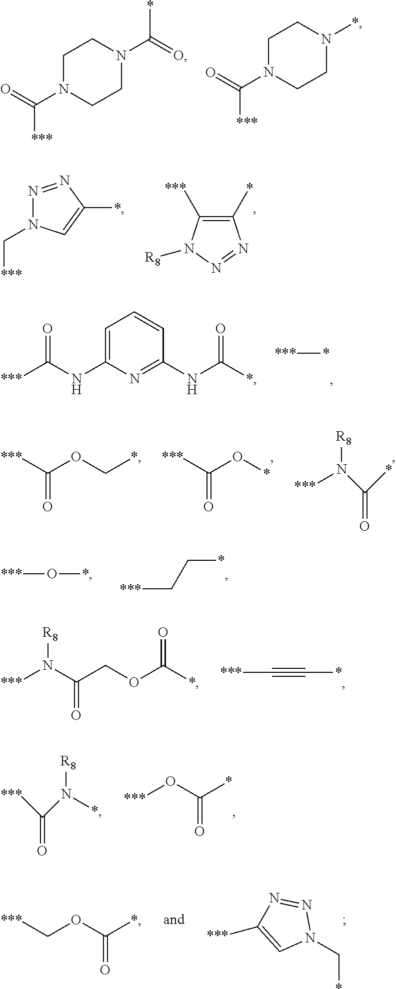
wherein R₈ is H or C₁₋₆alkyl.

18. The compound of any one of claims 1-12 or a salt thereof, wherein Y is selected from the groups consisting of 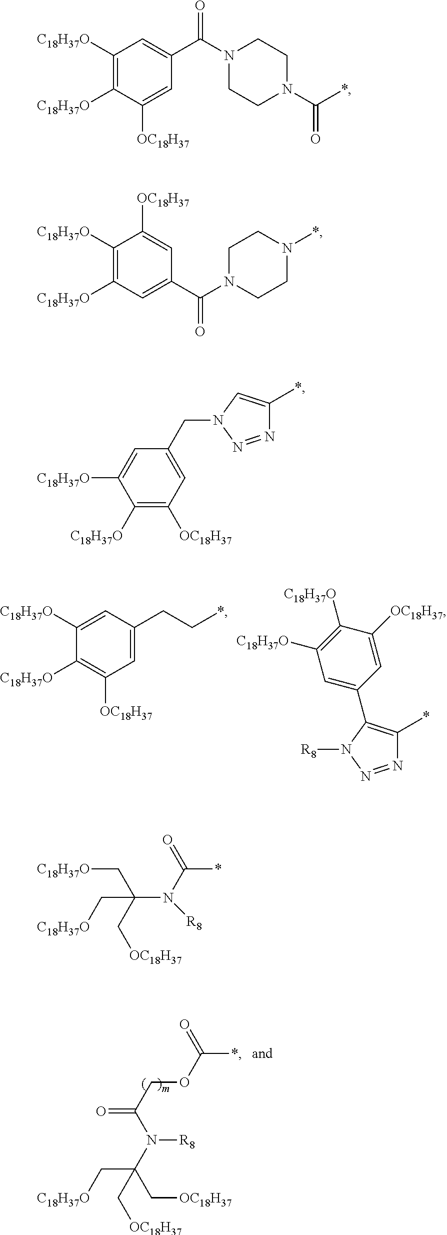 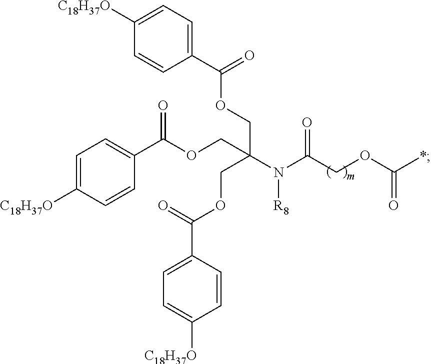
wherein
R₈ is H or C₁₋₆alkyl; and
m is an integer from 1 to 5.

19. The compound of any one of claims 1, 2, and 5-18 or a salt thereof, wherein R₁ and R₂ are independently H or CH₃.

20. The compound of any one of claims 1, 2, and 5-19 or a salt thereof, wherein e is 0, 1, or 2; and f is 0, 1, or 2.

21. The compound of any one of claims 10-20 or a salt thereof, wherein R₈ is H or C₁₋₄alkyl.

22. The compound of claim 1 or a salt thereof represented by Formula II or IIa 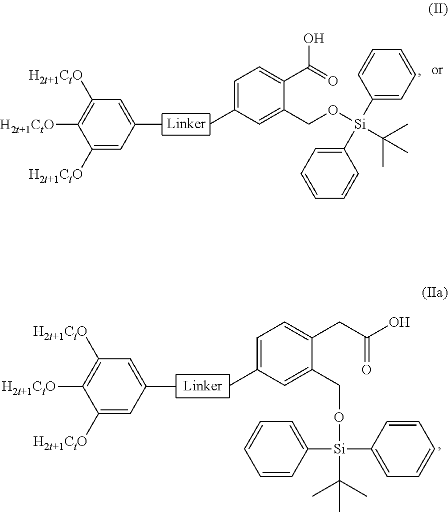
wherein
t is an integer from 10 to 30; [symbol]
is selected from the group consisting of 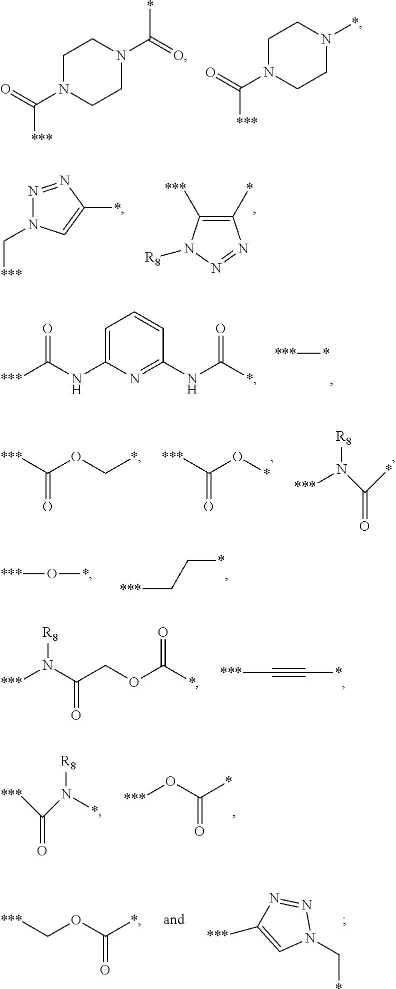
wherein R₈ is H or C₁₋₆alkyl.

23. The compound of claim 22 is selected from the group consisting of 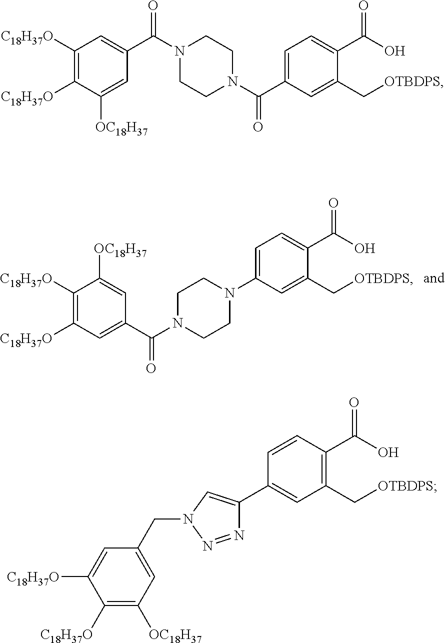
or a salt thereof.

24. The compound of claim 1, wherein the compound is 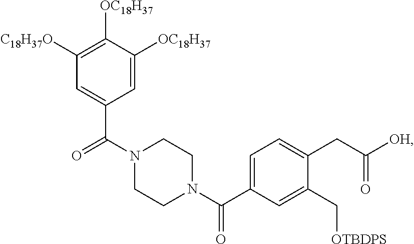
or a salt thereof.

25. The compound of claim 1, wherein the compound is selected from one of the following formulae: 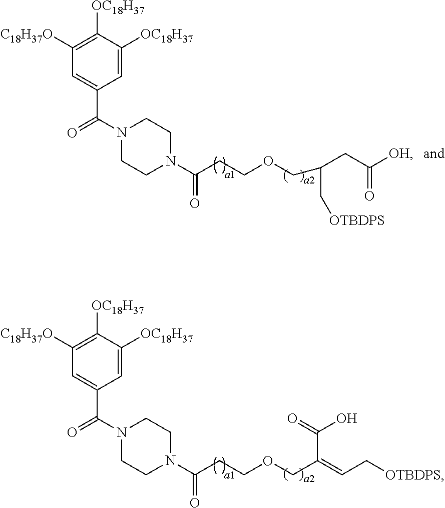
or a salt thereof.

26. A compound of Table 1 or a salt thereof.

27. A nucleotide or oligonucleotide represented by Formula III or IIIP, 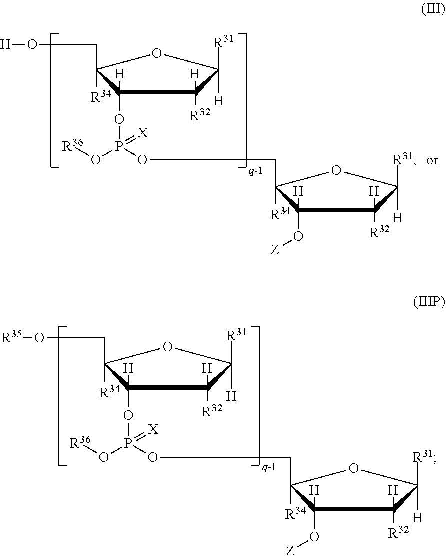
or a salt thereof, wherein
R³¹, for each occurrence, is independently a nucleobase, wherein the NH₂ of the nucleobase, if present, is optionally protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently H, C₁₋₆alkyl group, C₂₋₆alkenyl group, phenyl or benzyl group, each of which is optionally substituted with —CN, —NO₂ or halogen; or
R³⁶ is 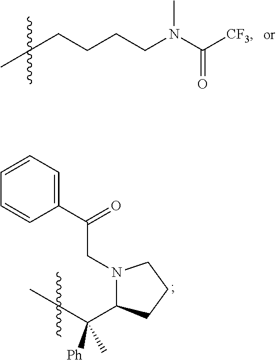 [SMILES: *CCCCN(C)C(=O)C(F)(F)F ; *[C@](C)(c1ccccc1)[C@@H]1CCCN1CC(=O)c1ccccc1]
q is an integer from 1 to 20;
X, for each occurrence, is independently O or S;
Z is a group represented by Formula I* or B*, 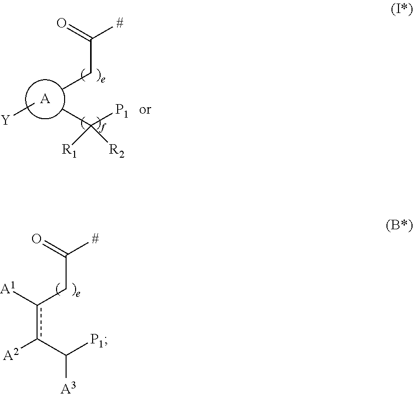
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

28. A nucleotide or oligonucleotide represented by Formula III′ or HIP′, 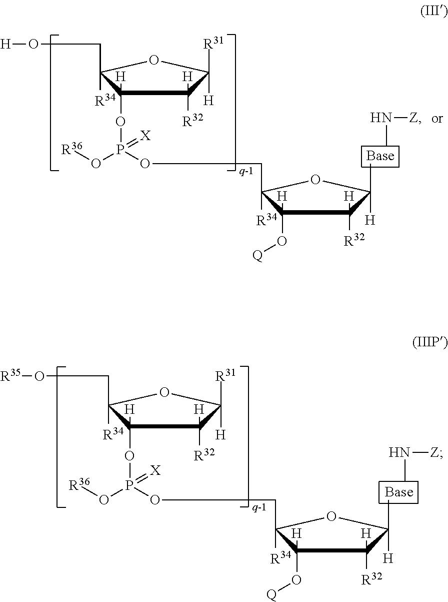
or a salt thereof, wherein
Q is a hydroxyl protecting group; [symbol]
is a nucleobase comprising a NH₂ group which is modified by Z;
R³¹, for each occurrence, is independently a nucleobase, wherein the NH₂ of the nucleobase, if present, is optionally protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently H, C₁₋₆alkyl group, C₂₋₆alkenyl group, phenyl or benzyl group, each of which is optionally substituted with —CN, —NO₂ or halogen; or
R³⁶ is 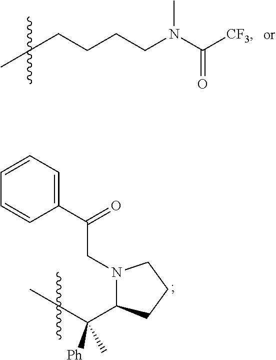 [SMILES: *CCCCN(C)C(=O)C(F)(F)F ; *[C@](C)(c1ccccc1)[C@@H]1CCCN1CC(=O)c1ccccc1]
q is an integer from 1 to 20;
X, for each occurrence, is independently O or S;
Z is a group represented by Formula I* or B*, 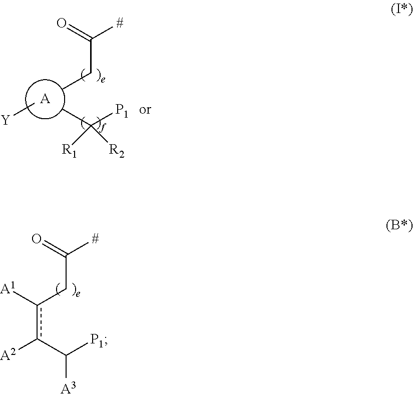
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

29. The nucleotide or oligonucleotide of claim 27 or 28 or a salt thereof, wherein Z is a group represented by Formula I*,

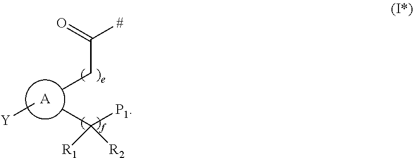

30. The nucleotide or oligonucleotide of claim 27 or 28 or a salt thereof, wherein Z is a group represented by Formula B*,

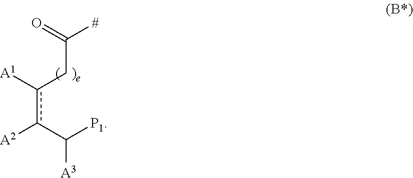

31. The nucleotide or oligonucleotide of claim 30, wherein Z is a group represented by Formula B-1* or B-2*:

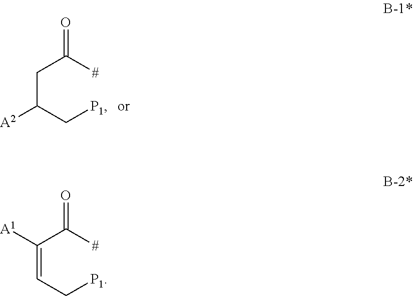

32. The nucleotide or oligonucleotide of any one of claims 27-31 or a salt thereof, wherein Y is a hydrophobic group comprising one or more aliphatic hydrocarbon group having 10 or more carbon atoms.

33. The nucleotide or oligonucleotide of claim 27, claim 28, or claim 32 or a salt thereof, wherein ring A is phenyl or naphthalenyl.

34. The nucleotide or oligonucleotide of any one of claims 27 to 33 or a salt thereof, wherein P₁ is a silyl hydroxyl protecting group selected from the following: 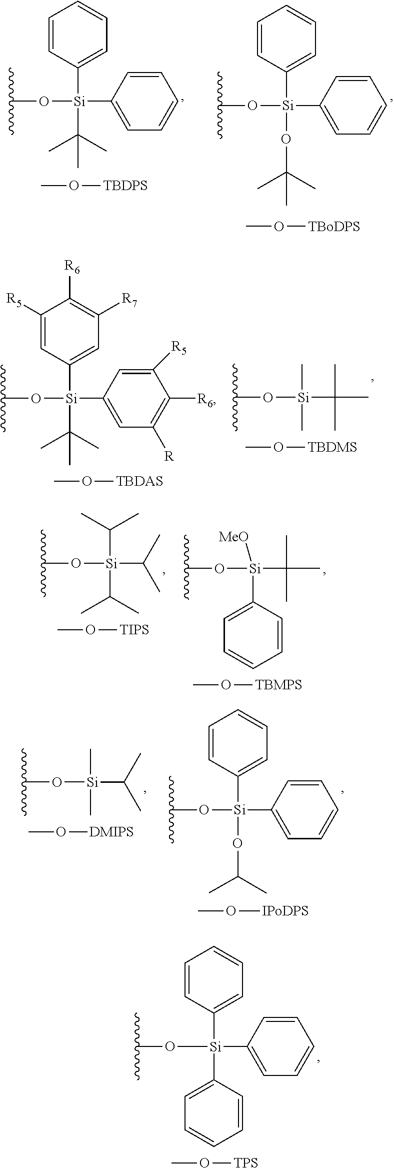 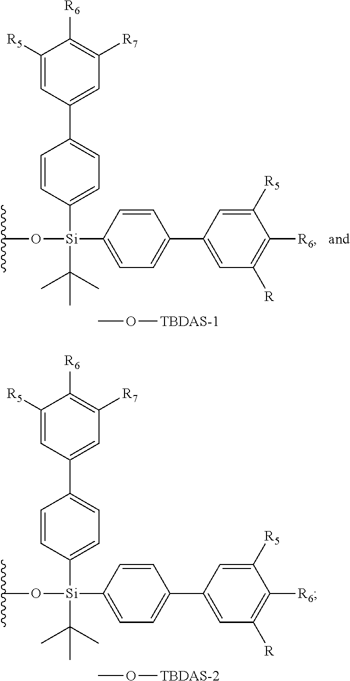
wherein 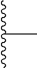
represents the point of attachment for P₁ and R₅, R₆ and R₇ are each independently H, C₁₋₃₀alkyl, or C₁₋₃₀alkoxy.

35. The compound of any one of claims 27 to 34, or a salt thereof, wherein P₁ is selected from the group consisting of —O-TBDMS, —O-TIPS, —O-TBDPS, —O-TBoDPS, and —O-TBDAS:

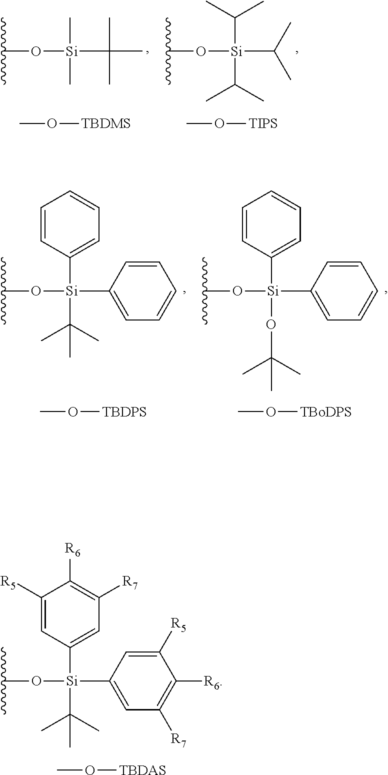

36. The nucleotide or oligonucleotide of any one of claims 27-35 or a salt thereof, wherein Z is a group represented by Formula I** or Ia**: 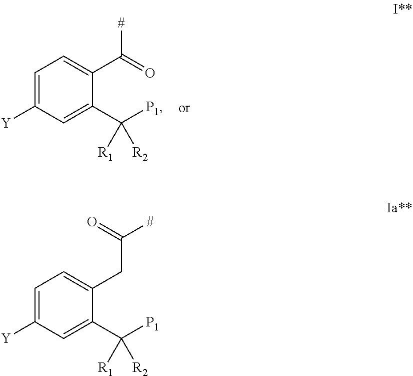
or a salt thereof;
wherein P₁ is selected from the group consisting of —O-TBDPS, —O-TBoDPS, and —O-TBDAS: 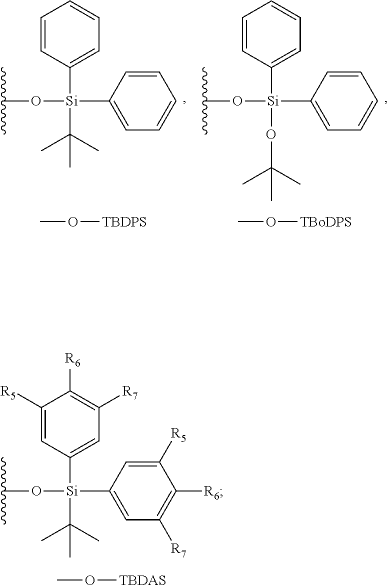
and
R₅, R₆ and R₇ are each independently H, C₁₋₃₀alkyl, or C₁₋₃₀alkoxy;

37. The nucleotide or oligonucleotide of any one of claims 27-36 or a salt thereof, wherein Y is represented by Formula A:
W—V—U—* (A)
wherein:
—* represents the point of attachment for Y;
W is represented by Formula A1, A2, A2-1, A2-2, A3, A3-1, or A3-2: 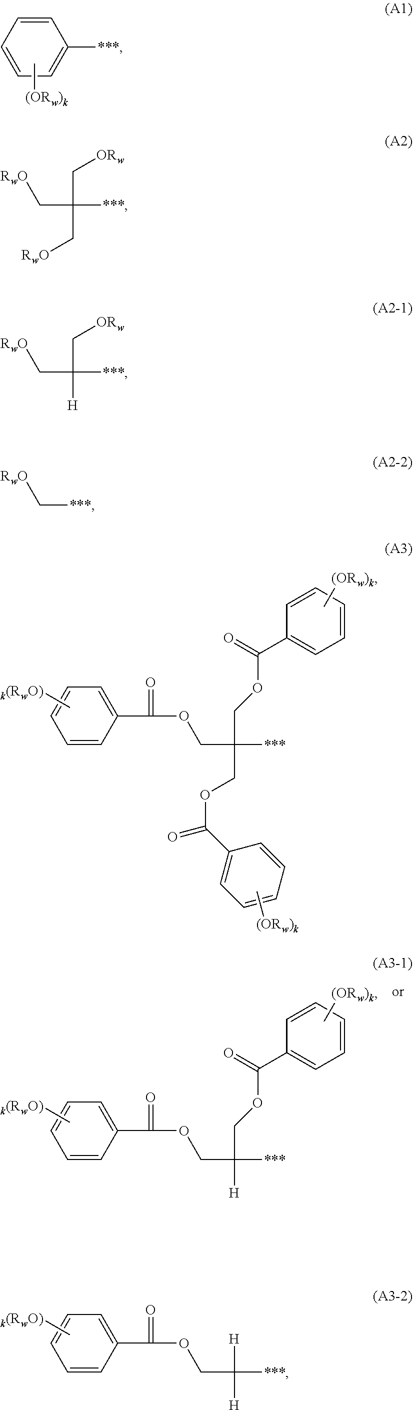
wherein
—*** represents the point where W and V connect;
each R_{w} is independently an aliphatic hydrocarbon group having 10 or more carbon atoms;
k is an integer from 1 to 5;
V is a bond, oxygen, C₁₋₂₀alkylene, C₁₋₆alkynylene, —C(═O)—, ***—C(═O)—O—**, ***—O—C(═O)—**, 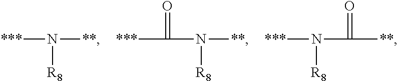
or 5 to 7 member heteroaryl having 1 to 3 heteroatoms selected from oxygen, nitrogen, and sulfur, wherein the heteroaryl is optionally substituted by 1-3 R₈; wherein —** represents the point where V and U connect; and R₈ is H or C₁₋₃₀alkyl; and
U is a bond, oxygen, C₁₋₂₀alkylene, carbonyl, ***—O—C(═O)—**, 5 to 7 member heterocyclyl having 1 to 3 heteroatoms selected from oxygen, nitrogen, and sulfur; 5 to 7 member heteroaryl having 1 to 3 heteroatoms selected from oxygen, nitrogen, and sulfur, wherein the heteroaryl is optionally substituted by 1-3 R₈; or a group represented by formula A4, A5, or A6: 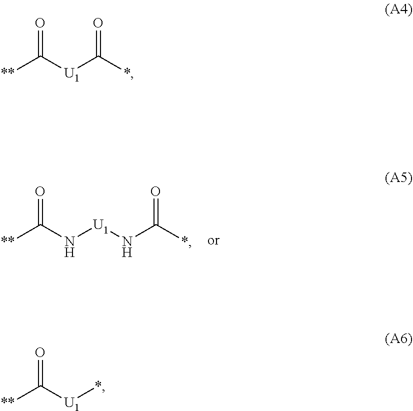
wherein U₁ is C₁₋₆alkylene, C₁₋₆alkyleneoxy, 5 to 7 member heterocyclyl having 1 to 3 heteroatoms selected from oxygen, nitrogen, and sulfur, or 5 to 7 member heteroaryl having 1 to 3 heteroatoms selected from oxygen, nitrogen, and sulfur.

38. The nucleotide or oligonucleotide of any one of claims 34-37 or a salt thereof, wherein the TBDAS group is: 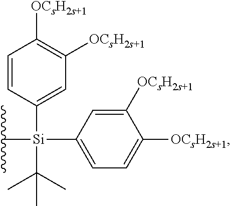
wherein s is an integer from 1 to 30.

39. The nucleotide or oligonucleotide of any one of claims 27-37 or a salt thereof, wherein P₁ is TBDPS.

40. The nucleotide or oligonucleotide of any one of claims 37-39 or a salt thereof, wherein W is represented by Formula A1: 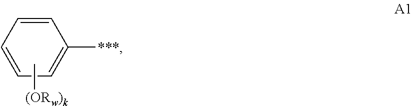
wherein R_{w} is CₙH₂ₙ₊₁;
n is an integer from 1 to 30.

41. The nucleotide or oligonucleotide of any one of claims 37-40 or a salt thereof, wherein R_{w} is selected from a group consisting of C₁₂H₂₅, C₁₈H₃₇, C₂₀H₄₁, C₂₂H₄₅, C₂₄H₄₉, C₂₆H₅₃, and C₂₈H₅₇.

42. The nucleotide or oligonucleotide of any one of claims 37-41 or a salt thereof, wherein V is a bond, CH₂, CH₂CH₂, C(═O)—, ***—C(═O)—O—**, or

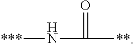

43. The nucleotide or oligonucleotide of any one of claims 37-42 or a salt thereof, wherein U is a bond, CH₂, CH₂CH₂, carbonyl, triazolylene, piperazinylene,

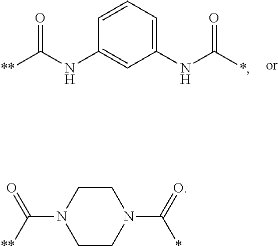

44. The nucleotide or oligonucleotide of any one of claims 37-41 or a salt thereof, wherein U—V is selected from the group consisting of 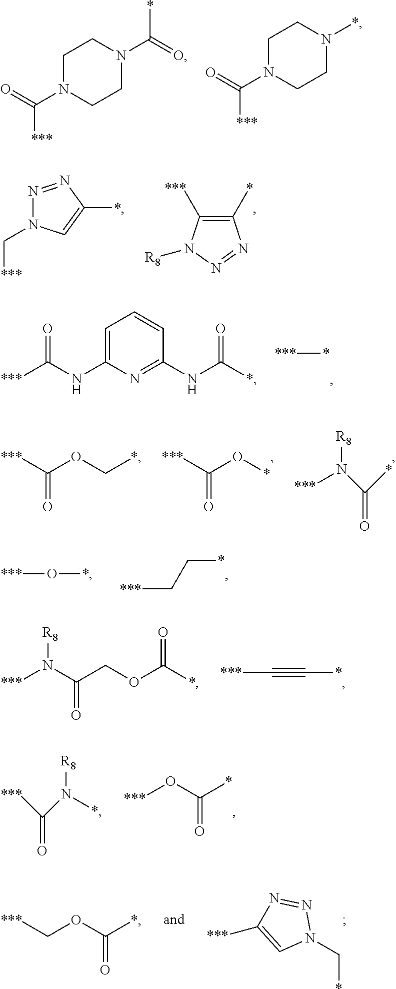
wherein R₈ is H or C₁₋₆alkyl.

45. The nucleotide or oligonucleotide of any one of claims 27-39 or a salt thereof, wherein Y is selected from the groups consisting of 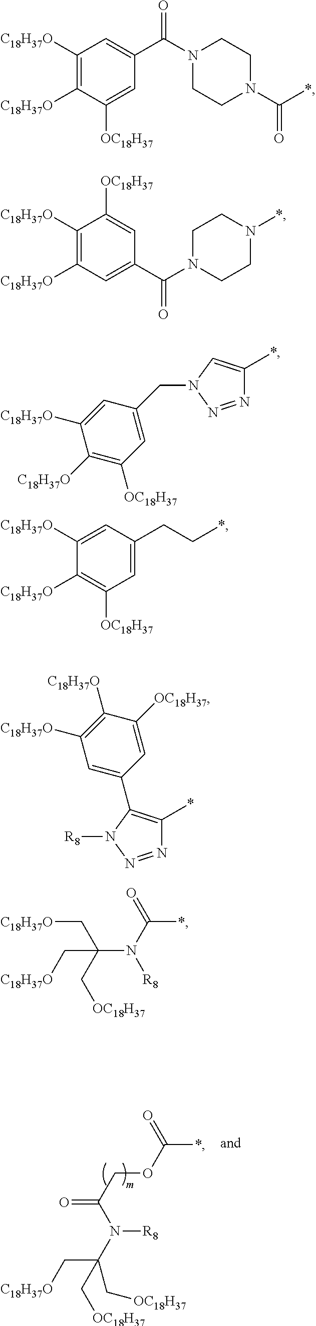 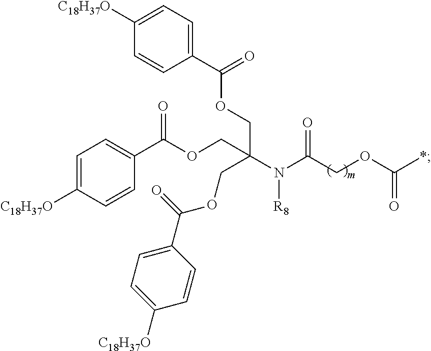
wherein
R₈ is H or C₁₋₆alkyl; and
m is an integer from 1 to 5.

46. The nucleotide or oligonucleotide of any one of claims 27-45 or a salt thereof, wherein R₁ and R₂ are independently H or CH₃.

47. The nucleotide or oligonucleotide of any one of claims 27-46 or a salt thereof, wherein e is 0, 1, or 2; and f is 0, 1, or 2.

48. The nucleotide or oligonucleotide of any one of claims 37-47 or a salt thereof, wherein R₈ is H or C₁₋₄alkyl.

49. The nucleotide or oligonucleotide of claim 27 or claim 28 or a salt thereof, wherein Z is represented by Formula II* or IIa*, 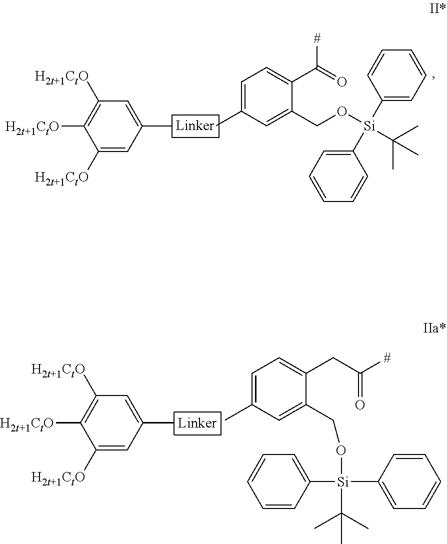
wherein
t is an integer from 10 to 30; [symbol]
is selected from the group consisting of 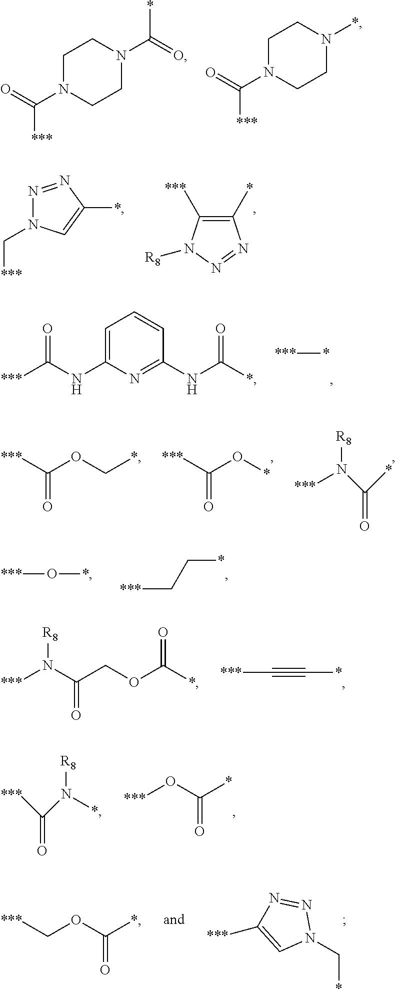
wherein R₈ is H or C₁₋₆alkyl.

50. The nucleotide or oligonucleotide of claim 49 or a salt thereof, wherein Z is

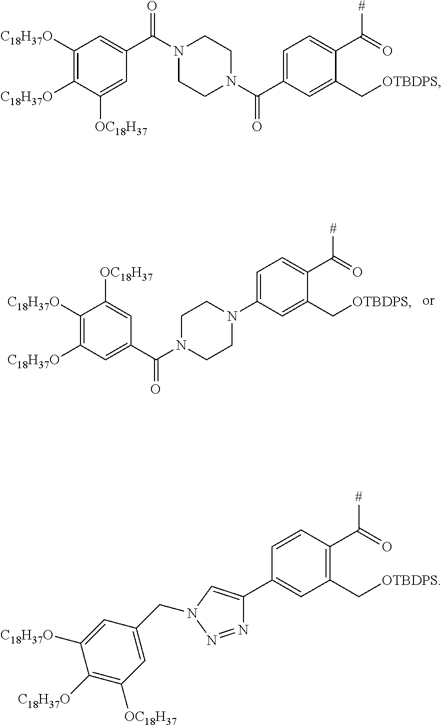

51. The nucleotide or oligonucleotide of claim 27 or 28, or a salt thereof, wherein Z is

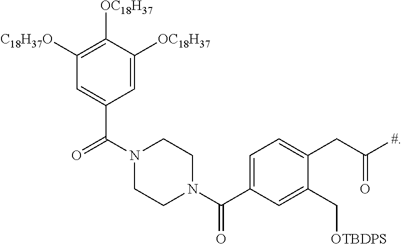

52. The nucleotide or oligonucleotide of claim 27 or 28, or a salt thereof, wherein Z is

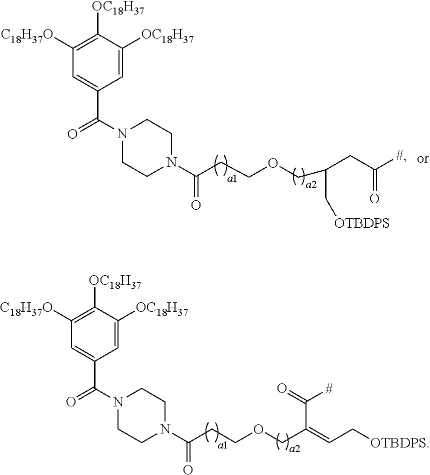

53. The nucleotide or oligonucleotide of any one of claims 27-52 or a salt thereof, wherein when X is S, the phosphorothiolate group has S-configuration as shown below: 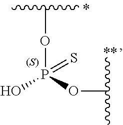
R-configuration as shown below: 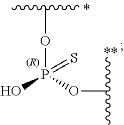
wherein [symbol] indicates the connection point to 3′-OH group and [symbol] indicates the connection point to 5′-OH group.

54. A process for preparing an oligonucleotide fragment of formula (V), 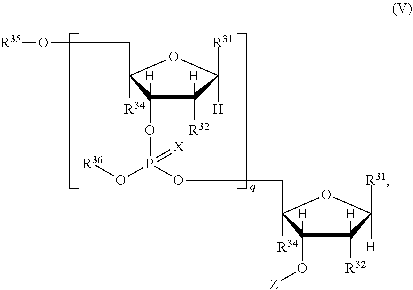
or a salt thereof, comprising the steps of:
1) deprotecting a compound of formula (VA): 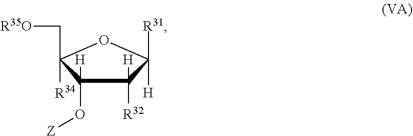
or a salt thereof, to form a compound of formula (VB): 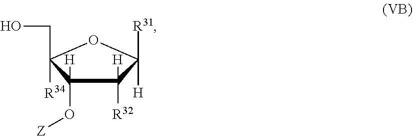
or a salt thereof;
2) reacting the compound of formula (VB), or a salt thereof, with a compound of formula (VC ): 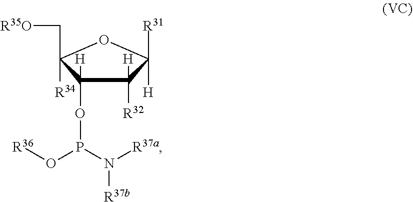
or a salt thereof, to form a compound of formula (VD), 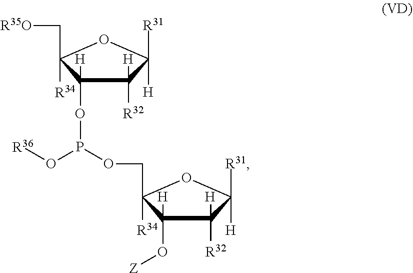
or a salt thereof;
3) sulfurizing or oxidizing the compound of formula (VD), or a salt thereof, with a sulfurization or oxidation agent to form a compound of formula (VE): 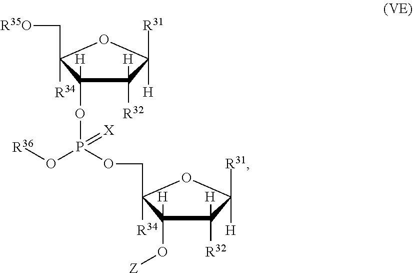
or a salt thereof;
4) deprotecting the compound of formula (VE), or a salt thereof to form a compound of formula (VF): 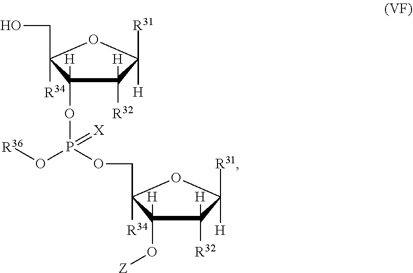
or a salt thereof;
5) when q is equal or greater than 2, starting with the compound of formula (VF), repeating steps 2), 3) and 4) for q-2 times, followed by steps 2) and 3) to yield the fragment of formula (V), or a salt thereof, wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 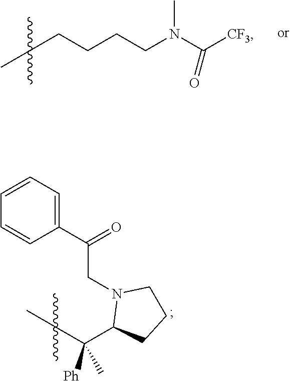 [SMILES: *CCCCN(C)C(=O)C(F)(F)F ; "or" ; *[C@](C)(c1ccccc1)[C@@H]1CCCN1CC(=O)c1ccccc1]
R^{37a} and R^{37b} are independently C₁₋₆alkyl;
q is an integer from 1 to 20;
X, for each occurrence, is independently O or S;
Z is a group represented by Formula I* or B*, 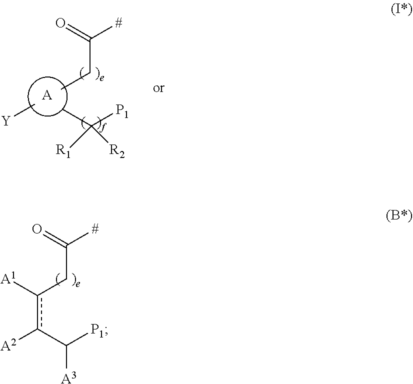
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

55. A process for preparing an oligonucleotide fragment of formula (V′), 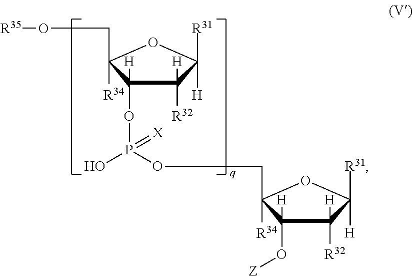
or a salt thereof, comprising the steps of:
1) deprotecting a compound of formula (VA): 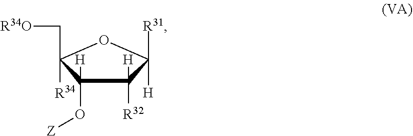
or a salt thereof, to form a compound of formula (VB): 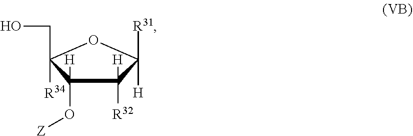
or a salt thereof;
2) reacting the compound of formula (VB), or a salt thereof, with a compound of formula (VC′): 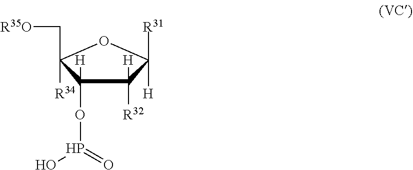
or a salt thereof, to form a compound of formula (VD′), 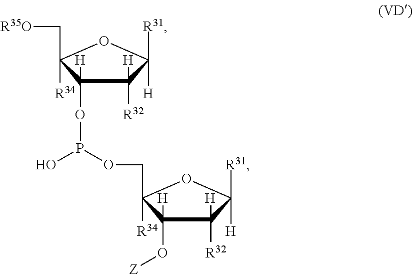
or a salt thereof;
3) sulfurizing or oxidizing the compound of formula (VD′), or a salt thereof, with a sulfurization or oxidation agent to form a compound of formula (VE′): 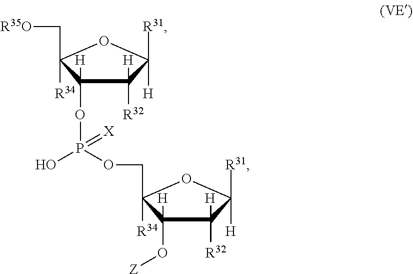
or a salt thereof;
4) deprotecting the compound of formula (VE′), or a salt thereof to form a compound of formula (VF′): 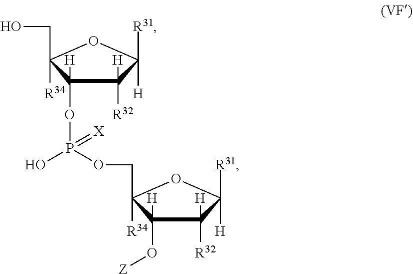
or a salt thereof;
5) when q is equal or greater than 2, starting with the compound of formula (VF′), repeating steps 2), 3) and 4) for q-2 times, followed by steps 2) and 3) to yield the fragment of formula (V′), or a salt thereof, wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
q is an integer from 1 to 20;
X, for each occurrence, is independently O or S;
Z is a group represented by Formula I* or B*, 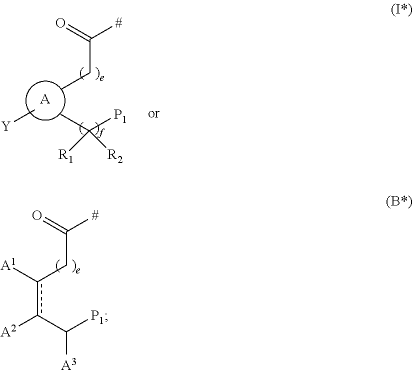
wherein
—#represents the point of attachment for Z;
one of A1, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

56. A process for preparing an oligonucleotide fragment of formula (V-C1) or (V-C2), 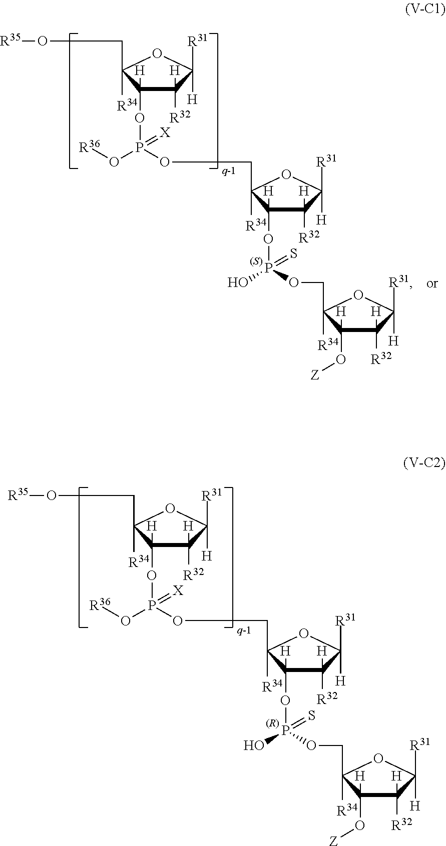
or a salt thereof, comprising the steps of:
1) reacting the compound of formula (VB), 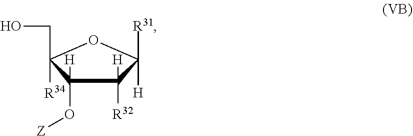
or a salt thereof, with a compound of formula (V-CR1) or (V-CR2), 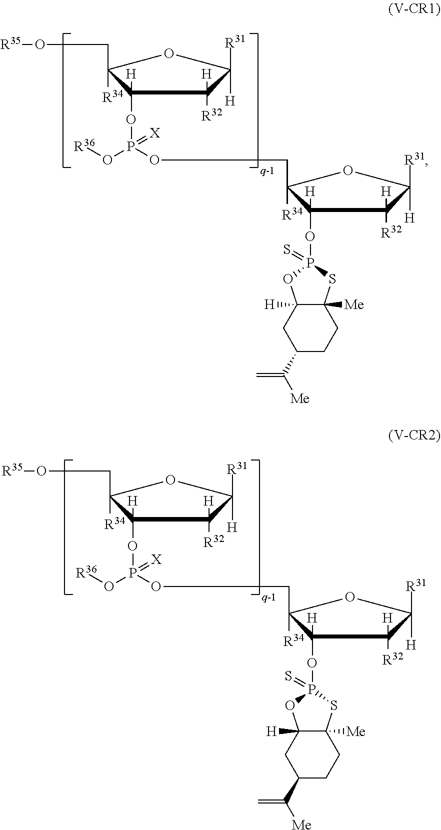
or a salt thereof, and a base, to form a compound of formula (V-C1) or (V-C2), wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 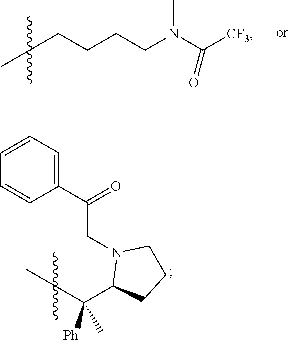 [SMILES: *CCCCN(C)C(=O)C(F)(F)F ; "or" ; *[C@](C)(c1ccccc1)[C@@H]1CCCN1CC(=O)c1ccccc1]
q is an integer from 1 to 20;
X, for each occurrence, is independently O or S, provided when X is S, the phosphorothiolate group has S-configuration, R-configuration or a mixture thereof (e.g., a racemic mixture);
Z is a group represented by Formula I* or B*, 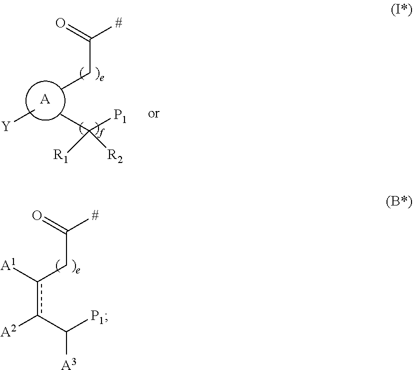
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

57. A process for preparing an oligonucleotide fragment of formula (V-C1) or (V-C2), 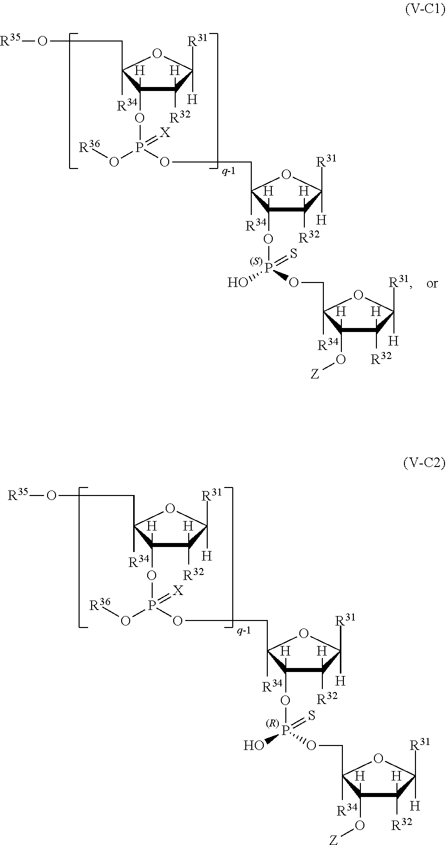
or a salt thereof, comprising the steps of:
1) reacting the compound of formula (VB), 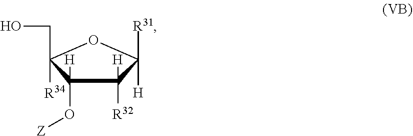
or a salt thereof, with a reagent of formula (VR1) or (VR2), 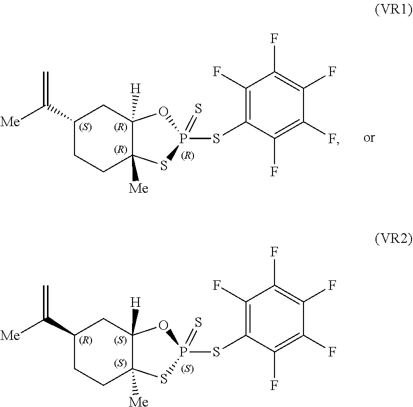
to form a compound of formula (V-CR3) or (V-CR4), 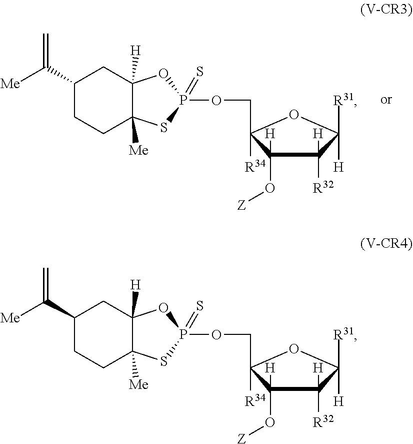
or a salt thereof;
2) reacting the compound of formula (V-CR3) or (V-CR4), or a salt thereof, with a compound of formula (VG): 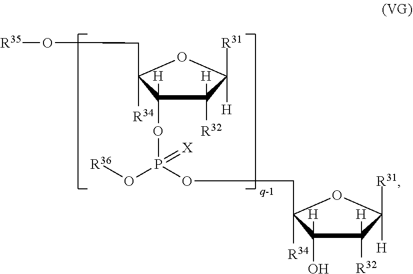
or a salt thereof, and a base, to form a compound of formula (V-C1) or (V-C2), wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 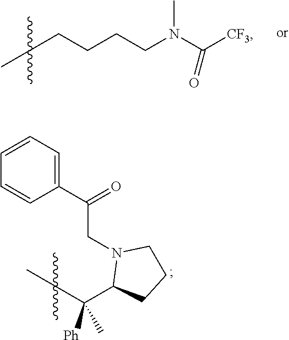 [SMILES: *CCCCN(C)C(=O)C(F)(F)F ; "or" ; *[C@](C)(c1ccccc1)[C@@H]1CCCN1CC(=O)c1ccccc1]
q is an integer from 1 to 20;
X, for each occurrence, is independently O or S, provided when X is S, the phosphorothiolate group has S-configuration, R-configuration or a mixture thereof (e.g., a racemic mixture);
Z is a group represented by Formula I* or B*, 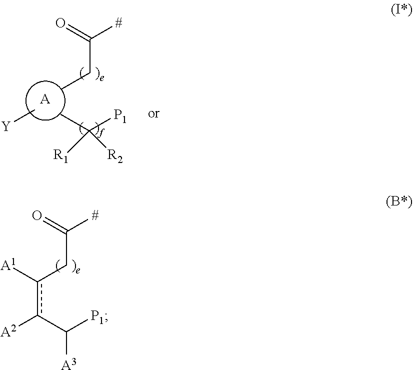
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

58. A process for preparing an oligonucleotide fragment of formula (VBZ), 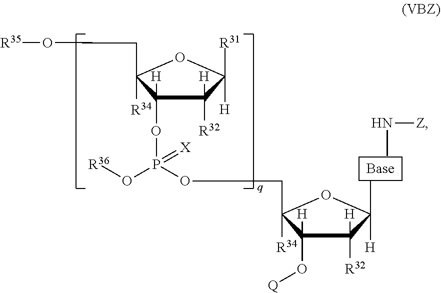
or a salt thereof, comprising the steps of:
1) reacting the compound of formula (VBZ-1), 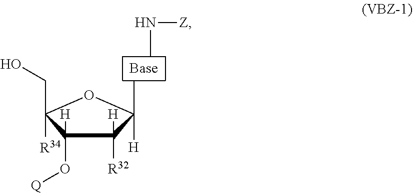
or a salt thereof, with a compound of formula (VBZ-2): 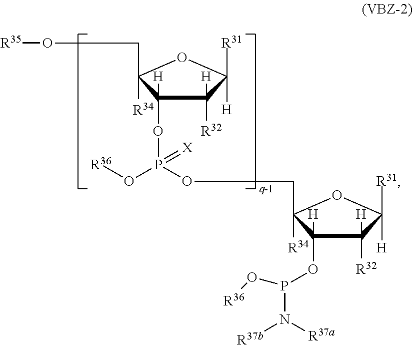
or a salt thereof, to form a compound of formula (VBZ-3), 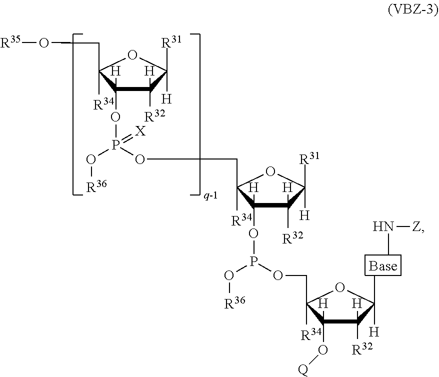
or a salt thereof;
2) sulfurizing or oxidizing the compound of formula (VBZ-3), or a salt thereof, with a sulfurization or oxidation agent to form a compound of formula (VBZ), or a salt thereof;
wherein:
Q is a hydroxyl protecting group; [symbol]
is a nucleobase comprising a NH₂ group which is modified by Z;
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 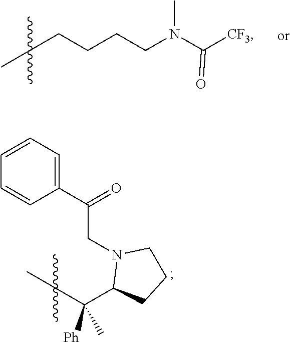 [SMILES: *CCCCN(C)C(=O)C(F)(F)F ; "or" ; *[C@](C)(c1ccccc1)[C@@H]1CCCN1CC(=O)c1ccccc1]
R^{37a} and R^{37b} are independently C₁₋₆alkyl;
q is an integer from 1 to 20;
X, for each occurrence, is independently O or S;
Z is a group represented by Formula I* or B*, 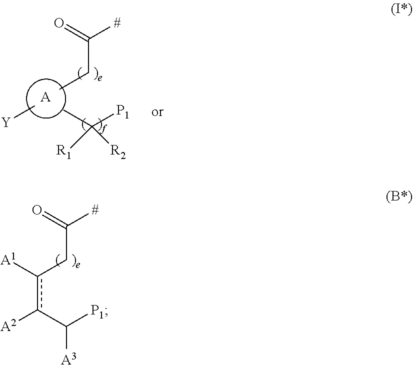
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

59. The process of claim 58, wherein the compound of formula VBZ-1 is prepared by
i) reacting the compound of formula (VBZ-4), 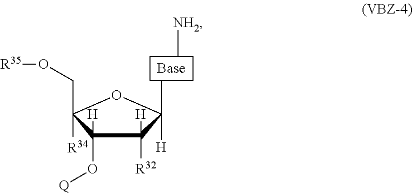
or a salt thereof, with Z—OH to form a compound of formula VBZ-5, 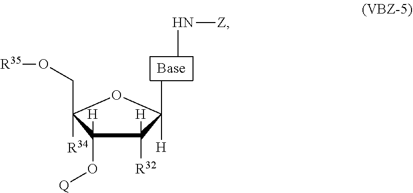
or a salt thereof; and
ii) deprotecting the compound of formula (VBZ-5) to form the compound of formula (VBZ-1).

60. The process of any one of claims 54-59, wherein Y is a hydrophobic group comprising one or more aliphatic hydrocarbon group having 10 or more carbon atoms.

61. The process of claim any one of claims 54-60, wherein no chromatography is used for purifying the reaction product of any one of steps 1), 2), 3) and 4).

62. The process of any one of claims 54 to 61, wherein the reaction product of any one of steps 1), 2), 3) and 4) is purified by selective precipitation.

63. A process for preparing an oligonucleotide fragment of formula (V), 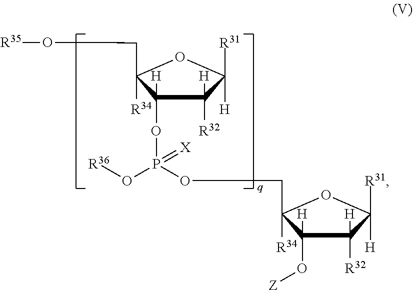
or a salt thereof, comprising the steps of:
c) coupling a nucleotide of formula (V-1): 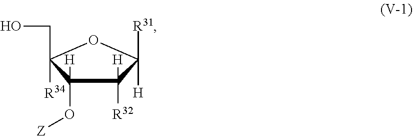
or a salt thereof, with an oligonucleotide fragment of formula (V-2): 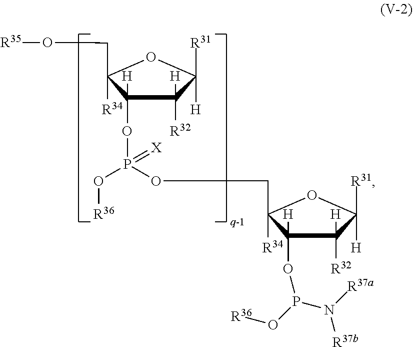
or a salt thereof, in a solution to form an oligonucleotide fragment of formula (V-3), 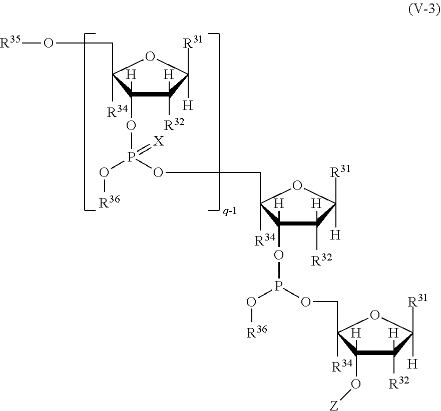
or a salt thereof; and
d) sulfurizing or oxidizing the oligonucleotide of formula (V-3), or a salt thereof, to form an oligonucleotide of formula (V): 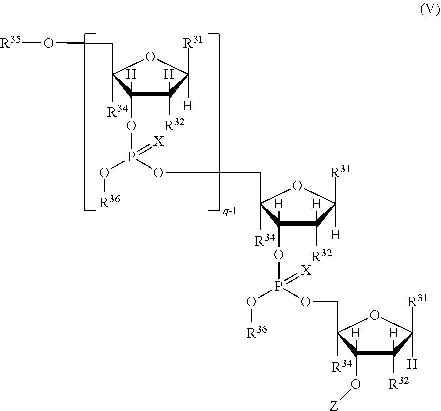
or a salt thereof;
wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 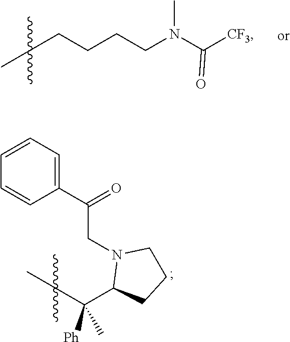 [SMILES: *CCCCN(C)C(=O)C(F)(F)F ; "or" ; *[C@](C)(c1ccccc1)[C@@H]1CCCN1CC(=O)c1ccccc1]
R^{37a} and R^{37b} are independently C₁₋₆alkyl;
q is an integer from 1 to 20;
X, for each occurrence, is independently O or S;
Z is a group represented by Formula I* or B*, 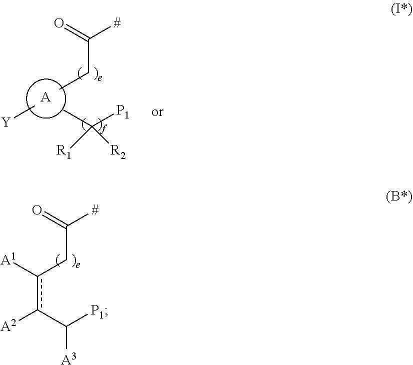
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

64. A process for preparing an oligonucleotide fragment of formula (V*), 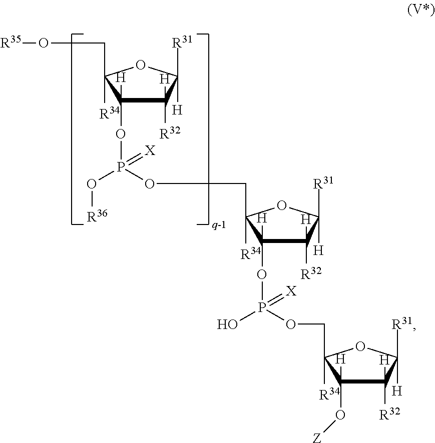
or a salt thereof, comprising the steps of:
a) coupling a nucleotide of formula (V-1): 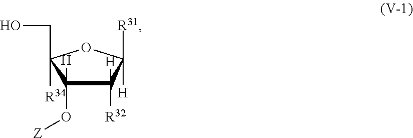
or a salt thereof, with an oligonucleotide fragment of formula (V-2′): 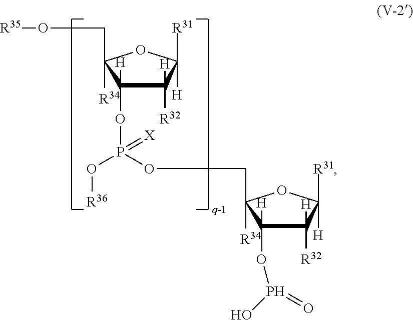
in a solution to form an oligonucleotide fragment of formula (V-3′), 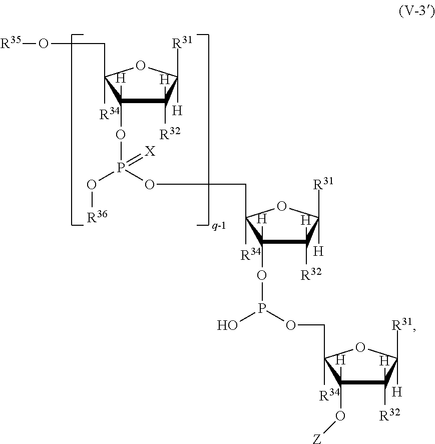
or a salt thereof; and
b) sulfurizing or oxidizing the oligonucleotide of formula (V-3′), or a salt thereof, to form the oligonucleotide of formula (V*) or a salt thereof;
wherein:
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently H, C₁₋₆alkyl group, C₂₋₆alkenyl group, phenyl or benzyl group, each of which is optionally substituted with —CN, —NO₂ or halogen; or
R³⁶ is 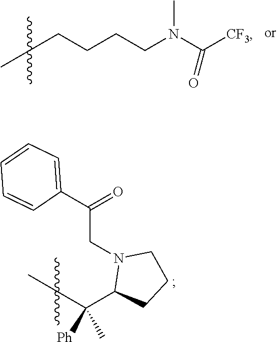 [SMILES: *CCCCN(C)C(=O)C(F)(F)F ; "or" ; *[C@](C)(c1ccccc1)[C@@H]1CCCN1CC(=O)c1ccccc1]
R^{37a} and R^{37b} are independently C₁₋₆alkyl;
q is an integer from 1 to 20;
X, for each occurrence, is independently O or S;
Z is a group represented by Formula I* or B*, 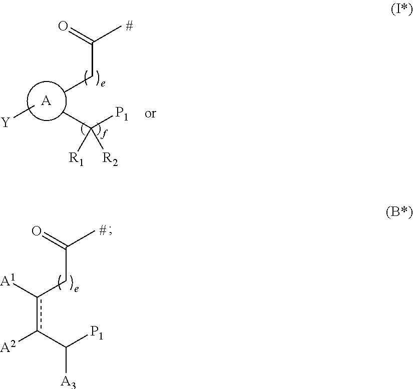
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

65. The process of claim 63 or 64, wherein Y is a hydrophobic group comprising one or more aliphatic hydrocarbon group having 10 or more carbon atoms.

66. The process of claim 54 or 63, further comprising deprotecting the fragment of formula (V) to form deprotected fragment of formula (VH): 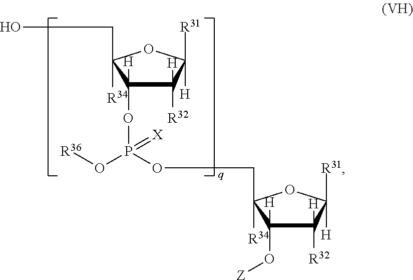
or a salt thereof.

67. The process of claim 55, further comprising deprotecting the fragment of formula (V′) to form deprotected fragment of formula (VH′): 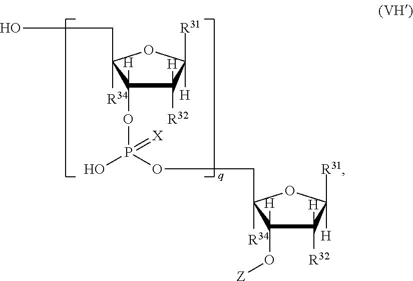
or a salt thereof.

68. The process of claim 56 or 57, further comprising deprotecting the fragment of formula (V-C1) or (V-C2) to form deprotected fragment of formula (V-C3) or (V-C4): 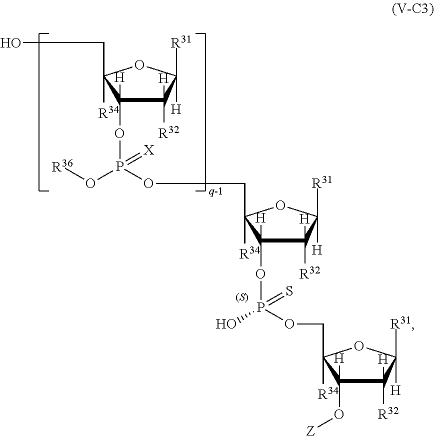
or a salt thereof, or 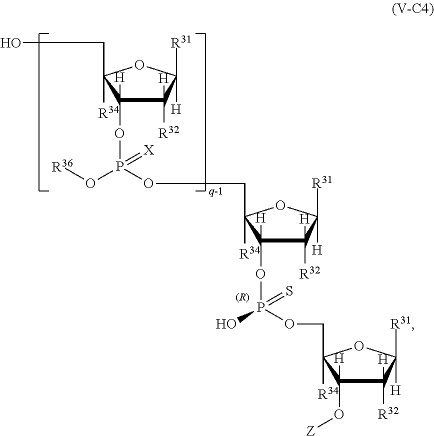
or a salt thereof.

69. The process of claim 58, further comprising deprotecting the fragment of formula (VBZ) to form deprotected fragment of formula (VBZ-6): 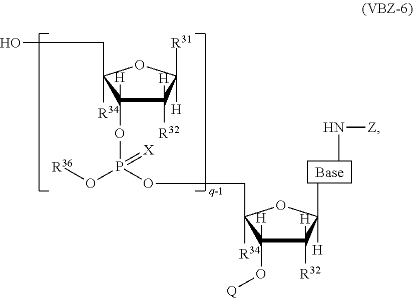
or a salt thereof.

70. The process of claim 64, further comprising deprotecting the fragment of formula (V*) to form deprotected fragment of formula (V*-1): 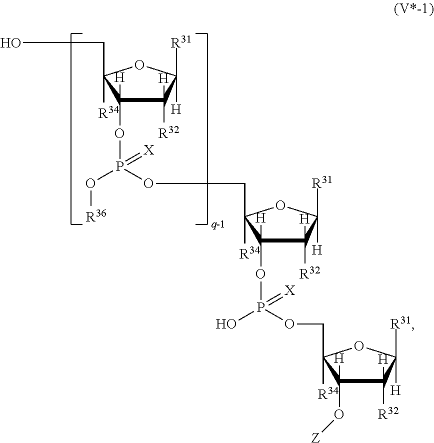
or a salt thereof.

71. The process of any one of claims 54-58, 63, and 64, wherein further comprising desilylation of the fragment of formula (V), (V′), (V-C1), (V-C2), (VBZ), or (V*) to form fragment of formula (VJ), (VJ′), (V-C5), (V-C6), (VBZ-7), or (V*-2): 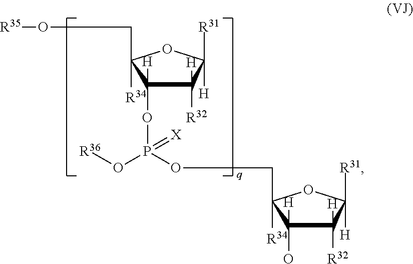
or a salt thereof, 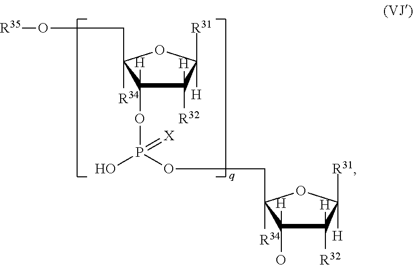
or a salt thereof, 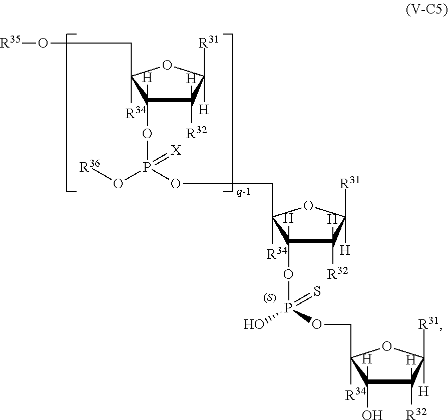
or a salt thereof, 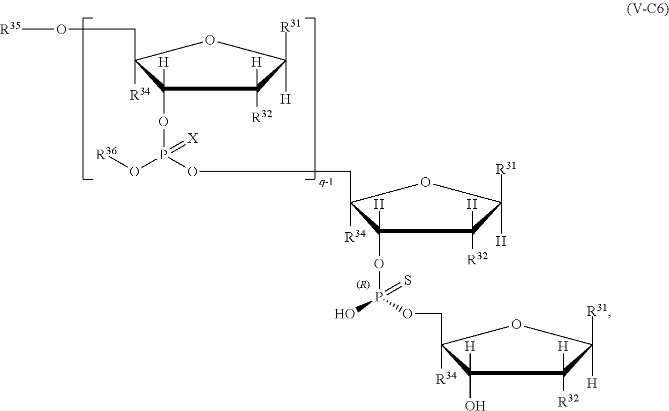
or a salt thereof, 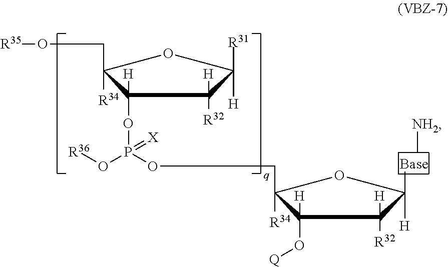
or a salt thereof, or 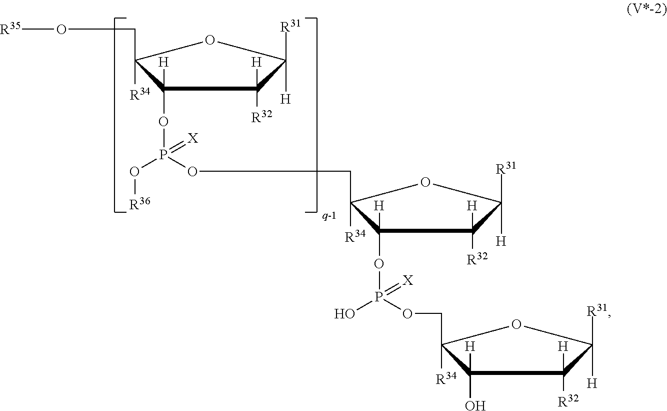
or a salt thereof, provided when Q and P₁ are the same, the desilylation of the fragment of (VBZ) forms a fragment of formula (VBZ-7′):

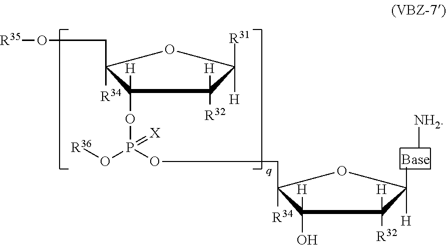

72. The process of claim 71, wherein the desilylation reaction is carried out by reacting the fragment of formula (V), (V′), (V-C1), (V-C2), (VBZ), or (V*) with HF in the presence of a base.

73. The process of claim 72, wherein the base is imidazole or pyridine, wherein the imidazole or pyridine are optionally substituted.

74. The process of 71, wherein the desilylation reaction is carried out by reacting the fragment of formula (V), (V′), (V-C1), (V-C2), (VBZ), or (V*) with HF in the presence of pyridine and imidazole.

75. The process of claim 74, wherein the molar ratio of imidazole to HF is in the range of 0.5:1 to 10:1.

76. The process of claim 75, wherein the molar ratio of imidazole to HF is in the range of 1.1:1 to 5:1.

77. The process of claim 76, wherein the molar ratio of imidazole to HF is 2:1.

78. The process of any one of claims 74-77, wherein the molar ratio of pyridine to HF is in the range of 100:1 to 1:1.

79. The process of any one of claims 74-77, wherein the molar ratio of pyridine to HF is 1:1.

80. The process of any one of claims 54-71, wherein the fragment for formula (V), (V′), (V-C1), (V-C2), (VBZ), (V*), (VH), (VH′), (V-C3), (V-C4), (VBZ-6), (V*-1), (VJ), (VJ′), (V-C5), (V-C6), (VBZ-7), (VBZ-7′) or (V*-2) is not purified by chromatography.

81. The process of claim 80, wherein the fragment of formula (V), (V′), (V-C1), (V-C2), (VBZ), (V*), (VH), (VH′), (V-C3), (V-C4), (VBZ-6), (V*-1), (VJ), (VJ′), (V-C5), (V-C6), (VBZ-7), (VBZ-7′) or (V*-2) is purified by selective precipitation and/or extraction.

82. The process of any one of claims 54-81, wherein q is 2 to 5.

83. The process of claim 82, wherein q is 4.

84. A process for preparing an oligonucleotide of formula (VI) or (VI-1), 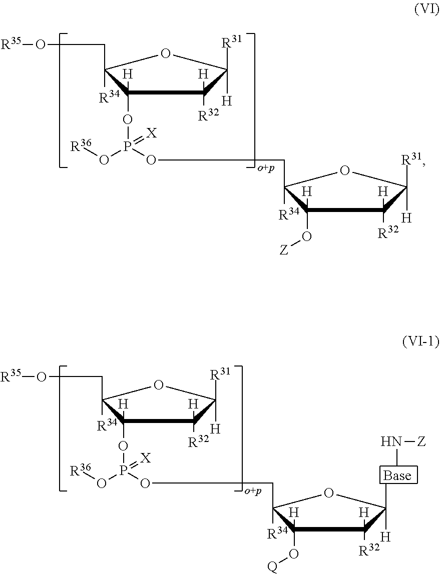
or a salt thereof, comprising
a) coupling an oligonucleotide fragment of formula (F1) or (F1-1): 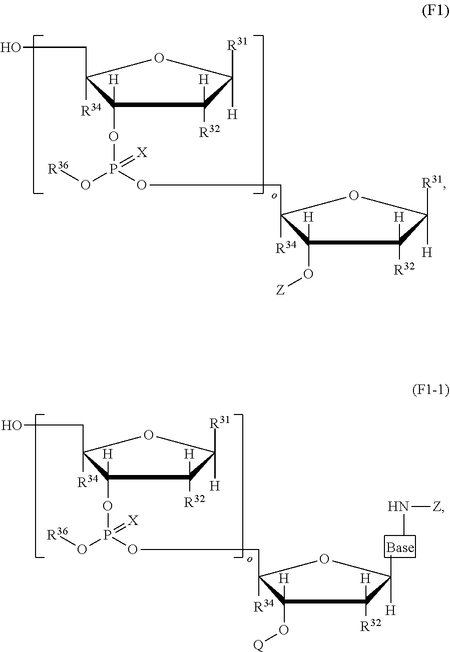
or a salt thereof, with an oligonucleotide fragment of formula (F2): 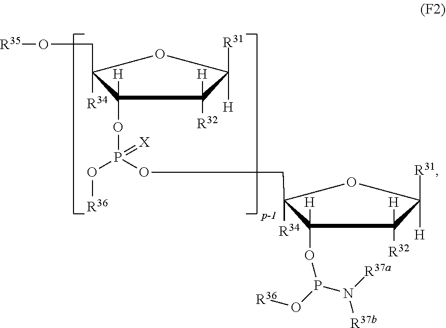
or a salt thereof, in a solution to form an oligonucleotide fragment of formula (F3) or (F3-1), 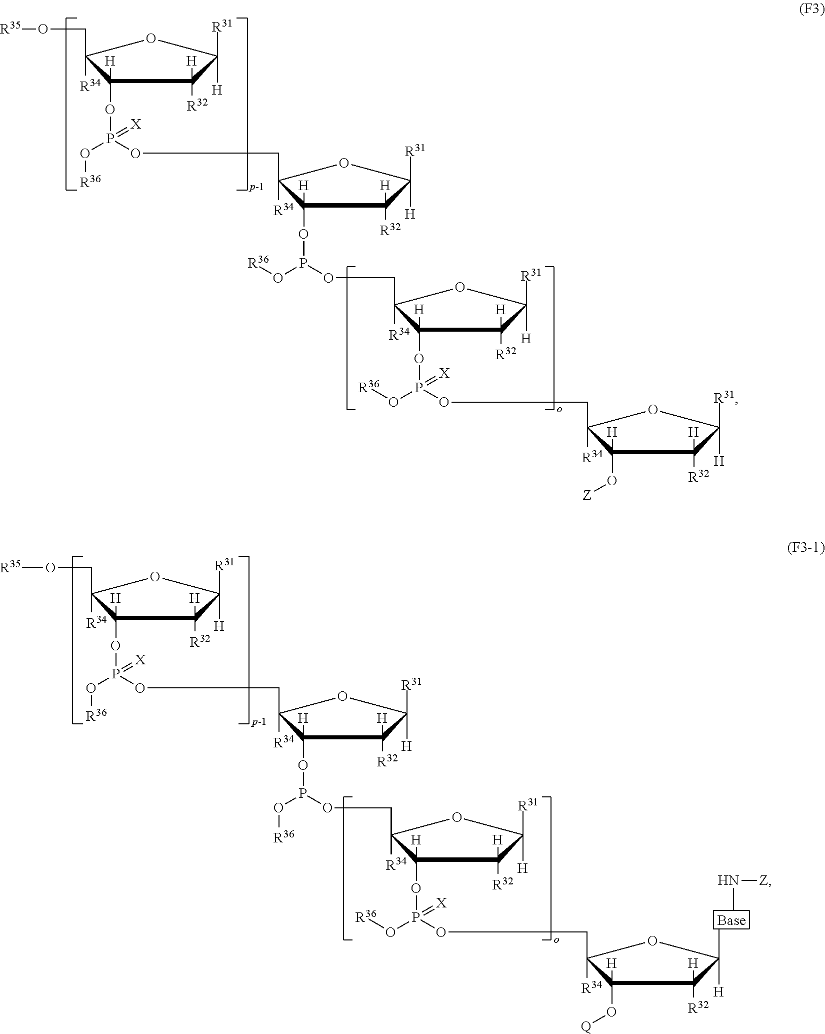
or a salt thereof; and
b) sulfurizing or oxidizing the oligonucleotide fragment of formula (F3) or (F3-1), or a salt thereof, to form the oligonucleotide of formula (VI) or (VI-1) or a salt thereof,
wherein:
Q is a hydroxyl protecting group; [symbol]
is a nucleobase comprising a NH₂ group which is modified by Z;
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 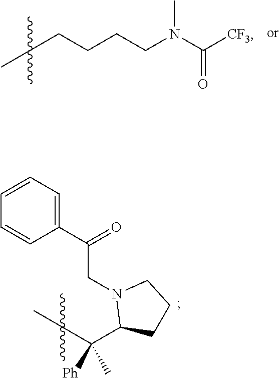 [SMILES: *CCCCN(C)C(=O)C(F)(F)F ; *[C@](C)(c1ccccc1)[C@@H]1CCCN1CC(=O)c1ccccc1]
R^{37a} and R^{37b} are independently C₁₋₆alkyl;
p is an integer from 2 to 20;
o is an integer from 1 to 200;
X, for each occurrence, is independently O or S;
Z is a group represented by Formula I* or B*, 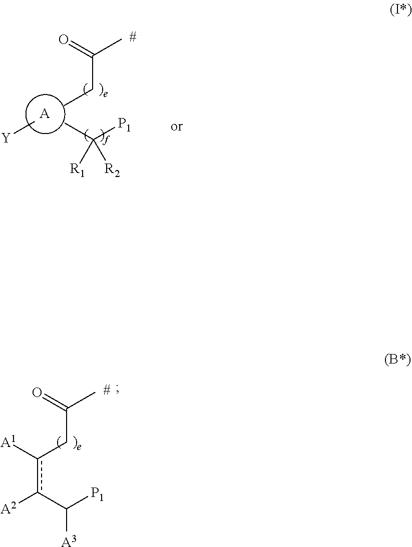
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

85. A process for preparing an oligonucleotide of formula (VI′) or (VI′-1), 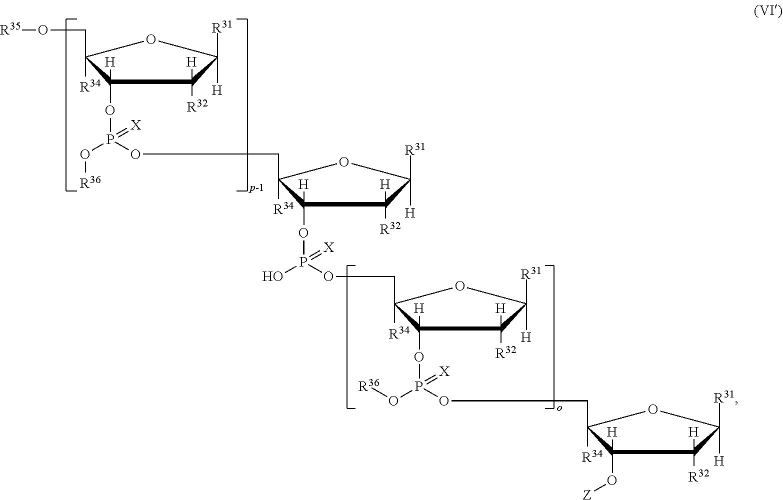 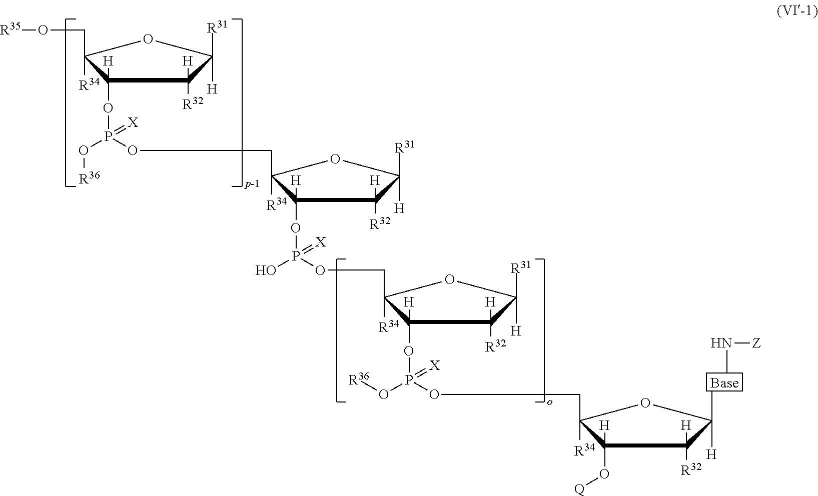
or a salt thereof, comprising
a) coupling an oligonucleotide fragment of formula (F1) or (F1-1): 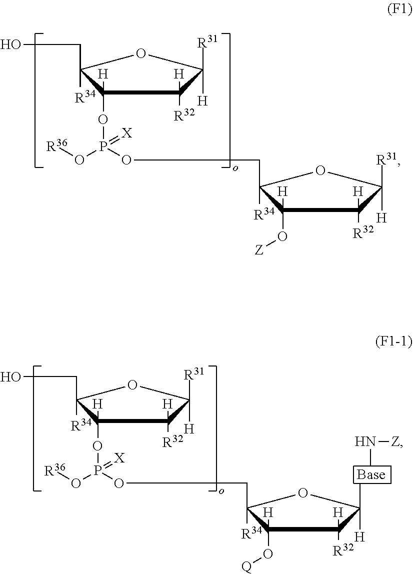
or a salt thereof, with an oligonucleotide fragment of formula (F2′): 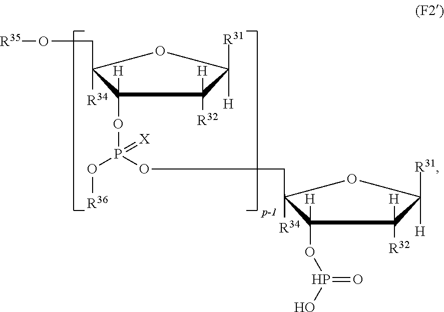
or a salt thereof, in a solution to form an oligonucleotide fragment of formula (F3′) or (F3′-1), 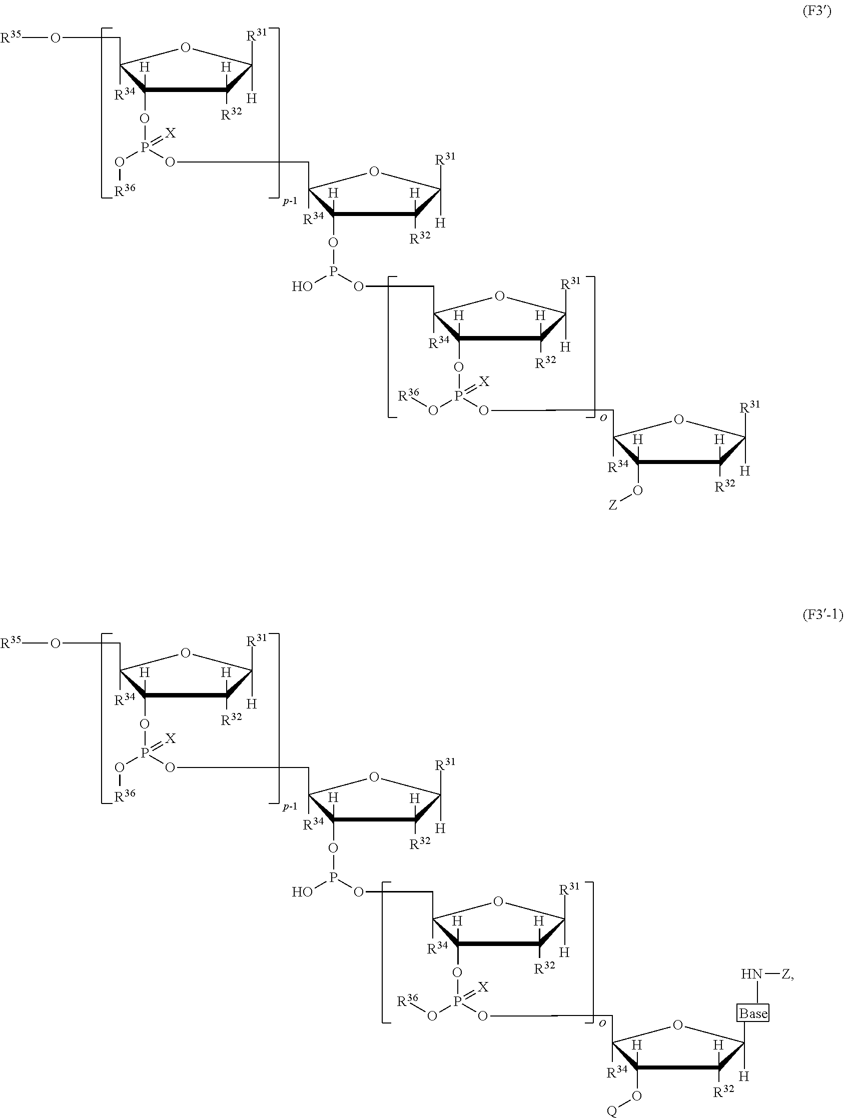
or a salt thereof; and
b) sulfurizing or oxidizing the oligonucleotide fragment of formula (F3′) or (F3′-1), or a salt thereof, to form the oligonucleotide of formula (VI′) or (VI′-1) or a salt thereof,
wherein:
Q is a hydroxyl protecting group; [symbol]
is a nucleobase comprising a NH₂ group which is modified by Z;
R³¹, for each occurrence, is independently a nucleobase, wherein the NH₂ of the nucleobase, if present, is protected by an amine protecting group;
R³², for each occurrence, is independently selected from the group consisting of H, halo, OH, and C₁₋₆alkoxy optionally substituted with C₁₋₆alkoxy; wherein the OH group is optionally protected by a hydroxyl protecting group;
R³⁴, for each occurrence, is independently H or forms a ring with the alkoxy group of R³²;
R³⁵ is a hydroxyl protecting group;
R³⁶, for each occurrence, is independently C₁₋₆alkyl group, C₂₋₆alkenyl group, phenyl or benzyl group, each of which is optionally substituted with —CN, —NO₂ or halogen; or
R³⁶ is 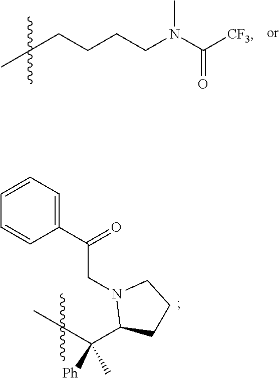 [SMILES: *CCCCN(C)C(=O)C(F)(F)F ; *[C@](C)(c1ccccc1)[C@@H]1CCCN1CC(=O)c1ccccc1]
R^{37a} and R^{37b} are independently C₁₋₆alkyl;
p is an integer from 2 to 20;
o is an integer from 1 to 200;
X, for each occurrence, is independently O or S;
Z is a group represented by Formula I* or B*, 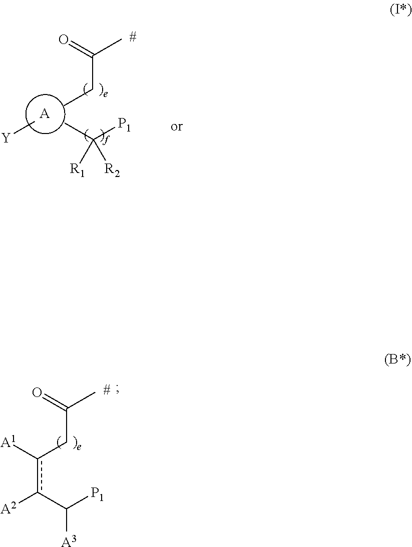
wherein
—#represents the point of attachment for Z;
one of A¹, A² and A³ is Y^{A} and the others are H; [symbol]
is a single bond or a double bond;
Y^{A} is Y—(CH₂)ₐ₁CH₂O(CH₂)ₐ₂—, wherein a1 and a2 are each independently 0 or an integer from 1 to 10;
ring A is phenyl, 8- to 10-membered bicyclic aryl, 5- to 6-membered heteroaryl having 1 to 3 heteroatoms independently selected from oxygen, nitrogen, and sulfur, or 7- to 10-membered bicyclic heteroaryl having 1-4 heteroatoms independently selected from oxygen, nitrogen, and sulfur;
Y is H, halogen, OR^{1A}, NR^{2A}R^{3A}, SR^{4A}, CR^{5A}R^{6A}R^{7A}, or a hydrophobic group comprising one or more aliphatic hydrocarbon group having 10 or more carbon atoms; wherein R^{1A}, R^{2A}, R^{3A}, R^{4A}, R^{5A}, R^{6A}, and R^{7A} are each independently C₁₋₆alkyl, C₁₋₆alkenyl, C₁₋₆alkynyl, phenyl, OR^{8A}, —OC(O)R^{8A}, —C(O)OR^{8A}, NR^{8A}R^{9A}, —NR^{8A}COR^{9A}, —CONR^{8A}R^{9A}, 3- to 7-membered saturated or partially unsaturated monocyclic carbocyclyl, or 3- to 7-membered saturated or partially unsaturated monocyclic heterocyclyl having 1-2 heteroatoms independently selected from oxygen, nitrogen, and sulfur; wherein R^{8A} and R^{9A}, for each occurrence, is independently H or C₁₋₆alkyl;
P₁ is NO₂ or a silyl hydroxyl protecting group;
R₁ and R₂ are independently H, C₁₋₆alkyl, or phenyl; wherein C₁₋₆alkyl and phenyl are optionally substituted by 1-3 R₃;
R₃ is C₁₋₃₀alkoxy;
e is an integer from 0 to 6; and
f is an integer from 0 to 6.

86. The process of claim 84 or claim 85, wherein Y is a hydrophobic group comprising one or more aliphatic hydrocarbon group having 10 or more carbon atoms.

87. The process of claim 84 or claim 85, further comprising step c) deprotecting the oligonucleotide of formula (VI), (VI′), (VI-1), or (VI′-1) to form an oligonucleotide of formula (VII), (VII-1), (VII′), or (VII′-1): 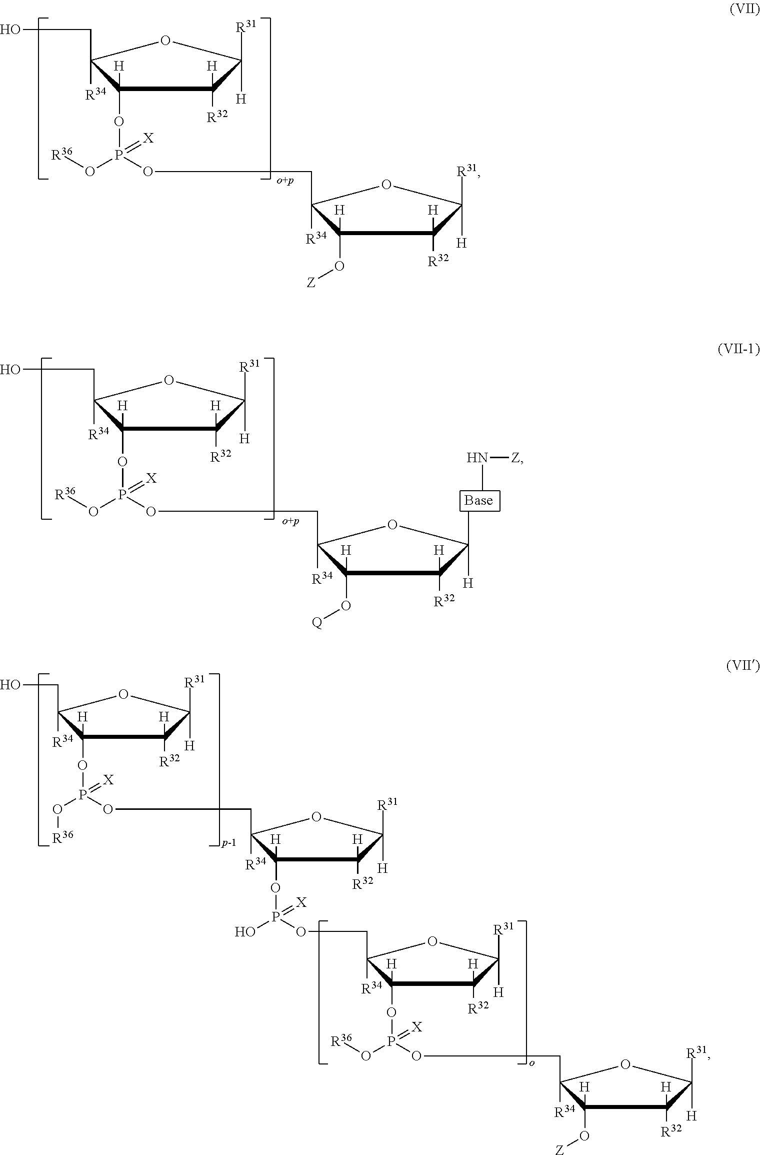 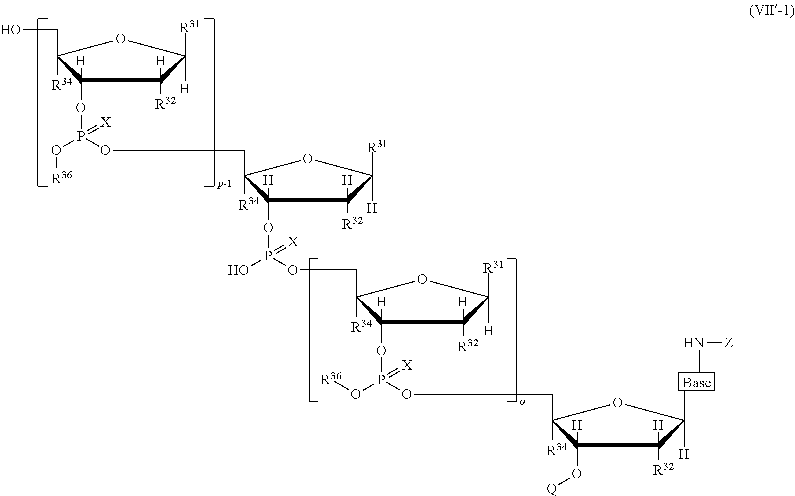
or a salt thereof.

88. The process of claim 87, wherein starting from oligonucleotide of formula (VII), (VII-1), (VII′), or (VII′-1), the process further comprises repeating steps a), b) and c) for 1 to 10 times, followed by steps a) and b).

89. The process of claim 88, wherein the process further comprises repeating steps a), b) and c) for 1 to 3 times followed by steps a) and b).

90. The process of any one of claims 84-89, wherein o is an integer from 2 to 20.

91. The process of claim 90, wherein o is 2 to 5.

92. The process of claim 91, wherein o is 4.

93. The process of claim of any one of claims 54-92, wherein Z is a group represented by Formula I*,

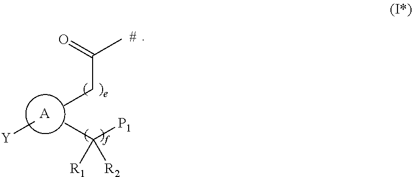

94. The process of claim of any one of claims 54-92, wherein Z is a group represented by Formula B*,

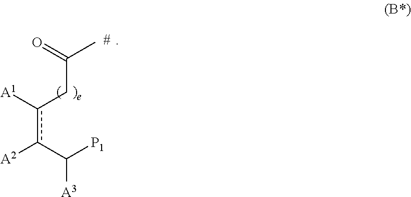

95. The process of claim of any one of claims 54-92, wherein Z is a group represented by Formula B-1* or B-2*:

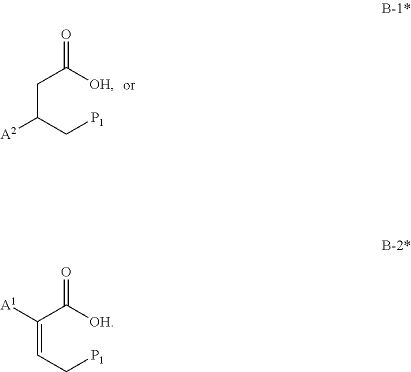

96. The process of any one of claims 54-92, wherein ring A is phenyl or naphthalenyl.

97. The process of any one of claims 54-96, wherein P₁ is a silyl hydroxyl protecting group selected from the following: 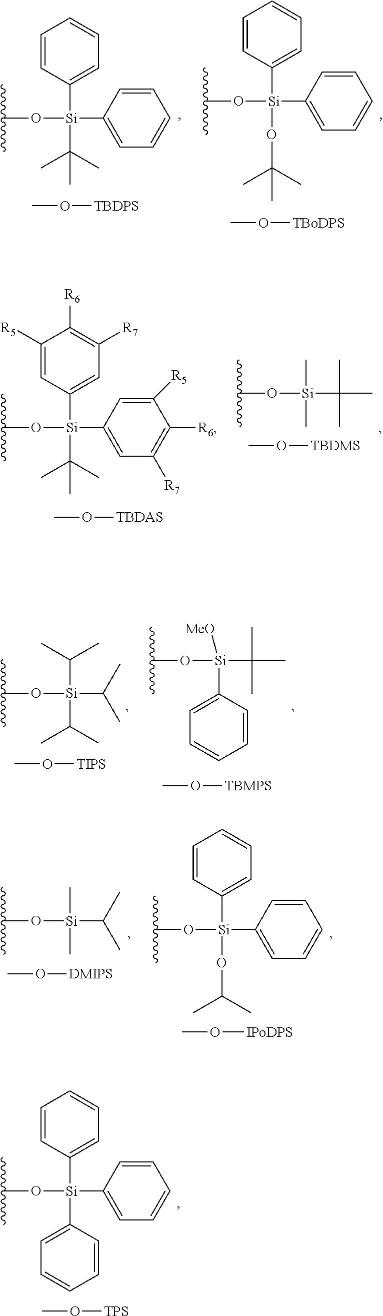 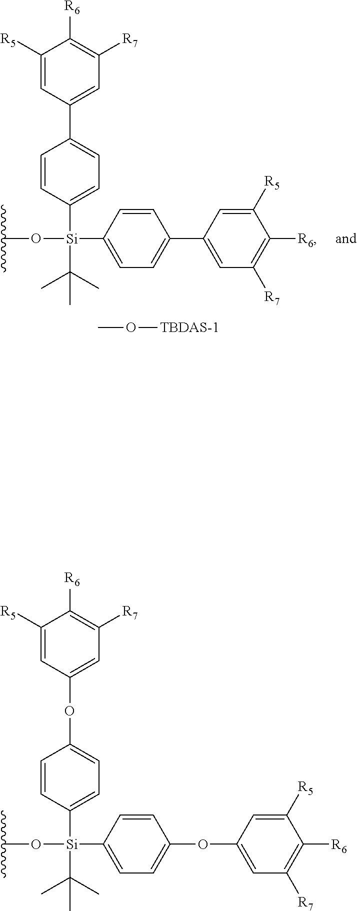
and —O-TBDAS-2; wherein 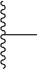
represents the point of attachment for P₁ and R₅, R₆ and R₇ are each independently H, C₁₋₃₀alkyl, or C₁₋₃₀alkoxy.

98. The process of claim 97, wherein P₁ is selected from the group consisting of —O-TBDMS, —O-TIPS, —O-TBDPS, —O-TBoDPS, and —O-TBDAS:

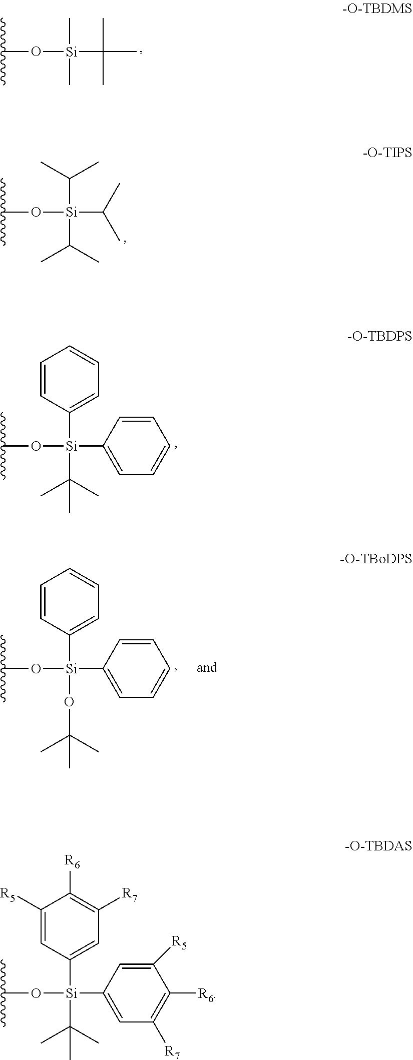

99. The process of claim 93, wherein Z is a group represented by Formula I** or Ia**: 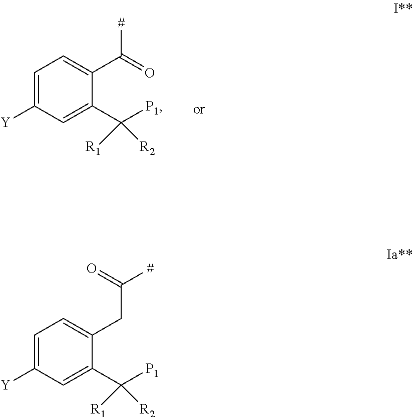
or a salt thereof;
wherein P₁ is selected from the group consisting of —O-TBDPS, —O-TBoDPS, and —O-TBDAS:

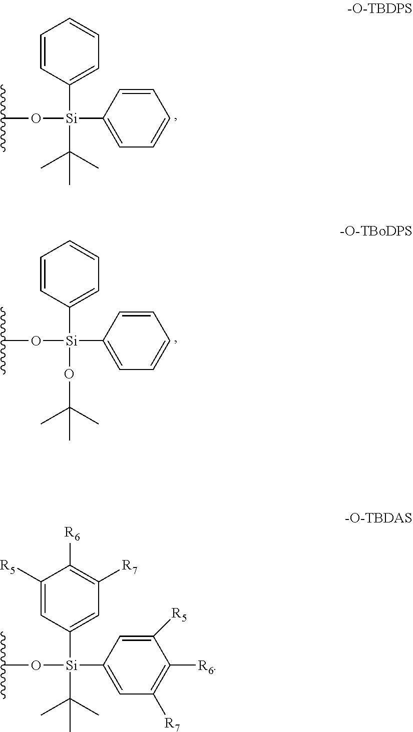

100. The process of any one of claims 54-99 or a salt thereof, wherein Y is represented by Formula A:
W—V—U* (A)
wherein:
—* represents the point of attachment for Y;
W is represented by Formula A1, A2, A2-1, A2-2, A3, A3-1, or A3-2: 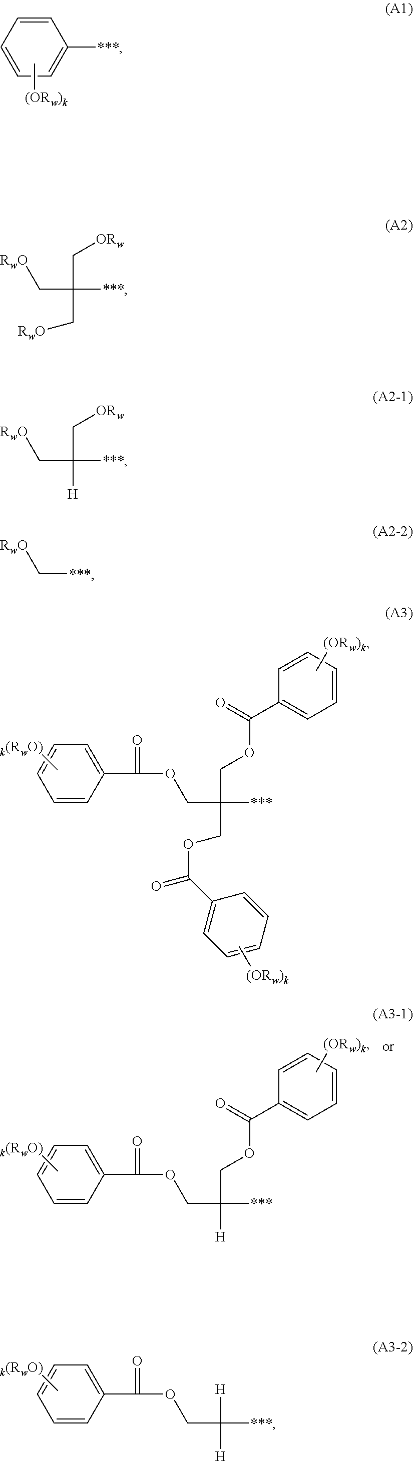
wherein
—*** represents the point where W and V connect;
each R_{w} is independently an aliphatic hydrocarbon group having 10 or more carbon atoms;
k is an integer from 1 to 5;
V is a bond, oxygen, C₁₋₂₀alkylene, C₁₋₆alkynylene, —C(═O)—, ***—C(═O)—O—**, ***—O—C(═O)—**, 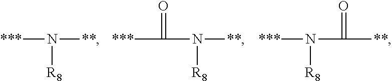
or 5 to 7 member heteroaryl having 1 to 3 heteroatoms selected from oxygen, nitrogen, and sulfur, wherein the heteroaryl is optionally substituted by 1-3 R₈; wherein —** represents the point where V and U connect; and R₈ is H or C₁₋₃₀alkyl; and
U is a bond, oxygen, C₁₋₂₀alkylene, carbonyl, ***—O—C(═O)—** 5 to 7 member heterocyclyl having 1 to 3 heteroatoms selected from oxygen, nitrogen, and sulfur; 5 to 7 member heteroaryl having 1 to 3 heteroatoms selected from oxygen, nitrogen, and sulfur, wherein the heteroaryl is optionally substituted by 1-3 R₈; or a group represented by formula A4, A5, or A6: 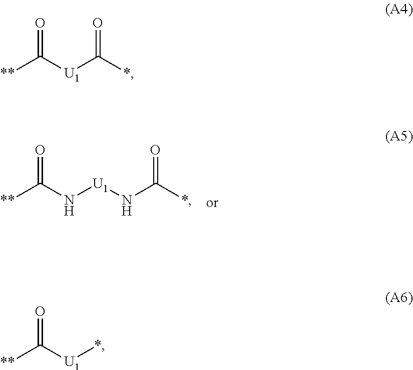
wherein U₁ is C₁₋₆alkylene, C₁₋₆alkyleneoxy, 5 to 7 member heterocyclyl having 1 to 3 heteroatoms selected from oxygen, nitrogen, and sulfur, or 5 to 7 member heteroaryl having 1 to 3 heteroatoms selected from oxygen, nitrogen, and sulfur.

101. The process of any one of claims 97-100, wherein the TBDAS group is: 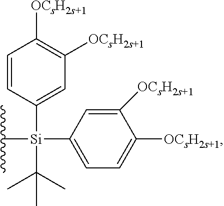
wherein s is an integer from 1 to 30.

102. The process of any one of claims 54-100, wherein P₁ is TBDPS.

103. The process of any one of claims 100-102, wherein W is represented by Formula A1: 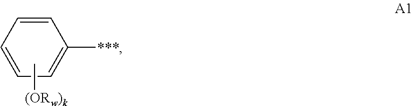
wherein R_{w} is CₙH₂ₙ₊₁;
n is an integer from 1 to 30.

104. The process of any one of claims 100-103, wherein R_{w} is selected from a group consisting of C₁₂H₂₅, C₁₈H₃₇, C₂₀H₄₁, C₂₂H₄₅, C₂₄H₄₉, C₂₆H₅₃, and C₂₈H₅₇.

105. The process of any one of claims 100-104, wherein V is a bond, CH₂, CH₂CH₂, C(═O)—, ***—C(═O)—O—**, or

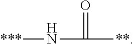

106. The process of any one of claims 54-100, wherein Y is selected from the groups consisting of 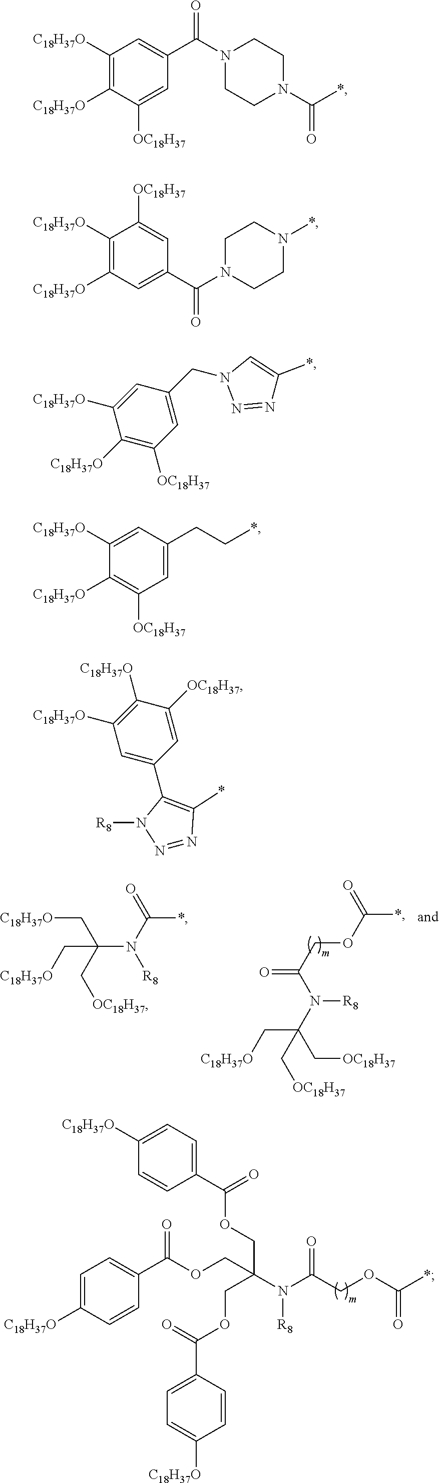
wherein
R₈ is H or C₁₋₆alkyl; and
m is an integer from 1 to 5.

107. The process of any one of claims 54-106, wherein R₁ and R₂ are independently H or CH₃.

108. The process of any one of claims 54-107, wherein e is 0, 1, or 2; and f is 0, 1, or 2.

109. The process of any one of claims 54-108, wherein e is 1; and f is 1.

110. The process of any one of claims 54-108, wherein e is 0; and f is 1 or e is 1; and f is 0.

111. The process of any one of claims 54-110, wherein R₈ is H or C₁₋₄alkyl.

112. The process of any one of claims 54-111, wherein Z is represented by Formula II* or IIa*, 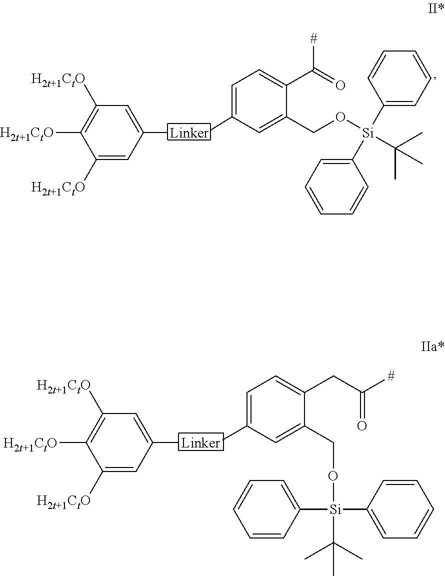
wherein
t is an integer from 10 to 30; [symbol]
is selected from the group consisting of 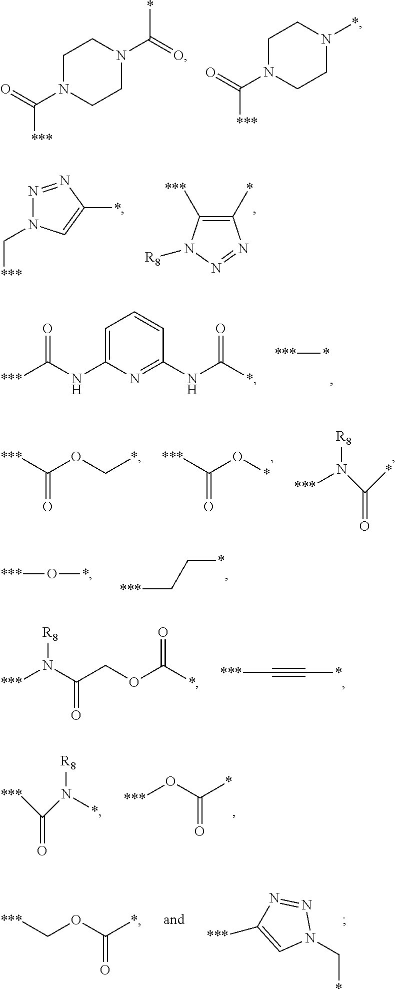
wherein R₈ is H or C₁₋₆alkyl.

113. The process of any one of claims 54-112, wherein Z is:

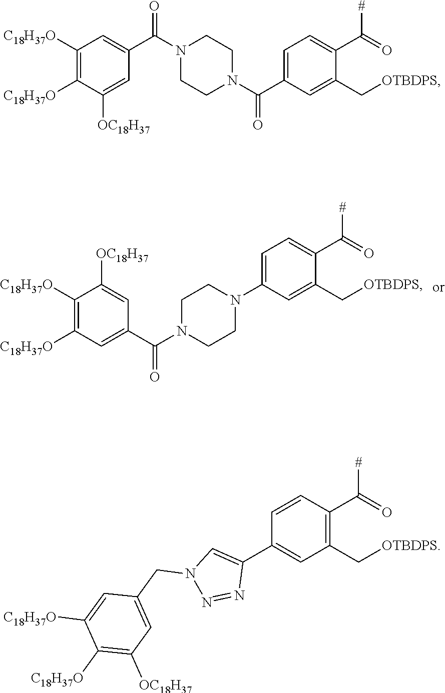

114. The process of any one of claims 54-93, or a salt thereof, wherein Z is

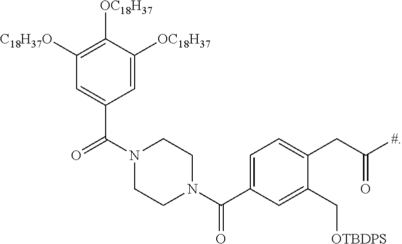

115. The process of any one of claims 54-93, or a salt thereof, wherein Z is

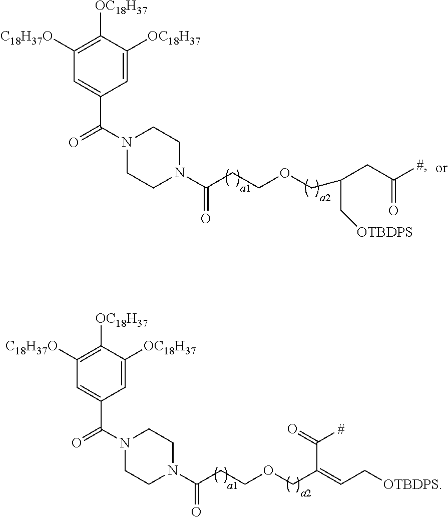

116. The nucleotide or oligonucleotide of any one of claims 27-53 or the process of any one of claims 54-115, wherein all of the P═X groups in the nucleotide or oligonucleotide are P═S.

117. The nucleotide or oligonucleotide of any one of claims 27-53 or the process of any one of claims 54-115, wherein all of the P═X groups in the nucleotide or oligonucleotide are P═O.

118. The nucleotide or oligonucleotide of any one of claims 27-53 or the process of any one of claims 54-115, wherein greater than 10%, 20%, 30%, 40%, 50%, 60%, 70%, 80% or 90% of the P═X groups in the compound or oligonucleotide are P═S.

119. The nucleotide or oligonucleotide of any one of claims 27-53 or the process of any one of claims 54-115, wherein 10-90%, 20-80%, 30-70% or 40-60% of the P═X groups in the compound or oligonucleotide are P═S.

120. The nucleotide or oligonucleotide of any one of claims 27-53 or the process of any one of claims 54-115, wherein the nucleobase is selected from the group consisting of cytosine, guanine, adenine, thymine, uracil, hypoxanthine, xanthine, 7-methylguanine, 5,6-dihydrouracil, 5-methylcytosine, and 5-hydroxymethylcytosine, wherein the NH₂ group of the nucleobase, if present, is protected by PhCO—, CH₃CO—, iPrCO—, Me₂N—CH═, or Me₂N—CMe═.

121. The nucleotide or oligonucleotide of any one of claims 27-53 or the process of any one of claims 54-115, wherein the nucleobase is selected from the group consisting of cytosine, guanine, adenine, thymine, uracil, and 5-methylcytosine, wherein the NH₂ group of the nucleobase, if present, is protected by PhCO—, CH₃CO—, iPrCO—, Me₂N—CH═, or Me₂N—CMe═.

122. The nucleotide or oligonucleotide of any one of claims 27-53 or the process of any one of claims 54-121, wherein
each R³² is independently selected from the group consisting of H, F, and C₁₋₄alkoxy optionally substituted with C₁₋₄alkoxy;
each R³⁴ is independently H or forms a ring with the alkoxy group of R², wherein the ring is a 5 or 6-membered ring optionally substituted with 1 to 3 C₁₋₄ alkyl groups;
each R³⁵ is a 4,4′-dimethoxytirtyl group;
R³⁶ is —CH₂CH₂CN; and
R^{37a} and R^{37b} are independently C₁₋₄alkyl.

123. The nucleotide or oligonucleotide of any one of claims 27-53 or the process of any one of claims 54-121, wherein
each R³² is independently selected from the group consisting of H, F, —OCH₃, —OCH₂CH₂OCH₃, and —OTBDMS; and
each R³⁴ is independently H or forms a ring with the alkoxy group of R³², wherein the ring is a 5-membered ring.

124. The nucleotide or oligonucleotide of any one of claims 27-53 or the process of any one of claims 54-121, wherein each R³⁴ is independently H or together with the alkoxy group of R³² form —CH₂—O—.

125. The nucleotide or oligonucleotide of any one of claims 27-53 or the process of any one of claims 54-121, wherein
each R³² is independently selected from H or —OCH₂CH₂OMe;
each R³⁴ is H;
each R³⁵ is a 4,4′-dimethoxytirtyl group;
R³⁶ is —CH₂CH₂CN; and
R^{37a} and R^{37b} are both —CH(CH₃)₂.

126. The process of any one of claims 55, 64, and 85, wherein the salt of the compound of formula (VD′), (V-2′), or (F2′) is selected from trimethyl amine salt, triethyl amine salt, and triisopropyl amine salt.

127. The process of claim 126, wherein the salt of the compound of formula (VD′), (V-2′), or (F2′) is triethyl amine salt.

128. The nucleotide or oligonucleotide of claim 28, or the process of any one of claims 58, 59, 69, and 71-92, wherein the [symbol] is adenine, cytosine, or guanine.

129. The nucleotide or oligonucleotide of claim 28, or the process of any one of claims 58, 59, 69, and 71-92, wherein the Q is a silyl protecting group.

130. The nucleotide or oligonucleotide of claim 28, or the process of any one of claims 58, 59, 69, and 71-92, wherein the Q is selected from the group consisting of trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl tri(trimethylsilyl)silyl, t-butylmethoxyphenylsilyl, and t-butoxydiphenylsilyl.

131. The nucleotide or oligonucleotide of claim 28, or the process of any one of claims 58, 59, 69, and 71-92, wherein the Q is t-butyldiphenylsilyl.
